(19) United States
(12) Patent Application Publication
(10) Pub. No.: US 2022/0251081 A1
(43) Pub. Date: Aug. 11, 2022

(54) HETEROBICYCLIC INHIBITORS OF MAT2A AND METHODS OF USE FOR TREATING CANCER

(71) Applicant: LES LABORATOIRES SERVIER, Suresnes Cedex (FR)

(72) Inventors: Zenon D. KONTEATIS, Cambridge, MA (US); Mingzong LI, Cambridge, MA (US); Samuel K. REZNIK, Cambridge, MA (US); Jeremy M. TRAVINS, Cambridge (GB); Zhihua SUI, Cambridge, MA (US)

(21) Appl. No.: 17/615,255

(22) PCT Filed: May 29, 2020
(86) PCT No.: PCT/US2020/035036

§ 371 Date: Nov. 30, 2021

Related U.S. Application Data
(60) Provisional application No. 62/855,395, filed on May 31, 2019.

(51) Int. Cl. C07D 471/04; C07D 519/00; A61P 35/00

(52) U.S. Cl. CPC C07D 471/04; A61P 35/00; C07D 519/00

(57) ABSTRACT

The present disclosure provides for compounds according to Formula I, Formula II, and their pharmaceutically acceptable salts, tautomers, and/or isotopologues as described in the disclosure. The compounds are inhibitors of methionine adenosyltransferase isoform 2A (MAT2A). Also provided are pharmaceutical compositions and methods of using the compounds for treating cancers, including some cancers in which the gene encoding methylthioadenosine phosphorylase (MTAP) is deleted.

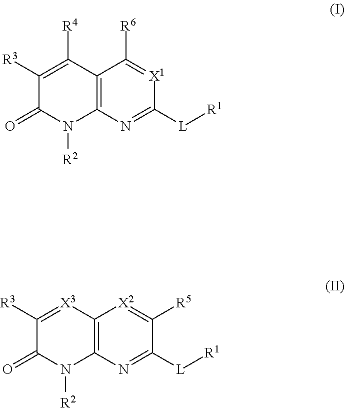

### CROSS-REFERENCE TO RELATED APPLICATIONS

This application claims priority to U.S. Provisional Patent Application No. 62/855,395, filed May 31, 2019, the disclosure of which is incorporated by reference herein.

### BACKGROUND

Methionine adenosyltransferase (MAT), which is also known as S-adenosylmethionine synthetase, is a cellular enzyme that catalyzes the synthesis of S-adenosyl methionine (SAM or AdoMet) from methionine and ATP; the catalysis is considered to be rate-limiting step of the methionine cycle. SAM is the propylamino donor in polyamine biosynthesis, the principal methyl donor for DNA methylation, and is involved in gene transcription and cellular proliferation as well as the production of secondary metabolites.

Two genes designated as MAT1A and MAT2A encode two distinct catalytic MAT isoforms, respectively. A third gene, MAT2B, encodes a MAT2A regulatory subunit. MAT1A is specifically expressed in the adult liver, whereas MAT2A is widely distributed. Because MAT isoforms differ in catalytic kinetics and regulatory properties, MAT1A-expressing cells have considerably higher SAM levels than do MAT2A-expressing cells. It has been found that hypomethylation of the MAT2A promoter and histone acetylation causes upregulation of MAT2A expression.

In hepatocellular carcinoma (HCC), the downregulation of MAT1A and the up-regulation of MAT2A occur, which is known as the MAT1A:MAT2A switch. The switch, accompanied with up-regulation of MAT2B, results in lower SAM contents, which provide a growth advantage to hepatoma cells. Because MAT2A plays a crucial role in facilitating the growth of hepatoma cells, it is a target for antineoplastic therapy. Recent studies have shown that silencing by using small interfering RNA substantially suppresses growth and induces apoptosis in hepatoma cells. See, e.g., T. Li et al., J. Cancer 7(10) (2016) 1317-1327.

Some cancer cell lines that are MTAP deficient are particularly sensitive to inhibition of MAT2A. Marjon et al. (Cell Reports 15(3) (2016) 574-587). MTAP (methylthioadenosine phosphorylase) is an enzyme widely expressed in normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

MAT2A is dysregulated in additional cancers that lack MTAP-deletion, including hepatocellular carcinoma and leukemia. J. Cai et al., Cancer Res. 58 (1998) 1444-1450; T. S. Jani et al., Cell. Res. 19 (2009) 358-369. Silencing of MAT2A expression via RNA-interference results in anti-proliferative effects in several cancer models. H. Chen et al., Gastroenterology 133 (2007) 207-218; Q. Liu et al. Hepatol. Res. 37 (2007) 376-388.

Many human and murine malignant cells lack MTAP activity. MTAP deficiency is found not only in tissue culture cells but the deficiency is also present in primary leukemias, gliomas, melanomas, pancreatic cancers, non-small cell lung cancers (NSCLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphoma, and mesotheliomas. The gene encoding for human MTAP maps to region 9p21 on human chromosome 9p. This region also contains the tumor suppressor genes p16INK4A (also known as CDKN2A) and p15INK4B. These genes code for p16 and p15, which are inhibitors of the cyclin D-dependent kinases cdk4 and cdk6, respectively.

The p16INK4A transcript can alternatively be alternative reading frame (ARF) spliced into a transcript encoding p14ARF. p14ARF binds to MDM2 and prevents degradation of p53 (Pomerantz et al. (1998) Cell 92:713-723). The 9p21 chromosomal region is of interest because it is frequently homozygously deleted in a variety of cancers, including leukemias, NSLC, pancreatic cancers, gliomas, melanomas, and mesothelioma. The deletions often inactivate more than one gene. For example, Cairns et al. ((1995) Nat. Gen. 11:210-212) reported that after studying more than 500 primary tumors, almost all the deletions identified in such tumors involved a 170 kb region containing MTAP, p14ARF and P16INK4A. Carson et al. (WO 99/67634) reported that a correlation exists between the stage of tumor development and loss of homozygosity of the gene encoding MTAP and the gene encoding p16. For example, deletion of the MTAP gene, but not p16INK4A was reported to be indicative of a cancer at an early stage of development, whereas deletion of the genes encoding for p16 and MTAP was reported to be indicative of a cancer at a more advanced stage of tumor development. In some osteosarcoma patients, the MTAP gene was present at diagnosis but was deleted at a later time point (Garcia-Castellano et al., Clin. Cancer Res. 8(3) 2002 782-787).

### SUMMARY

The present disclosure provides compounds that inhibit MAT2A. The compounds and their pharmaceutical compositions are useful in methods for treating various cancers, including those that are refractory to standard treatments, such as surgery, radiation therapy, chemotherapy, hormonal therapy, antibody therapy, and combinations thereof.

Thus, in accordance with some embodiments, the present disclosure provides compounds according to Formula I or pharmaceutically acceptable salts, tautomers, and/or isotopologues thereof:

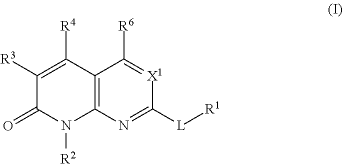

In Formula I, X¹ is N or CR⁵, L is O, S, S(O)₂, NR, or a bond, and R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. In some embodiments, R¹ is optionally substituted by 1-6 halo or 1-6 deuterium. When X¹ is N, L is NR, R is H, and R¹ is C₁-C₆-alkyl, then R¹ is substituted by 1-6 halo.

In some embodiments, wherein L is NR, R and R¹ can be taken together in combination with L to form a 3- to 6-membered heterocycloalkyl (wherein 1-4 ring members are independently N, O, or S) optionally substituted by one or more R^{A}.

R² and R³ are independently selected from the group consisting of C₆-C₁₀-aryl and 5- to 10-membered heteroaryl (wherein 1-4 heteroaryl members are independently N, O, or S). R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN.

R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D}.

R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S). Each alkyl, alkoxy, alkenyl, alkynyl, aryl, carbocyclyl, heterocycloalkyl, and heteroaryl moiety of R^{A} and R^{B} is, independently, optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S) and —(C₁-C₆-alkyl)-(3- to 14-membered heterocycle) (wherein 1-4 heterocycle members are independently N, O, or S), and —O(C₆-C₁₄-aryl). Each alkyl, alkenyl, aryl, and heterocycloalkyl described above is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are, independently, H or C₁-C₆-alkyl.

Notwithstanding the scope of Formula I as described herein, it should be understood that Formula I does not include the compounds:
6-(2-chlorophenyl)-8-(4-fluorophenyl)-2-(methylthio)pyrido[2,3-d]pyrimidin-7(8H)-one;
2-(methylthio)-8-phenyl-6-(o-tolyl)pyrido[2,3-d]pyrimidin-7(8H)-one;
6-(4-hydroxyphenyl)-2-(methylthio)-8-phenylpyrido[2,3-d]pyrimidin-7(8H)-one;
6-(4-methoxyphenyl)-2-(methylthio)-8-phenylpyrido[2,3-d]pyrimidin-7(8H)-one;
6-(2,5-dimethoxyphenyl)-2-(methylthio)-8-phenylpyrido[2,3-d]pyrimidin-7(8H)-one;
6-(4-(tert-butyl)phenyl)-2-(methylthio)-8-phenylpyrido[2,3-d]pyrimidin-7(8H)-one; and
6-(2,6-dichlorophenyl)-2-(methylthio)-8-phenylpyrido[2,3-d]pyrimidin-7(8H)-one.

Additional embodiments of the present disclosure provide compounds according to Formula II or pharmaceutically acceptable salts, tautomers, and/or isotopologues thereof:

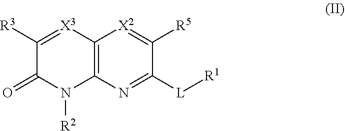

L is O, S, NR, or a bond. In some embodiments, X² is CR⁶ and X³ is N, and in other embodiments X² is N and X³ is CR⁴.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. In some embodiments, R¹ is optionally substituted by 1-6 halo.

In some embodiments wherein L is NR, R and R¹ can be taken together in combination with L to form a 3- to 6-membered heterocycloalkyl (wherein 1-4 ring members are independently N, O, or S) optionally substituted by one or more R^{A}.

R² and R³ are independently selected from the group consisting of C₆-C₁₀-aryl and 5- to 10-membered heteroaryl (wherein 1-4 heteroaryl members are independently N, O, or S). In various embodiments, R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN.

R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D}.

R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are, independently, N, O, or S). Each alkyl, alkoxy, alkenyl, alkynyl, aryl, carbocyclyl, heterocycloalkyl, and heteroaryl moiety of R^{A} and R^{B} is optionally and independently substituted with one or more substituents selected from the group consisting of hydroxy, deuterium, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S), —(C₁-C₆-alkyl)-(3- to 14-membered heterocycle) (wherein 1-4 heterocycle members are independently N, O, or S), and —O(C₆-C₁₄-aryl). Each alkyl, alkenyl, aryl, and heterocycloalkyl described above is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are, independently, H or C₁-C₆-alkyl.

The disclosure provides in another embodiment a pharmaceutical composition comprising a therapeutically effective amount of a compound as described herein or a pharmaceutically acceptable salt, tautomer, and/or isotopologue thereof, and a pharmaceutically acceptable carrier.

In accordance with an additional embodiment, the disclosure provides a method for treating a cancer in a subject suffering therefrom, comprising administering to the subject an effective amount of a MAT2A inhibitor, such as a compound, or a pharmaceutically acceptable salt, tautomer, and/or isotopologue as described herein.

The disclosure also provides in a further embodiment a method for inhibiting the synthesis of S-adenosyl methionine (SAM) in a cell, comprising introducing into the cell an effective amount of a compound, or a pharmaceutically acceptable salt, tautomer, and/or isotopologue thereof, as described herein.

The disclosure also provides in a further embodiment a method for inhibiting the synthesis of S-adenosyl methionine (SAM) in a subject, comprising administering to the subject an effective amount of a compound, or a pharmaceutically acceptable salt, tautomer, and/or isotopologue thereof, as described herein.

In another embodiment, the disclosure provides a method for treating a cancer in a subject suffering therefrom, comprising administering to the subject an effective amount of a compound or a pharmaceutically acceptable salt, tautomer, and/or isotopologue thereof, as described herein.

In accordance with still another embodiment, the disclosure provides a method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, as compared to cancers where the MTAP gene or protein is present and/or fully functioning. The method comprises administering to the subject a therapeutically effective amount of a compound, or a pharmaceutically acceptable salt, tautomer, and/or isotopologue thereof, as described herein.

The disclosure provides in an embodiment a compound as described herein, or a pharmaceutically acceptable salt, tautomer, and/or isotopologue thereof, for use in inhibiting the synthesis of S-adenosyl methionine (SAM).

Another embodiment is a compound as described herein, or a pharmaceutically acceptable salt, tautomer, and/or isotopologue thereof, for use in treating a cancer in a subject suffering therefrom.

### DETAILED DESCRIPTION

The compounds described herein are inhibitors of MAT2A. The present disclosure thus relates not only to such compounds in conformity with Formula I or Formula II, but also to their pharmaceutically acceptable salts, pharmaceutical compositions, tautomers, and/or isotopologues. The compounds and compositions are useful in treating cancers. Some cancers include various MTAP-deleted cancers, i.e., those cancers characterized by the absence or deletion of the MTAP gene or reduced function of the MTAP protein.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein below.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein below.

“Substituted alkenyl” refers to alkenyl substituted at 1 or more, e.g., 1, 2, 3, 4, 5, or even 6 positions, which substituents are attached at any available atom to produce a stable compound, with substitution as described herein. “Optionally substituted alkenyl” refers to alkenyl or substituted alkenyl.

“Alkyne or “alkynyl” refers to a straight or branched chain unsaturated hydrocarbon having the indicated number of carbon atoms and at least one triple bond. Examples of a (C₂-C₅)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

“Substituted alkynyl” refers to an alkynyl substituted at 1 or more, e.g., 1, 2, 3, 4, 5, or even 6 positions, which substituents are attached at any available atom to produce a stable compound, with substitution as described herein. “Optionally substituted alkynyl” refers to alkynyl or substituted alkynyl.

The term “alkoxy” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “carbocyclyl” refers to a monocyclic, bicyclic, tricyclic, or polycyclic, 3- to 14-membered ring system, which is either saturated, such as “cycloalkyl,” or partially unsaturated, such as “cycloalkenyl.” The term “cycloalkenyl” refers specifically to cyclic alkenyl, such as C₃-C₆-cycloalkenyl. The carbocyclyl may be attached via any atom. A carbocyclyl also includes a carbocyclyl that is fused to an aryl or heteroaryl ring as defined herein. Representative examples of carbocyclyl include, but are not limited to cyclopropyl, cyclobutyl, cyclopentyl, cyclohexyl, cyclopropenyl, cyclobutenyl, cyclopentenyl, cyclohexenyl, phenyl, naphthyl, anthracyl, benzofuranyl, and benzothiophenyl. A carbocyclyl group can be unsubstituted or optionally substituted with one or more substituents as described herein below.

“Substituted carbocyclyl” refers to carbocyclyl substituted at 1 or more, e.g., 1, 2, 3, 4, 5, or even 6 positions, which substituents are attached at any available atom to produce a stable compound, with substitution as described herein. “Optionally substituted carbocyclyl” refers to carbocyclyl or substituted carbocyclyl.

“Aryl” when used alone or as part of another term means a carbocyclic aromatic group whether or not fused having the number of carbon atoms designated or if no number is designated, up to 14 carbon atoms, such as a C₆-C₁₄-aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. “Aryl” can be optionally fused with a carbocyclyl ring, as herein defined. An aryl group can be unsubstituted or optionally substituted with one or more substituents as described herein below.

A “substituted aryl” is an aryl that is independently substituted with one or more substituents attached at any available atom to produce a stable compound, wherein the substituents are as described herein. “Optionally substituted aryl” refers to aryl or substituted aryl.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, refers to a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms that are, independently, O, S, or N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein below.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

“Heterocycloalkyl” means a saturated or partially unsaturated non-aromatic monocyclic, bicyclic, tricyclic or polycyclic ring system that has from 3 to 14, such as 3 to 6, atoms in which from 1 to 3 carbon atoms in the ring are replaced by heteroatoms of 0, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein below.

“Optionally substituted heterocycloalkyl” denotes a heterocycloalkyl that is substituted with 1 to 3 substituents, e.g., 1, 2 or 3 substituents, attached at any available atom to produce a stable compound, wherein the substituents are as described herein.

The term “nitrile” or “cyano” can be used interchangeably and refer to a —CN group which is bound to a carbon atom of a heteroaryl ring, aryl ring and a heterocycloalkyl ring.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

The substituent —CO₂H may be replaced with bioisosteric replacements such as:

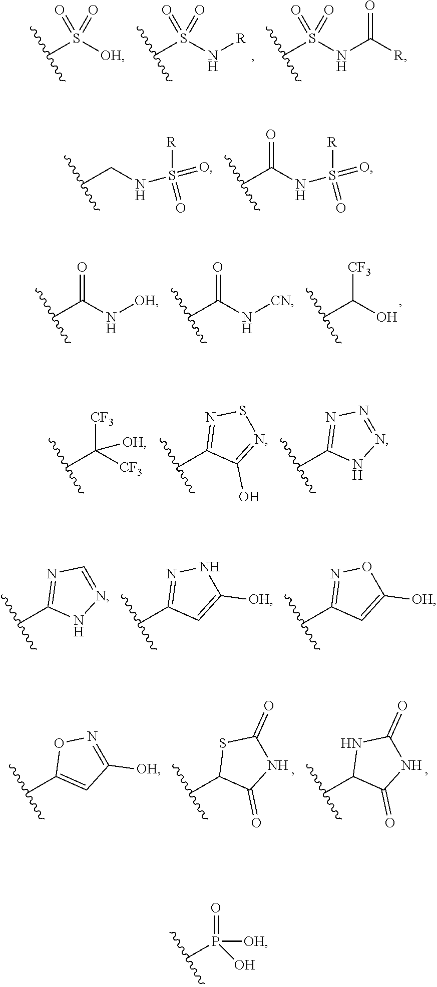

and the like, wherein R has the same definition as R^{A} as defined herein. See, e.g., THE PRACTICE OF MEDICINAL CHEMISTRY (Academic Press. New York, 1996), at page 203.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” may also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D” or “²H” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound of Formula I or Formula II includes a pharmaceutically acceptable salt of an isotopologue of the compound. Similarly, a compound of Formula I or Formula II includes a pharmaceutically acceptable salt of a tautomer of the compound.

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

“Inhibitor” means a compound which prevents or reduces the amount of synthesis of SAM. In an embodiment, an inhibitor binds to MAT2A.

### Compounds

As described generally above, the present disclosure provides compounds according to Formula I, pharmaceutically acceptable salts, tautomers, and/or isotopologues thereof:

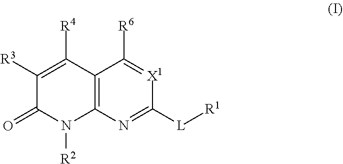

In Formula I, X¹ is N or CR⁵, L is O, S, S(O)₂, NR, or a bond, and R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. In some embodiments, R¹ is optionally substituted by 1-6 halo or 1-6 deuterium. When X¹ is N, L is NR, R is H, and R¹ is C₁-C₆-alkyl, then R¹ is substituted by 1-6 halo. In other embodiments, R¹ is C₁-C₆-alkyl. In further embodiments, R¹ is C₂-C₆-alkenyl. In yet other embodiments, R¹ is C₃-C₆-carbocyclyl. In still further embodiments, R¹ is —(C₁-C₆-alkyl)(C₃-C₆-carbocyclyl). In certain embodiments, R¹—(C₁-C₆-alkyl)(C₃-C₆-cycloalkenyl). In other embodiments, any alkyl in R¹ is straight. In further embodiments, R¹ branched. In yet other embodiments, R¹ is optionally substituted by 1-6 fluoro.

In some embodiments, wherein L is NR, R and R¹ can be taken together in combination with L to form a 3- to 6-membered heterocycloalkyl (wherein 1-4 ring members are independently N, O, or S) optionally substituted by one or more R^{A}.

R² and R³ are independently selected from the group consisting of C₆-C₁₀-aryl and 5- to 10-membered heteroaryl (wherein 1-4 heteroaryl members are independently N, O, or S). In some embodiments, R² and R³ are C₆-C₁₀-aryl. In other embodiments, R² and R³ are 5- to 10-membered heteroaryl (wherein 1-4 heteroaryl members are independently N, O, or S). In further embodiments, R² is C₆-C₁₀-aryl and R³ is 5- to 10-membered heteroaryl (wherein 1-4 heteroaryl members are independently N, O, or S). In yet other embodiments, R³ is C₆-C₁₀-aryl and R² is 5- to 10-membered heteroaryl (wherein 1-4 heteroaryl members are independently N, O, or S). R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN. In some embodiments, the substituent is R^{A}. In other embodiments, the substituent is OR^{A}. In further embodiments, the substituent is halo such as fluoro. In certain embodiments, the substituent is —N═N—R^{A}. In yet other embodiments, the substituent is NR^{A}R^{B} such as NH₂. In still further embodiments, the substituent is —(C₁-C₆-alkyl)NR^{A}R^{B} such as —(C₁-C₆-alkyl)NH₂. In some embodiments, the substituent is —C(O)OR^{A} such as —C(O)OH. In other embodiments, the substituent is —C(O)NR^{A}R^{B} such as —C(O)NH₂. In still further embodiments, the substituent is —OC(O)R^{A} such as —OC(O)OH. In certain embodiments, the substituent is —CN.

R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D}. In some embodiments, R⁴ is H. In other embodiments, R⁴ is C₁-C₆-alkyl. In further embodiments, R⁴ is C₁-C₆-alkoxy. In certain embodiments, R⁴ is C₂-C₆-alkenyl. In yet other embodiments, R⁴ is C₂-C₆-alkynyl. In still further embodiments, R⁴ is halo such as fluoro. In some embodiments, R⁴ is oxo. In other embodiments, R⁴ is —CN. In further embodiments, R⁴ is NR^{C}R^{D} such as NH₂.

R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D}. In some embodiments, R⁵ is H. In other embodiments, R⁵ is C₁-C₆-alkyl. In further embodiments, R⁵ is C₁-C₆-alkoxy. In certain embodiments, R⁵ is C₂-C₆-alkenyl. In yet other embodiments, R⁵ is C₂-C₆-alkynyl. In still further embodiments, R⁵ is halo such as fluoro. In some embodiments, R⁵ is —CN. In further embodiments, R⁵ is NR^{C}R^{D} such as NH₂.

R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B}. In some embodiments, R⁶ is H. In other embodiments, R⁶ is C₁-C₆-alkyl. In yet other embodiments, R⁶ is C₁-C₆-alkyl substituted by halo such as fluoro. In further embodiments, R⁶ is —OH. In certain embodiments, R⁶ is halo such as fluoro. In yet other embodiments, R⁶ is —CN. In some embodiments, R⁶ is —(C₁-C₆-alkyl)NR^{A}R^{B} such as —(C₁-C₆-alkyl)NH₂. In further embodiments, R⁶ is NR^{C}R^{D} such as NH₂.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S). In some embodiments, R^{A} is H. In other embodiments, R^{A} is —CN. In further embodiments, R^{A} is -hydroxy. In certain embodiments, R^{A} is oxo. In still other embodiments, R^{A} is C₁-C₆-alkyl. In yet further embodiments, R^{A} is C₁-C₆-alkoxy. In some embodiments, R^{A} is C₂-C₆-alkenyl. In other embodiments, R^{A} is C₂-C₆-alkynyl. In further embodiments, R^{A} is NH₂. In certain embodiments, R^{A} is —S(O)₀₋₂—(C₁-C₆-alkyl). In yet other embodiments, R^{A} is —S(O)₀₋₂—(C₆-C₁₀-aryl). In still further embodiments, R^{A} is —C(O)(C₁-C₆-alkyl). In some embodiments, R^{A} is —C(O)(C₃-C₁₄-carbocyclyl). In other embodiments, R^{A} is —C₃-C₁₄-carbocyclyl. In further embodiments, R^{A} is —(C₁-C₆-alkyl)(C₃-C₁₄-carbocyclyl). In certain embodiments, R^{A} is C₆-C₁₀-aryl. In still other embodiments, R^{A} is 3- to 14-membered heterocycloalkyl (wherein 1-4 ring members are, independently, N, O, or S). In still further embodiments, R^{A} is —(C₁-C₆-alkyl)-(3- to 14-membered heterocycloalkyl) (wherein 1-4 ring members are independently N, O, or S). In some embodiments, R^{A} is 5- to 10-membered heteroaryl (wherein 1-4 heteroaryl members are independently N, O, or S). In some embodiments, R^{B} is H. In other embodiments, R^{B} is —CN. In further embodiments, R^{B} is -hydroxy. In certain embodiments, R^{B} is oxo. In still other embodiments, R^{B} is C₁-C₆-alkyl. In yet further embodiments, R^{B} is C₁-C₆-alkoxy. In some embodiments, R^{B} is C₂-C₆-alkenyl. In other embodiments, R^{B} is C₂-C₆-alkynyl. In further embodiments, R^{B} is NH₂. In certain embodiments, R^{B} is —S(O)₀₋₂—(C₁-C₆-alkyl). In yet other embodiments, R^{B} is —S(O)₀₋₂—(C₆-C₁₀-aryl). In still further embodiments, R^{B} is —C(O)(C₁-C₆-alkyl). In some embodiments, R^{B} is —C(O)(C₃-C₁₄-carbocyclyl). In other embodiments, R^{B} is —C₃-C₁₄-carbocyclyl. In further embodiments, R^{B} is —(C₁-C₆-alkyl)(C₃-C₁₄-carbocyclyl). In certain embodiments, R^{B} is C₆-C₁₀-aryl. In still other embodiments, R^{B} is 3- to 14-membered heterocycloalkyl (wherein 1-4 ring members are, independently, N, O, or S). In still further embodiments, R^{B} is —(C₁-C₆-alkyl)-(3- to 14-membered heterocycloalkyl) (wherein 1-4 ring members are independently N, O, or S). In some embodiments, R^{B} is 5- to 10-membered heteroaryl (wherein 1-4 heteroaryl members are independently N, O, or S).

Each alkyl, alkoxy, alkenyl, alkynyl, aryl, carbocyclyl, heterocycloalkyl, and heteroaryl moiety of R^{A} and R^{B} is independently, optionally substituted with one or more substituents selected from the group consisting of hydroxy, deuterium, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S), —(C₁-C₆-alkyl)-(3- to 14-membered heterocycle) (wherein 1-4 heterocycle members are independently N, O, or S), and —O(C₆-C₁₄-aryl). Each alkyl, alkenyl, aryl, and heterocycloalkyl described above is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are, independently, H or C₁-C₆-alkyl. In some embodiments, R^{C} is H. In other embodiments, R^{D} is H. In other embodiments, R^{C} is C₁-C₆-alkyl. In further embodiments, R^{D} is C₁-C₆-alkyl. In yet other embodiments, R^{C} and R^{D} are C₁-C₆-alkyl. In still further embodiments, R^{C} and R^{D} are H.

Notwithstanding the scope of Formula I as described herein, it should be understood that Formula I does not include the following compounds:
6-(2-chlorophenyl)-8-(4-fluorophenyl)-2-(methylthio)pyrido[2,3-d]pyrimidin-7(8H)-one;
2-(methylthio)-8-phenyl-6-(o-tolyl)pyrido[2,3-d]pyrimidin-7(8H)-one;
6-(4-hydroxyphenyl)-2-(methylthio)-8-phenylpyrido[2,3-d]pyrimidin-7(8H)-one;
6-(4-methoxyphenyl)-2-(methylthio)-8-phenylpyrido[2,3-d]pyrimidin-7(8H)-one;
6-(2,5-dimethoxyphenyl)-2-(methylthio)-8-phenylpyrido[2,3-d]pyrimidin-7(8H)-one;
6-(4-(tert-butyl)phenyl)-2-(methylthio)-8-phenylpyrido[2,3-d]pyrimidin-7(8H)-one; and
6-(2,6-dichlorophenyl)-2-(methylthio)-8-phenylpyrido[2,3-d]pyrimidin-7(8H)-one.

As described generally above, the present disclosure also provides compounds according to Formula II, pharmaceutically acceptable salts, tautomers, and/or isotopologues thereof:

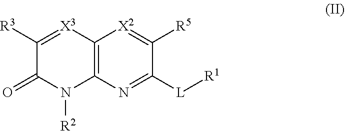

In Formula II, L is O, S, NR, or a bond. In some embodiments, X² is CR⁶ and X³ is N, and in other embodiments X² is N and X³ is CR⁴. R is H or C₁-C₆-alkyl.

Further, R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. In some embodiments, R¹ is optionally substituted by 1-6 halo. In other embodiments, R¹ is C₁-C₆-alkyl. In further embodiments, R¹ is C₂-C₆-alkenyl. In yet other embodiments, R¹ is C₃-C₆-carbocyclyl. In still further embodiments, R¹ is —(C₁-C₆-alkyl)(C₃-C₆-carbocyclyl). In certain embodiments, R¹—(C₁-C₆-alkyl)(C₃-C₆-cycloalkenyl). In other embodiments, any alkyl in R¹ is straight. In further embodiments, R¹ branched. R¹ is optionally substituted by 1-6 fluoro.

In some Formula II embodiments wherein L is NR, R and R¹ can be taken together in combination with L to form a 3- to 6-membered heterocycloalkyl (wherein 1-4 ring members are, independently, N, O, or S) optionally substituted by one or more R^{A}.

In Formula II, R² and R³ are independently selected from the group consisting of C₆-C₁₀-aryl and 5- to 10-membered heteroaryl (wherein 1-4 heteroaryl members are independently N, O, or S). In some embodiments, R² and R³ are C₆-C₁₀-aryl. In other embodiments, R² and R³ are 5- to 10-membered heteroaryl (wherein 1-4 heteroaryl members are independently N, O, or S). In further embodiments, R² is C₆-C₁₀-aryl and R³ is 5- to 10-membered heteroaryl (wherein 1-4 heteroaryl members are independently N, O, or S). In yet other embodiments, R³ is C₆-C₁₀-aryl and R² is 5- to 10-membered heteroaryl (wherein 1-4 heteroaryl members are independently N, O, or S). In various embodiments, R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN. In some embodiments, the substituent is R^{A}. In other embodiments, the substituent is OR^{A}. In further embodiments, the substituent is halo such as fluoro. In certain embodiments, the substituent is —N═N—R^{A}. In yet other embodiments, the substituent is NR^{A}R^{B} such as NH₂. In still further embodiments, the substituent is —(C₁-C₆-alkyl)NR^{A}R^{B} such as —(C₁-C₆-alkyl)NH₂. In some embodiments, the substituent is —C(O)OR^{A} such as —C(O)OH. In other embodiments, the substituent is —C(O)NR^{A}R^{B} such as —C(O)NH₂. In still further embodiments, the substituent is —OC(O)R^{A} such as —OC(O)OH. In certain embodiments, the substituent is —CN.

Additionally, R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D}. In some embodiments, R⁴ is H. In other embodiments, R⁴ is C₁-C₆-alkyl. In further embodiments, R⁴ is C₁-C₆-alkoxy. In certain embodiments, R⁴ is C₂-C₆-alkenyl. In yet other embodiments, R⁴ is C₂-C₆-alkynyl. In still further embodiments, R⁴ is halo such as fluoro. In some embodiments, R⁴ is oxo. In other embodiments, R⁴ is —CN. In further embodiments, R⁴ is NR^{C}R^{D} such as NH₂.

In Formula II, R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D}. In some embodiments, R⁵ is H. In other embodiments, R⁵ is C₁-C₆-alkyl. In further embodiments, R⁵ is C₁-C₆-alkoxy. In certain embodiments, R⁵ is C₂-C₆-alkenyl. In yet other embodiments, R⁵ is C₂-C₆-alkynyl. In still further embodiments, R⁵ is halo such as fluoro. In some embodiments, R⁵ is —CN. In further embodiments, R⁵ is NR^{C}R^{D} such as NH₂.

Further, R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B}. In some embodiments, R⁶ is H. In other embodiments, R⁶ is C₁-C₆-alkyl. In yet other embodiments, R⁶ is C₁-C₆-alkyl substituted by halo such as fluoro. In further embodiments, R⁶ is —OH. In certain embodiments, R⁶ is halo such as fluoro. In yet other embodiments, R⁶ is —CN. In some embodiments, R⁶ is —(C₁-C₆-alkyl)NR^{A}R^{B} such as —(C₁-C₆-alkyl)NH₂. In further embodiments, R⁶ is NR^{C}R^{D} such as NH₂.

In Formula II, R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S). In some embodiments, R^{A} is H. In other embodiments, R^{A} is —CN. In further embodiments, R^{A} is -hydroxy. In certain embodiments, R^{A} is oxo. In still other embodiments, R^{A} is C₁-C₆-alkyl. In yet further embodiments, R^{A} is C₁-C₆-alkoxy. In some embodiments, R^{A} is C₂-C₆-alkenyl. In other embodiments, R^{A} is C₂-C₆-alkynyl. In further embodiments, R^{A} is NH₂. In certain embodiments, R^{A} is —S(O)₀₋₂—(C₁-C₆-alkyl). In yet other embodiments, R^{A} is —S(O)₀₋₂—(C₆-C₁₀-aryl). In still further embodiments, R^{A} is —C(O)(C₁-C₆-alkyl). In some embodiments, R^{A} is —C(O)(C₃-C₁₄-carbocyclyl). In other embodiments, R^{A} is —C₃-C₁₄-carbocyclyl. In further embodiments, R^{A} is —(C₁-C₆-alkyl)(C₃-C₁₄-carbocyclyl). In certain embodiments, R^{A} is C₆-C₁₀-aryl. In still other embodiments, R^{A} is 3- to 14-membered heterocycloalkyl (wherein 1-4 ring members are, independently, N, O, or S). In still further embodiments, R^{A} is —(C₁-C₆-alkyl)-(3- to 14-membered heterocycloalkyl) (wherein 1-4 ring members are independently N, O, or S). In some embodiments, R^{A} is 5- to 10-membered heteroaryl (wherein 1-4 heteroaryl members are independently N, O, or S). In some embodiments, R^{B} is H. In other embodiments, R^{B} is —CN. In further embodiments, R^{B} is -hydroxy. In certain embodiments, R^{B} is oxo. In still other embodiments, R^{B} is C₁-C₆-alkyl. In yet further embodiments, R^{B} is C₁-C₆-alkoxy. In some embodiments, R^{B} is C₂-C₆-alkenyl. In other embodiments, R^{B} is C₂-C₆-alkynyl. In further embodiments, R^{B} is N12. In certain embodiments, R^{B} is —S(O)₀₋₂—(C₁-C₆-alkyl). In yet other embodiments, R^{B} is —S(O)₀₋₂—(C₆-C₁₀-aryl). In still further embodiments, R^{B} is —C(O)(C₁-C₆-alkyl). In some embodiments, R^{B} is —C(O)(C₃-C₁₄-carbocyclyl). In other embodiments, R^{B} is —C₃-C₁₄-carbocyclyl. In further embodiments, R^{B} is —(C₁-C₆-alkyl)(C₃-C₁₄-carbocyclyl). In certain embodiments, R^{B} is C₆-C₁₀-aryl. In still other embodiments, R^{B} is 3- to 14-membered heterocycloalkyl (wherein 1-4 ring members are, independently, N, O, or S). In still further embodiments, R^{B} is —(C₁-C₆-alkyl)-(3- to 14-membered heterocycloalkyl) (wherein 1-4 ring members are independently N, O, or S). In some embodiments, R^{B} is 5- to 10-membered heteroaryl (wherein 1-4 heteroaryl members are independently N, O, or S).

Each alkyl, alkoxy, alkenyl, alkynyl, aryl, carbocyclyl, heterocycloalkyl, and heteroaryl moiety of R^{A} and R^{B} is optionally and independently substituted with one or more substituents selected from the group consisting of hydroxy, deuterium, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are independently N, O, or S), and 5- to 10-membered heteroaryl (wherein 1-4 heteroaryl members are independently N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S), —(C₁-C₆-alkyl)-(3- to 14-membered heterocycle) (wherein 1-4 heterocycle members are independently N, O, or S), and —O(C₆-C₁₄-aryl). Each alkyl, alkenyl, aryl, and heterocycloalkyl described above is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo.

In Formula II, R^{C} and R^{D} are, independently, H or C₁-C₆-alkyl. In some embodiments, R^{C} is H. In other embodiments, R^{D} is H. In other embodiments, R^{C} is C₁-C₆-alkyl. In further embodiments, R^{D} is C₁-C₆-alkyl. In yet other embodiments, R^{C} and R^{D} are C₁-C₆-alkyl. In still further embodiments, R^{C} and R^{D} are H.

In some Formula I compounds, per various embodiments, X¹ is N. In other embodiments, X¹ is CR⁵.

In some Formula II compounds, in accordance with various embodiments, X² is CR⁶ and X³ is N. In other embodiments, X² is N and X³ is CR⁴.

In some embodiments for Formula I or Formula II, optionally in combination with any other embodiment described herein, each of R⁴ and R⁵ is, independently, H or C₁-C₆-alkyl. Further, R⁶ is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are, independently, H or C₁-C₆-alkyl).

In other embodiments for Formula I or Formula II, at least one of R⁴, R⁵, and R⁶ is H. For example, R⁴ is H, R⁵ is H, or R⁶ is H. The present disclosure provides compounds, per various embodiments, wherein each of R⁴, R⁵, and R⁶ is H. In some embodiments, R⁴ is H. In other embodiments, R⁵ is H. In further embodiments, R⁶ is H. In yet other embodiments, each of R⁴, R⁵, and R⁶ is H.

In additional embodiments for Formula I or Formula II, R² is C₆-C₁₀-aryl or 5- to 10-membered heteroaryl. Thus, for example, R² can be C₆-C₁₀-aryl, such as phenyl. In other embodiments, R² is 5- to 10-membered heteroaryl wherein 1 ring member is N. An exemplary embodiment is one wherein R² is pyridyl.

In some embodiments for Formula I or Formula II, R³ is 5- to 10-membered heteroaryl. For example, non-limiting examples of R³ include benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

In other embodiments for Formula I or Formula II, R³ is C₆-C₁₀-aryl. For example, in an embodiment, R³ is phenyl.

The present disclosure also includes embodiments for Formula I or Formula II wherein R² is phenyl and R³ is 5- to 10-membered heteroaryl. In other embodiments, each of R² and R³ is C₆-C₁₀-aryl. For example, each of R² and R³ can be phenyl.

In still further embodiments relating to Formula I or Formula II, L is O or NR. In an embodiment, L is NR. In other embodiments, L is O. In further embodiments, L is S. In still other embodiments, L is NR (wherein in some aspects R is H or C₁-C₆-alkyl or in other aspects, R is H, or in further aspects R is C₁-C₆-alkyl). In yet further embodiments, L is a bond.

In some embodiments relating to Formula I, L is S(O)₂.

In various embodiments for Formula I or Formula II, R¹ is C₁-C₆-alkyl or C₃-C₅-carbocyclyl. Thus, for example, R¹ can be C₁-C₃-alkyl. In some embodiments, C₁-C₃-alkyl is optionally substituted by 1-3 fluoro.

Other embodiments of the disclosure provide Formula I or Formula II compounds wherein
L is O or NR and R is H;
R¹ is C₁-C₃-alkyl that is optionally substituted by 1-3 fluoro;
R² is 5- to 10-membered heteroaryl (wherein 1 heteroaryl member is N) or C₆-C₁₀-aryl;
R³ is 5- to 10-membered heteroaryl (wherein 1 to 3 heteroaryl members are, independently, N, O, or S), or C₆-C₁₀-aryl; and
each of R⁴, R⁵, and R⁶ is H.

In these embodiments, for example, L can be NR. In some embodiments, R² is optionally substituted phenyl; and R³ is an optionally substituted 5- to 10-membered heteroaryl wherein 1 to 3 heteroaryl members are, independently, N, O, or S. Thus, in some exemplary embodiments, R³ is selected from the group consisting of optionally substituted benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

In other such embodiments, R² is an optionally substituted 5- to 10-membered heteroaryl wherein 1 to 3 heteroaryl members are, independently, N, O, or S, and R³ is optionally substituted phenyl. In further embodiments, R² is an optionally substituted 5- to 10-membered heteroaryl wherein 1 heteroaryl member is N and R³ is optionally substituted phenyl.

In still other such embodiments, R² and R³ independently are optionally substituted phenyl.

Some embodiments are Formula I or Formula II compounds wherein L is O or NR and R is H; X¹ is CR⁵; R¹ is C₁-C₃-alkyl that is optionally substituted by 1-3 fluoro; R² is substituted phenyl or substituted pyridyl; R³ is selected from the group consisting of substituted phenyl, substituted benzimidazolyl, and triazolopyridinyl; and each of R⁴, R⁵, and R⁶ is H.

In yet further embodiments, X¹ is CR⁵ and R⁵ is H.

In various embodiments, the disclosure provides specific examples of Formula I and Formula II compounds, and their pharmaceutically acceptable salts, tautomers, and/or isotopologues thereof as set forth in Tables 1-6 below.

TABLE 1
Examples of Formula I Compounds

| Ex | |
|---|---|
| No | Structure |
| 101 | 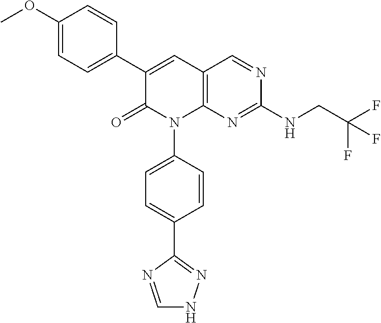 [SMILES: COc1ccc(-c2cc3cnc(NCC(F)(F)F)nc3n(-c3ccc(-c4nc[nH]n4)cc3)c2=O)cc1] |
| 102 | 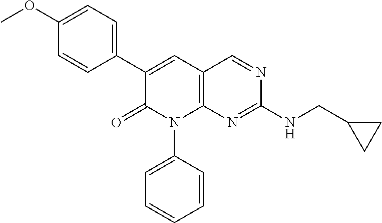 [SMILES: COc1ccc(-c2cc3cnc(NCC4CC4)nc3n(-c3ccccc3)c2=O)cc1] |
| 103 | 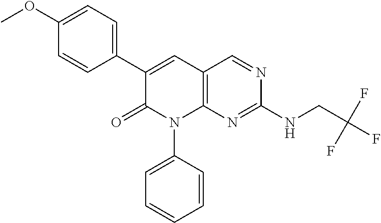 [SMILES: COc1ccc(-c2cc3cnc(NCC(F)(F)F)nc3n(-c3ccccc3)c2=O)cc1] |
| 104 | 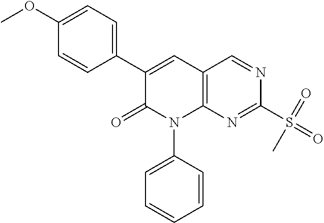 [SMILES: COc1ccc(-c2cc3cnc(S(C)(=O)=O)nc3n(-c3ccccc3)c2=O)cc1] |
| 105 | 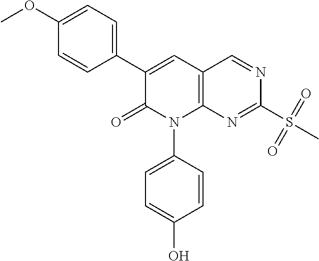 |
| 106 | 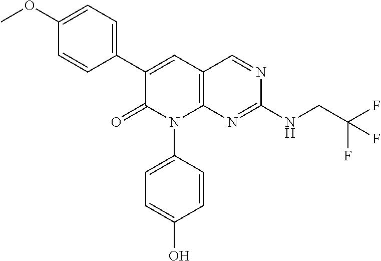 [SMILES: COc1ccc(-c2cc3cnc(NCC(F)(F)F)nc3n(-c3ccc(O)cc3)c2=O)cc1] |
| 107 | 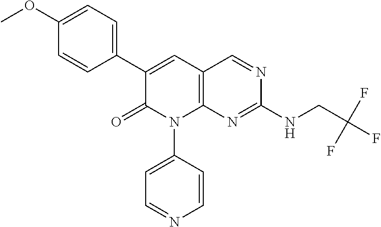 [SMILES: COc1ccc(-c2cc3cnc(NCC(F)(F)F)nc3n(-c3ccncc3)c2=O)cc1] |
| 108 | 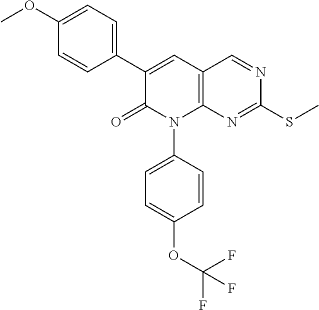 |
| 109 | 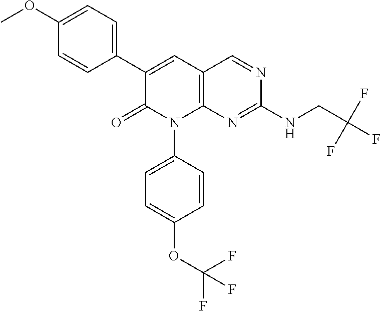 [SMILES: COc1ccc(-c2cc3cnc(NCC(F)(F)F)nc3n(-c3ccc(OC(F)(F)F)cc3)c2=O)cc1] |
| 110 | 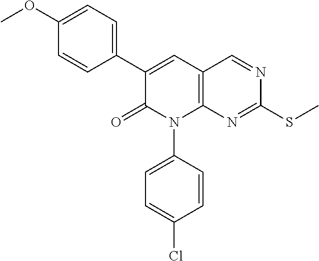 |
| 111 | 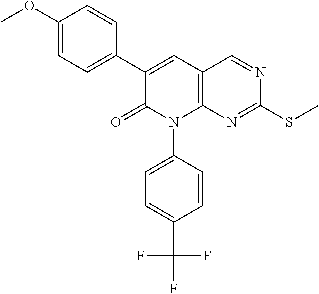 |
| 112 | 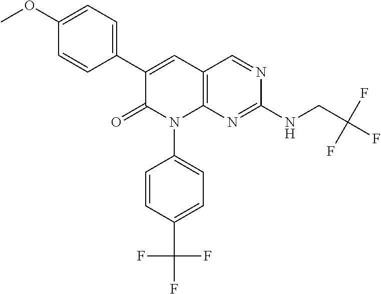 [SMILES: COc1ccc(-c2cc3cnc(NCC(F)(F)F)nc3n(-c3ccc(C(F)(F)F)cc3)c2=O)cc1] |
| 113 | 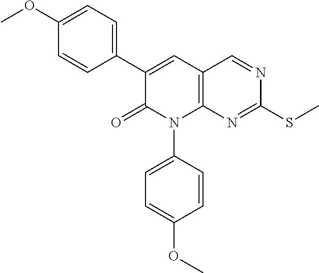 |
| 114 | 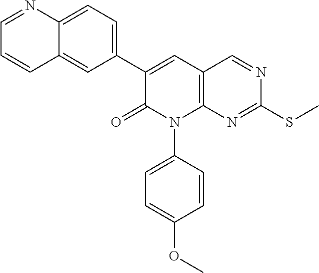 [SMILES: COc1ccc(-n2c(=O)c(-c3ccc4ncccc4c3)cc3cnc(SC)nc32)cc1] |
| 115 | 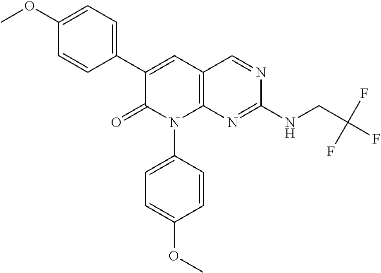 [SMILES: COc1ccc(-c2cc3cnc(NCC(F)(F)F)nc3n(-c3ccc(OC)cc3)c2=O)cc1] |
| 116 | 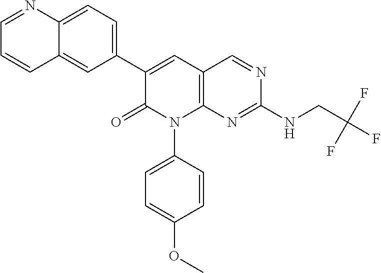 [SMILES: COc1ccc(-n2c(=O)c(-c3ccc4ncccc4c3)cc3cnc(NCC(F)(F)F)nc32)cc1] |
| 117 | 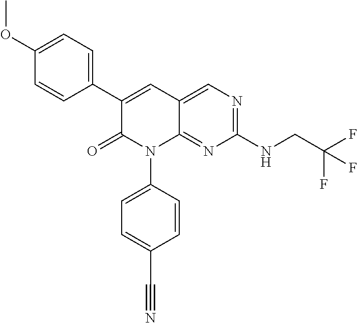 [SMILES: COc1ccc(-c2cc3cnc(NCC(F)(F)F)nc3n(-c3ccc(C#N)cc3)c2=O)cc1] |
| 118 | 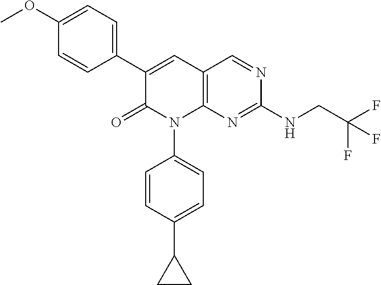 [SMILES: COc1ccc(-c2cc3cnc(NCC(F)(F)F)nc3n(-c3ccc(C4CC4)cc3)c2=O)cc1] |
| 119 | 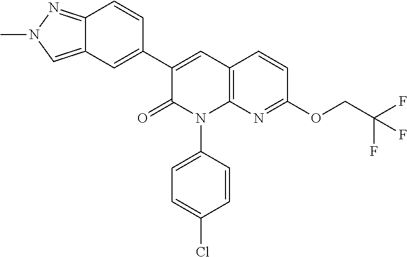 [SMILES: Cn1cc2cc(-c3cc4ccc(OCC(F)(F)F)nc4n(-c4ccc(Cl)cc4)c3=O)ccc2n1] |
| 120 | 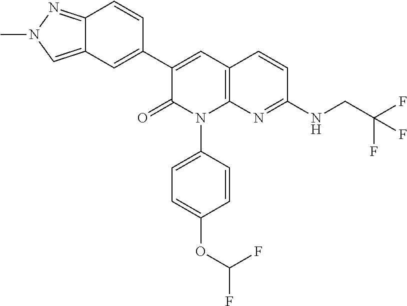 [SMILES: Cn1cc2cc(-c3cc4ccc(NCC(F)(F)F)nc4n(-c4ccc(OC(F)F)cc4)c3=O)ccc2n1] |
| 121 | 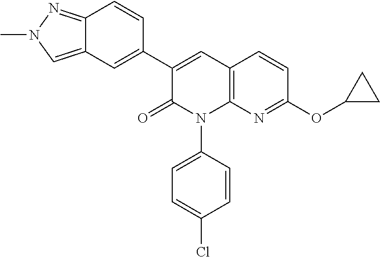 [SMILES: Cn1cc2cc(-c3cc4ccc(OC5CC5)nc4n(-c4ccc(Cl)cc4)c3=O)ccc2n1] |
| 122 | 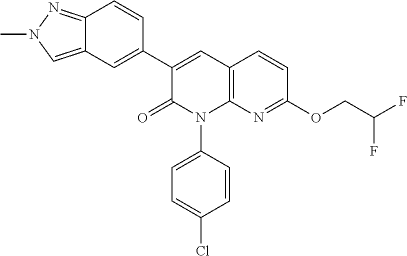 [SMILES: Cn1cc2cc(-c3cc4ccc(OCC(F)F)nc4n(-c4ccc(Cl)cc4)c3=O)ccc2n1] |
| 123 | 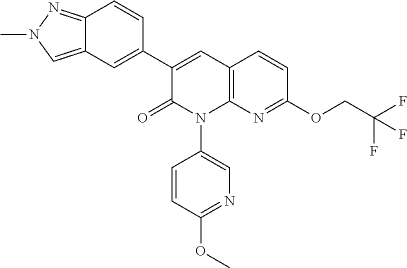 [SMILES: COc1ccc(-n2c(=O)c(-c3ccc4nn(C)cc4c3)cc3ccc(OCC(F)(F)F)nc32)cn1] |
| 124 | 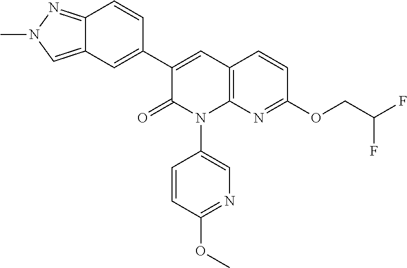 [SMILES: COc1ccc(-n2c(=O)c(-c3ccc4nn(C)cc4c3)cc3ccc(OCC(F)F)nc32)cn1] |
| 125 | 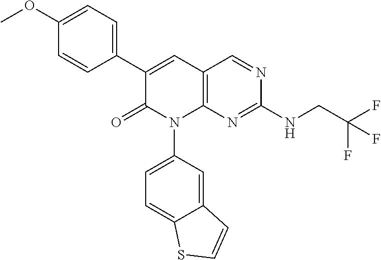 [SMILES: COc1ccc(-c2cc3cnc(NCC(F)(F)F)nc3n(-c3ccc4sccc4c3)c2=O)cc1] |
| 126 | 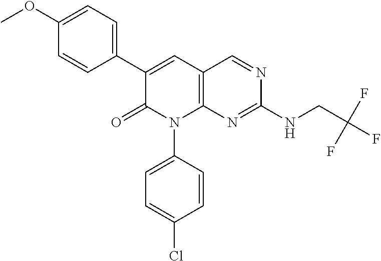 [SMILES: COc1ccc(-c2cc3cnc(NCC(F)(F)F)nc3n(-c3ccc(Cl)cc3)c2=O)cc1] |
| 127 | 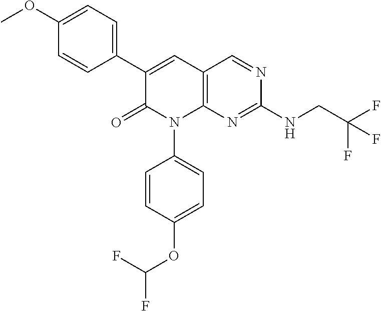 [SMILES: COc1ccc(-c2cc3cnc(NCC(F)(F)F)nc3n(-c3ccc(OC(F)F)cc3)c2=O)cc1] |
| 128 | 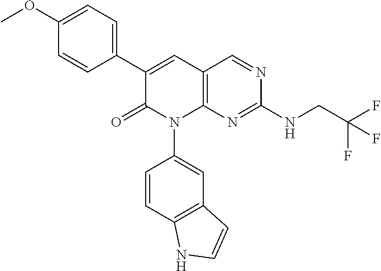 [SMILES: COc1ccc(-c2cc3cnc(NCC(F)(F)F)nc3n(-c3ccc4[nH]ccc4c3)c2=O)cc1] |
| 129 | 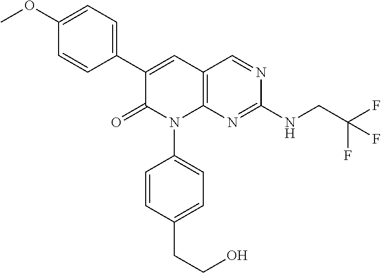 [SMILES: COc1ccc(-c2cc3cnc(NCC(F)(F)F)nc3n(-c3ccc(CCO)cc3)c2=O)cc1] |
| 130 | 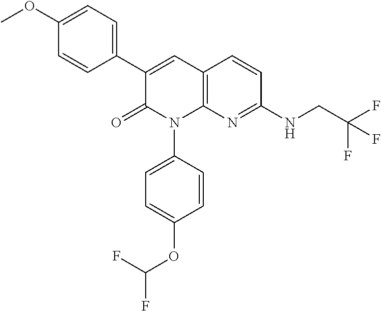 [SMILES: COc1ccc(-c2cc3ccc(NCC(F)(F)F)nc3n(-c3ccc(OC(F)F)cc3)c2=O)cc1] |
| 131 | 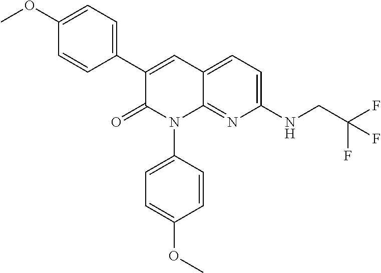 [SMILES: COc1ccc(-c2cc3ccc(NCC(F)(F)F)nc3n(-c3ccc(OC)cc3)c2=O)cc1] |
| 132 | 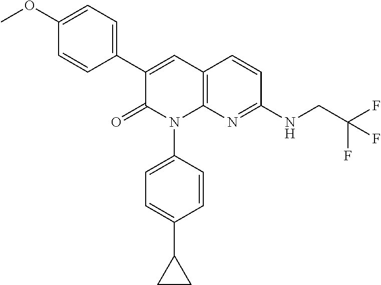 [SMILES: COc1ccc(-c2cc3ccc(NCC(F)(F)F)nc3n(-c3ccc(C4CC4)cc3)c2=O)cc1] |
| 133 | 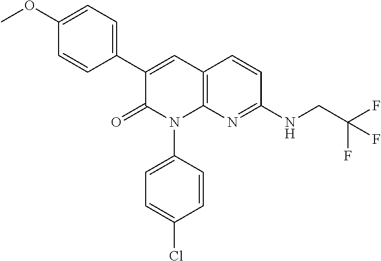 [SMILES: COc1ccc(-c2cc3ccc(NCC(F)(F)F)nc3n(-c3ccc(Cl)cc3)c2=O)cc1] |
| 134 | 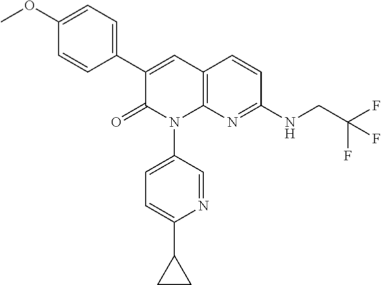 [SMILES: COc1ccc(-c2cc3ccc(NCC(F)(F)F)nc3n(-c3ccc(C4CC4)nc3)c2=O)cc1] |
| 135 | 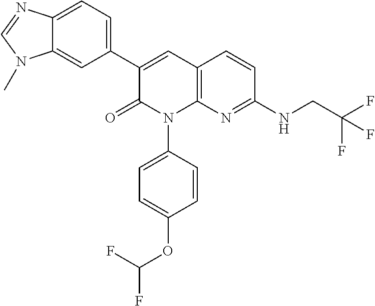 [SMILES: Cn1cnc2ccc(-c3cc4ccc(NCC(F)(F)F)nc4n(-c4ccc(OC(F)F)cc4)c3=O)cc21] |
| 136 | 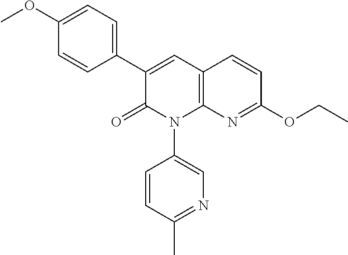 [SMILES: CCOc1ccc2cc(-c3ccc(OC)cc3)c(=O)n(-c3ccc(C)nc3)c2n1] |
| 137 | 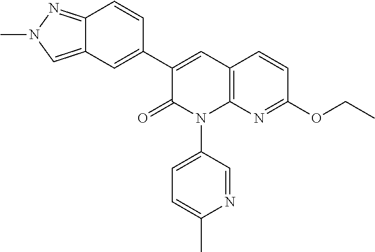 [SMILES: CCOc1ccc2cc(-c3ccc4nn(C)cc4c3)c(=O)n(-c3ccc(C)nc3)c2n1] |
| 138 | 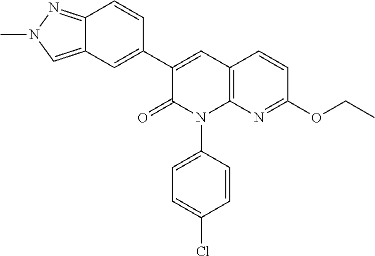 [SMILES: CCOc1ccc2cc(-c3ccc4nn(C)cc4c3)c(=O)n(-c3ccc(Cl)cc3)c2n1] |
| 139 | 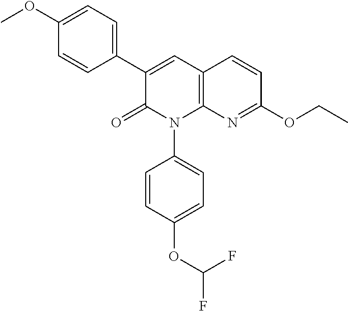 [SMILES: CCOc1ccc2cc(-c3ccc(OC)cc3)c(=O)n(-c3ccc(OC(F)F)cc3)c2n1] |
| 140 | 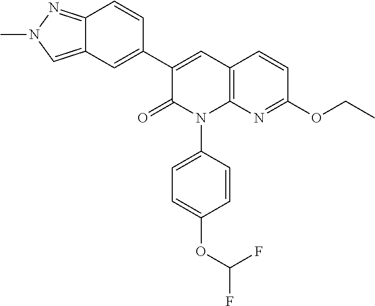 [SMILES: CCOc1ccc2cc(-c3ccc4nn(C)cc4c3)c(=O)n(-c3ccc(OC(F)F)cc3)c2n1] |
| 141 | 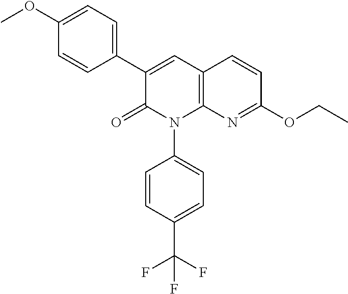 [SMILES: CCOc1ccc2cc(-c3ccc(OC)cc3)c(=O)n(-c3ccc(C(F)(F)F)cc3)c2n1] |
| 142 | 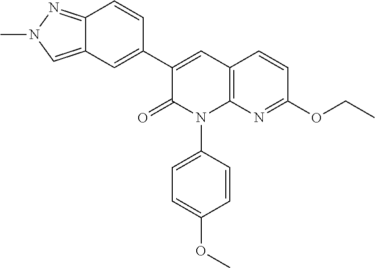 [SMILES: CCOc1ccc2cc(-c3ccc4nn(C)cc4c3)c(=O)n(-c3ccc(OC)cc3)c2n1] |
| 143 | 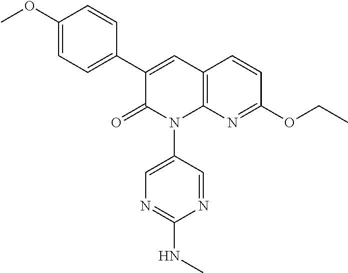 [SMILES: CCOc1ccc2cc(-c3ccc(OC)cc3)c(=O)n(-c3cnc(NC)nc3)c2n1] |
| 144 | 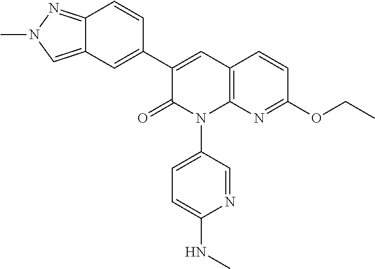 [SMILES: CCOc1ccc2cc(-c3ccc4nn(C)cc4c3)c(=O)n(-c3ccc(NC)nc3)c2n1] |
| 145 | 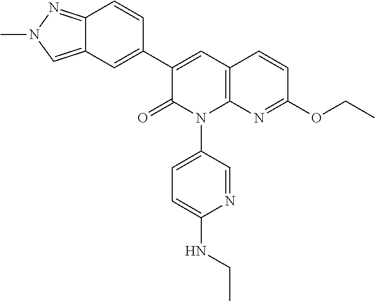 [SMILES: CCNc1ccc(-n2c(=O)c(-c3ccc4nn(C)cc4c3)cc3ccc(OCC)nc32)cn1] |
| 146 | 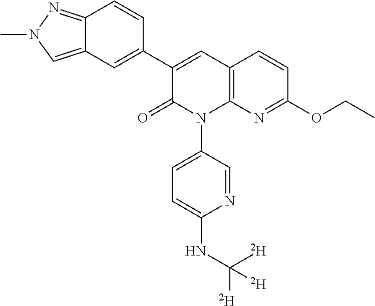 [SMILES: [2H]C([2H])([2H])Nc1ccc(-n2c(=O)c(-c3ccc4nn(C)cc4c3)cc3ccc(OCC)nc32)cn1] |
| 147 | 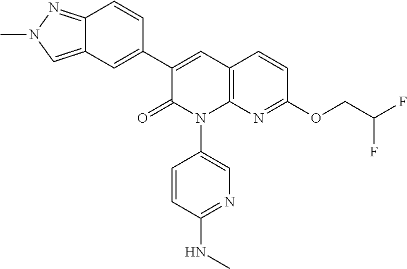 [SMILES: CNc1ccc(-n2c(=O)c(-c3ccc4nn(C)cc4c3)cc3ccc(OCC(F)F)nc32)cn1] |
| 148 | 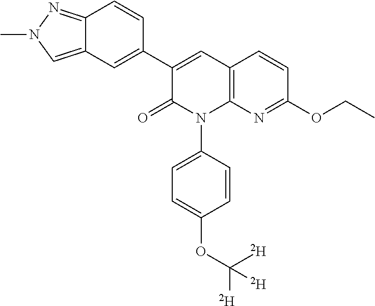 [SMILES: [2H]C([2H])([2H])Oc1ccc(-n2c(=O)c(-c3ccc4nn(C)cc4c3)cc3ccc(OCC)nc32)cc1] |
| 149 | 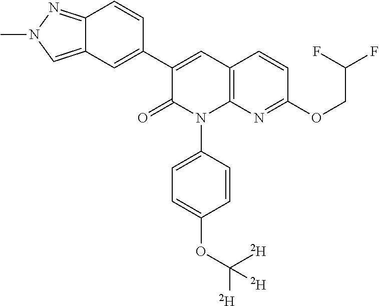 [SMILES: [2H]C([2H])([2H])Oc1ccc(-n2c(=O)c(-c3ccc4nn(C)cc4c3)cc3ccc(OCC(F)F)nc32)cc1] |
| 150 | 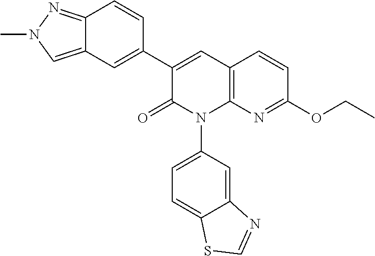 [SMILES: CCOc1ccc2cc(-c3ccc4nn(C)cc4c3)c(=O)n(-c3ccc4scnc4c3)c2n1] |
| 151 | 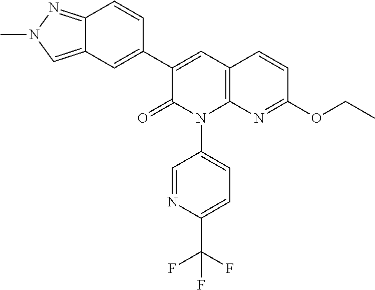 [SMILES: CCOc1ccc2cc(-c3ccc4nn(C)cc4c3)c(=O)n(-c3ccc(C(F)(F)F)nc3)c2n1] |
| 152 | 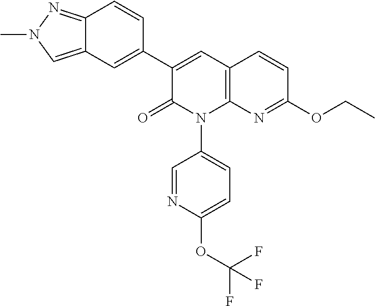 [SMILES: CCOc1ccc2cc(-c3ccc4nn(C)cc4c3)c(=O)n(-c3ccc(OC(F)(F)F)nc3)c2n1] |
| 153 | 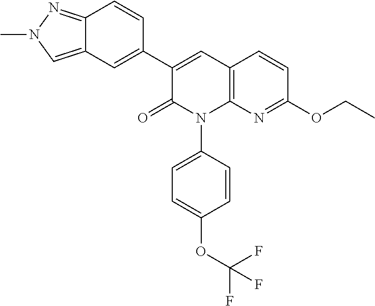 [SMILES: CCOc1ccc2cc(-c3ccc4nn(C)cc4c3)c(=O)n(-c3ccc(OC(F)(F)F)cc3)c2n1] |
| 154 | 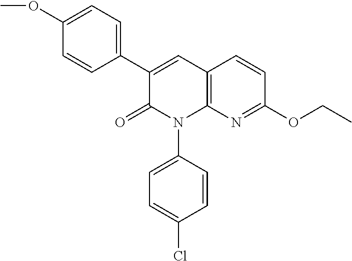 [SMILES: CCOc1ccc2cc(-c3ccc(OC)cc3)c(=O)n(-c3ccc(Cl)cc3)c2n1] |
| 155 | 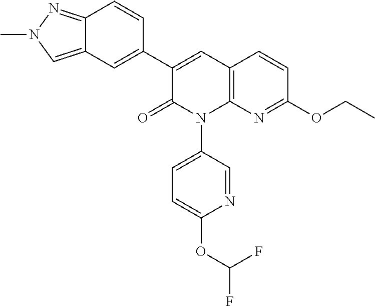 [SMILES: CCOc1ccc2cc(-c3ccc4nn(C)cc4c3)c(=O)n(-c3ccc(OC(F)F)nc3)c2n1] |
| 156 | 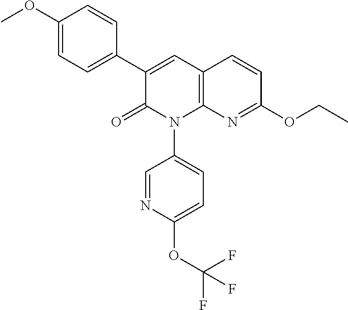 [SMILES: CCOc1ccc2cc(-c3ccc(OC)cc3)c(=O)n(-c3ccc(OC(F)(F)F)nc3)c2n1] |
| 157 | 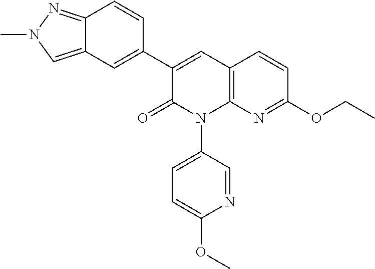 [SMILES: CCOc1ccc2cc(-c3ccc4nn(C)cc4c3)c(=O)n(-c3ccc(OC)nc3)c2n1] |
| 158 | 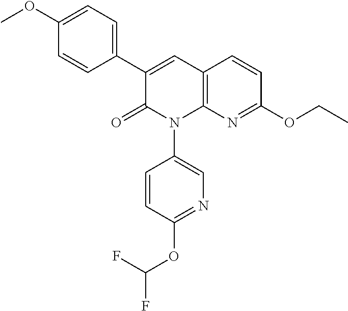 [SMILES: CCOc1ccc2cc(-c3ccc(OC)cc3)c(=O)n(-c3ccc(OC(F)F)nc3)c2n1] |
| 159 | 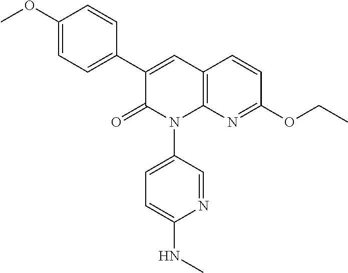 [SMILES: CCOc1ccc2cc(-c3ccc(OC)cc3)c(=O)n(-c3ccc(NC)nc3)c2n1] |
| 160 | 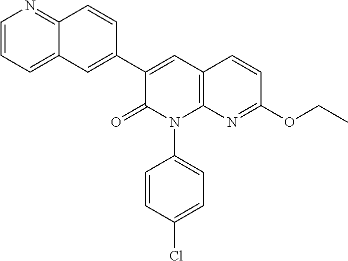 [SMILES: CCOc1ccc2cc(-c3ccc4ncccc4c3)c(=O)n(-c3ccc(Cl)cc3)c2n1] |
| 161 | 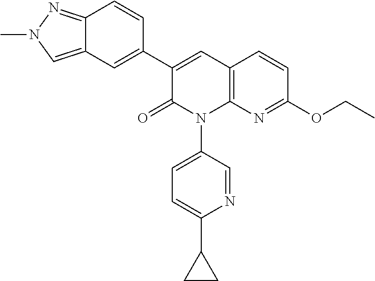 [SMILES: CCOc1ccc2cc(-c3ccc4nn(C)cc4c3)c(=O)n(-c3ccc(C4CC4)nc3)c2n1] |
| 162 | 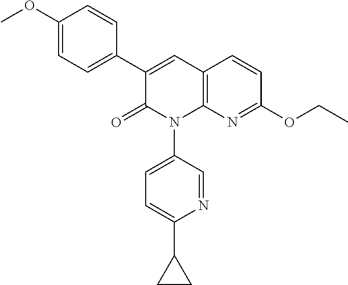 [SMILES: CCOc1ccc2cc(-c3ccc(OC)cc3)c(=O)n(-c3ccc(C4CC4)nc3)c2n1] |
| 163 | 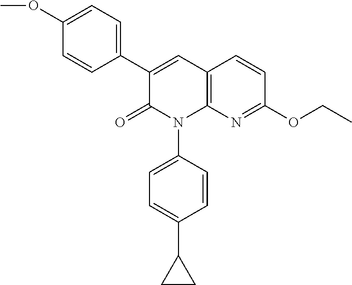 [SMILES: CCOc1ccc2cc(-c3ccc(OC)cc3)c(=O)n(-c3ccc(C4CC4)cc3)c2n1] |
| 164 | 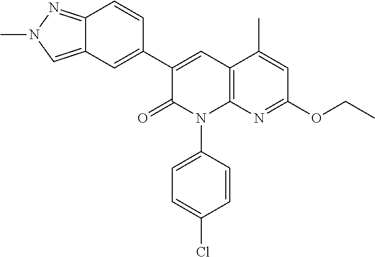 [SMILES: CCOc1cc(C)c2cc(-c3ccc4nn(C)cc4c3)c(=O)n(-c3ccc(Cl)cc3)c2n1] |
| 165 | 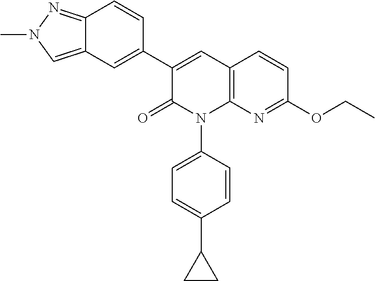 [SMILES: CCOc1ccc2cc(-c3ccc4nn(C)cc4c3)c(=O)n(-c3ccc(C4CC4)cc3)c2n1] |
| 166 | 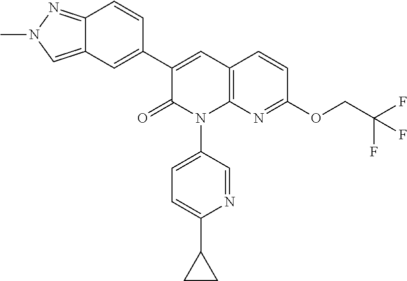 [SMILES: Cn1cc2cc(-c3cc4ccc(OCC(F)(F)F)nc4n(-c4ccc(C5CC5)nc4)c3=O)ccc2n1] |
| 167 | 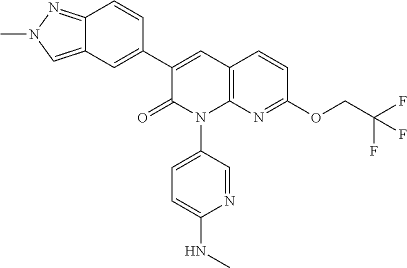 [SMILES: CNc1ccc(-n2c(=O)c(-c3ccc4nn(C)cc4c3)cc3ccc(OCC(F)(F)F)nc32)cn1] |
| 168 | 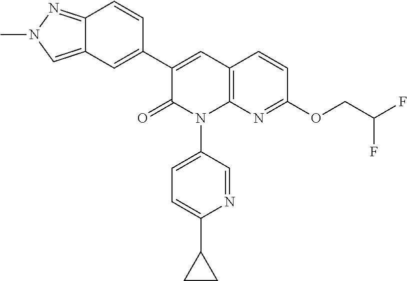 [SMILES: Cn1cc2cc(-c3cc4ccc(OCC(F)F)nc4n(-c4ccc(C5CC5)nc4)c3=O)ccc2n1] |
| 169 | 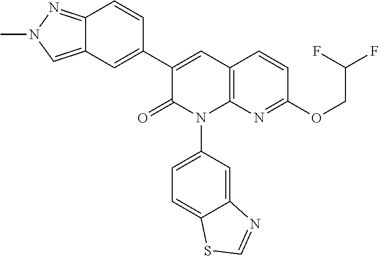 [SMILES: Cn1cc2cc(-c3cc4ccc(OCC(F)F)nc4n(-c4ccc5scnc5c4)c3=O)ccc2n1] |
| 170 | 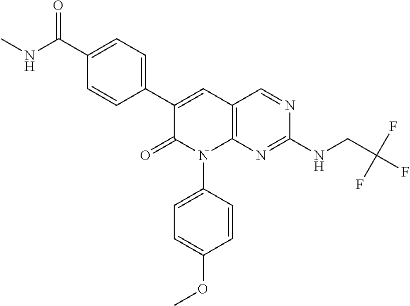 [SMILES: CNC(=O)c1ccc(-c2cc3cnc(NCC(F)(F)F)nc3n(-c3ccc(OC)cc3)c2=O)cc1] |
| 171 | 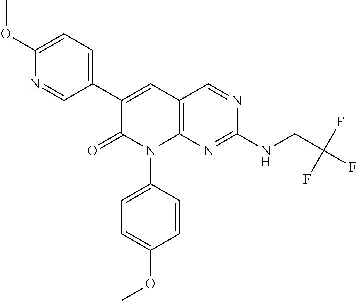 [SMILES: COc1ccc(-n2c(=O)c(-c3ccc(OC)nc3)cc3cnc(NCC(F)(F)F)nc32)cc1] |
| 172 | 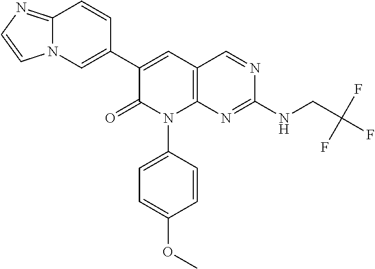 [SMILES: COc1ccc(-n2c(=O)c(-c3ccc4nccn4c3)cc3cnc(NCC(F)(F)F)nc32)cc1] |
| 173 | 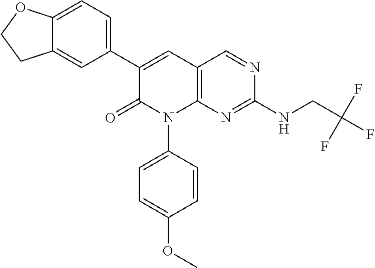 [SMILES: COc1ccc(-n2c(=O)c(-c3ccc4c(c3)CCO4)cc3cnc(NCC(F)(F)F)nc32)cc1] |
| 174 | 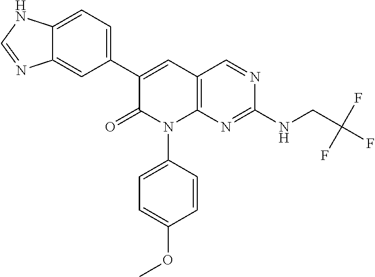 [SMILES: COc1ccc(-n2c(=O)c(-c3ccc4[nH]cnc4c3)cc3cnc(NCC(F)(F)F)nc32)cc1] |
| 175 | 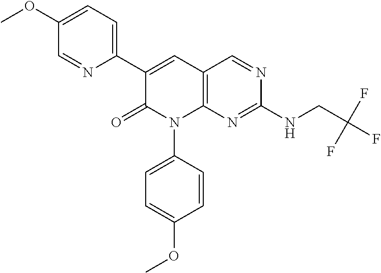 [SMILES: COc1ccc(-n2c(=O)c(-c3ccc(OC)cn3)cc3cnc(NCC(F)(F)F)nc32)cc1] |
| 176 | 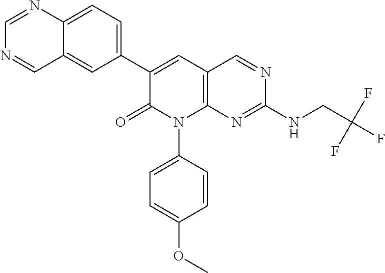 [SMILES: COc1ccc(-n2c(=O)c(-c3ccc4ncncc4c3)cc3cnc(NCC(F)(F)F)nc32)cc1] |
| 177 | 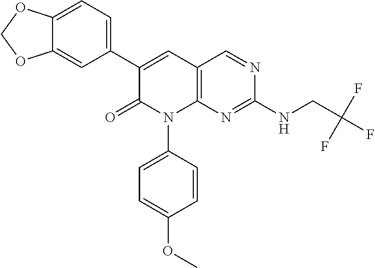 [SMILES: COc1ccc(-n2c(=O)c(-c3ccc4c(c3)OCO4)cc3cnc(NCC(F)(F)F)nc32)cc1] |
| 178 | 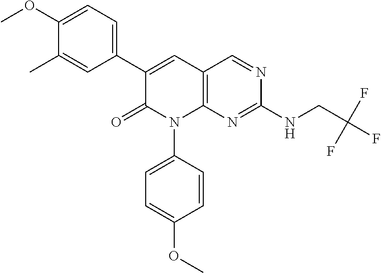 [SMILES: COc1ccc(-n2c(=O)c(-c3ccc(OC)c(C)c3)cc3cnc(NCC(F)(F)F)nc32)cc1] |
| 179 | 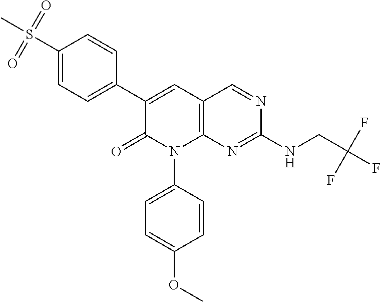 [SMILES: COc1ccc(-n2c(=O)c(-c3ccc(S(C)(=O)=O)cc3)cc3cnc(NCC(F)(F)F)nc32)cc1] |
| 180 | 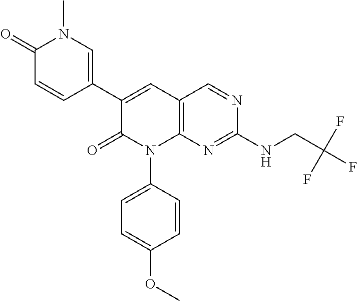 [SMILES: COc1ccc(-n2c(=O)c(-c3ccc(=O)n(C)c3)cc3cnc(NCC(F)(F)F)nc32)cc1] |
| 181 | 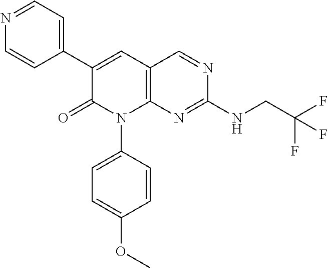 [SMILES: COc1ccc(-n2c(=O)c(-c3ccncc3)cc3cnc(NCC(F)(F)F)nc32)cc1] |
| 182 | 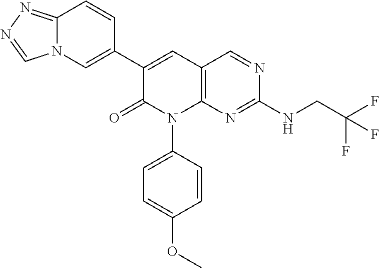 [SMILES: COc1ccc(-n2c(=O)c(-c3ccc4nncn4c3)cc3cnc(NCC(F)(F)F)nc32)cc1] |
| 183 | 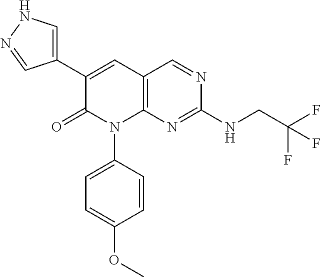 [SMILES: COc1ccc(-n2c(=O)c(-c3cn[nH]c3)cc3cnc(NCC(F)(F)F)nc32)cc1] |
| 184 | 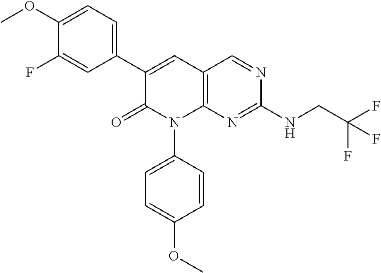 [SMILES: COc1ccc(-n2c(=O)c(-c3ccc(OC)c(F)c3)cc3cnc(NCC(F)(F)F)nc32)cc1] |
| 185 | 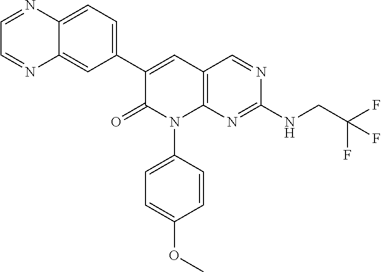 [SMILES: COc1ccc(-n2c(=O)c(-c3ccc4nccnc4c3)cc3cnc(NCC(F)(F)F)nc32)cc1] |
| 186 | 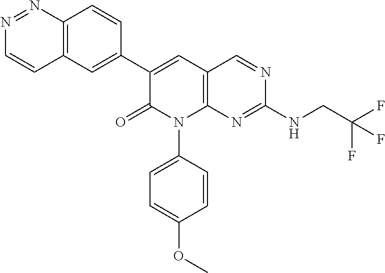 [SMILES: COc1ccc(-n2c(=O)c(-c3ccc4nnccc4c3)cc3cnc(NCC(F)(F)F)nc32)cc1] |
| 187 | 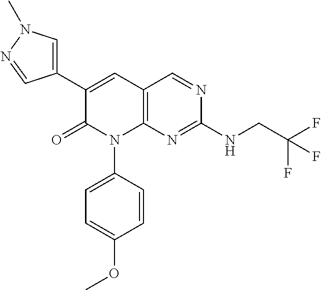 [SMILES: COc1ccc(-n2c(=O)c(-c3cnn(C)c3)cc3cnc(NCC(F)(F)F)nc32)cc1] |
| 188 | 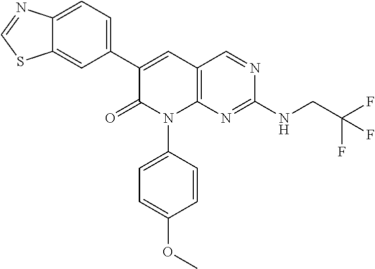 [SMILES: COc1ccc(-n2c(=O)c(-c3ccc4ncsc4c3)cc3cnc(NCC(F)(F)F)nc32)cc1] |
| 189 | 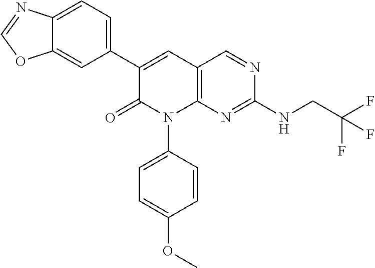 [SMILES: COc1ccc(-n2c(=O)c(-c3ccc4ncoc4c3)cc3cnc(NCC(F)(F)F)nc32)cc1] |
| 190 | 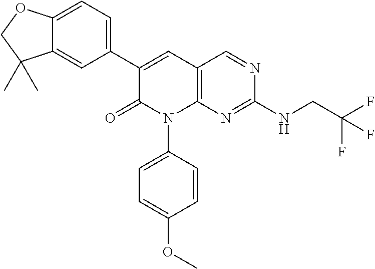 [SMILES: COc1ccc(-n2c(=O)c(-c3ccc4c(c3)C(C)(C)CO4)cc3cnc(NCC(F)(F)F)nc32)cc1] |
| 191 | 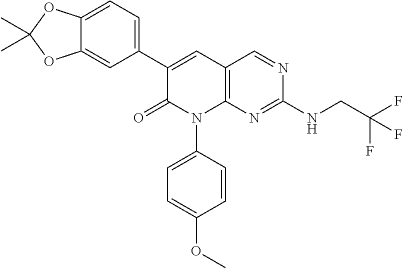 [SMILES: COc1ccc(-n2c(=O)c(-c3ccc4c(c3)OC(C)(C)O4)cc3cnc(NCC(F)(F)F)nc32)cc1] |
| 192 | 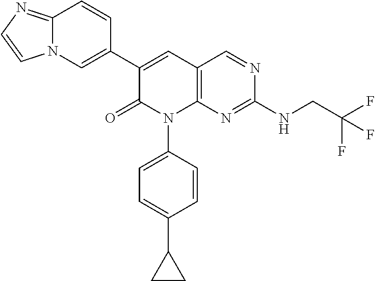 [SMILES: O=c1c(-c2ccc3nccn3c2)cc2cnc(NCC(F)(F)F)nc2n1-c1ccc(C2CC2)cc1] |
| 193 | 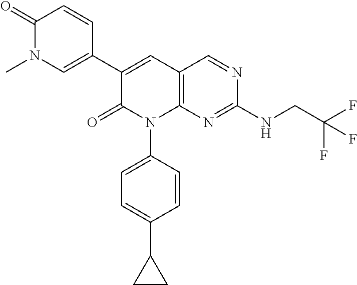 [SMILES: Cn1cc(-c2cc3cnc(NCC(F)(F)F)nc3n(-c3ccc(C4CC4)cc3)c2=O)ccc1=O] |
| 194 | 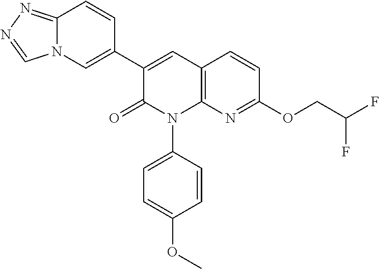 [SMILES: COc1ccc(-n2c(=O)c(-c3ccc4nncn4c3)cc3ccc(OCC(F)F)nc32)cc1] |
| 195 | 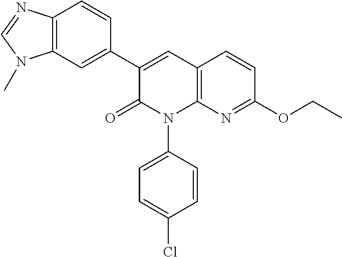 [SMILES: CCOc1ccc2cc(-c3ccc4ncn(C)c4c3)c(=O)n(-c3ccc(Cl)cc3)c2n1] |
| 196 | 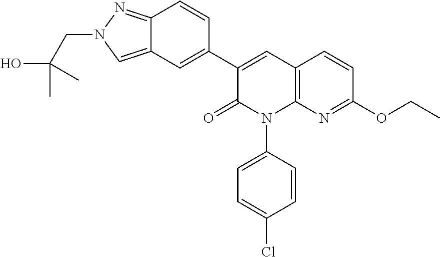 [SMILES: CCOc1ccc2cc(-c3ccc4nn(CC(C)(C)O)cc4c3)c(=O)n(-c3ccc(Cl)cc3)c2n1] |
| 197 | 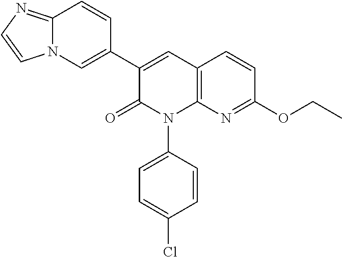 [SMILES: CCOc1ccc2cc(-c3ccc4nccn4c3)c(=O)n(-c3ccc(Cl)cc3)c2n1] |
| 198 | 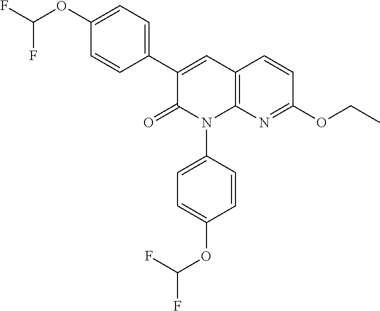 [SMILES: CCOc1ccc2cc(-c3ccc(OC(F)F)cc3)c(=O)n(-c3ccc(OC(F)F)cc3)c2n1] |
| 199 | 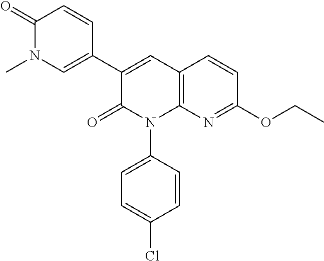 [SMILES: CCOc1ccc2cc(-c3ccc(=O)n(C)c3)c(=O)n(-c3ccc(Cl)cc3)c2n1] |
| 200 | 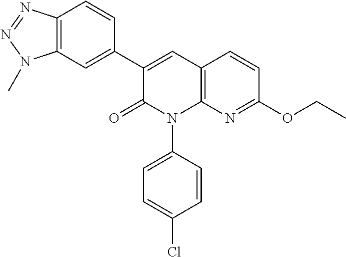 [SMILES: CCOc1ccc2cc(-c3ccc4nnn(C)c4c3)c(=O)n(-c3ccc(Cl)cc3)c2n1] |
| 201 | 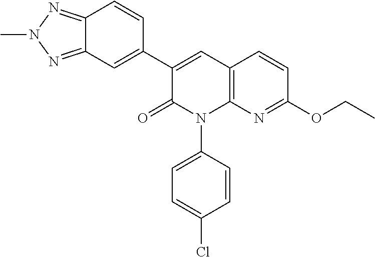 [SMILES: CCOc1ccc2cc(-c3ccc4nn(C)nc4c3)c(=O)n(-c3ccc(Cl)cc3)c2n1] |
| 202 | 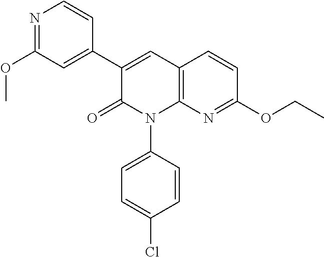 [SMILES: CCOc1ccc2cc(-c3ccnc(OC)c3)c(=O)n(-c3ccc(Cl)cc3)c2n1] |
| 203 | 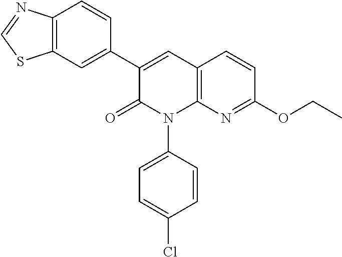 [SMILES: CCOc1ccc2cc(-c3ccc4ncsc4c3)c(=O)n(-c3ccc(Cl)cc3)c2n1] |
| 204 | 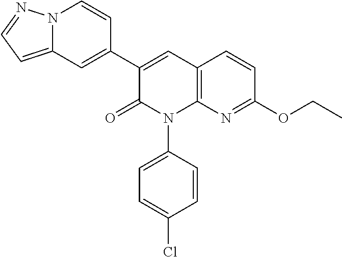 [SMILES: CCOc1ccc2cc(-c3ccn4nccc4c3)c(=O)n(-c3ccc(Cl)cc3)c2n1] |
| 205 | 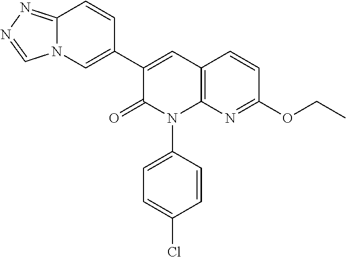 [SMILES: CCOc1ccc2cc(-c3ccc4nncn4c3)c(=O)n(-c3ccc(Cl)cc3)c2n1] |
| 206 | 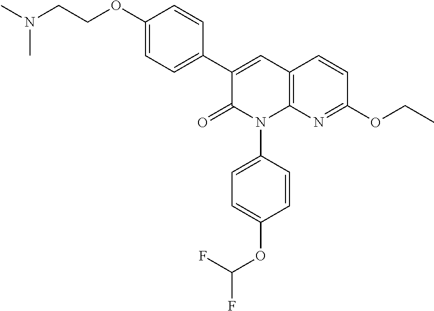 [SMILES: CCOc1ccc2cc(-c3ccc(OCCN(C)C)cc3)c(=O)n(-c3ccc(OC(F)F)cc3)c2n1] |
| 207 | 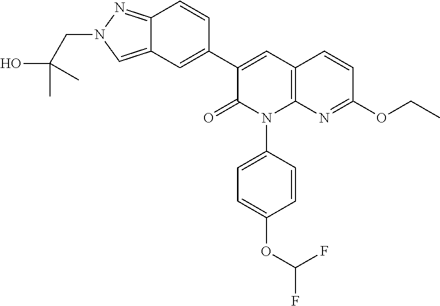 [SMILES: CCOc1ccc2cc(-c3ccc4nn(CC(C)(C)O)cc4c3)c(=O)n(-c3ccc(OC(F)F)cc3)c2n1] |
| 208 | 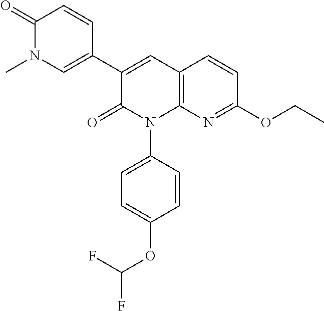 [SMILES: CCOc1ccc2cc(-c3ccc(=O)n(C)c3)c(=O)n(-c3ccc(OC(F)F)cc3)c2n1] |
| 209 | 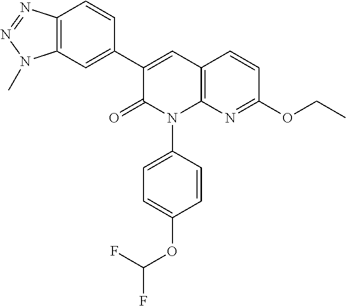 [SMILES: CCOc1ccc2cc(-c3ccc4nnn(C)c4c3)c(=O)n(-c3ccc(OC(F)F)cc3)c2n1] |
| 210 | 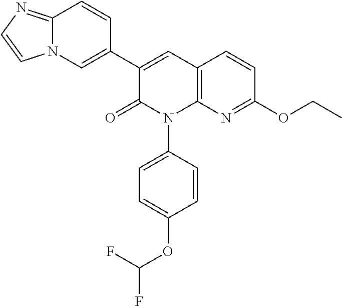 [SMILES: CCOc1ccc2cc(-c3ccc4nccn4c3)c(=O)n(-c3ccc(OC(F)F)cc3)c2n1] |
| 211 | 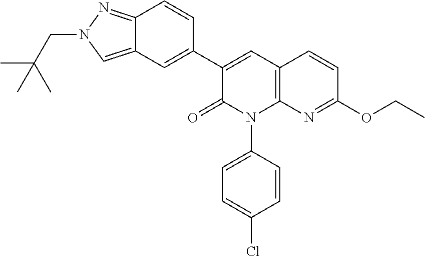 [SMILES: CCOc1ccc2cc(-c3ccc4nn(CC(C)(C)C)cc4c3)c(=O)n(-c3ccc(Cl)cc3)c2n1] |
| 212 | 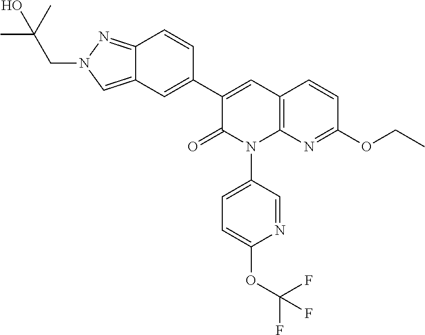 [SMILES: CCOc1ccc2cc(-c3ccc4nn(CC(C)(C)O)cc4c3)c(=O)n(-c3ccc(OC(F)(F)F)nc3)c2n1] |
| 213 | 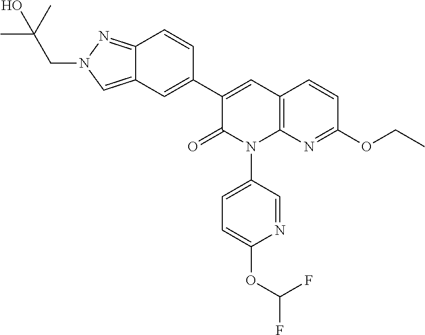 [SMILES: CCOc1ccc2cc(-c3ccc4nn(CC(C)(C)O)cc4c3)c(=O)n(-c3ccc(OC(F)F)nc3)c2n1] |
| 214 | 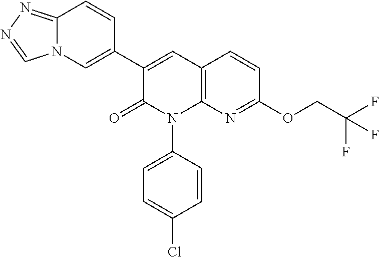 [SMILES: O=c1c(-c2ccc3nncn3c2)cc2ccc(OCC(F)(F)F)nc2n1-c1ccc(Cl)cc1] |
| 215 | 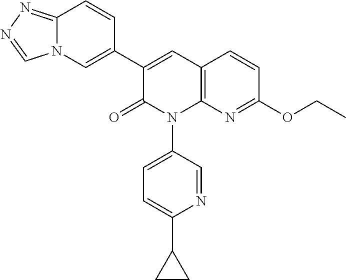 [SMILES: CCOc1ccc2cc(-c3ccc4nncn4c3)c(=O)n(-c3ccc(C4CC4)nc3)c2n1] |
| 216 | 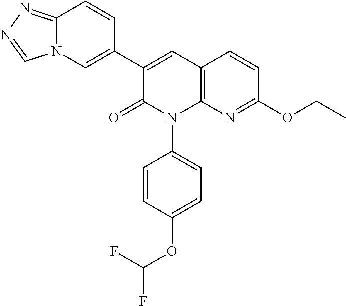 [SMILES: CCOc1ccc2cc(-c3ccc4nncn4c3)c(=O)n(-c3ccc(OC(F)F)cc3)c2n1] |
| 217 | 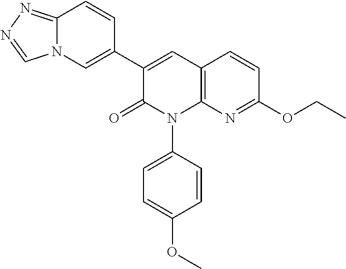 [SMILES: CCOc1ccc2cc(-c3ccc4nncn4c3)c(=O)n(-c3ccc(OC)cc3)c2n1] |
| 218 | 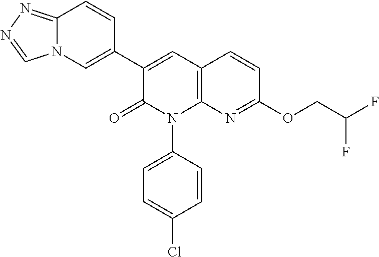 [SMILES: O=c1c(-c2ccc3nncn3c2)cc2ccc(OCC(F)F)nc2n1-c1ccc(Cl)cc1] |
| 219 | 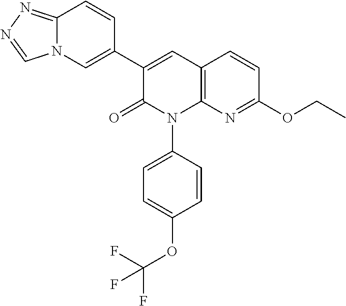 [SMILES: CCOc1ccc2cc(-c3ccc4nncn4c3)c(=O)n(-c3ccc(OC(F)(F)F)cc3)c2n1] |
| 220 | 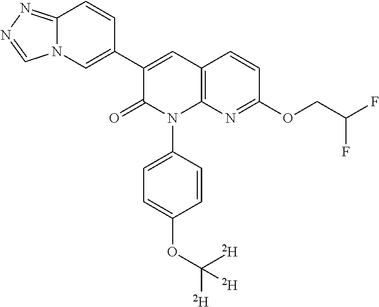 [SMILES: [2H]C([2H])([2H])Oc1ccc(-n2c(=O)c(-c3ccc4nncn4c3)cc3ccc(OCC(F)F)nc32)cc1] |
| 221 | 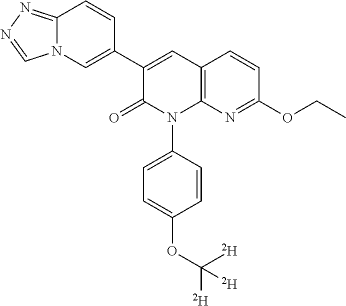 [SMILES: [2H]C([2H])([2H])Oc1ccc(-n2c(=O)c(-c3ccc4nncn4c3)cc3ccc(OCC)nc32)cc1] |
| 232 | 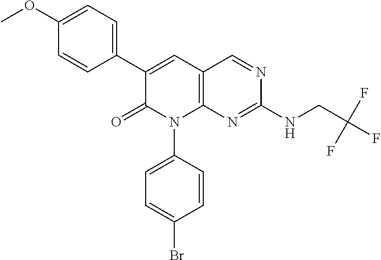 [SMILES: COc1ccc(-c2cc3cnc(NCC(F)(F)F)nc3n(-c3ccc(Br)cc3)c2=O)cc1] |
| 233 | 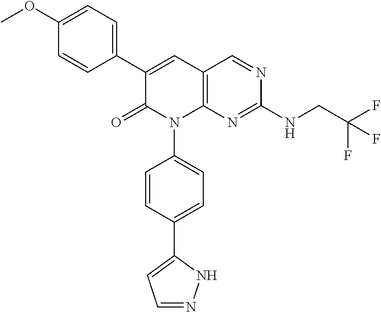 [SMILES: COc1ccc(-c2cc3cnc(NCC(F)(F)F)nc3n(-c3ccc(-c4ccn[nH]4)cc3)c2=O)cc1] |
| 234 | 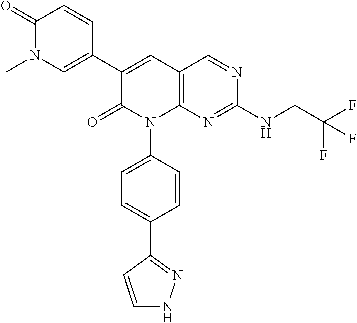 [SMILES: Cn1cc(-c2cc3cnc(NCC(F)(F)F)nc3n(-c3ccc(-c4cc[nH]n4)cc3)c2=O)ccc1=O] |
| 235 | 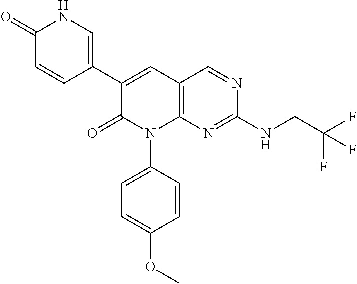 [SMILES: COc1ccc(-n2c(=O)c(-c3ccc(=O)[nH]c3)cc3cnc(NCC(F)(F)F)nc32)cc1] |
| 236 | 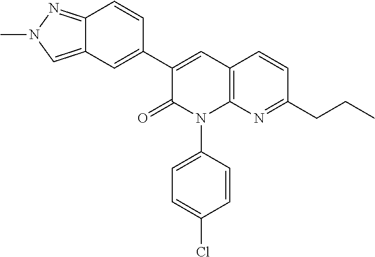 [SMILES: CCCc1ccc2cc(-c3ccc4nn(C)cc4c3)c(=O)n(-c3ccc(Cl)cc3)c2n1] |

TABLE 2
Examples of Formula II Compounds

| Ex | |
|---|---|
| No | Structure |
| 222 | 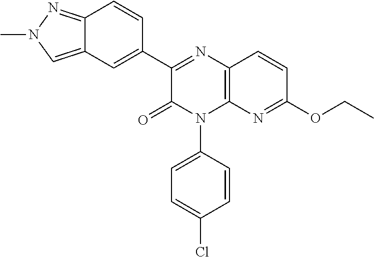 [SMILES: CCOc1ccc2nc(-c3ccc4nn(C)cc4c3)c(=O)n(-c3ccc(Cl)cc3)c2n1] |
| 223 | 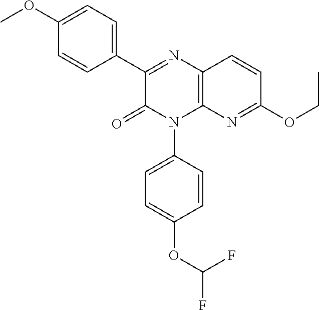 [SMILES: CCOc1ccc2nc(-c3ccc(OC)cc3)c(=O)n(-c3ccc(OC(F)F)cc3)c2n1] |
| 224 | 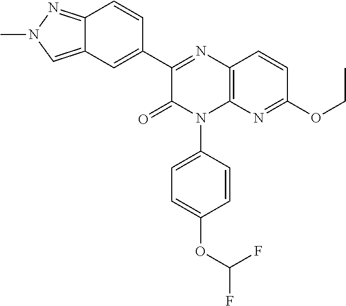 [SMILES: CCOc1ccc2nc(-c3ccc4nn(C)cc4c3)c(=O)n(-c3ccc(OC(F)F)cc3)c2n1] |
| 225 | 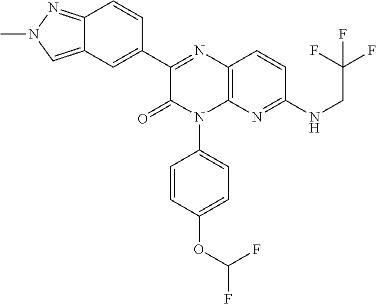 [SMILES: Cn1cc2cc(-c3nc4ccc(NCC(F)(F)F)nc4n(-c4ccc(OC(F)F)cc4)c3=O)ccc2n1] |
| 226 | 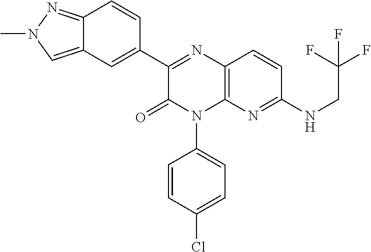 [SMILES: Cn1cc2cc(-c3nc4ccc(NCC(F)(F)F)nc4n(-c4ccc(Cl)cc4)c3=O)ccc2n1] |
| 227 | 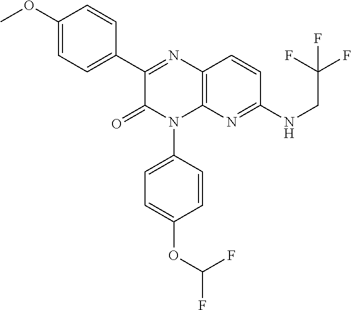 [SMILES: COc1ccc(-c2nc3ccc(NCC(F)(F)F)nc3n(-c3ccc(OC(F)F)cc3)c2=O)cc1] |
| 228 | 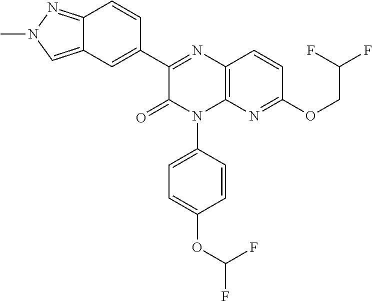 [SMILES: Cn1cc2cc(-c3nc4ccc(OCC(F)F)nc4n(-c4ccc(OC(F)F)cc4)c3=O)ccc2n1] |
| 229 | 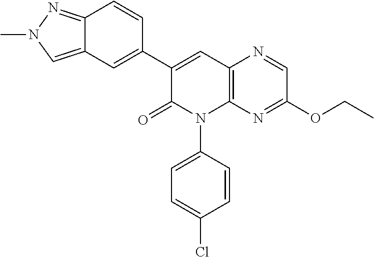 [SMILES: CCOc1cnc2cc(-c3ccc4nn(C)cc4c3)c(=O)n(-c3ccc(Cl)cc3)c2n1] |
| 230 | 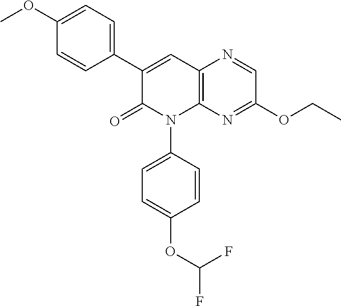 [SMILES: CCOc1cnc2cc(-c3ccc(OC)cc3)c(=O)n(-c3ccc(OC(F)F)cc3)c2n1] |
| 231 | 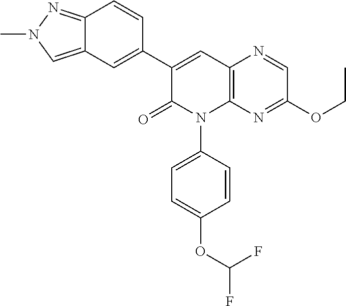 [SMILES: CCOc1cnc2cc(-c3ccc4nn(C)cc4c3)c(=O)n(-c3ccc(OC(F)F)cc3)c2n1] |
| 237 | 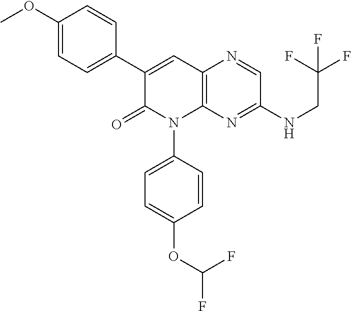 [SMILES: COc1ccc(-c2cc3ncc(NCC(F)(F)F)nc3n(-c3ccc(OC(F)F)cc3)c2=O)cc1] |

In various embodiments, the disclosure provides Formula I compounds and their pharmaceutically acceptable salts, tautomers, and/or isotopologues thereof, as set forth in the following table:

TABLE 3

| Ex | |
|---|---|
| No | Structure |
| 124 | 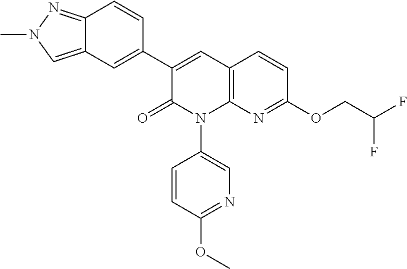 [SMILES: COc1ccc(-n2c(=O)c(-c3ccc4nn(C)cc4c3)cc3ccc(OCC(F)F)nc32)cn1] |
| 166 | 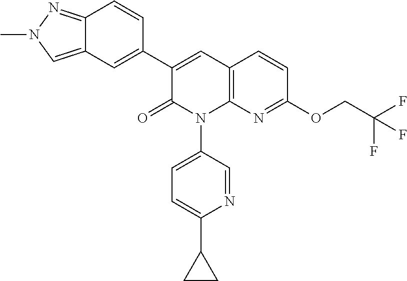 [SMILES: Cn1cc2cc(-c3cc4ccc(OCC(F)(F)F)nc4n(-c4ccc(C5CC5)nc4)c3=O)ccc2n1] |
| 122 | 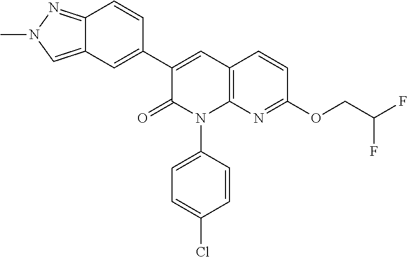 [SMILES: Cn1cc2cc(-c3cc4ccc(OCC(F)F)nc4n(-c4ccc(Cl)cc4)c3=O)ccc2n1] |
| 161 | 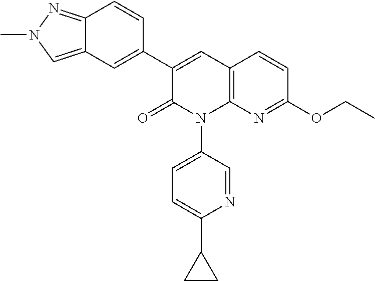 [SMILES: CCOc1ccc2cc(-c3ccc4nn(C)cc4c3)c(=O)n(-c3ccc(C4CC4)nc3)c2n1] |
| 165 | 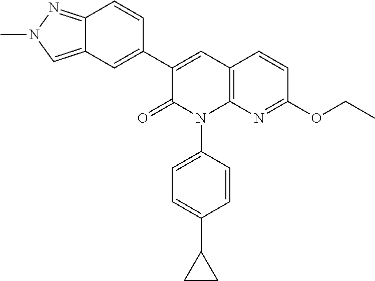 [SMILES: CCOc1ccc2cc(-c3ccc4nn(C)cc4c3)c(=O)n(-c3ccc(C4CC4)cc3)c2n1] |
| 138 | 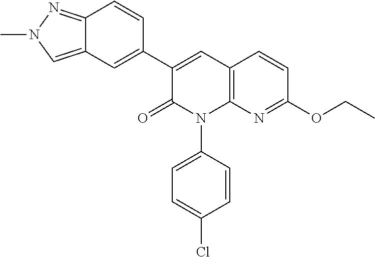 [SMILES: CCOc1ccc2cc(-c3ccc4nn(C)cc4c3)c(=O)n(-c3ccc(Cl)cc3)c2n1] |
| 196 | 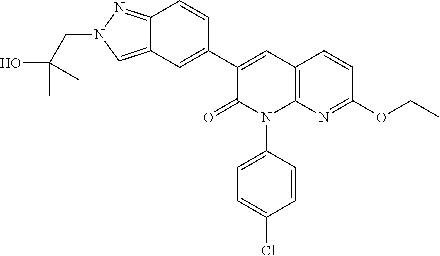 [SMILES: CCOc1ccc2cc(-c3ccc4nn(CC(C)(C)O)cc4c3)c(=O)n(-c3ccc(Cl)cc3)c2n1] |
| 149 | 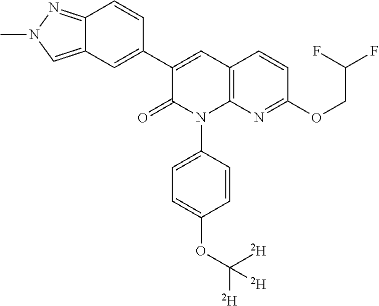 [SMILES: [2H]C([2H])([2H])Oc1ccc(-n2c(=O)c(-c3ccc4nn(C)cc4c3)cc3ccc(OCC(F)F)nc32)cc1] |
| 322 | 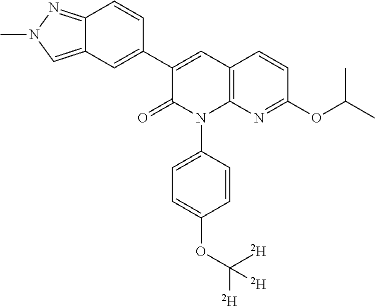 [SMILES: [2H]C([2H])([2H])Oc1ccc(-n2c(=O)c(-c3ccc4nn(C)cc4c3)cc3ccc(OC(C)C)nc32)cc1] |
| 303 | 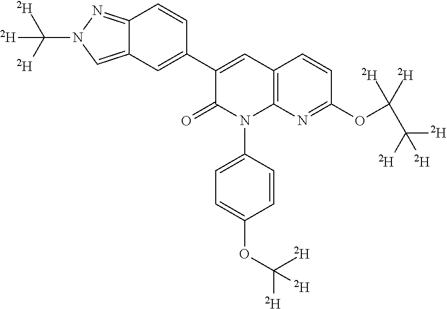 [SMILES: [2H]C([2H])([2H])Oc1ccc(-n2c(=O)c(-c3ccc4nn(C([2H])([2H])[2H])cc4c3)cc3ccc(OC([2H])([2H])C([2H])([2H])[2H])nc32)cc1] |
| 313 | 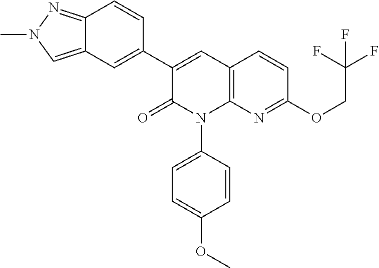 [SMILES: COc1ccc(-n2c(=O)c(-c3ccc4nn(C)cc4c3)cc3ccc(OCC(F)(F)F)nc32)cc1] |
| 142 | 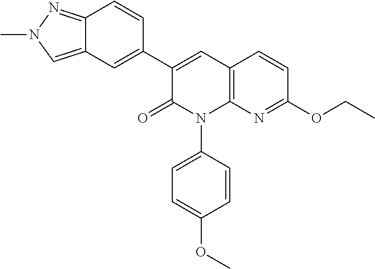 [SMILES: CCOc1ccc2cc(-c3ccc4nn(C)cc4c3)c(=O)n(-c3ccc(OC)cc3)c2n1] |
| 302 | 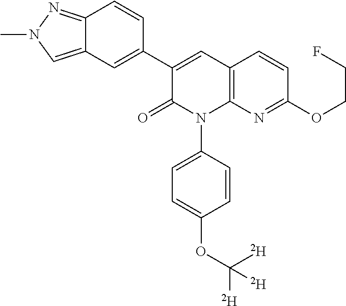 [SMILES: [2H]C([2H])([2H])Oc1ccc(-n2c(=O)c(-c3ccc4nn(C)cc4c3)cc3ccc(OCCF)nc32)cc1] |
| 195 | 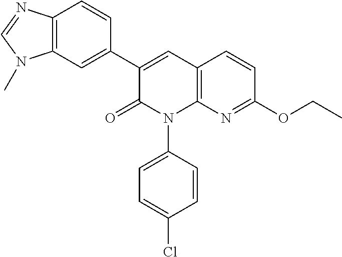 [SMILES: CCOc1ccc2cc(-c3ccc4ncn(C)c4c3)c(=O)n(-c3ccc(Cl)cc3)c2n1] |
| 314 | 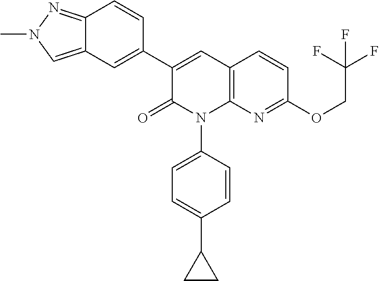 [SMILES: Cn1cc2cc(-c3cc4ccc(OCC(F)(F)F)nc4n(-c4ccc(C5CC5)cc4)c3=O)ccc2n1] |
| 210 | 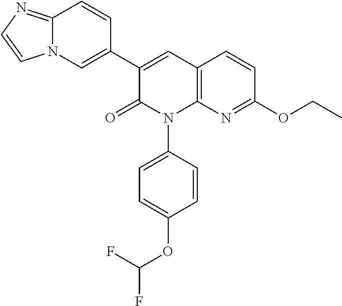 [SMILES: CCOc1ccc2cc(-c3ccc4nccn4c3)c(=O)n(-c3ccc(OC(F)F)cc3)c2n1] |
| 219 | 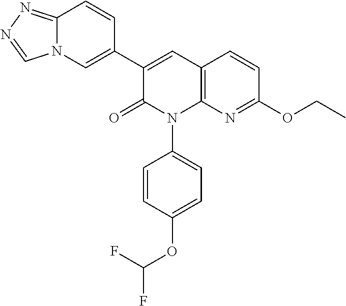 [SMILES: CCOc1ccc2cc(-c3ccc4nncn4c3)c(=O)n(-c3ccc(OC(F)F)cc3)c2n1] |
| 305 | 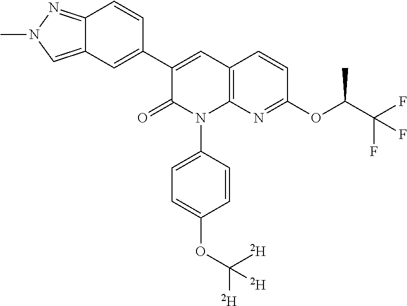 [SMILES: [2H]C([2H])([2H])Oc1ccc(-n2c(=O)c(-c3ccc4nn(C)cc4c3)cc3ccc(O[C@@H](C)C(F)(F)F)nc32)cc1] |
| 168 | 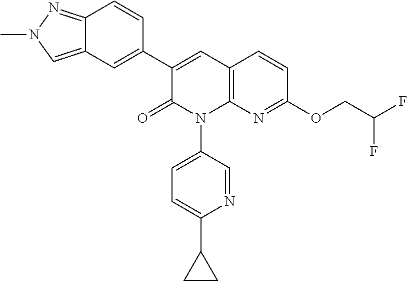 [SMILES: Cn1cc2cc(-c3cc4ccc(OCC(F)F)nc4n(-c4ccc(C5CC5)nc4)c3=O)ccc2n1] |
| 157 | 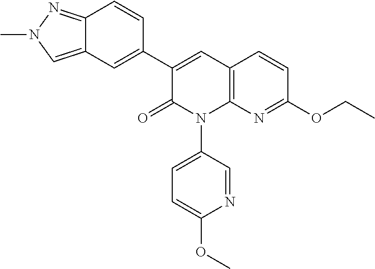 [SMILES: CCOc1ccc2cc(-c3ccc4nn(C)cc4c3)c(=O)n(-c3ccc(OC)nc3)c2n1] |
| 144 | 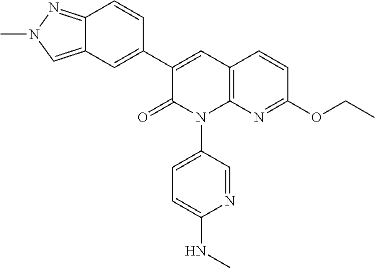 [SMILES: CCOc1ccc2cc(-c3ccc4nn(C)cc4c3)c(=O)n(-c3ccc(NC)nc3)c2n1] |
| 221 | 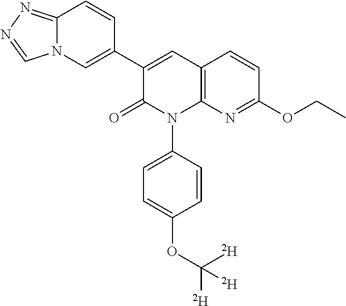 [SMILES: [2H]C([2H])([2H])Oc1ccc(-n2c(=O)c(-c3ccc4nncn4c3)cc3ccc(OCC)nc32)cc1] |
| 123 | 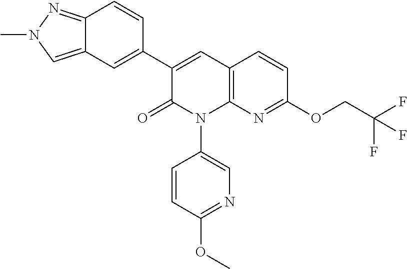 [SMILES: COc1ccc(-n2c(=O)c(-c3ccc4nn(C)cc4c3)cc3ccc(OCC(F)(F)F)nc32)cn1] |
| 205 | 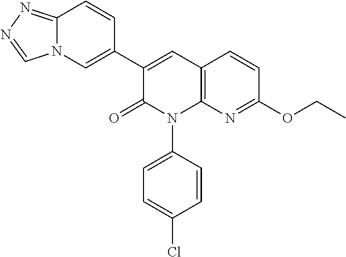 [SMILES: CCOc1ccc2cc(-c3ccc4nncn4c3)c(=O)n(-c3ccc(Cl)cc3)c2n1] |
| 148 | 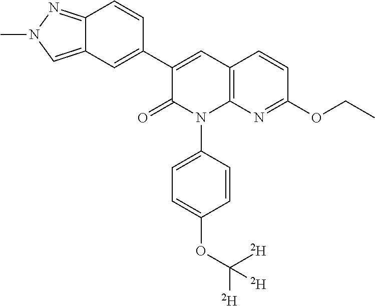 [SMILES: [2H]C([2H])([2H])Oc1ccc(-n2c(=O)c(-c3ccc4nn(C)cc4c3)cc3ccc(OCC)nc32)cc1] |
| 220 | 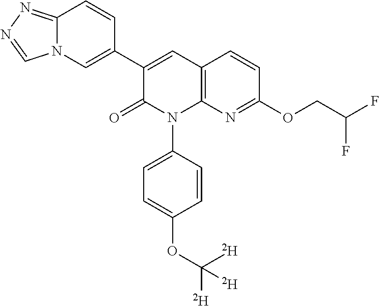 [SMILES: [2H]C([2H])([2H])Oc1ccc(-n2c(=O)c(-c3ccc4nncn4c3)cc3ccc(OCC(F)F)nc32)cc1] |
| 301 | 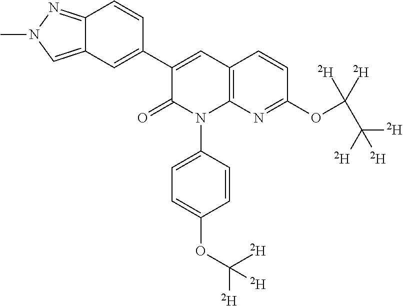 [SMILES: [2H]C([2H])([2H])Oc1ccc(-n2c(=O)c(-c3ccc4nn(C)cc4c3)cc3ccc(OC([2H])([2H])C([2H])([2H])[2H])nc32)cc1] |
| 318 | 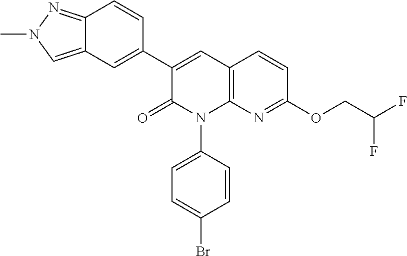 [SMILES: Cn1cc2cc(-c3cc4ccc(OCC(F)F)nc4n(-c4ccc(Br)cc4)c3=O)ccc2n1] |
| 194 | 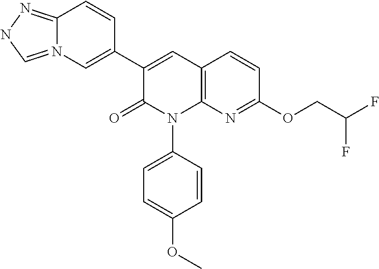 |
| 316 | 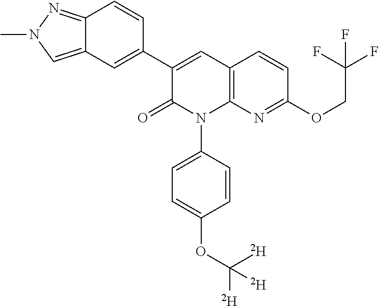 [SMILES: [2H]C([2H])([2H])Oc1ccc(-n2c(=O)c(-c3ccc4nn(C)cc4c3)cc3ccc(OCC(F)(F)F)nc32)cc1] |
| 319 | 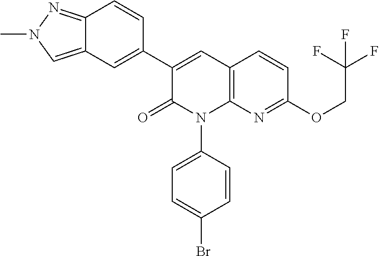 [SMILES: Cn1cc2cc(-c3cc4ccc(OCC(F)(F)F)nc4n(-c4ccc(Br)cc4)c3=O)ccc2n1] |
| 217 | 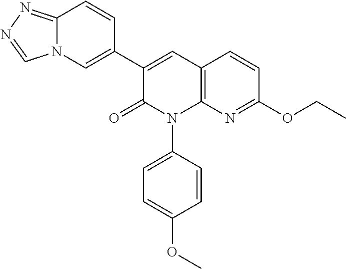 [SMILES: CCOc1ccc2cc(-c3ccc4nncn4c3)c(=O)n(-c3ccc(OC)cc3)c2n1] |
| 204 | 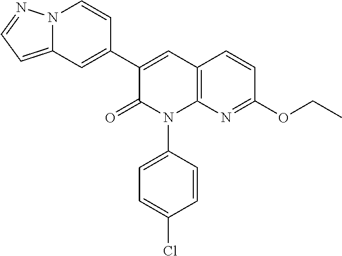 [SMILES: CCOc1ccc2cc(-c3ccn4nccc4c3)c(=O)n(-c3ccc(Cl)cc3)c2n1] |
| 200 | 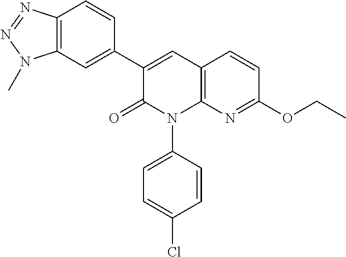 [SMILES: CCOc1ccc2cc(-c3ccc4nnn(C)c4c3)c(=O)n(-c3ccc(Cl)cc3)c2n1] |
| 218 | 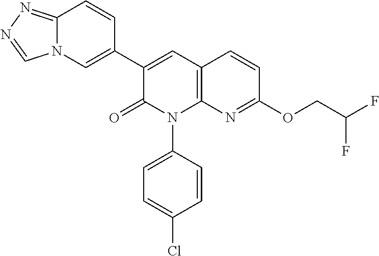 [SMILES: O=c1c(-c2ccc3nncn3c2)cc2ccc(OCC(F)F)nc2n1-c1ccc(Cl)cc1] |
| 306 | 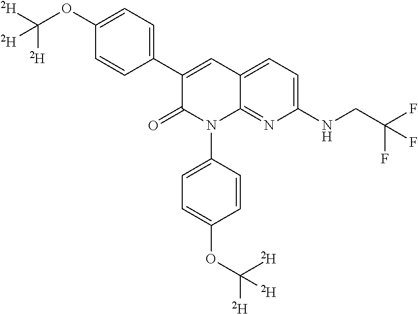 [SMILES: [2H]C([2H])([2H])Oc1ccc(-c2cc3ccc(NCC(F)(F)F)nc3n(-c3ccc(OC([2H])([2H])[2H])cc3)c2=O)cc1] |

In further embodiments, the disclosure provides Formula I compounds and their pharmaceutically acceptable salts, tautomers, and/or isotopologues thereof, as set forth in the following table:

TABLE 4

| Ex | |
|---|---|
| No | Structure |
| 304 | 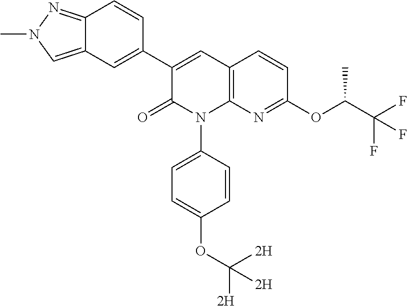 |
| 305 | 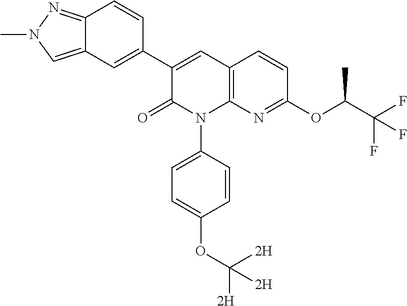 |
| 307 | 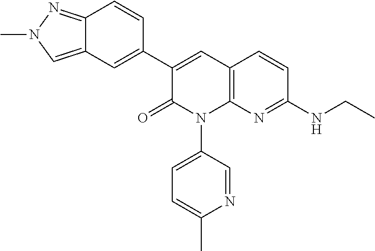 [SMILES: CCNc1ccc2cc(-c3ccc4nn(C)cc4c3)c(=O)n(-c3ccc(C)nc3)c2n1] |
| 308 | 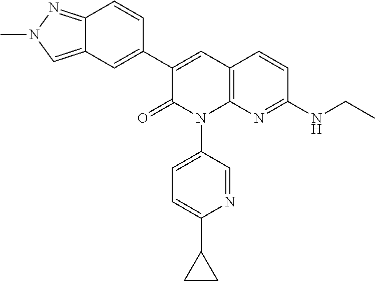 [SMILES: CCNc1ccc2cc(-c3ccc4nn(C)cc4c3)c(=O)n(-c3ccc(C4CC4)nc3)c2n1] |
| 309 | 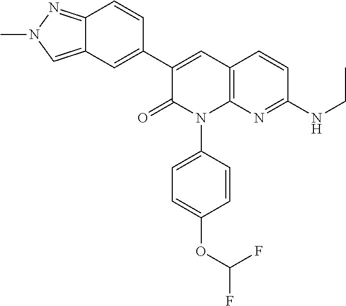 [SMILES: CCNc1ccc2cc(-c3ccc4nn(C)cc4c3)c(=O)n(-c3ccc(OC(F)F)cc3)c2n1] |
| 310 | 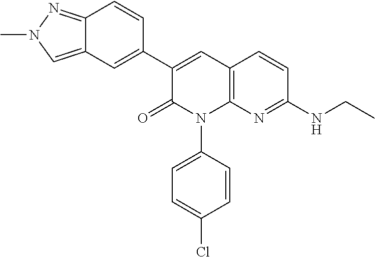 [SMILES: CCNc1ccc2cc(-c3ccc4nn(C)cc4c3)c(=O)n(-c3ccc(Cl)cc3)c2n1] |
| 311 | 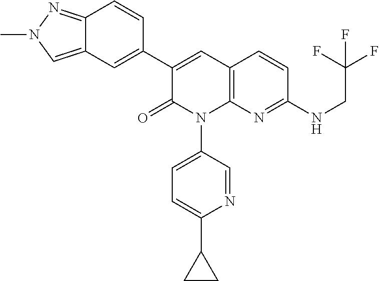 [SMILES: Cn1cc2cc(-c3cc4ccc(NCC(F)(F)F)nc4n(-c4ccc(C5CC5)nc4)c3=O)ccc2n1] |
| 312 | 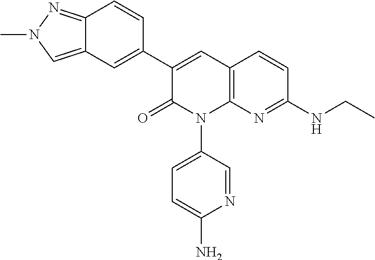 [SMILES: CCNc1ccc2cc(-c3ccc4nn(C)cc4c3)c(=O)n(-c3ccc(N)nc3)c2n1] |
| 315 | 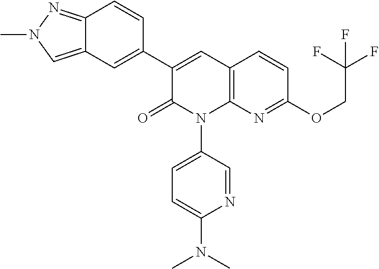 [SMILES: CN(C)c1ccc(-n2c(=O)c(-c3ccc4nn(C)cc4c3)cc3ccc(OCC(F)(F)F)nc32)cn1] |
| 317 | 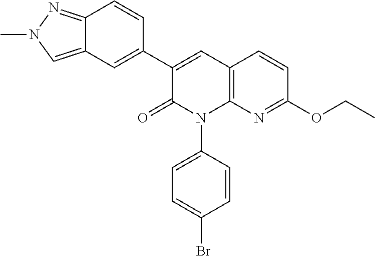 [SMILES: CCOc1ccc2cc(-c3ccc4nn(C)cc4c3)c(=O)n(-c3ccc(Br)cc3)c2n1] |
| 320 | 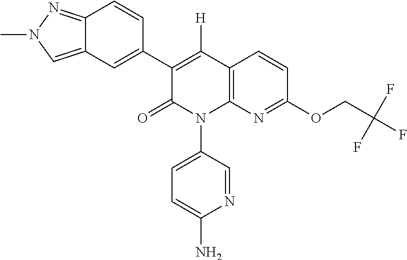 [SMILES: Cn1cc2cc(-c3cc4ccc(OCC(F)(F)F)nc4n(-c4ccc(N)nc4)c3=O)ccc2n1] |
| 321 | 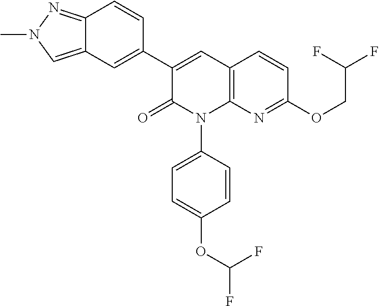 [SMILES: Cn1cc2cc(-c3cc4ccc(OCC(F)F)nc4n(-c4ccc(OC(F)F)cc4)c3=O)ccc2n1] |
| 323 | 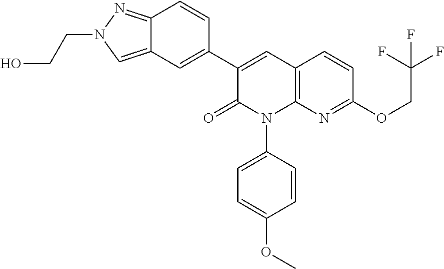 [SMILES: COc1ccc(-n2c(=O)c(-c3ccc4nn(CCO)cc4c3)cc3ccc(OCC(F)(F)F)nc32)cc1] |
| 353 | 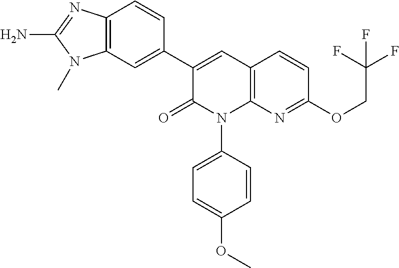 [SMILES: COc1ccc(-n2c(=O)c(-c3ccc4nc(N)n(C)c4c3)cc3ccc(OCC(F)(F)F)nc32)cc1] |

In other embodiments, the disclosure provides Formula II compounds and their pharmaceutically acceptable salts, tautomers, and/or isotopologues thereof, as set forth in the following table:

TABLE 5

| | |
|---|---|
| 326 | 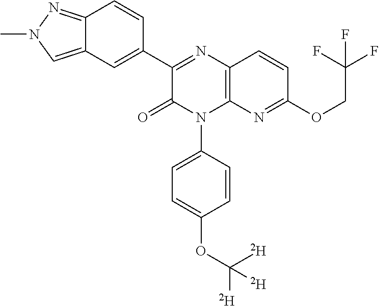 [SMILES: [2H]C([2H])([2H])Oc1ccc(-n2c(=O)c(-c3ccc4nn(C)cc4c3)nc3ccc(OCC(F)(F)F)nc32)cc1] |
| 344 | 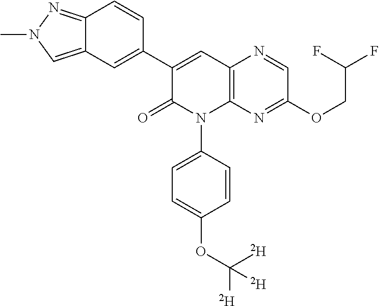 [SMILES: [2H]C([2H])([2H])Oc1ccc(-n2c(=O)c(-c3ccc4nn(C)cc4c3)cc3ncc(OCC(F)F)nc32)cc1] |
| 329 | 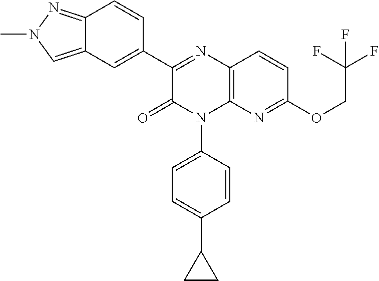 [SMILES: Cn1cc2cc(-c3nc4ccc(OCC(F)(F)F)nc4n(-c4ccc(C5CC5)cc4)c3=O)ccc2n1] |
| 330 | 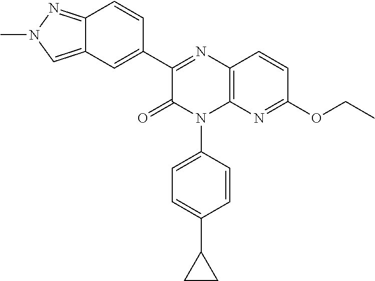 [SMILES: CCOc1ccc2nc(-c3ccc4nn(C)cc4c3)c(=O)n(-c3ccc(C4CC4)cc3)c2n1] |
| 343 | 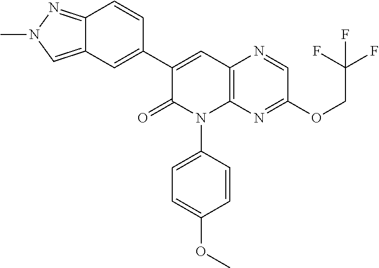 [SMILES: COc1ccc(-n2c(=O)c(-c3ccc4nn(C)cc4c3)cc3ncc(OCC(F)(F)F)nc32)cc1] |
| 342 | 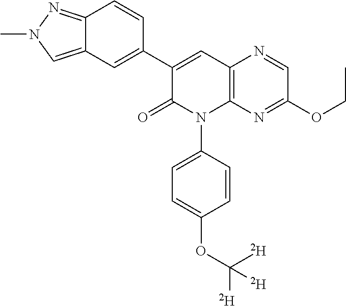 [SMILES: [2H]C([2H])([2H])Oc1ccc(-n2c(=O)c(-c3ccc4nn(C)cc4c3)cc3ncc(OCC)nc32)cc1] |
| 331 | 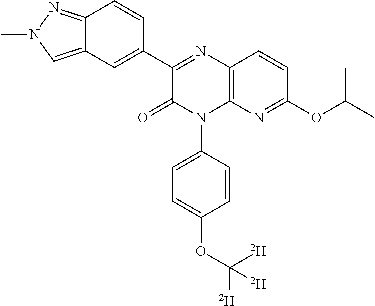 [SMILES: [2H]C([2H])([2H])Oc1ccc(-n2c(=O)c(-c3ccc4nn(C)cc4c3)nc3ccc(OC(C)C)nc32)cc1] |
| 328 | 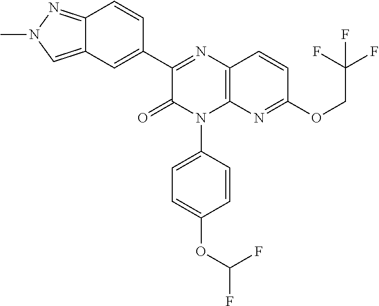 [SMILES: Cn1cc2cc(-c3nc4ccc(OCC(F)(F)F)nc4n(-c4ccc(OC(F)F)cc4)c3=O)ccc2n1] |
| 334 | 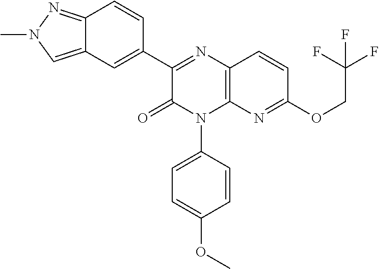 [SMILES: COc1ccc(-n2c(=O)c(-c3ccc4nn(C)cc4c3)nc3ccc(OCC(F)(F)F)nc32)cc1] |
| 345 | 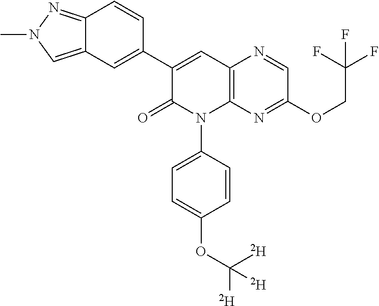 [SMILES: [2H]C([2H])([2H])Oc1ccc(-n2c(=O)c(-c3ccc4nn(C)cc4c3)cc3ncc(OCC(F)(F)F)nc32)cc1] |

In yet further embodiments, the disclosure provides Formula II compounds and their pharmaceutically acceptable salts, tautomers, and/or isotopologues thereof, as set forth in the following table:

TABLE 6

| | |
|---|---|
| 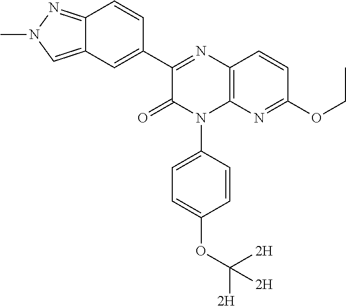 | 324 |
| 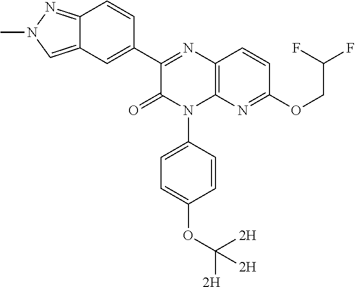 | 325 |
| 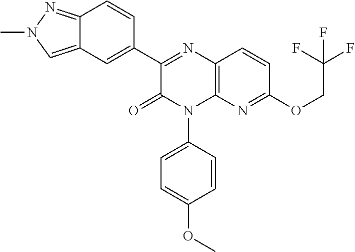 [SMILES: COc1ccc(-n2c(=O)c(-c3ccc4nn(C)cc4c3)nc3ccc(OCC(F)(F)F)nc32)cc1] | 327 |
| 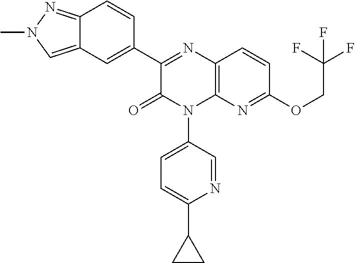 [SMILES: Cn1cc2cc(-c3nc4ccc(OCC(F)(F)F)nc4n(-c4ccc(C5CC5)nc4)c3=O)ccc2n1] | 332 |
| 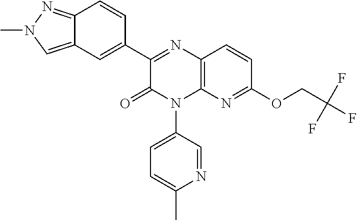 [SMILES: Cc1ccc(-n2c(=O)c(-c3ccc4nn(C)cc4c3)nc3ccc(OCC(F)(F)F)nc32)cn1] | 333 |
| 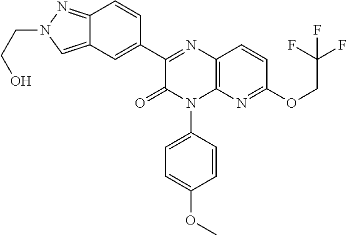 [SMILES: COc1ccc(-n2c(=O)c(-c3ccc4nn(CCO)cc4c3)nc3ccc(OCC(F)(F)F)nc32)cc1] | 335 |
| 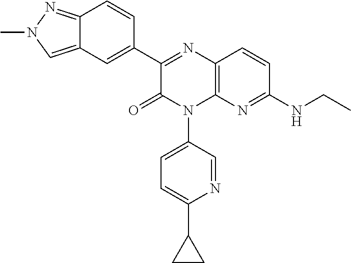 [SMILES: CCNc1ccc2nc(-c3ccc4nn(C)cc4c3)c(=O)n(-c3ccc(C4CC4)nc3)c2n1] | 336 |
| 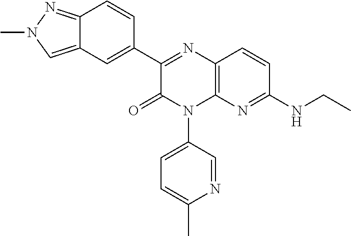 [SMILES: CCNc1ccc2nc(-c3ccc4nn(C)cc4c3)c(=O)n(-c3ccc(C)nc3)c2n1] | 337 |
| 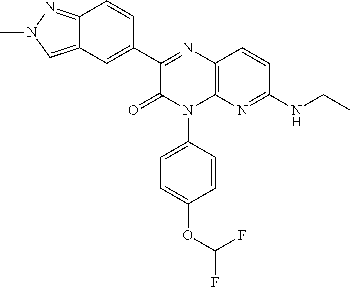 [SMILES: CCNc1ccc2nc(-c3ccc4nn(C)cc4c3)c(=O)n(-c3ccc(OC(F)F)cc3)c2n1] | 338 |
| 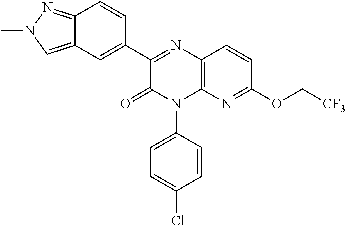 [SMILES: Cn1cc2cc(-c3nc4ccc(OCC(F)(F)F)nc4n(-c4ccc(Cl)cc4)c3=O)ccc2n1] | 339 |
| 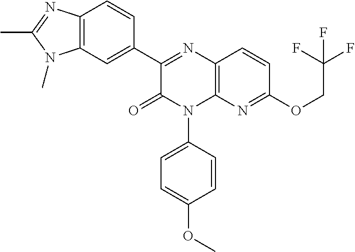 [SMILES: COc1ccc(-n2c(=O)c(-c3ccc4nc(C)n(C)c4c3)nc3ccc(OCC(F)(F)F)nc32)cc1] | 340 |
| 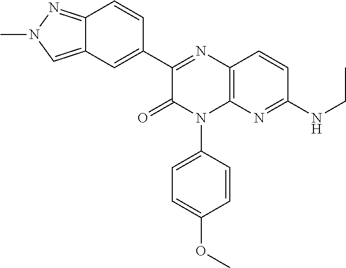 [SMILES: CCNc1ccc2nc(-c3ccc4nn(C)cc4c3)c(=O)n(-c3ccc(OC)cc3)c2n1] | 341 |
| 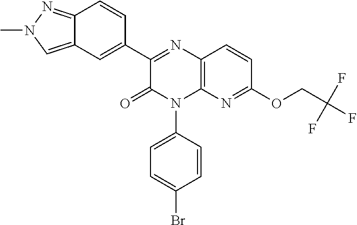 [SMILES: Cn1cc2cc(-c3nc4ccc(OCC(F)(F)F)nc4n(-c4ccc(Br)cc4)c3=O)ccc2n1] | 346 |
| 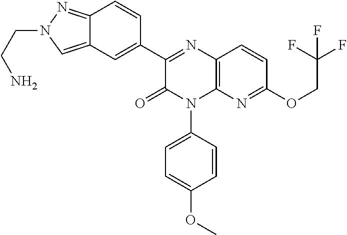 [SMILES: COc1ccc(-n2c(=O)c(-c3ccc4nn(CCN)cc4c3)nc3ccc(OCC(F)(F)F)nc32)cc1] | 347 |
| 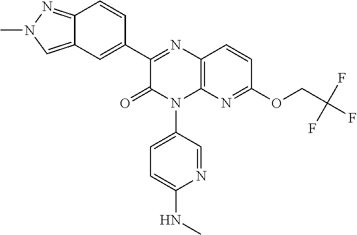 [SMILES: CNc1ccc(-n2c(=O)c(-c3ccc4nn(C)cc4c3)nc3ccc(OCC(F)(F)F)nc32)cn1] | 348 |
| 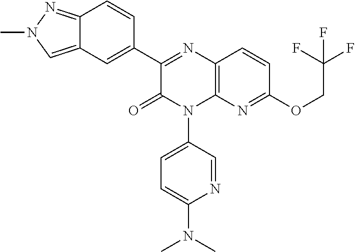 [SMILES: CN(C)c1ccc(-n2c(=O)c(-c3ccc4nn(C)cc4c3)nc3ccc(OCC(F)(F)F)nc32)cn1] | 349 |
| 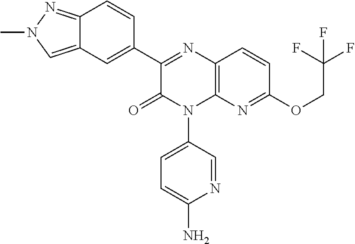 [SMILES: Cn1cc2cc(-c3nc4ccc(OCC(F)(F)F)nc4n(-c4ccc(N)nc4)c3=O)ccc2n1] | 350 |
| 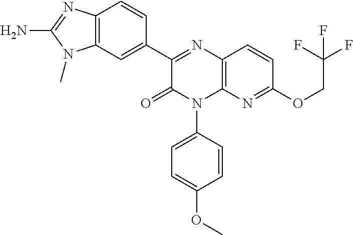 [SMILES: COc1ccc(-n2c(=O)c(-c3ccc4nc(N)n(C)c4c3)nc3ccc(OCC(F)(F)F)nc32)cc1] | 351 |
| 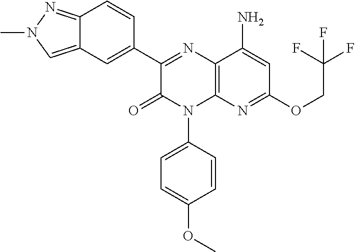 [SMILES: COc1ccc(-n2c(=O)c(-c3ccc4nn(C)cc4c3)nc3c(N)cc(OCC(F)(F)F)nc32)cc1] | 352 |

### Pharmaceutical Composition

The disclosure also provides a pharmaceutical composition comprising a therapeutically effective amount of one or more compounds according to Formula I, Formula II, or a pharmaceutically acceptable salt, stereoisomer, tautomer, and/or isotopologue thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Tables 1 and 2 or a pharmaceutically acceptable salt, stereoisomer, tautomer, and/or isotopologue thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, tautomer, and/or isotopologue thereof that is administered is governed by such considerations, and is the minimum amount necessary to exert a cytotoxic effect on a cancer, or to inhibit MAT2A activity, or both. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the MAT2A inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soybean, lecithin, and esters or partial esters derived from fatty acids and hexitol, anhydrides, for example sorbitan monooleate, and condensation reaction products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I or Formula II may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The MAT2A enzyme catalyzes the synthesis of S-adenosyl methionine (SAM) from methionine and ATP in cells. Accordingly, in another embodiment of the present disclosure there is provided a method of inhibiting in a cell the synthesis of SAM comprising introducing into the cell an effective amount of a compound of Formula I or Formula II or a pharmaceutically acceptable salt, stereoisomer, tautomer, and/or isotopologue thereof. In some embodiments, the cell is in a subject. In some embodiments, a Formula I or Formula II compound is used to identify other compounds that are inhibitors of MAT2A, for example, in a competition assay for binding to MAT2A or for the inhibition of SAM production. Binding to MAT2A or the inhibition of SAM production by a test compound having a detectable label can be measured with and without the presence of an unlabeled compound of the present disclosure.

The present disclosure also provides a method for treating a cancer in a subject suffering therefrom, comprising administering to the subject an effective amount of a compound of Formula I or Formula II or a pharmaceutically acceptable salt, stereoisomer, tautomer, and/or isotopologue thereof as described herein. In an embodiment, optionally in combination with any other embodiment, the subject is a mammal, such as a human.

In an embodiment, the cancer is an MTAP-deleted cancer. In some embodiments, the cancer as one selected from the group consisting of mesothelioma, neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, head and neck cancer, lymphoma, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma (SCLC), non-small cell lung carcinoma (NSCLC), multiple myeloma (MM), basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

In other embodiments, the cancer is selected from lung cancer, non-small cell lung cancer, bronchioloalveolar cell lung cancer, bone cancer, pancreatic cancer, skin cancer, head and neck cancer, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphoma, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including resistant and/or refractory versions of any of the above cancers, and a combination of one or more of the above cancers.

In some embodiments, the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma and breast cancer.

In other embodiments the lung cancer is non-small cell lung cancer, small cell lung cancer, adenocarcinoma of the lung, and squamous cell carcinoma of the lung.

In other embodiments the breast cancer is triple negative breast cancer (TNBC).

In other embodiments, the brain cancer is a brain tumor selected from the group consisting of glioma, glioblastoma, astrocytoma, meningioma, medulloblastoma, peripheral neuroectodermal tumors, and craniopharyngioma.

In still other embodiments, the cancer is a lymphoma selected from the group consisting of mantle cell lymphoma, Hodgkin lymphoma, non-Hodgkin lymphoma, Burkitt lymphoma, diffuse large B-cell lymphoma (DLBCL), and adult T-cell leukemia/lymphoma (ATLL). As used herein, the expression adult T-cell leukemia/lymphoma refers to a rare and often aggressive T-cell lymphoma that can be found in the blood (leukemia), lymph nodes (lymphoma), skin, or multiple areas of the body.

As described generally above, methylthioadenosine phosphorylase (MTAP) is an enzyme found in all normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine. Many human and murine malignant cells lack MTAP activity. MTAP deficiency is not only found in tissue culture cells but the deficiency is also present in primary leukemias, gliomas, melanomas, pancreatic cancers, non-small cell lung cancers (NSCLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas. For example, proliferation of cancer cells that are MTAP null, i.e., MTAP-deleted, is inhibited by knocking down MAT2A expression with shRNA which was confirmed using small molecule inhibitors of MAT2A. K. Marjon et al., Cell Reports 15 (2016) 574-587, incorporated herein by reference. An MTAP null or MTAP-deleted cancer is a cancer in which the MTAP gene has been deleted or lost or otherwise deactivated or a cancer in which the MTAP protein has a reduced or impaired function, or a reduced presence.

Accordingly, in an embodiment of the present disclosure there is provided a method for treating a cancer in a subject wherein the cancer is characterized by a reduction or absence of MTAP expression or absence of the MTAP gene or reduced function of MTAP protein as compared to cancers where the MTAP gene and/or protein is present and fully functioning, or as compared to cancers with the wild type MTAP gene. The method comprises administering to the subject a therapeutically effective amount of a compound of Formula I or Formula II or a pharmaceutically acceptable salt, stereoisomer, or tautomer thereof.

In another embodiment, there is provided a method of treating an MTAP deleted cancer in a subject comprising administering to the subject an effective amount of a compound of Formula I, Formula II, or a pharmaceutically acceptable salt, stereoisomer, tautomer, and/or isotopologue thereof. In an embodiment, the MTAP deleted cancer is selected from leukemia, glioma, melanoma, pancreatic cancer, non-small cell lung cancer (NSCLC), bladder cancer, astrocytoma, osteosarcoma, head and neck cancer, myxoid chondrosarcoma, ovarian cancer, endometrial cancer, breast cancer, soft tissue sarcoma, lymphoma, and mesothelioma.

In an embodiment, the MTAP deleted cancer is pancreatic cancer. In another embodiment, the MTAP deleted cancer is selected from bladder cancer, melanoma, brain cancer, lung cancer, pancreatic cancer, breast cancer, liver cancer, esophageal cancer, gastric cancer, colon cancer, head and neck cancer, kidney cancer, colon cancer, diffuse large B cell lymphoma (DLBCL), acute lymphoblastic leukemia (ALL), mantle cell lymphoma (MCL), glioblastoma multiforme (GBM), and non-small cell lung cancer (NSCLC).

Genomic analysis of MTAP null cell lines revealed that cell lines incorporating a KRAS mutation or a p53 mutation were sensitive to MAT2A inhibition. Accordingly, an embodiment of the present disclosure provides a method for treating a cancer in a subject wherein the cancer is characterized by reduction or absence of MTAP expression or absence of the MTAP gene or reduced function of MTAP protein, the method comprising administering to the subject a therapeutically effective amount of a compound of Formula I or Formula II, or a pharmaceutically acceptable salt, stereoisomer, tautomer, and/or isotopologue thereof, wherein said cancer is further characterized by the presence of mutant KRAS or mutant p53. In an embodiment, there is provided a method of treating an MTAP null cancer having a mutant KRAS or mutant p53 in a subject, comprising administering to the subject an effective amount of a compound of Formula I or Formula II or a pharmaceutically acceptable salt, stereoisomer, tautomer, and/or isotopologue thereof. For example, the cancer is MTAP null and KRAS mutant, MTAP null and p53 mutant, or each of MTAP null, KRAS mutant and p53 mutant.

The term “mutant KRAS” or “KRAS mutation” refers to a KRAS protein incorporating an activating mutation that alters its normal function and the gene encoding such a protein. For example, a mutant KRAS protein may incorporate a single amino acid substitution at position 12 or 13. In a particular embodiment, the KRAS mutant incorporates a G12X or G13X substitution, wherein X represents any amino acid change at the indicated position. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. In an embodiment, said p53 mutation is, Y126_splice, K132Q, M133K, R174fs, R175H, R196*, C238S, C242Y, G245S, R248W, R248Q, I255T, D259V, S261_splice, R267P, R273C, R282W, A159V or R280K. In an embodiment, the foregoing cancer is non-small cell lung cancer (NSCLC), pancreatic cancer, head and neck cancer, gastric cancer, breast cancer, colon cancer or ovarian cancer.

In another embodiment, the compounds disclosed herein are useful as ligands for degradation of disease-associated proteins. An example of this approach is PROTACs (PROteolysis TArgeting Chimeras). PROTACs are bifunctional molecules that comprise both a ligand moiety selected from one of the compounds disclosed herein, which is capable of binding the target protein, and a ligase targeting moiety, such as a peptide portion (referred to as the degron) that is recognized and polyubiquitinated by E3 ligase. Thus, the PROTAC non-covalently binds to a target protein, and recruits E3 ligase via the degron, which results in polyubiquination and degradation of the bound target. A number of publications describe the pre-clinical use of PROTACs in a variety of therapeutic areas including oncology. See, e.g., Lu et al. Chemistry & Biology 22 (2015) 755-763.

Aspects

Aspect 1. A compound according to Formula I.

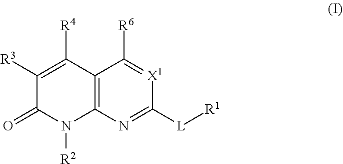

wherein

X¹ is N or CR⁵;

L is O, S, S(O), S(O)₂, NR, or a bond;

R is H or C₁-C₆-alkyl;

R¹ is selected from the group consisting of C₁-C₆-alkyl, C₂-C₆-alkenyl, C₃-C₅-carbocyclyl, —(C₁-C₆-alkyl)(C₃-C₆-carbocyclyl), and —(C₁-C₆-alkyl)(C₃-C₆-cycloalkenyl), wherein

any alkyl in R¹ is straight or branched,

R¹ is optionally substituted by 1-6 halo; and

when X¹ is N, L is NR, R is H, and R¹ is C₁-C₆-alkyl, then R¹ is substituted by 1-6 halo;

or when L is NR, then R and R¹ can be taken together in combination with L to form a 3- to 6-membered heterocycloalkyl (wherein 1-4 ring members are independently selected from N, O, and S) optionally substituted by one or more R^{A};

R² and R³ are independently selected from the group consisting of C₆-C₁₀-aryl and 5- to 10-membered heteroaryl (wherein 1-4 heteroaryl members are independently selected from N, O, and S),

wherein R² and R³ are independently and optionally substituted by one or more substituents that are selected from the group consisting of R^{A}, OR^{A}halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN;

R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D};

R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D};

R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B};

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S);

wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl),

wherein each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo,

R^{C} and R^{D} are each independently selected from H and C₁-C₆-alkyl; and

wherein the compound is not:

6-(2-chlorophenyl)-8-(4-fluorophenyl)-2-(methylthio)pyrido[2,3-d]pyrimidin-7(8H)-one;

2-(methylthio)-8-phenyl-6-(o-tolyl)pyrido[2,3-d]pyrimidin-7(8H)-one;

6-(4-hydroxyphenyl)-2-(methylthio)-8-phenylpyrido[2,3-d]pyrimidin-7(8H)-one;

6-(4-methoxyphenyl)-2-(methylthio)-8-phenylpyrido[2,3-d]pyrimidin-7(8H)-one;

6-(2,5-dimethoxyphenyl)-2-(methylthio)-8-phenylpyrido[2,3-d]pyrimidin-7(8H)-one;

6-(4-(tert-butyl)phenyl)-2-(methylthio)-8-phenylpyrido[2,3-d]pyrimidin-7(8H)-one; or

6-(2,6-dichlorophenyl)-2-(methylthio)-8-phenylpyrido[2,3-d]pyrimidin-7(8H)-one;

or a pharmaceutically acceptable salt thereof.

Aspect 2. A compound according to Formula II:

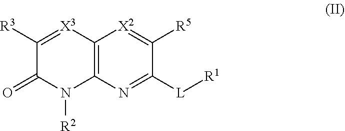

wherein

X² is CR⁶ and X³ is N, or X² is N and X³ is CR⁴;

L is O, S, NR, or a bond;

R is H or C₁-C₆-alkyl;

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein

any alkyl in R¹ is straight or branched,

R¹ is optionally substituted by 1-6 halo;

or when L is NR, then R and R¹ can be taken together in combination with L to form a 3- to 6-membered heterocycloalkyl (wherein 1-4 ring members are independently selected from N, O, and S) optionally substituted by one or more R^{A};

R² and R³ are independently selected from the group consisting of C₆-C₁₀-aryl and 5- to 10-membered heteroaryl (wherein 1-4 heteroaryl members are independently selected from N, O, and S),

wherein R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN;

R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D};

R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D};

R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B};

R^{A} and R^{B} are independently selected from the group consisting of H, —CN, -hydroxy, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S);

wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl),

wherein each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo,

R^{C} and R^{D} are each independently selected from H and C₁-C₆-alkyl;

or a pharmaceutically acceptable salt thereof.

Aspect 3. The compound or pharmaceutically salt thereof according to Aspect 1, wherein X¹ is N.

Aspect 4. The compound or pharmaceutically salt thereof according to Aspect 1, wherein X¹ is CR⁵.

Aspect 5. The compound or pharmaceutically salt thereof according to Aspect 2, wherein X² is CR⁶ and X³ is N.

Aspect 6. The compound or pharmaceutically salt thereof according to Aspect 2, wherein X² is N and X³ is CR⁴.

Aspect 7. The compound or pharmaceutically salt thereof according to any one of Aspects 1-6, wherein

each of R⁴ and R⁵ is independently selected from H and C₁-C₆-alkyl; and

R⁶ is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are independently selected from H and C₁-C₆-alkyl).

Aspect 8. The compound or pharmaceutically salt thereof according to any one of Aspects 1 to 7, wherein at least one of R⁴, R⁵, and R⁶ is H.

Aspect 9. The compound or pharmaceutically salt thereof according to any one of Aspects 1 to 8, wherein R⁴ is H.

Aspect 10. The compound or pharmaceutically salt thereof according to any one of Aspects 1 to 8, wherein R⁵ is H.

Aspect 11. The compound or pharmaceutically salt thereof according to any one of Aspects 1 to 8, wherein R⁶ is H.

Aspect 12. The compound or pharmaceutically salt thereof according to any one of Aspects 1 to 11, wherein each of R⁴, R⁵, and R⁶ is H.

Aspect 13. The compound or pharmaceutically salt thereof according to any one of Aspects 1 to 12, wherein R² is C₆-C₁₀-aryl.

Aspect 14. The compound or pharmaceutically salt thereof according to Aspect 13, wherein R² is phenyl.

Aspect 15. The compound or pharmaceutically salt thereof according to any one of Aspects 1 to 12, wherein R² is 5- to 10-membered heteroaryl, and wherein 1 ring member is N.

Aspect 16. The compound or pharmaceutically salt thereof according to Aspect 15, wherein R² is pyridyl.

Aspect 17. The compound or pharmaceutically salt thereof according to any one of Aspects 1 to 16, wherein R³ is 5- to 10-membered heteroaryl.

Aspect 18. The compound or pharmaceutically salt thereof according to Aspect 17, wherein R³ is selected from the group consisting of benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

Aspect 19. The compound or pharmaceutically salt thereof according to any one of Aspects 1 to 16, wherein R³ is C₆-C₁₀-aryl.

Aspect 20. The compound or pharmaceutically salt thereof according to Aspect 19, wherein R³ is phenyl.

Aspect 21. The compound or pharmaceutically salt thereof according to any one of Aspects 1 to 12, wherein R² is phenyl and R³ is 5- to 10-membered heteroaryl.

Aspect 22. The compound or pharmaceutically salt thereof according to any one of Aspects 1 to 12, wherein each of R² and R³ is C₆-C₁₀-aryl.

Aspect 23. The compound or pharmaceutically salt thereof according to Aspect 22, wherein each of R² and R³ is phenyl.

Aspect 24. The compound or pharmaceutically salt thereof according to any one of Aspects 1 to 23, wherein L is O or NR.

Aspect 25. The compound or pharmaceutically salt thereof according to any one of Aspects 1 to 24, wherein L is NR.

Aspect 26. The compound or pharmaceutically salt thereof according to any one of Aspects 1 to 25, wherein R¹ is C₁-C₆-alkyl or C₃-C₅-carbocyclyl.

Aspect 27. The compound or pharmaceutically salt thereof according to any one of Aspects 1 to 26, wherein R¹ is C₁-C₃-alkyl that is optionally substituted by 1-3 fluoro.

Aspect 28. The compound or pharmaceutically salt thereof according to any one of Aspects 1 to 7, wherein

L is O or NR and R is H;

R¹ is C₁-C₃-alkyl that is optionally substituted by 1-3 fluoro;

R² is 5- to 10-membered heteroaryl (wherein 1 heteroaryl member is N) or C₆-C₁₀-aryl;

R³ is 5- to 10-membered heteroaryl wherein 1 to 3 heteroaryl members are independently selected from N, O, and S, or C₆-C₁₀-aryl; and

each of R⁴, R⁵, and R⁶ is H.

Aspect 29. The compound or pharmaceutically salt thereof according to Aspect 28, wherein L is NR.

Aspect 30. The compound or pharmaceutically salt thereof according to Aspect 28 or 29, wherein

R² is optionally substituted phenyl; and

R³ is an optionally substituted 5- to 10-membered heteroaryl wherein 1 to 3 heteroaryl members are independently selected from N, O, and S.

Aspect 31. The compound or pharmaceutically salt thereof according to Aspect 28 or 29, wherein

R² is an optionally substituted 5- to 10-membered heteroaryl wherein 1 heteroaryl member is N; and

R³ is optionally substituted phenyl.

Aspect 32. The compound or pharmaceutically salt thereof according to Aspect 30, wherein R³ is selected from the group consisting of optionally substituted benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

Aspect 33. The compound or pharmaceutically salt thereof according to Aspect 28 or 29, wherein R² and R³ independently are optionally substituted phenyl.

Aspect 34. The compound or pharmaceutically salt thereof according to Aspect 1 or 2, wherein

L is O or NR and R is H;

X¹ is CR⁵;

R¹ is C₁-C₃-alkyl that is optionally substituted by 1-3 fluoro;

R² is substituted phenyl or substituted pyridyl;

R³ is selected from the group consisting of substituted phenyl, substituted benzimidazolyl, and triazolopyridinyl; and

each of R⁴, R⁵, and R⁶ is H.

Aspect 35. The compound or pharmaceutically salt thereof according to Aspect 1, wherein the compound is selected from the following table:

| | |
|---|---|
| 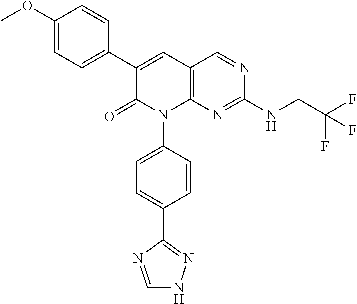 [SMILES: COc1ccc(-c2cc3cnc(NCC(F)(F)F)nc3n(-c3ccc(-c4nc[nH]n4)cc3)c2=O)cc1] | 101 |
| 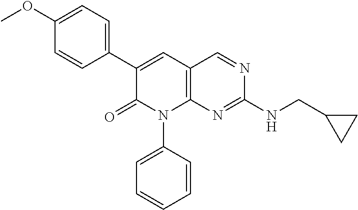 [SMILES: COc1ccc(-c2cc3cnc(NCC4CC4)nc3n(-c3ccccc3)c2=O)cc1] | 102 |
| 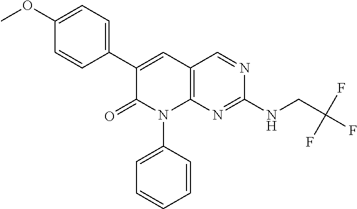 [SMILES: COc1ccc(-c2cc3cnc(NCC(F)(F)F)nc3n(-c3ccccc3)c2=O)cc1] | 103 |
| 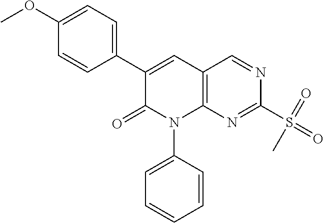 [SMILES: COc1ccc(-c2cc3cnc(S(C)(=O)=O)nc3n(-c3ccccc3)c2=O)cc1] | 104 |
| 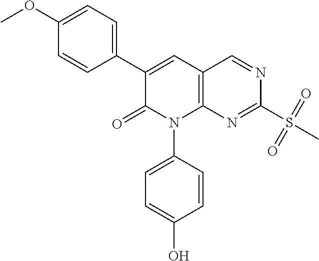 | 105 |
| 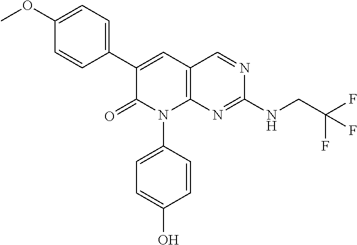 [SMILES: COc1ccc(-c2cc3cnc(NCC(F)(F)F)nc3n(-c3ccc(O)cc3)c2=O)cc1] | 106 |
| 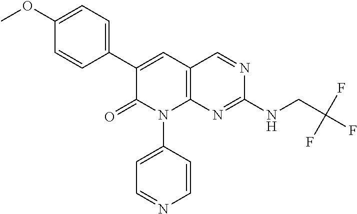 [SMILES: COc1ccc(-c2cc3cnc(NCC(F)(F)F)nc3n(-c3ccncc3)c2=O)cc1] | 107 |
| 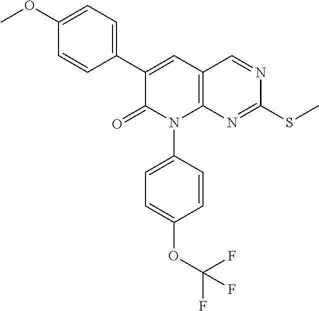 | 108 |
| 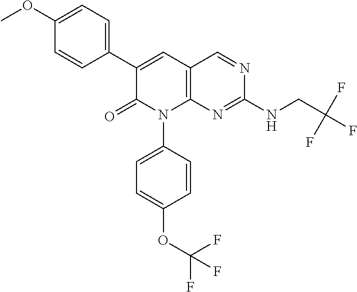 [SMILES: COc1ccc(-c2cc3cnc(NCC(F)(F)F)nc3n(-c3ccc(OC(F)(F)F)cc3)c2=O)cc1] | 109 |
| 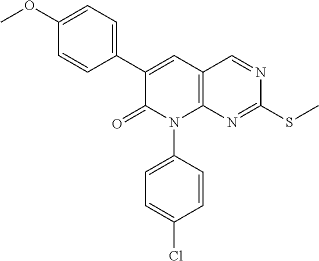 | 110 |
| 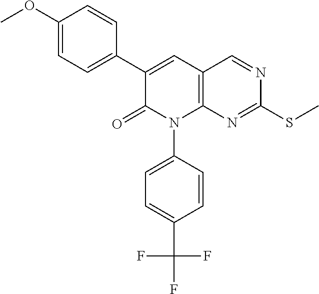 | 111 |
| 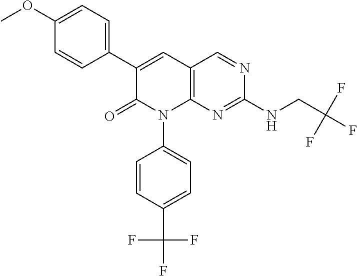 [SMILES: COc1ccc(-c2cc3cnc(NCC(F)(F)F)nc3n(-c3ccc(C(F)(F)F)cc3)c2=O)cc1] | 112 |
| 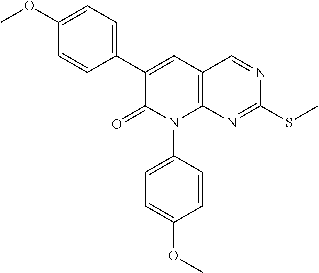 | 113 |
| 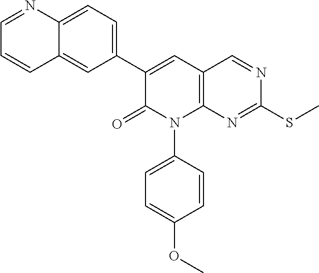 [SMILES: COc1ccc(-n2c(=O)c(-c3ccc4ncccc4c3)cc3cnc(SC)nc32)cc1] | 114 |
| 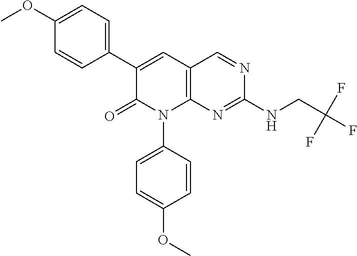 [SMILES: COc1ccc(-c2cc3cnc(NCC(F)(F)F)nc3n(-c3ccc(OC)cc3)c2=O)cc1] | 115 |
| 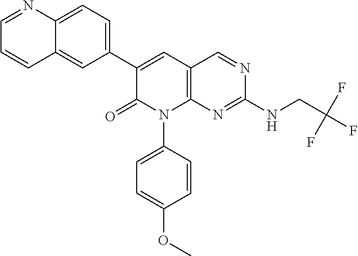 [SMILES: COc1ccc(-n2c(=O)c(-c3ccc4ncccc4c3)cc3cnc(NCC(F)(F)F)nc32)cc1] | 116 |
| 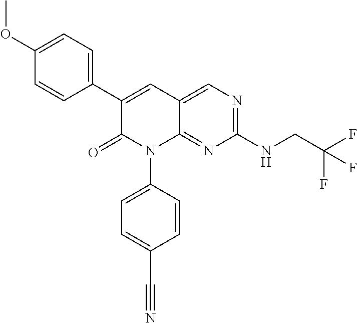 [SMILES: COc1ccc(-c2cc3cnc(NCC(F)(F)F)nc3n(-c3ccc(C#N)cc3)c2=O)cc1] | 117 |
| 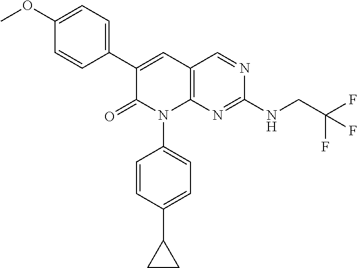 [SMILES: COc1ccc(-c2cc3cnc(NCC(F)(F)F)nc3n(-c3ccc(C4CC4)cc3)c2=O)cc1] | 118 |
| 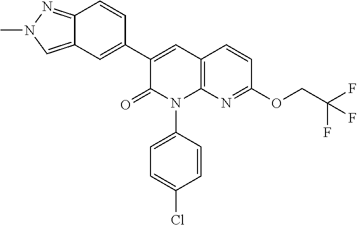 [SMILES: Cn1cc2cc(-c3cc4ccc(OCC(F)(F)F)nc4n(-c4ccc(Cl)cc4)c3=O)ccc2n1] | 119 |
| 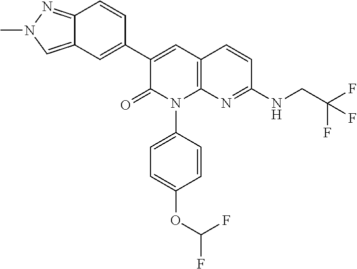 [SMILES: Cn1cc2cc(-c3cc4ccc(NCC(F)(F)F)nc4n(-c4ccc(OC(F)F)cc4)c3=O)ccc2n1] | 120 |
| 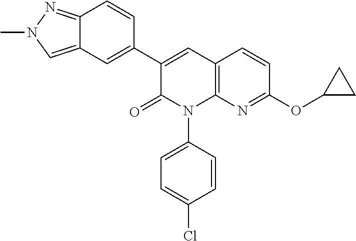 [SMILES: Cn1cc2cc(-c3cc4ccc(OC5CC5)nc4n(-c4ccc(Cl)cc4)c3=O)ccc2n1] | 121 |
| 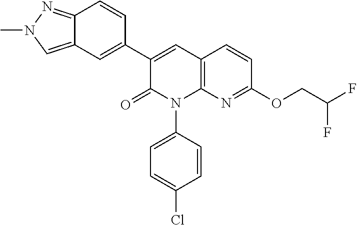 [SMILES: Cn1cc2cc(-c3cc4ccc(OCC(F)F)nc4n(-c4ccc(Cl)cc4)c3=O)ccc2n1] | 122 |
| 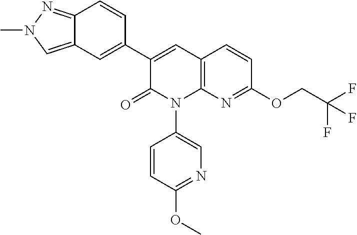 [SMILES: COc1ccc(-n2c(=O)c(-c3ccc4nn(C)cc4c3)cc3ccc(OCC(F)(F)F)nc32)cn1] | 123 |
| 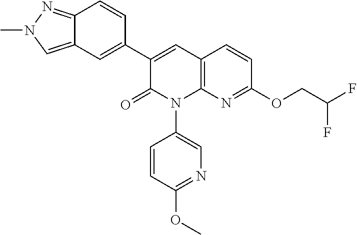 [SMILES: COc1ccc(-n2c(=O)c(-c3ccc4nn(C)cc4c3)cc3ccc(OCC(F)F)nc32)cn1] | 124 |
| 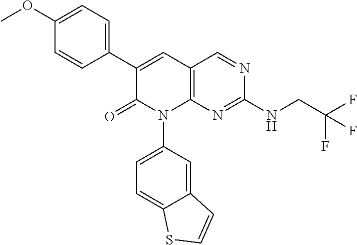 [SMILES: COc1ccc(-c2cc3cnc(NCC(F)(F)F)nc3n(-c3ccc4sccc4c3)c2=O)cc1] | 125 |
| 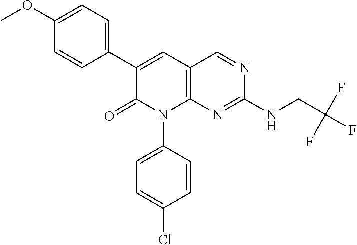 [SMILES: COc1ccc(-c2cc3cnc(NCC(F)(F)F)nc3n(-c3ccc(Cl)cc3)c2=O)cc1] | 126 |
| 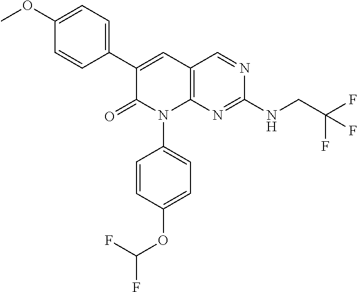 [SMILES: COc1ccc(-c2cc3cnc(NCC(F)(F)F)nc3n(-c3ccc(OC(F)F)cc3)c2=O)cc1] | 127 |
| 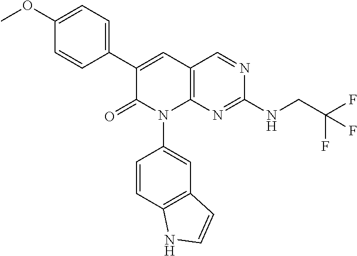 [SMILES: COc1ccc(-c2cc3cnc(NCC(F)(F)F)nc3n(-c3ccc4[nH]ccc4c3)c2=O)cc1] | 128 |
| 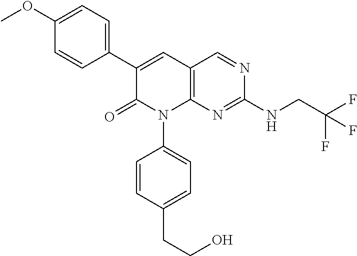 [SMILES: COc1ccc(-c2cc3cnc(NCC(F)(F)F)nc3n(-c3ccc(CCO)cc3)c2=O)cc1] | 129 |
| 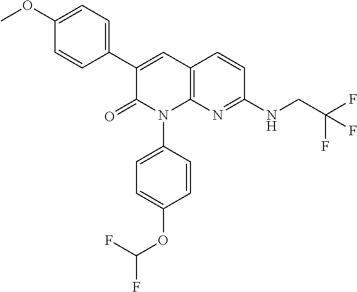 [SMILES: COc1ccc(-c2cc3ccc(NCC(F)(F)F)nc3n(-c3ccc(OC(F)F)cc3)c2=O)cc1] | 130 |
| 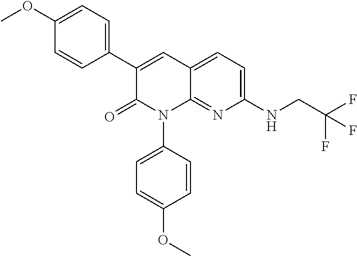 [SMILES: COc1ccc(-c2cc3ccc(NCC(F)(F)F)nc3n(-c3ccc(OC)cc3)c2=O)cc1] | 131 |
| 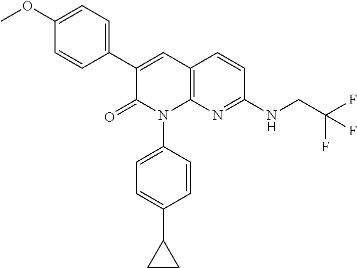 [SMILES: COc1ccc(-c2cc3ccc(NCC(F)(F)F)nc3n(-c3ccc(C4CC4)cc3)c2=O)cc1] | 132 |
| 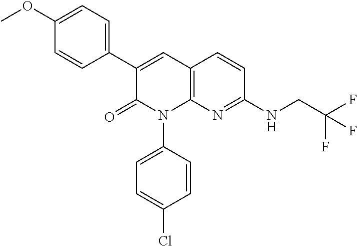 [SMILES: COc1ccc(-c2cc3ccc(NCC(F)(F)F)nc3n(-c3ccc(Cl)cc3)c2=O)cc1] | 133 |
| 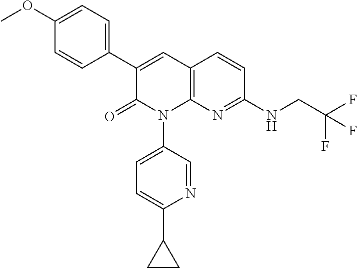 [SMILES: COc1ccc(-c2cc3ccc(NCC(F)(F)F)nc3n(-c3ccc(C4CC4)nc3)c2=O)cc1] | 134 |
| 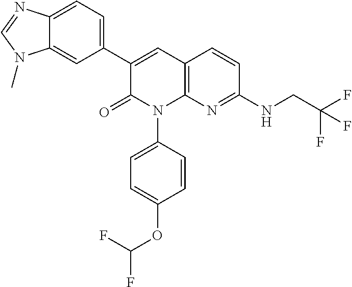 [SMILES: Cn1cnc2ccc(-c3cc4ccc(NCC(F)(F)F)nc4n(-c4ccc(OC(F)F)cc4)c3=O)cc21] | 135 |
| 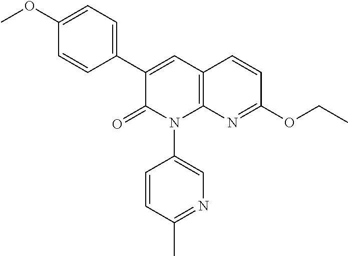 [SMILES: CCOc1ccc2cc(-c3ccc(OC)cc3)c(=O)n(-c3ccc(C)nc3)c2n1] | 136 |
| 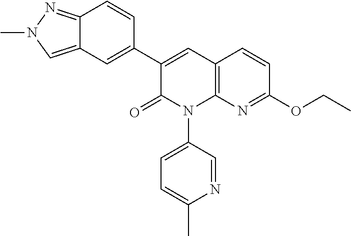 [SMILES: CCOc1ccc2cc(-c3ccc4nn(C)cc4c3)c(=O)n(-c3ccc(C)nc3)c2n1] | 137 |
| 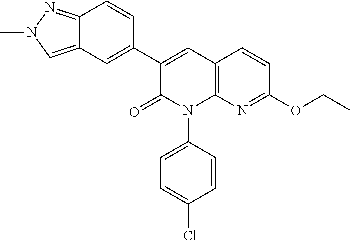 [SMILES: CCOc1ccc2cc(-c3ccc4nn(C)cc4c3)c(=O)n(-c3ccc(Cl)cc3)c2n1] | 138 |
| 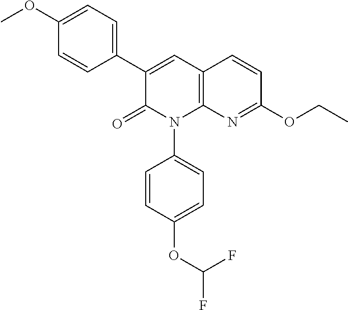 [SMILES: CCOc1ccc2cc(-c3ccc(OC)cc3)c(=O)n(-c3ccc(OC(F)F)cc3)c2n1] | 139 |
| 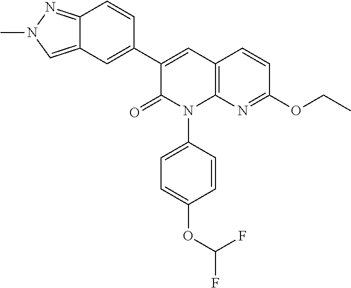 [SMILES: CCOc1ccc2cc(-c3ccc4nn(C)cc4c3)c(=O)n(-c3ccc(OC(F)F)cc3)c2n1] | 140 |
| 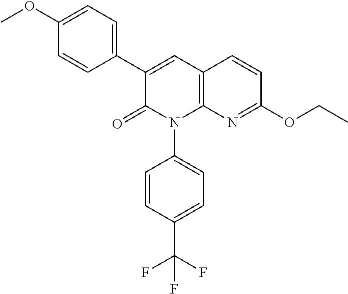 [SMILES: CCOc1ccc2cc(-c3ccc(OC)cc3)c(=O)n(-c3ccc(C(F)(F)F)cc3)c2n1] | 141 |
| 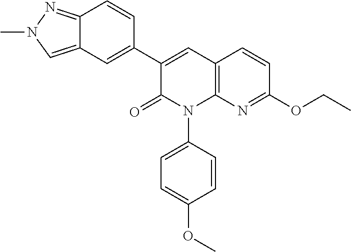 [SMILES: CCOc1ccc2cc(-c3ccc4nn(C)cc4c3)c(=O)n(-c3ccc(OC)cc3)c2n1] | 142 |
| 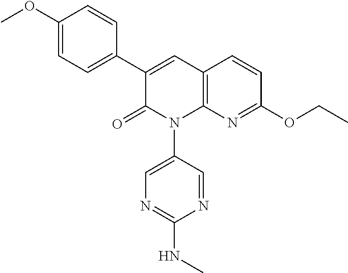 [SMILES: CCOc1ccc2cc(-c3ccc(OC)cc3)c(=O)n(-c3cnc(NC)nc3)c2n1] | 143 |
| 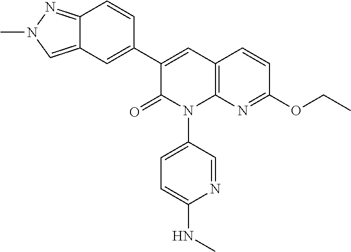 [SMILES: CCOc1ccc2cc(-c3ccc4nn(C)cc4c3)c(=O)n(-c3ccc(NC)nc3)c2n1] | 144 |
| 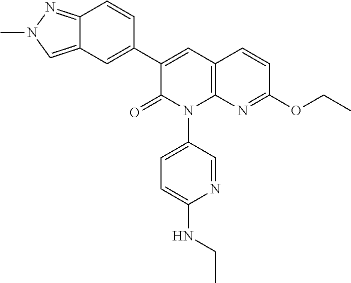 [SMILES: CCNc1ccc(-n2c(=O)c(-c3ccc4nn(C)cc4c3)cc3ccc(OCC)nc32)cn1] | 145 |
| 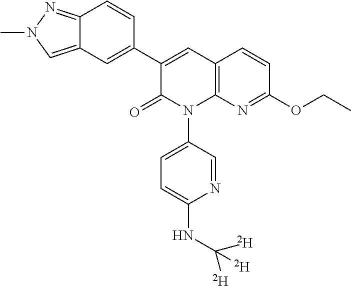 [SMILES: [2H]C([2H])([2H])Nc1ccc(-n2c(=O)c(-c3ccc4nn(C)cc4c3)cc3ccc(OCC)nc32)cn1] | 146 |
| 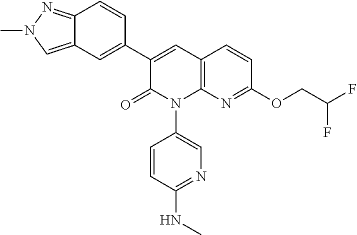 [SMILES: CNc1ccc(-n2c(=O)c(-c3ccc4nn(C)cc4c3)cc3ccc(OCC(F)F)nc32)cn1] | 147 |
| 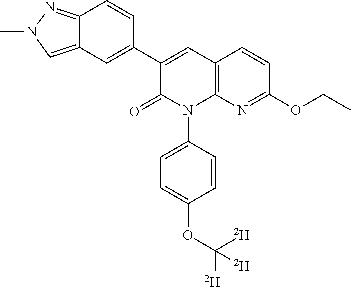 [SMILES: [2H]C([2H])([2H])Oc1ccc(-n2c(=O)c(-c3ccc4nn(C)cc4c3)cc3ccc(OCC)nc32)cc1] | 148 |
| 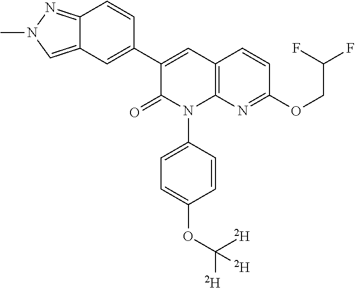 [SMILES: [2H]C([2H])([2H])Oc1ccc(-n2c(=O)c(-c3ccc4nn(C)cc4c3)cc3ccc(OCC(F)F)nc32)cc1] | 149 |
| 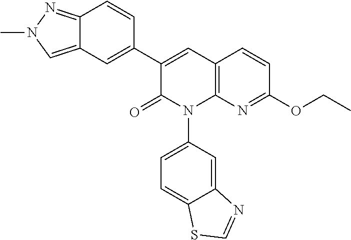 [SMILES: CCOc1ccc2cc(-c3ccc4nn(C)cc4c3)c(=O)n(-c3ccc4scnc4c3)c2n1] | 150 |
| 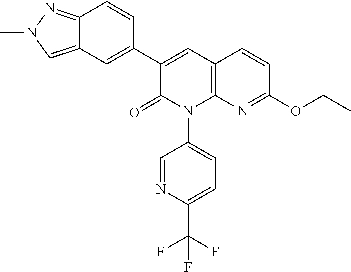 [SMILES: CCOc1ccc2cc(-c3ccc4nn(C)cc4c3)c(=O)n(-c3ccc(C(F)(F)F)nc3)c2n1] | 151 |
| 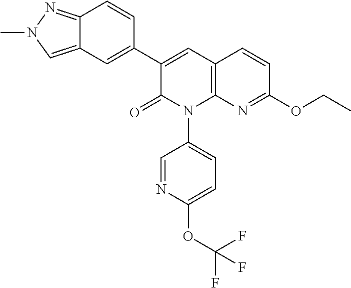 [SMILES: CCOc1ccc2cc(-c3ccc4nn(C)cc4c3)c(=O)n(-c3ccc(OC(F)(F)F)nc3)c2n1] | 152 |
| 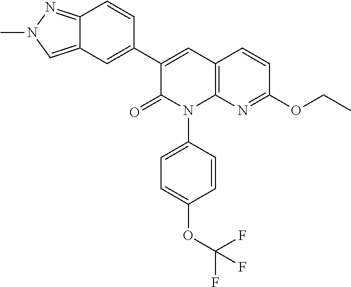 [SMILES: CCOc1ccc2cc(-c3ccc4nn(C)cc4c3)c(=O)n(-c3ccc(OC(F)(F)F)cc3)c2n1] | 153 |
| 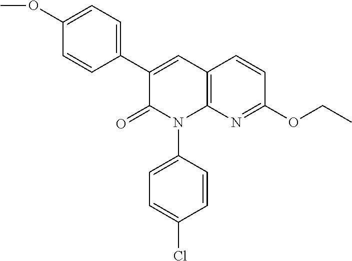 [SMILES: CCOc1ccc2cc(-c3ccc(OC)cc3)c(=O)n(-c3ccc(Cl)cc3)c2n1] | 154 |
| 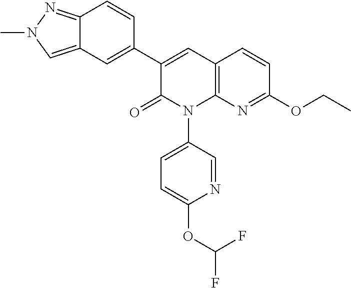 [SMILES: CCOc1ccc2cc(-c3ccc4nn(C)cc4c3)c(=O)n(-c3ccc(OC(F)F)nc3)c2n1] | 155 |
| 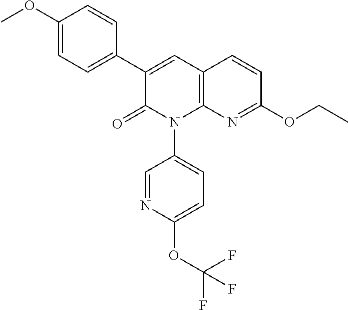 [SMILES: CCOc1ccc2cc(-c3ccc(OC)cc3)c(=O)n(-c3ccc(OC(F)(F)F)nc3)c2n1] | 156 |
| 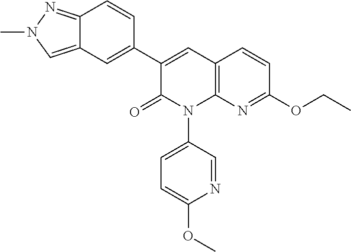 [SMILES: CCOc1ccc2cc(-c3ccc4nn(C)cc4c3)c(=O)n(-c3ccc(OC)nc3)c2n1] | 157 |
| 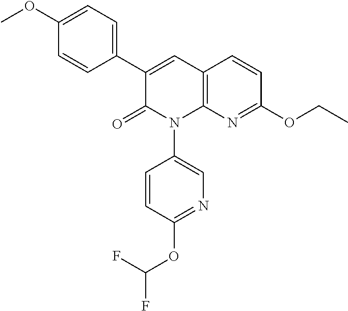 [SMILES: CCOc1ccc2cc(-c3ccc(OC)cc3)c(=O)n(-c3ccc(OC(F)F)nc3)c2n1] | 158 |
| 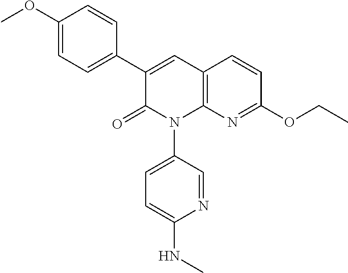 [SMILES: CCOc1ccc2cc(-c3ccc(OC)cc3)c(=O)n(-c3ccc(NC)nc3)c2n1] | 159 |
| 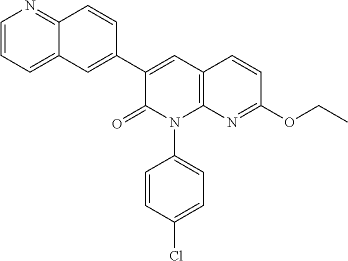 [SMILES: CCOc1ccc2cc(-c3ccc4ncccc4c3)c(=O)n(-c3ccc(Cl)cc3)c2n1] | 160 |
| 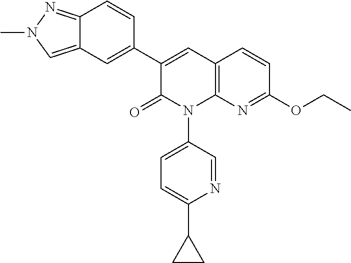 [SMILES: CCOc1ccc2cc(-c3ccc4nn(C)cc4c3)c(=O)n(-c3ccc(C4CC4)nc3)c2n1] | 161 |
| 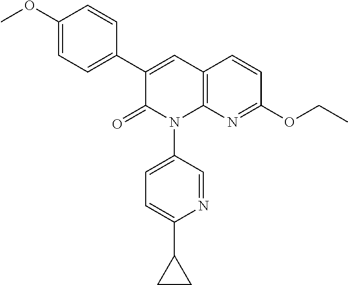 [SMILES: CCOc1ccc2cc(-c3ccc(OC)cc3)c(=O)n(-c3ccc(C4CC4)nc3)c2n1] | 162 |
| 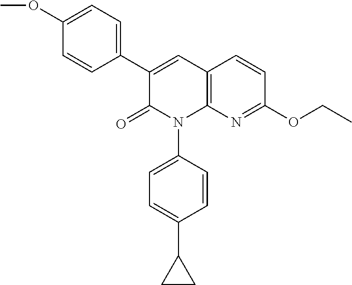 [SMILES: CCOc1ccc2cc(-c3ccc(OC)cc3)c(=O)n(-c3ccc(C4CC4)cc3)c2n1] | 163 |
| 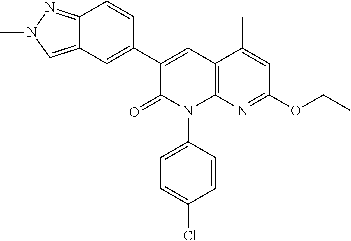 [SMILES: CCOc1cc(C)c2cc(-c3ccc4nn(C)cc4c3)c(=O)n(-c3ccc(Cl)cc3)c2n1] | 164 |
| 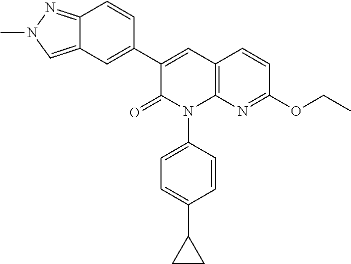 [SMILES: CCOc1ccc2cc(-c3ccc4nn(C)cc4c3)c(=O)n(-c3ccc(C4CC4)cc3)c2n1] | 165 |
| 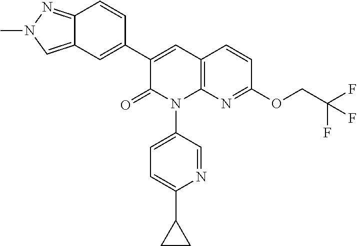 [SMILES: Cn1cc2cc(-c3cc4ccc(OCC(F)(F)F)nc4n(-c4ccc(C5CC5)nc4)c3=O)ccc2n1] | 166 |
| 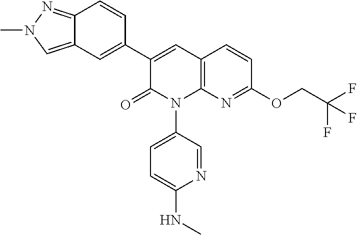 [SMILES: CNc1ccc(-n2c(=O)c(-c3ccc4nn(C)cc4c3)cc3ccc(OCC(F)(F)F)nc32)cn1] | 167 |
| 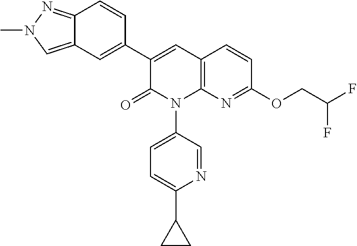 [SMILES: Cn1cc2cc(-c3cc4ccc(OCC(F)F)nc4n(-c4ccc(C5CC5)nc4)c3=O)ccc2n1] | 168 |
| 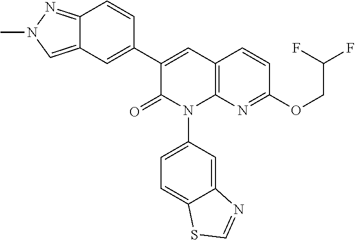 [SMILES: Cn1cc2cc(-c3cc4ccc(OCC(F)F)nc4n(-c4ccc5scnc5c4)c3=O)ccc2n1] | 169 |
| 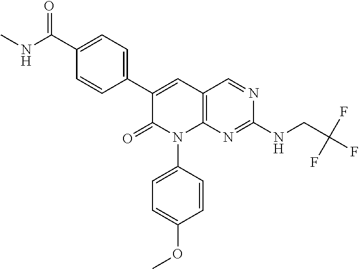 [SMILES: CNC(=O)c1ccc(-c2cc3cnc(NCC(F)(F)F)nc3n(-c3ccc(OC)cc3)c2=O)cc1] | 170 |
| 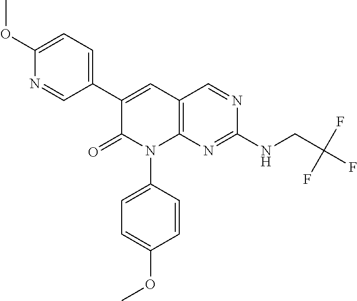 [SMILES: COc1ccc(-n2c(=O)c(-c3ccc(OC)nc3)cc3cnc(NCC(F)(F)F)nc32)cc1] | 171 |
| 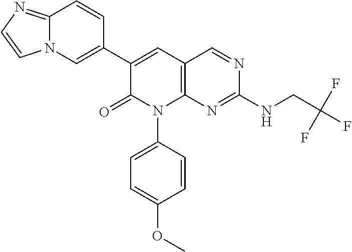 [SMILES: COc1ccc(-n2c(=O)c(-c3ccc4nccn4c3)cc3cnc(NCC(F)(F)F)nc32)cc1] | 172 |
| 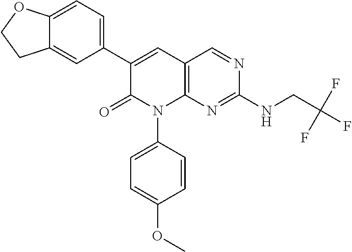 [SMILES: COc1ccc(-n2c(=O)c(-c3ccc4c(c3)CCO4)cc3cnc(NCC(F)(F)F)nc32)cc1] | 173 |
| 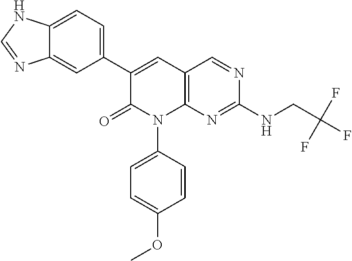 [SMILES: COc1ccc(-n2c(=O)c(-c3ccc4[nH]cnc4c3)cc3cnc(NCC(F)(F)F)nc32)cc1] | 174 |
| 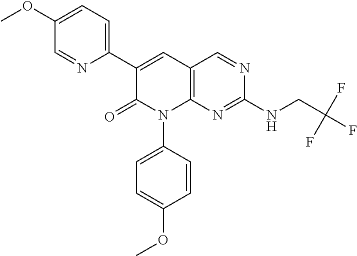 [SMILES: COc1ccc(-n2c(=O)c(-c3ccc(OC)cn3)cc3cnc(NCC(F)(F)F)nc32)cc1] | 175 |
| 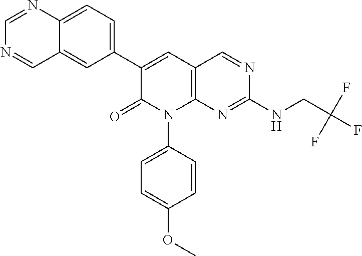 [SMILES: COc1ccc(-n2c(=O)c(-c3ccc4ncncc4c3)cc3cnc(NCC(F)(F)F)nc32)cc1] | 176 |
| 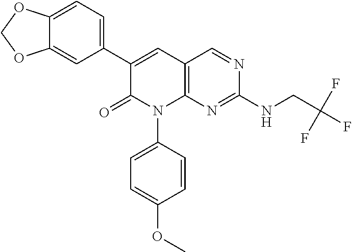 [SMILES: COc1ccc(-n2c(=O)c(-c3ccc4c(c3)OCO4)cc3cnc(NCC(F)(F)F)nc32)cc1] | 177 |
| 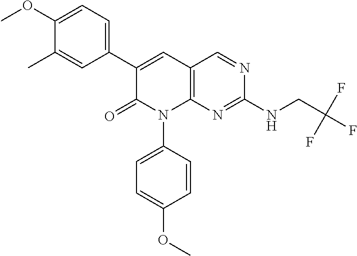 [SMILES: COc1ccc(-n2c(=O)c(-c3ccc(OC)c(C)c3)cc3cnc(NCC(F)(F)F)nc32)cc1] | 178 |
| 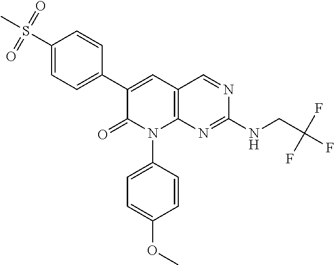 [SMILES: COc1ccc(-n2c(=O)c(-c3ccc(S(C)(=O)=O)cc3)cc3cnc(NCC(F)(F)F)nc32)cc1] | 179 |
| 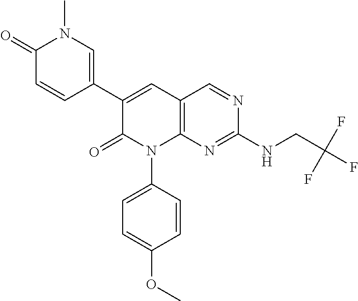 [SMILES: COc1ccc(-n2c(=O)c(-c3ccc(=O)n(C)c3)cc3cnc(NCC(F)(F)F)nc32)cc1] | 180 |
| 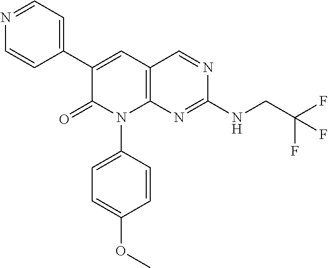 [SMILES: COc1ccc(-n2c(=O)c(-c3ccncc3)cc3cnc(NCC(F)(F)F)nc32)cc1] | 181 |
| 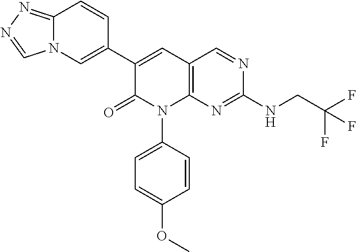 [SMILES: COc1ccc(-n2c(=O)c(-c3ccc4nncn4c3)cc3cnc(NCC(F)(F)F)nc32)cc1] | 182 |
| 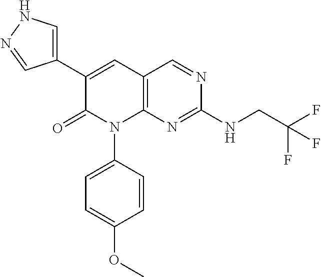 [SMILES: COc1ccc(-n2c(=O)c(-c3cn[nH]c3)cc3cnc(NCC(F)(F)F)nc32)cc1] | 183 |
| 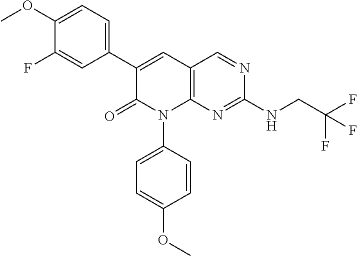 [SMILES: COc1ccc(-n2c(=O)c(-c3ccc(OC)c(F)c3)cc3cnc(NCC(F)(F)F)nc32)cc1] | 184 |
| 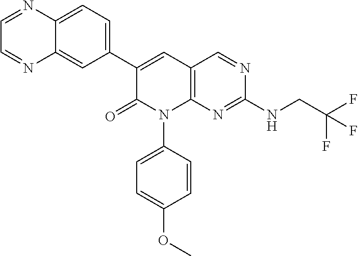 [SMILES: COc1ccc(-n2c(=O)c(-c3ccc4nccnc4c3)cc3cnc(NCC(F)(F)F)nc32)cc1] | 185 |
| 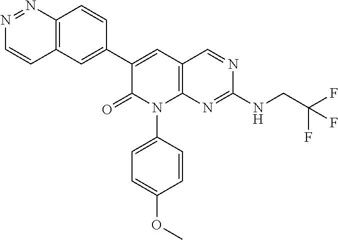 [SMILES: COc1ccc(-n2c(=O)c(-c3ccc4nnccc4c3)cc3cnc(NCC(F)(F)F)nc32)cc1] | 186 |
| 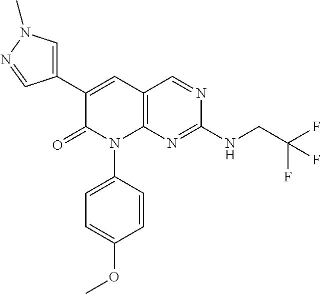 [SMILES: COc1ccc(-n2c(=O)c(-c3cnn(C)c3)cc3cnc(NCC(F)(F)F)nc32)cc1] | 187 |
| 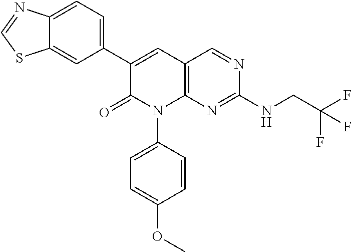 [SMILES: COc1ccc(-n2c(=O)c(-c3ccc4ncsc4c3)cc3cnc(NCC(F)(F)F)nc32)cc1] | 188 |
| 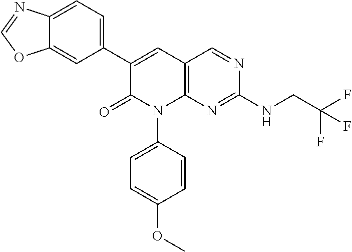 [SMILES: COc1ccc(-n2c(=O)c(-c3ccc4ncoc4c3)cc3cnc(NCC(F)(F)F)nc32)cc1] | 189 |
| 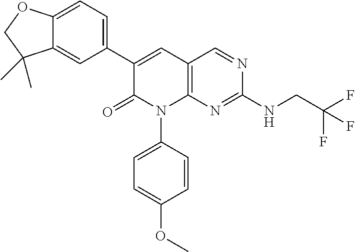 [SMILES: COc1ccc(-n2c(=O)c(-c3ccc4c(c3)C(C)(C)CO4)cc3cnc(NCC(F)(F)F)nc32)cc1] | 190 |
| 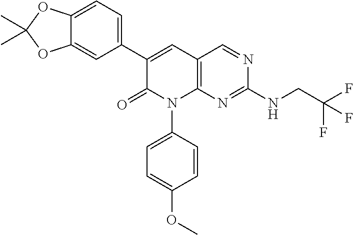 [SMILES: COc1ccc(-n2c(=O)c(-c3ccc4c(c3)OC(C)(C)O4)cc3cnc(NCC(F)(F)F)nc32)cc1] | 191 |
| 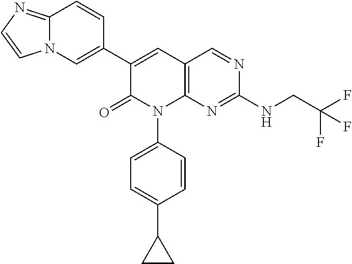 [SMILES: O=c1c(-c2ccc3nccn3c2)cc2cnc(NCC(F)(F)F)nc2n1-c1ccc(C2CC2)cc1] | 192 |
| 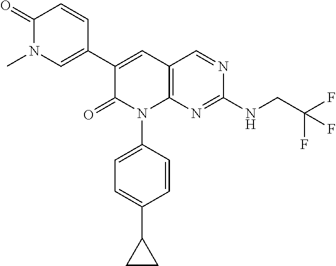 [SMILES: Cn1cc(-c2cc3cnc(NCC(F)(F)F)nc3n(-c3ccc(C4CC4)cc3)c2=O)ccc1=O] | 193 |
| 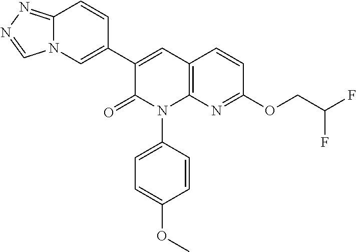 [SMILES: COc1ccc(-n2c(=O)c(-c3ccc4nncn4c3)cc3ccc(OCC(F)F)nc32)cc1] | 194 |
| 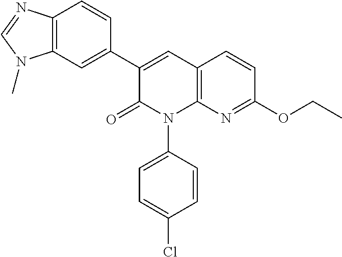 [SMILES: CCOc1ccc2cc(-c3ccc4ncn(C)c4c3)c(=O)n(-c3ccc(Cl)cc3)c2n1] | 195 |
| 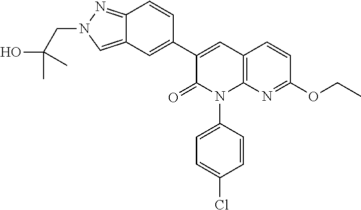 [SMILES: CCOc1ccc2cc(-c3ccc4nn(CC(C)(C)O)cc4c3)c(=O)n(-c3ccc(Cl)cc3)c2n1] | 196 |
| 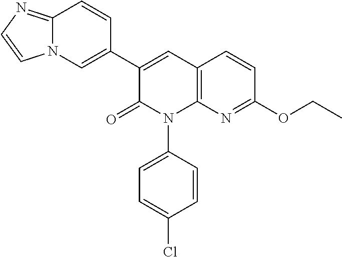 [SMILES: CCOc1ccc2cc(-c3ccc4nccn4c3)c(=O)n(-c3ccc(Cl)cc3)c2n1] | 197 |
| 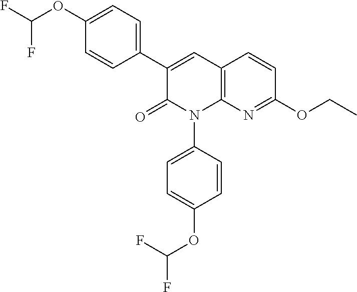 [SMILES: CCOc1ccc2cc(-c3ccc(OC(F)F)cc3)c(=O)n(-c3ccc(OC(F)F)cc3)c2n1] | 198 |
| 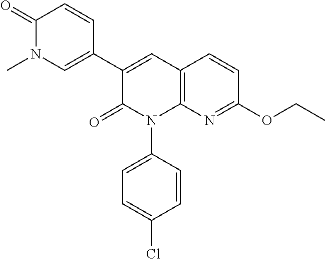 [SMILES: CCOc1ccc2cc(-c3ccc(=O)n(C)c3)c(=O)n(-c3ccc(Cl)cc3)c2n1] | 199 |
| 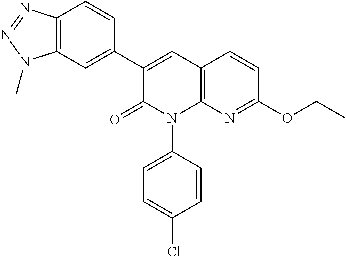 [SMILES: CCOc1ccc2cc(-c3ccc4nnn(C)c4c3)c(=O)n(-c3ccc(Cl)cc3)c2n1] | 200 |
| 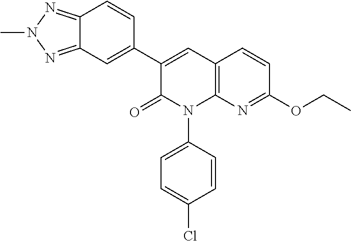 [SMILES: CCOc1ccc2cc(-c3ccc4nn(C)nc4c3)c(=O)n(-c3ccc(Cl)cc3)c2n1] | 201 |
| 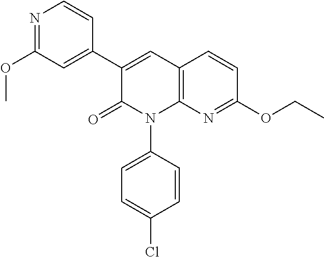 [SMILES: CCOc1ccc2cc(-c3ccnc(OC)c3)c(=O)n(-c3ccc(Cl)cc3)c2n1] | 202 |
| 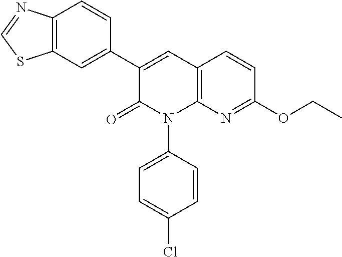 [SMILES: CCOc1ccc2cc(-c3ccc4ncsc4c3)c(=O)n(-c3ccc(Cl)cc3)c2n1] | 203 |
| 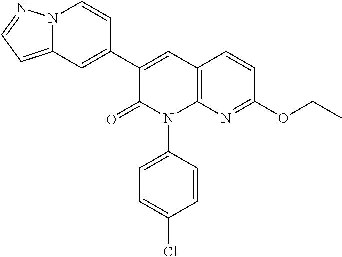 [SMILES: CCOc1ccc2cc(-c3ccn4nccc4c3)c(=O)n(-c3ccc(Cl)cc3)c2n1] | 204 |
| 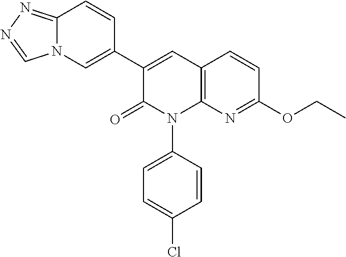 [SMILES: CCOc1ccc2cc(-c3ccc4nncn4c3)c(=O)n(-c3ccc(Cl)cc3)c2n1] | 205 |
| 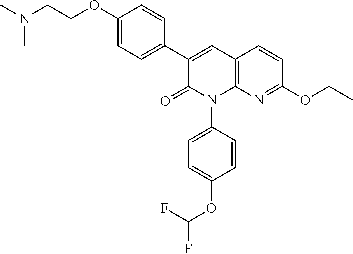 [SMILES: CCOc1ccc2cc(-c3ccc(OCCN(C)C)cc3)c(=O)n(-c3ccc(OC(F)F)cc3)c2n1] | 206 |
| 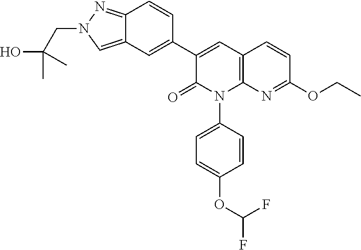 [SMILES: CCOc1ccc2cc(-c3ccc4nn(CC(C)(C)O)cc4c3)c(=O)n(-c3ccc(OC(F)F)cc3)c2n1] | 207 |
| 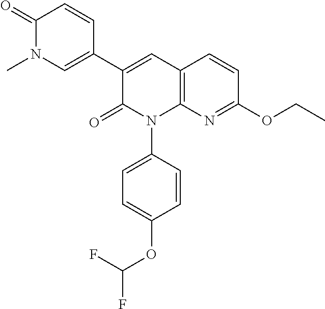 [SMILES: CCOc1ccc2cc(-c3ccc(=O)n(C)c3)c(=O)n(-c3ccc(OC(F)F)cc3)c2n1] | 208 |
| 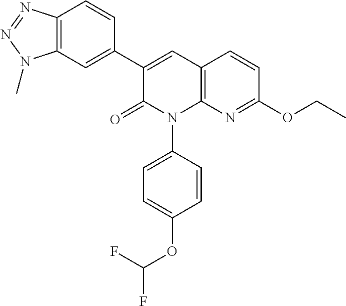 [SMILES: CCOc1ccc2cc(-c3ccc4nnn(C)c4c3)c(=O)n(-c3ccc(OC(F)F)cc3)c2n1] | 209 |
| 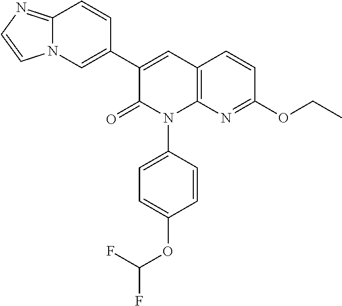 [SMILES: CCOc1ccc2cc(-c3ccc4nccn4c3)c(=O)n(-c3ccc(OC(F)F)cc3)c2n1] | 210 |
| 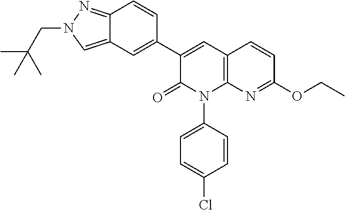 [SMILES: CCOc1ccc2cc(-c3ccc4nn(CC(C)(C)C)cc4c3)c(=O)n(-c3ccc(Cl)cc3)c2n1] | 211 |
| 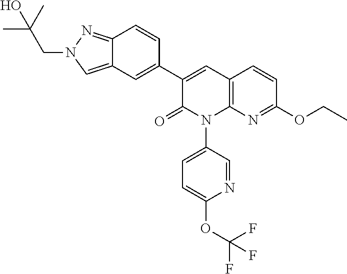 [SMILES: CCOc1ccc2cc(-c3ccc4nn(CC(C)(C)O)cc4c3)c(=O)n(-c3ccc(OC(F)(F)F)nc3)c2n1] | 212 |
| 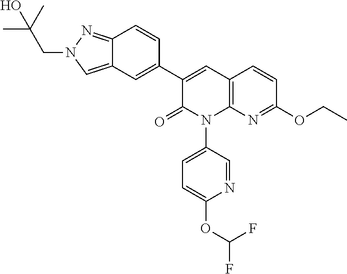 [SMILES: CCOc1ccc2cc(-c3ccc4nn(CC(C)(C)O)cc4c3)c(=O)n(-c3ccc(OC(F)F)nc3)c2n1] | 213 |
| 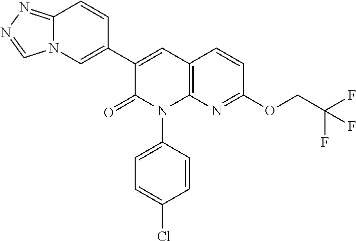 [SMILES: O=c1c(-c2ccc3nncn3c2)cc2ccc(OCC(F)(F)F)nc2n1-c1ccc(Cl)cc1] | 214 |
| 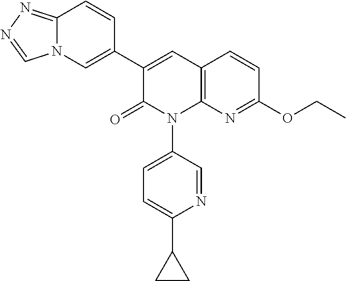 [SMILES: CCOc1ccc2cc(-c3ccc4nncn4c3)c(=O)n(-c3ccc(C4CC4)nc3)c2n1] | 215 |
| 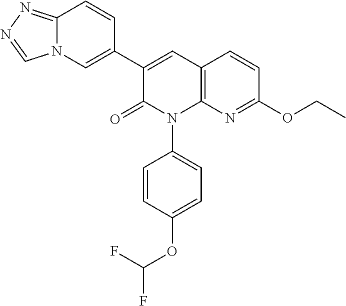 [SMILES: CCOc1ccc2cc(-c3ccc4nncn4c3)c(=O)n(-c3ccc(OC(F)F)cc3)c2n1] | 216 |
| 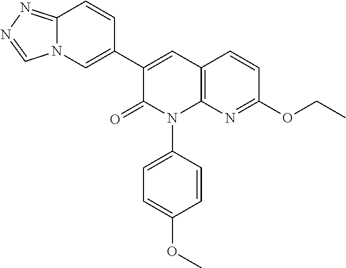 [SMILES: CCOc1ccc2cc(-c3ccc4nncn4c3)c(=O)n(-c3ccc(OC)cc3)c2n1] | 217 |
| 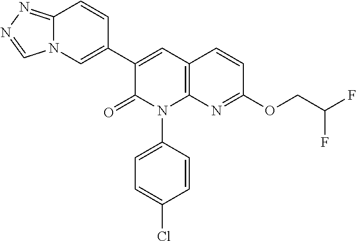 [SMILES: O=c1c(-c2ccc3nncn3c2)cc2ccc(OCC(F)F)nc2n1-c1ccc(Cl)cc1] | 218 |
| 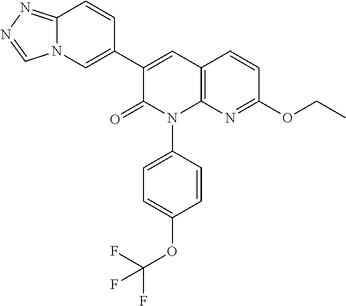 [SMILES: CCOc1ccc2cc(-c3ccc4nncn4c3)c(=O)n(-c3ccc(OC(F)(F)F)cc3)c2n1] | 219 |
| 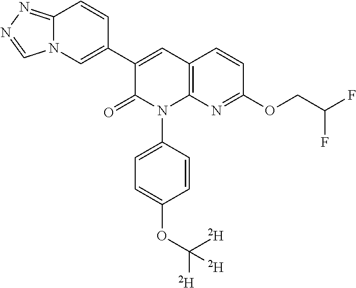 [SMILES: [2H]C([2H])([2H])Oc1ccc(-n2c(=O)c(-c3ccc4nncn4c3)cc3ccc(OCC(F)F)nc32)cc1] | 220 |
| 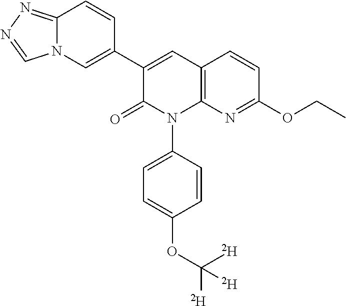 [SMILES: [2H]C([2H])([2H])Oc1ccc(-n2c(=O)c(-c3ccc4nncn4c3)cc3ccc(OCC)nc32)cc1] | 221 |
| 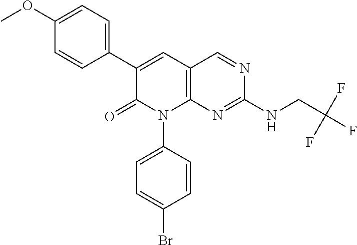 [SMILES: COc1ccc(-c2cc3cnc(NCC(F)(F)F)nc3n(-c3ccc(Br)cc3)c2=O)cc1] | 232 |
| 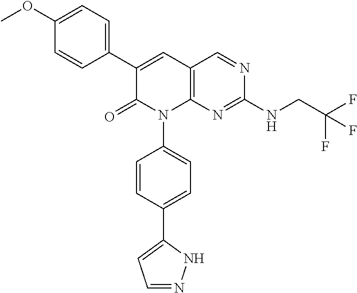 [SMILES: COc1ccc(-c2cc3cnc(NCC(F)(F)F)nc3n(-c3ccc(-c4ccn[nH]4)cc3)c2=O)cc1] | 233 |
| 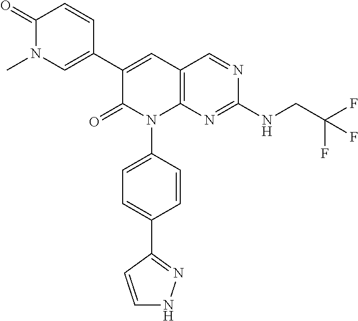 [SMILES: Cn1cc(-c2cc3cnc(NCC(F)(F)F)nc3n(-c3ccc(-c4cc[nH]n4)cc3)c2=O)ccc1=O] | 234 |
| 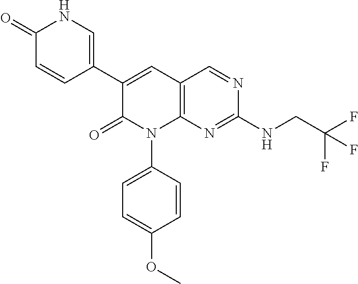 [SMILES: COc1ccc(-n2c(=O)c(-c3ccc(=O)[nH]c3)cc3cnc(NCC(F)(F)F)nc32)cc1] | 235 |
| 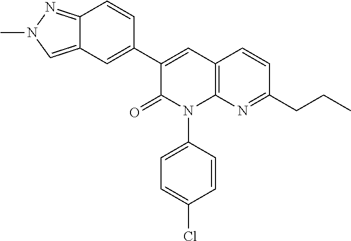 [SMILES: CCCc1ccc2cc(-c3ccc4nn(C)cc4c3)c(=O)n(-c3ccc(Cl)cc3)c2n1] | 236 |

36. The compound or pharmaceutically salt thereof according to Aspect 1, wherein the compound is selected from the following table:

| | |
|---|---|
| 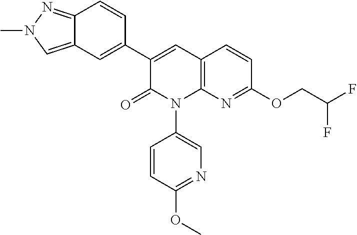 [SMILES: COc1ccc(-n2c(=O)c(-c3ccc4nn(C)cc4c3)cc3ccc(OCC(F)F)nc32)cn1] | 124 |
| 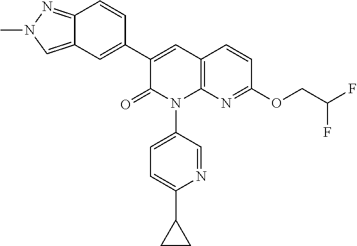 [SMILES: Cn1cc2cc(-c3cc4ccc(OCC(F)F)nc4n(-c4ccc(C5CC5)nc4)c3=O)ccc2n1] | 168 |
| 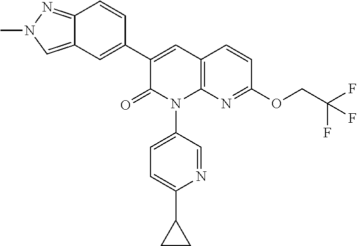 [SMILES: Cn1cc2cc(-c3cc4ccc(OCC(F)(F)F)nc4n(-c4ccc(C5CC5)nc4)c3=O)ccc2n1] | 166 |
| 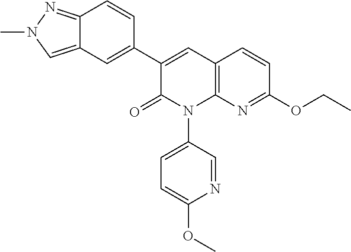 [SMILES: CCOc1ccc2cc(-c3ccc4nn(C)cc4c3)c(=O)n(-c3ccc(OC)nc3)c2n1] | 157 |
| 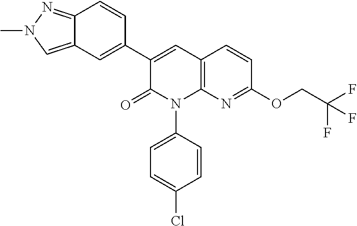 [SMILES: Cn1cc2cc(-c3cc4ccc(OCC(F)(F)F)nc4n(-c4ccc(Cl)cc4)c3=O)ccc2n1] | 122 |
| 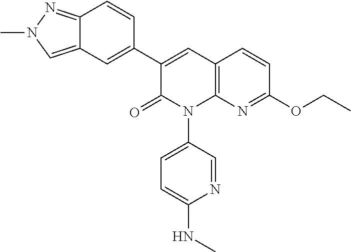 [SMILES: CCOc1ccc2cc(-c3ccc4nn(C)cc4c3)c(=O)n(-c3ccc(NC)nc3)c2n1] | 144 |
| 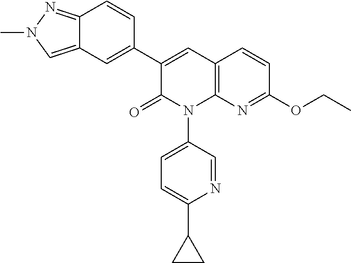 [SMILES: CCOc1ccc2cc(-c3ccc4nn(C)cc4c3)c(=O)n(-c3ccc(C4CC4)nc3)c2n1] | 161 |
| 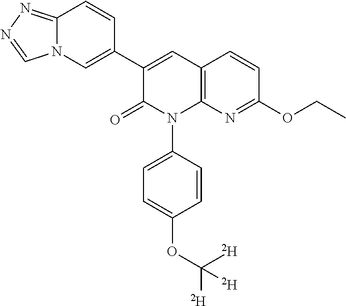 [SMILES: [2H]C([2H])([2H])Oc1ccc(-n2c(=O)c(-c3ccc4nncn4c3)cc3ccc(OCC)nc32)cc1] | 221 |
| 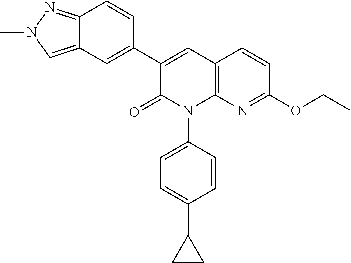 [SMILES: CCOc1ccc2cc(-c3ccc4nn(C)cc4c3)c(=O)n(-c3ccc(C4CC4)cc3)c2n1] | 165 |
| 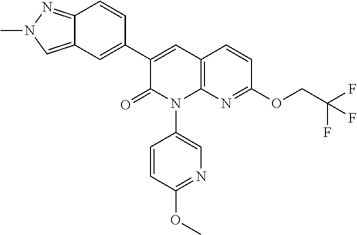 [SMILES: COc1ccc(-n2c(=O)c(-c3ccc4nn(C)cc4c3)cc3ccc(OCC(F)(F)F)nc32)cn1] | 123 |
| 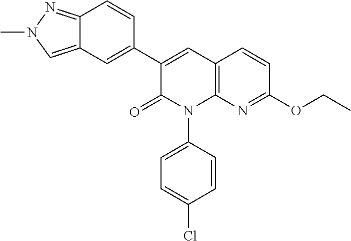 [SMILES: CCOc1ccc2cc(-c3ccc4nn(C)cc4c3)c(=O)n(-c3ccc(Cl)cc3)c2n1] | 138 |
| 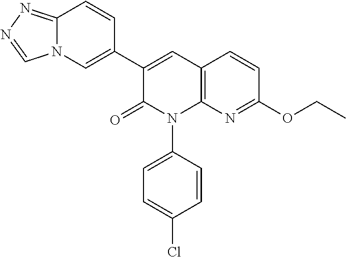 [SMILES: CCOc1ccc2cc(-c3ccc4nncn4c3)c(=O)n(-c3ccc(Cl)cc3)c2n1] | 205 |
| 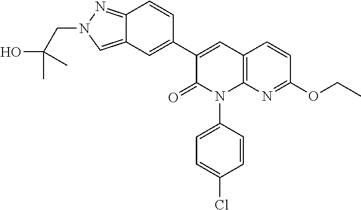 [SMILES: CCOc1ccc2cc(-c3ccc4nn(CC(C)(C)O)cc4c3)c(=O)n(-c3ccc(Cl)cc3)c2n1] | 196 |
| 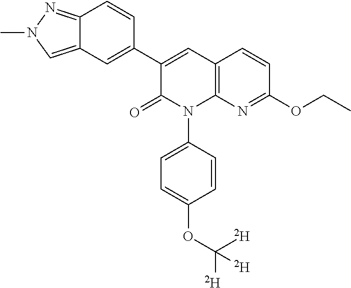 [SMILES: [2H]C([2H])([2H])Oc1ccc(-n2c(=O)c(-c3ccc4nn(C)cc4c3)cc3ccc(OCC)nc32)cc1] | 148 |
| 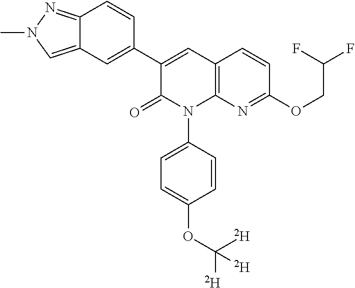 [SMILES: [2H]C([2H])([2H])Oc1ccc(-n2c(=O)c(-c3ccc4nn(C)cc4c3)cc3ccc(OCC(F)F)nc32)cc1] | 149 |
| 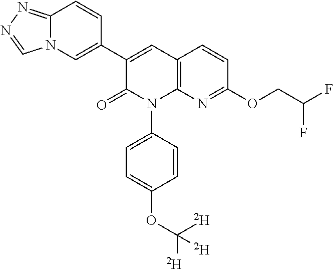 [SMILES: [2H]C([2H])([2H])Oc1ccc(-n2c(=O)c(-c3ccc4nncn4c3)cc3ccc(OCC(F)F)nc32)cc1] | 220 |
| 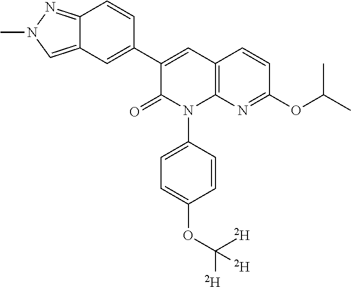 [SMILES: [2H]C([2H])([2H])Oc1ccc(-n2c(=O)c(-c3ccc4nn(C)cc4c3)cc3ccc(OC(C)C)nc32)cc1] | |
| 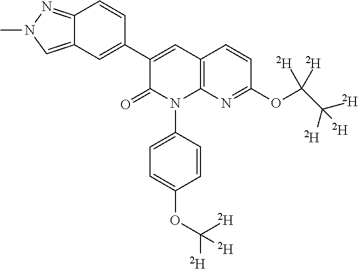 [SMILES: [2H]C([2H])([2H])Oc1ccc(-n2c(=O)c(-c3ccc4nn(C)cc4c3)cc3ccc(OC([2H])([2H])C([2H])([2H])[2H])nc32)cc1] | |
| 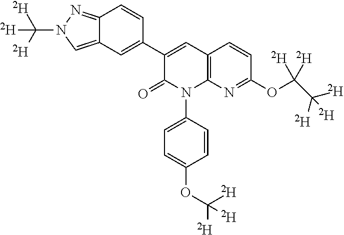 [SMILES: [2H]C([2H])([2H])Oc1ccc(-n2c(=O)c(-c3ccc4nn(C([2H])([2H])[2H])cc4c3)cc3ccc(OC([2H])([2H])C([2H])([2H])[2H])nc32)cc1] | |
| 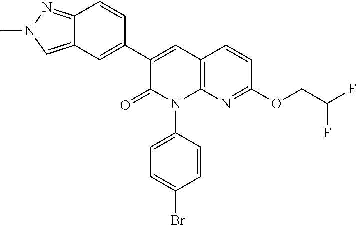 [SMILES: Cn1cc2cc(-c3cc4ccc(OCC(F)F)nc4n(-c4ccc(Br)cc4)c3=O)ccc2n1] | |
| 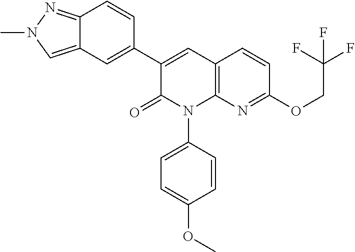 [SMILES: COc1ccc(-n2c(=O)c(-c3ccc4nn(C)cc4c3)cc3ccc(OCC(F)(F)F)nc32)cc1] | |
| 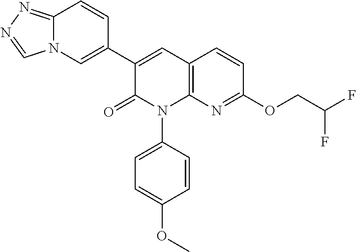 [SMILES: COc1ccc(-n2c(=O)c(-c3ccc4nncn4c3)cc3ccc(OCC(F)F)nc32)cc1] | |
| 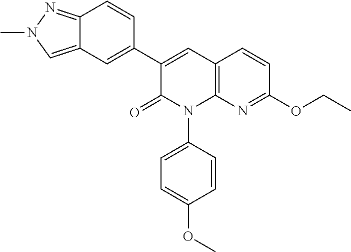 [SMILES: CCOc1ccc2cc(-c3ccc4nn(C)cc4c3)c(=O)n(-c3ccc(OC)cc3)c2n1] | |
| 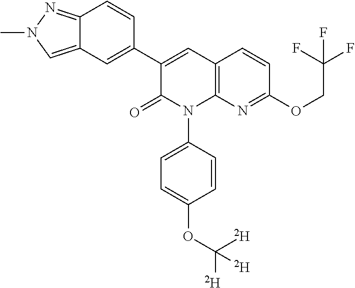 [SMILES: [2H]C([2H])([2H])Oc1ccc(-n2c(=O)c(-c3ccc4nn(C)cc4c3)cc3ccc(OCC(F)(F)F)nc32)cc1] | |
| 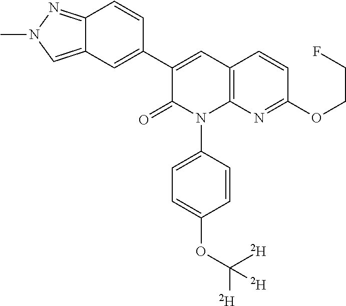 [SMILES: [2H]C([2H])([2H])Oc1ccc(-n2c(=O)c(-c3ccc4nn(C)cc4c3)cc3ccc(OCCF)nc32)cc1] | |
| 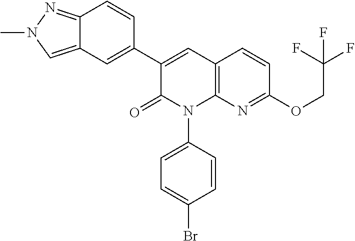 [SMILES: Cn1cc2cc(-c3cc4ccc(OCC(F)(F)F)nc4n(-c4ccc(Br)cc4)c3=O)ccc2n1] | |
| 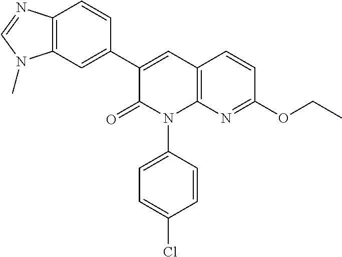 [SMILES: CCOc1ccc2cc(-c3ccc4ncn(C)c4c3)c(=O)n(-c3ccc(Cl)cc3)c2n1] | |
| 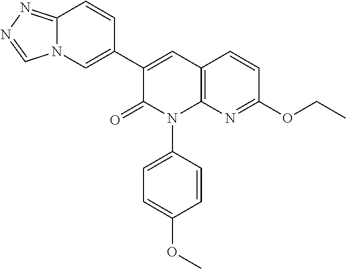 [SMILES: CCOc1ccc2cc(-c3ccc4nncn4c3)c(=O)n(-c3ccc(OC)cc3)c2n1] | |
| 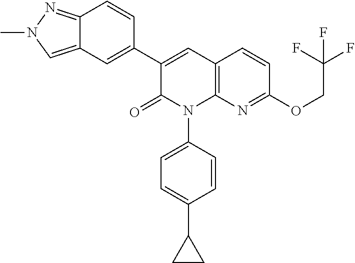 [SMILES: Cn1cc2cc(-c3cc4ccc(OCC(F)(F)F)nc4n(-c4ccc(C5CC5)cc4)c3=O)ccc2n1] | |
| 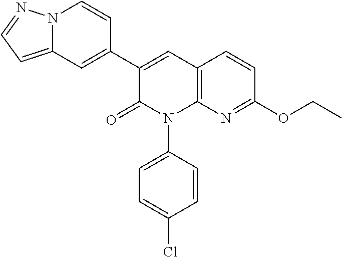 [SMILES: CCOc1ccc2cc(-c3ccn4nccc4c3)c(=O)n(-c3ccc(Cl)cc3)c2n1] | |
| 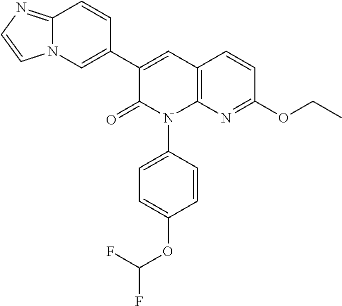 [SMILES: CCOc1ccc2cc(-c3ccc4nccn4c3)c(=O)n(-c3ccc(OC(F)F)cc3)c2n1] | |
| 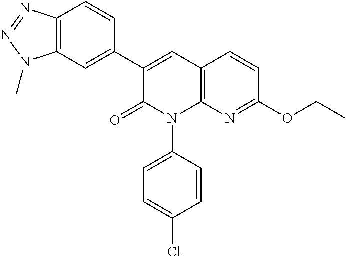 [SMILES: CCOc1ccc2cc(-c3ccc4nnn(C)c4c3)c(=O)n(-c3ccc(Cl)cc3)c2n1] | |
| 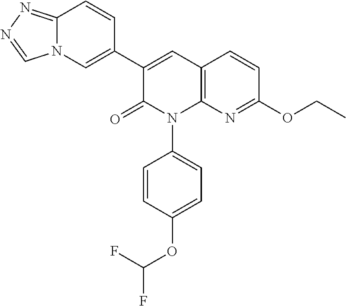 [SMILES: CCOc1ccc2cc(-c3ccc4nncn4c3)c(=O)n(-c3ccc(OC(F)F)cc3)c2n1] | |
| 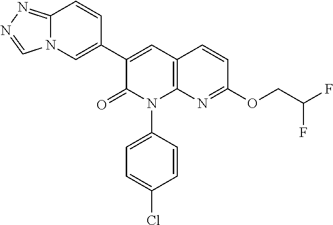 [SMILES: O=c1c(-c2ccc3nncn3c2)cc2ccc(OCC(F)F)nc2n1-c1ccc(Cl)cc1] | |
| 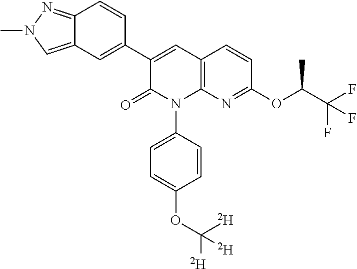 [SMILES: [2H]C([2H])([2H])Oc1ccc(-n2c(=O)c(-c3ccc4nn(C)cc4c3)cc3ccc(O[C@@H](C)C(F)(F)F)nc32)cc1] | |
| 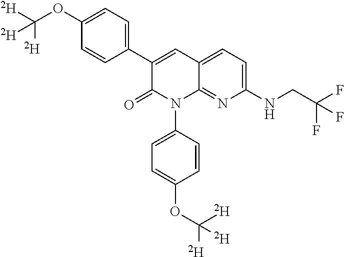 [SMILES: [2H]C([2H])([2H])Oc1ccc(-c2cc3ccc(NCC(F)(F)F)nc3n(-c3ccc(OC([2H])([2H])[2H])cc3)c2=O)cc1] | |

37. The compound or pharmaceutically acceptable salt thereof according to Aspect 2, wherein the compound is selected from the following table:

| | |
|---|---|
| 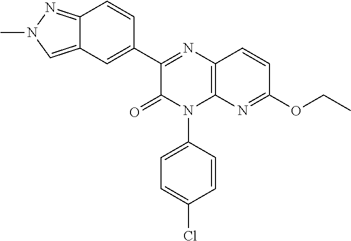 [SMILES: CCOc1ccc2nc(-c3ccc4nn(C)cc4c3)c(=O)n(-c3ccc(Cl)cc3)c2n1] | 222 |
| 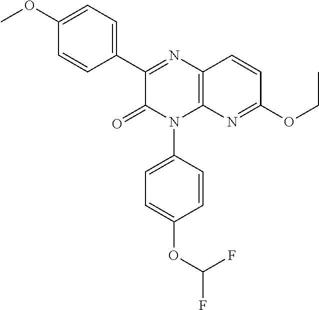 [SMILES: CCOc1ccc2nc(-c3ccc(OC)cc3)c(=O)n(-c3ccc(OC(F)F)cc3)c2n1] | 223 |
| 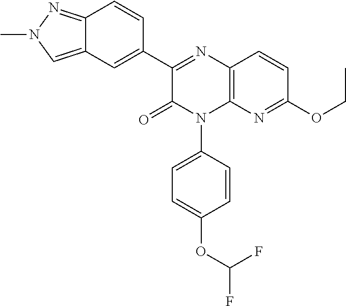 [SMILES: CCOc1ccc2nc(-c3ccc4nn(C)cc4c3)c(=O)n(-c3ccc(OC(F)F)cc3)c2n1] | 224 |
| 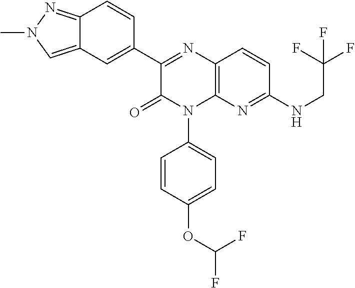 [SMILES: Cn1cc2cc(-c3nc4ccc(NCC(F)(F)F)nc4n(-c4ccc(OC(F)F)cc4)c3=O)ccc2n1] | 225 |
| 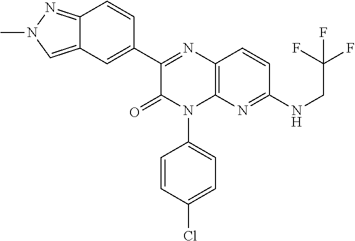 [SMILES: Cn1cc2cc(-c3nc4ccc(NCC(F)(F)F)nc4n(-c4ccc(Cl)cc4)c3=O)ccc2n1] | 226 |
| 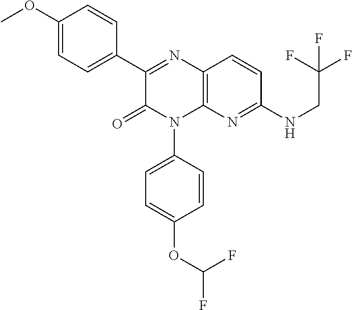 [SMILES: COc1ccc(-c2nc3ccc(NCC(F)(F)F)nc3n(-c3ccc(OC(F)F)cc3)c2=O)cc1] | 227 |
| 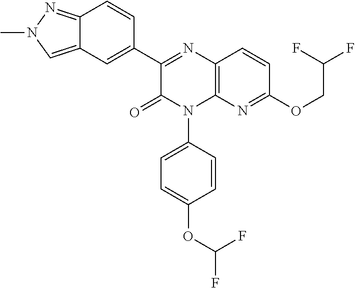 [SMILES: Cn1cc2cc(-c3nc4ccc(OCC(F)F)nc4n(-c4ccc(OC(F)F)cc4)c3=O)ccc2n1] | 228 |
| 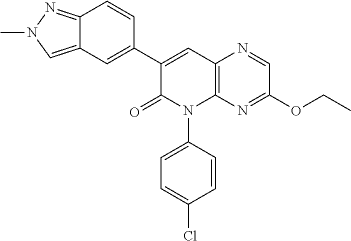 [SMILES: CCOc1cnc2cc(-c3ccc4nn(C)cc4c3)c(=O)n(-c3ccc(Cl)cc3)c2n1] | 229 |
| 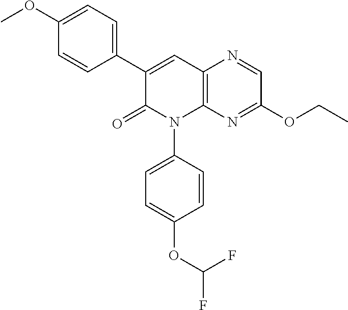 [SMILES: CCOc1cnc2cc(-c3ccc(OC)cc3)c(=O)n(-c3ccc(OC(F)F)cc3)c2n1] | 230 |
| 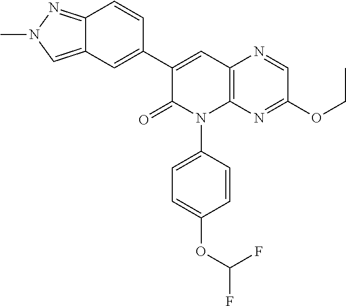 [SMILES: CCOc1cnc2cc(-c3ccc4nn(C)cc4c3)c(=O)n(-c3ccc(OC(F)F)cc3)c2n1] | 231 |
| 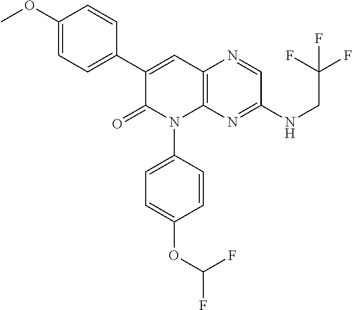 [SMILES: COc1ccc(-c2cc3ncc(NCC(F)(F)F)nc3n(-c3ccc(OC(F)F)cc3)c2=O)cc1] | 232 |

Aspect 38. The compound or pharmaceutically acceptable salt thereof according to Aspect 2, wherein the compound is selected from the following table:

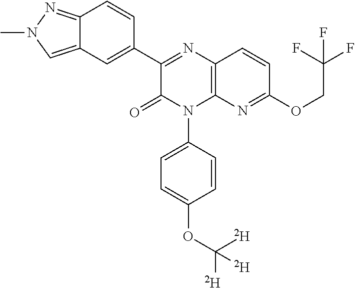 [SMILES: [2H]C([2H])([2H])Oc1ccc(-n2c(=O)c(-c3ccc4nn(C)cc4c3)nc3ccc(OCC(F)(F)F)nc32)cc1]

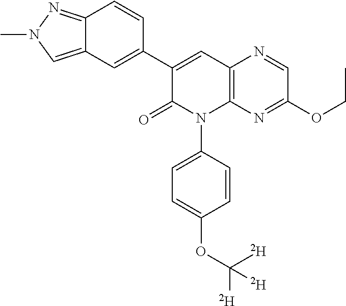 [SMILES: [2H]C([2H])([2H])Oc1ccc(-n2c(=O)c(-c3ccc4nn(C)cc4c3)cc3ncc(OCC)nc32)cc1]

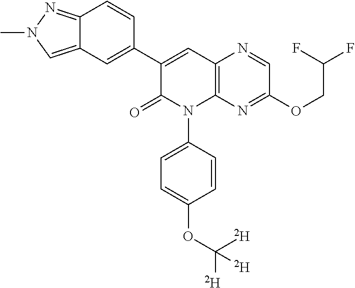 [SMILES: [2H]C([2H])([2H])Oc1ccc(-n2c(=O)c(-c3ccc4nn(C)cc4c3)cc3ncc(OCC(F)F)nc32)cc1]

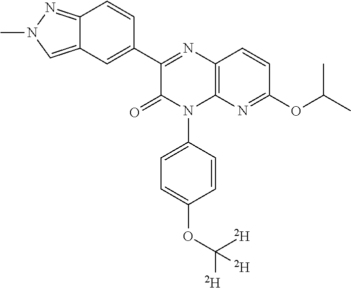 [SMILES: [2H]C([2H])([2H])Oc1ccc(-n2c(=O)c(-c3ccc4nn(C)cc4c3)nc3ccc(OC(C)C)nc32)cc1]

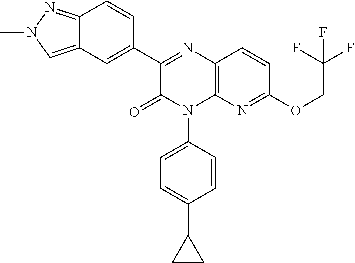 [SMILES: Cn1cc2cc(-c3nc4ccc(OCC(F)(F)F)nc4n(-c4ccc(C5CC5)cc4)c3=O)ccc2n1]

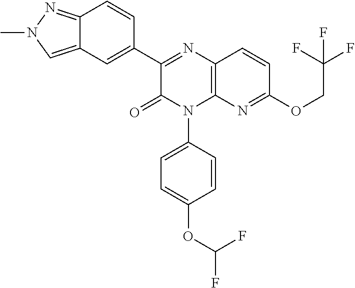 [SMILES: Cn1cc2cc(-c3nc4ccc(OCC(F)(F)F)nc4n(-c4ccc(OC(F)F)cc4)c3=O)ccc2n1]

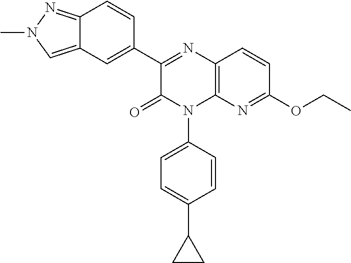 [SMILES: CCOc1ccc2nc(-c3ccc4nn(C)cc4c3)c(=O)n(-c3ccc(C4CC4)cc3)c2n1]

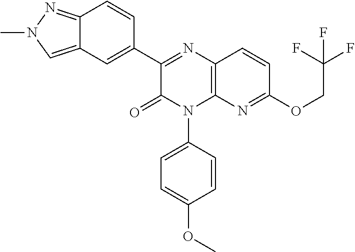 [SMILES: COc1ccc(-n2c(=O)c(-c3ccc4nn(C)cc4c3)nc3ccc(OCC(F)(F)F)nc32)cc1]

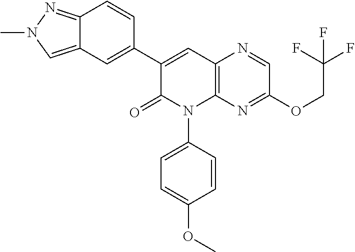 [SMILES: COc1ccc(-n2c(=O)c(-c3ccc4nn(C)cc4c3)cc3ncc(OCC(F)(F)F)nc32)cc1]

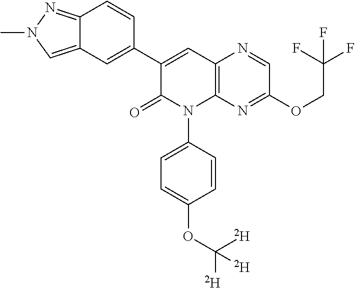 [SMILES: [2H]C([2H])([2H])Oc1ccc(-n2c(=O)c(-c3ccc4nn(C)cc4c3)cc3ncc(OCC(F)(F)F)nc32)cc1]

Aspect 39. A pharmaceutical composition comprising a therapeutically effective amount of a compound or pharmaceutically acceptable salt thereof according to any one of Aspects 1 to 37 or a pharmaceutically acceptable salt thereof, and a pharmaceutically acceptable carrier.

Aspect 40. A method for treating a cancer in a subject suffering therefrom, comprising administering to the subject an effective amount of a MAT2A inhibitor.

Aspect 41. The method according to Aspect 40, wherein the cancer is an MTAP-deleted cancer.

Aspect 42. A method for inhibiting the synthesis of S-adenosyl methionine (SAM) in a cell, comprising introducing into the cell an effective amount of a compound, or a pharmaceutically acceptable salt thereof, according to any one of Aspects 1 to 37.

Aspect 43. A method for inhibiting the synthesis of S-adenosyl methionine (SAM) in a subject, comprising administering to the subject an effective amount of at least one compound or pharmaceutically acceptable salt thereof according to any one of Aspects 1 to 37.

Aspect 44. A method for treating a cancer in a subject suffering therefrom, comprising administering to the subject an effective amount of a compound or pharmaceutically acceptable salt thereof according to any one of Aspects 1 to 37.

Aspect 45. The method according to Aspect 44, wherein the cancer is an MTAP-deleted cancer.

Aspect 46. The method according to Aspect 40, 44, or 45, wherein the cancer is selected from the group consisting of mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

Aspect 47. The method according to Aspect 40, 44, or 45, wherein the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, and breast cancer.

Aspect 48. The method according to Aspect 47, wherein the cancer is a lung cancer is selected from the group consisting of non-small cell lung cancer, small cell lung cancer, adenocarcinoma of the lung, and squamous cell carcinoma of the lung.

Aspect 49. The method according to Aspect 47, wherein the cancer is a brain tumor selected from the group consisting of glioma, glioblastoma, astrocytoma, meningioma, medulloblastoma, peripheral neuroectodermal tumors, and craniopharyngioma.

Aspect 50. The method according to Aspect 47, wherein the cancer is triple negative breast cancer (TNBC).

Aspect 51. The method according to Aspect 47, wherein the cancer is a lymphoma selected from the group consisting of mantle cell lymphoma, Hodgkin lymphoma, non-Hodgkin lymphoma, Burkitt lymphoma, diffuse large B-cell lymphoma, and adult T-cell leukemia/lymphoma.

Aspect 52. A method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, as compared to cancers where the MTAP gene or protein is present and/or fully functioning, the method comprising administering to the subject a therapeutically effective amount of a compound, or a pharmaceutically acceptable salt thereof, according to any one of Aspects 1 to 38.

Aspect 53. A compound according to any one of Aspects 1 to 38, or a pharmaceutically acceptable salt thereof, for use in inhibiting the synthesis of S-adenosyl methionine (SAM).

Aspect 54. A compound according to any one of Aspects 1 to 38, or a pharmaceutically acceptable salt thereof, for use intreating a cancer in a subject suffering therefrom.

Aspect 55. The compound or pharmaceutically acceptable salt thereof according to Aspect 54, wherein the cancer is an MTAP-deleted cancer.

Aspect 56. The compound or pharmaceutically acceptable salt thereof according to Aspect 54 or 55, wherein the cancer is selected from the group consisting of mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

Aspect 57. The compound or pharmaceutically acceptable salt thereof according to Aspect 54 or 55, wherein the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, and breast cancer.

Aspect 58. The compound or pharmaceutically acceptable salt thereof according to Aspect 57, wherein the cancer is a lung cancer is selected from the group consisting of non-small cell lung cancer, small cell lung cancer, adenocarcinoma of the lung, and squamous cell carcinoma of the lung.

Aspect 59. The compound or pharmaceutically acceptable salt thereof according to Aspect 57, wherein the cancer is triple negative breast cancer (TNBC).

Aspect 60. The compound or pharmaceutically acceptable salt thereof according to Aspect 57, wherein the cancer is a brain tumor selected from the group consisting of glioma, glioblastoma, astrocytoma, meningioma, medulloblastoma, peripheral neuroectodermal tumors, and craniopharyngioma.

Aspect 61. The compound or pharmaceutically acceptable salt thereof according to Aspect 57, wherein the cancer is a lymphoma selected from the group consisting of mantle cell lymphoma, Hodgkin lymphoma, non-Hodgkin lymphoma, Burkitt lymphoma, diffuse large B-cell lymphoma (DLBCL), and adult T-cell leukemia/lymphoma.

### EXAMPLES

The following non-limiting examples are additional embodiments for illustrating the present disclosure.

Units and Terms List:
anhy. anhydrous
aq. aqueous
min minute(s)
mL milliliter
mmol millimole(s)
mol mole(s)
MS mass spectrometry
NMR nuclear magnetic resonance
TLC thin layer chromatography
HPLC high-performance liquid chromatography
RT(r.t.) room temperature

NMR Spectra
Hz hertz
δ chemical shift
J coupling constant
s singlet
d doublet
t triplet
q quartet
m multiplet
br broad
qd quartet of doublets
dquin doublet of quintets
dd doublet of doublets
dt doublet of triplets

Solvents and Reagents:
CHCl₃ chloroform
DCM dichloromethane
DMF dimethylformamide
Et₂O diethyl ether
EtOH ethyl alcohol
EtOAc ethyl acetate
EA ethyl acetate
MeOH methyl alcohol
MeCN acetonitrile
PE petroleum ether
THE tetrahydrofuran
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃ sodium bicarbonate
LiHMDS lithium hexamethyldisilylamide
NaHMDS sodium hexamethyldisilylamide
LAH lithium aluminum hydride
NaBH₄ sodium borohydride
LDA lithium diisopropylamide
Et₃N triethylamine
DMAP 4-(dimethylamino)pyridine
DIPEA N,N-diisopropylethylamine
NH₄OH ammonium hydroxide
EDCI 1-ethyl-3-(3-dimethylaminopropyl)carbodiimide
HOBt 1-hydroxybenzotriazole
HATU O-(7-azabenzotriazol-1-yl)-N,N,N′,N′-tetra-methyluronium
Xphos 2-Dicyclohexylphosphino-2′,4′,6′-triisopropylbiphenyl
BINAP 2,2′-bis(diphenylphosphanyl)-1,1′-binaphthyl

General Experimental

In the following examples, the reagents and solvents were purchased from commercial sources (such as Alfa, Acros, Sigma Aldrich, TCI and Shanghai Chemical Reagent Company), and used without further purification unless otherwise specified. Flash chromatography was performed on an Ez Purifier III using column with silica gel particles of 200-300 mesh. Analytical and preparative thin layer chromatography (TLC) plates were HSGF 254 (0.15-0.2 mm thickness, Shanghai Anbang Company, China). Nuclear magnetic resonance (NMR) spectra were obtained on a Brucker AMX-400 NMR (Brucker, Switzerland). Chemical shifts were reported in parts per million (ppm, δ) downfield from tetramethylsilane. Mass spectra were given with electrospray ionization (ESI) from a Waters LCT TOF Mass Spectrometer (Waters, USA). HPLC chromatographs were record on an Agilent 1200 Liquid Chromatography (Agilent, USA, column: Ultimate 4.6 mm×50 mm, 5 μm, mobile phase A: 0.1% formic acid in water; mobile phase B: acetonitrile). Microwave reactions were run on an Initiator 2.5 Microwave Synthesizer (Biotage, Sweden).

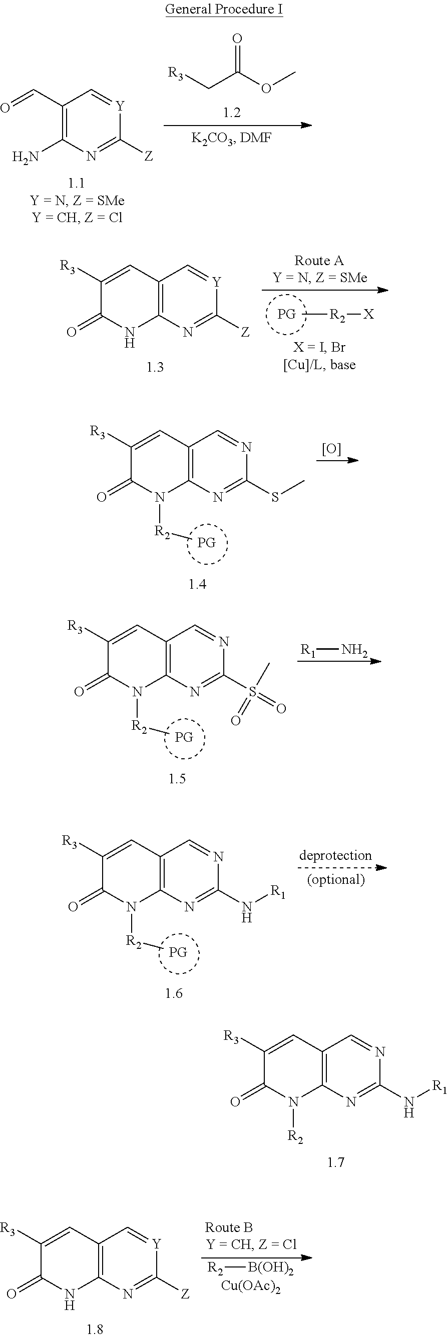

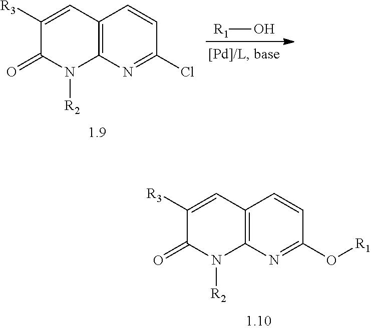

Compounds of structure 1.7 and 1.10 were obtained through the scheme depicted as General Procedure I. Beginning with heterocycle 1.1 (optionally substituted at positions Y and Z as shown), the desired core structure 1.3 was generated through an amidation-condensation sequence with ester 1.2. For Route A (where Y=N and Z=SMe), the desired and optionally protected R2 group was introduced through a copper mediated Ullmann coupling to obtain compound 1.4. Thioether 1.4 was then oxidized to sulfoxide 1.5, allowing for the desired R₁ to be installed using a nucleophilic aromatic substitution reaction. If necessary, compound 1.6 was then deprotected to afford compounds of structure 1.7.

Alternatively, when Route B was employed (where Y=CH and Z=Cl), the desired R₂ group was introduced using a copper mediated Chan-Lam coupling to generate compound 1.9. The desired R₁ group was then introduced using a palladium mediated Buchwald-Hartwig coupling to afford compounds of structure 1.10.

### Preparation of Example 101 via General Procedure I (Route A, Y=N, Z=SMe

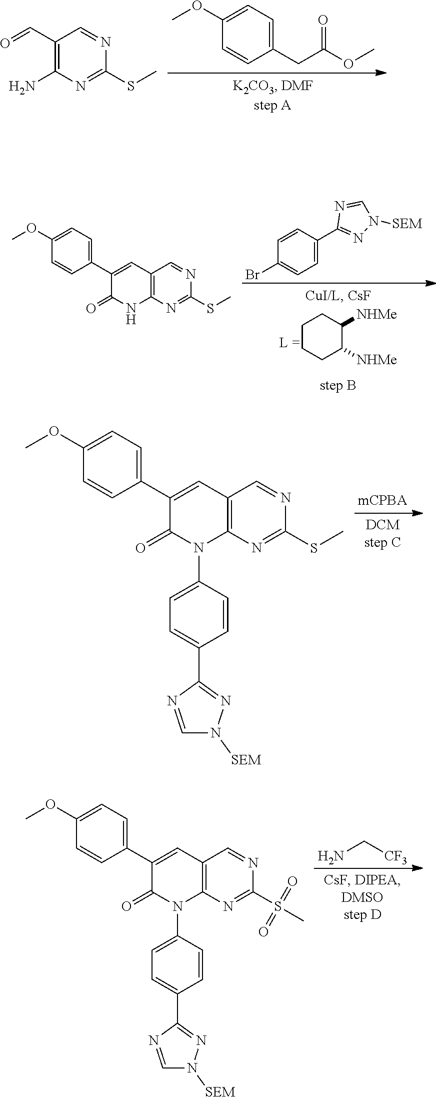

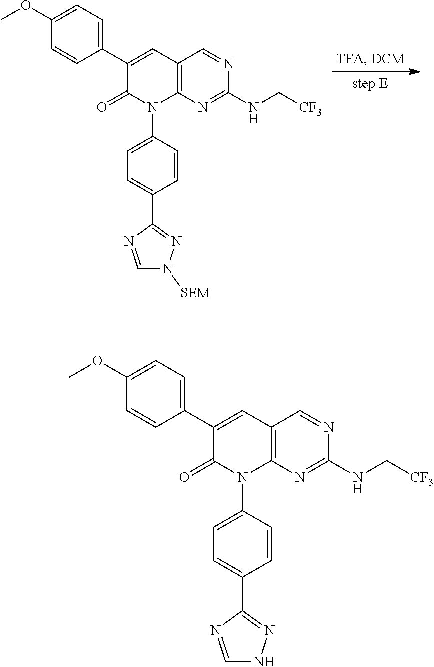 [SMILES: COc1ccc(-c2cc3cnc(NCC(F)(F)F)nc3n(-c3ccc(-c4ncn(COCC[Si](C)(C)C)n4)cc3)c2=O)cc1 ; COc1ccc(-c2cc3cnc(NCC(F)(F)F)nc3n(-c3ccc(-c4nc[nH]n4)cc3)c2=O)cc1]

### Step A: 6-(4-methoxyphenyl)-2-(methylthio)pyrido[2,3-d]pyrimidin-7(8H)-one

To a solution of 4-amino-2-(methylthio)pyrimidine-5-carbaldehyde (900 mg, 5.3 mmol, 1.0 eq.) in DMF (10 mL) was added K₂CO₃ (2.2 g, 15.9 mmol, 3.0 eq.) and methyl 2-(4-methoxyphenyl)acetate (1.2 g, 6.7 mmol, 1.2 eq.) at room temperature. The resulting mixture was stirred at 100° C. for 2 hrs. Then the reaction was quenched with ice water (20 mL), the resulting precipitate was filtered, the filter cake was collected and dried under reduced pressure to afford 6-(4-methoxyphenyl)-2-(methylthio)pyrido[2,3-d]pyrimidin-7(8H)-one (1.4 g, 88% yield) as a white solid. LC-MS (ESI): m/z 300 [M+H]⁺.

### Step B: 6-(4-methoxyphenyl)-2-(methylthio)-8-(4-(1-((2-(trimethylsilyl)ethoxy)methyl)-1H-1,2,4-triazol-3-yl)phenyl)pyrido[2,3-d]pyrimidin-7(8H)-one

To a solution of 6-(4-methoxyphenyl)-2-(methylthio)pyrido[2,3-d]pyrimidin-7(8H)-one (200 mg, 0.67 mmol, 1.0 eq.) in MeCN (10 mL) was added CuI (127 mg, 0.67 mmol, 1.0 eq.), (1R,2R)—N1,N2-dimethylcyclohexane-1,2-diamine (190 mg, 1.34 mmol, 2.0 eq.), CsF (305 mg, 2.0 mmol, 3.0 eq.) and 3-(4-bromophenyl)-1-((2-(trimethylsilyl)ethoxy)methyl)-1H-1,2,4-triazole (355 mg, 1.0 mmol, 1.5 eq.) (Ref: WO 2008/156726A1) at room temperature. The resulting mixture was stirred at 90° C. for 14 hrs. The reaction mixture was quenched with ice water (30 mL) and extracted with ethyl acetate (20 mL×3). The combined organic layers were washed with brine (20 mL), dried over Na₂SO₄ and concentrated under reduced pressure, the residue was purified by flash column chromatography on silica gel to afford 6-(4-methoxyphenyl)-2-(methylthio)-8-(4-(1-((2-(trimethylsilyl)ethoxy)methyl)-1H-1,2,4-triazol-3-yl)phenyl)pyrido[2,3-d]pyrimidin-7(8H)-one (300 mg, 78% yield) as a white solid. LC-MS (ESI): m/z 573 [M+H]⁺.

### Step C: 6-(4-methoxyphenyl)-2-(methylsulfonyl)-8-(4-(1-((2-(trimethylsilyl)ethoxy)methyl)-1H-1,2,4-triazol-3-yl)phenyl)pyrido[2,3-d]pyrimidin-7(8H)-one

A mixture of 6-(4-methoxyphenyl)-2-(methylthio)-8-(4-(1-((2-(trimethylsilyl)ethoxy)methyl)-1H-1,2,4-triazol-3-yl)phenyl)pyrido[2,3-d]pyrimidin-7(8H)-one (150 mg, 0.26 mmol, 1.0 eq.) and mCPBA (135 mg, 0.78 mmol, 3.0 eq.) in DCM (10 mL) was stirred at room temperature for 2 hrs. The reaction mixture was quenched with ice water (20 mL) and extracted with DCM (10 mL×3). The combined organic layers were washed with brine (20 mL), dried over Na₂SO₄ and concentrated under reduced pressure. The residue was purified by flash column chromatography on silica gel to afford 6-(4-methoxyphenyl)-2-(methylsulfonyl)-8-(4-(1-((2-(trimethylsilyl)ethoxy)methyl)-1H-1,2,4-triazol-3-yl)phenyl)pyrido[2,3-d]pyrimidin-7(8H)-one (150 mg, 95% yield) as a white solid. LC-MS (ESI): m/z 605 [M+H]⁺.

### Step D: 6-(4-methoxyphenyl)-2-((2,2,2-trifluoroethyl)amino)-8-(4-(1-((2-(trimethylsilyl)ethoxy)methyl)-1H-1,2,4-triazol-3-yl)phenyl)pyrido[2,3-d]pyrimidin-7(8H)-one

A mixture of 6-(4-methoxyphenyl)-2-(methylsulfonyl)-8-(4-(1-((2-(trimethylsilyl)ethoxy)methyl)-1H-1,2,4-triazol-3-yl)phenyl)pyrido[2,3-d]pyrimidin-7(8H)-one (50 mg, 0.08 mmol, 1.0 eq.), CsF (12 mg, 0.08 mmol, 1.0 eq.), DIPEA (31 mg, 0.24 mmol, 3.0 eq.) and 2,2,2-trifluoroethanamine (40 mg, 0.4 mmol, 5.0 eq.) in DMSO (1 mL) was stirred in a sealed tube for 14 hrs at 80° C. The reaction mixture was quenched with ice water (10 mL) and extracted with DCM (10 mL×3). The combined organic layers were washed with brine (20 mL), dried over Na₂SO₄ and concentrated under reduced pressure. The residue was purified by RP-prep-HPLC to afford 6-(4-methoxyphenyl)-2-((2,2,2-trifluoroethyl)amino)-8-(4-(1-((2-(trimethylsilyl)ethoxy)methyl)-1H-1,2,4-triazol-3-yl)phenyl)pyrido[2,3-d]pyrimidin-7(8H)-one (20 mg, 40% yield) as a white solid. LC-MS (ESI): m/z 624 [M+H]⁺.

### Step E: 8-(4-(1H-1,2,4-triazol-3-yl)phenyl)-6-(4-methoxyphenyl)-2-(2,2,2-trifluoroethylamino)pyrido[2,3-d]pyrimidin-7(8H)-one

To a solution of 6-(4-methoxyphenyl)-2-((2,2,2-trifluoroethyl)amino)-8-(4-(1-((2-(trimethylsilyl)ethoxy)methyl)-1H-1,2,4-triazol-3-yl)phenyl)pyrido[2,3-d]pyrimidin-7(8H)-one (20 mg, 0.03 mmol, 1.0 eq.) in DCM (1 mL) was added TFA (0.2 mL) at room temperature. The resulting mixture was stirred for 1 hr before being neutralized with NaHCO₃ (sat. aq.) at 0° C. to a final pH=8. The resulting mixture was extracted with DCM (5 mL×3), the combined organic layers were washed with brine (10 mL), dried over Na₂SO₄ and concentrated under reduced pressure. The residue was purified by RP-prep-HPLC to afford 8-(4-(1H-1,2,4-triazol-3-yl)phenyl)-6-(4-methoxyphenyl)-2-(2,2,2-trifluoroethylamino)pyrido[2,3-d]pyrimidin-7(8H)-one (Example 101).

¹H NMR (400 hz, DMSO-d₆) (the ratio of two tautomers 1:1) δ: 14.21 (br, 1H), 8.85-8.77 (m, 1H), 8.69-8.51 (m, 1H), 8.33-8.25 (m, 0.5H), 8.15 (d, J=8.4 Hz, 2H), 8.06 (s, 1H), 8.03-7.95 (m, 0.5H), 7.66 (d, J=8.8 Hz, 2H), 7.51-7.40 (m, 2H), 6.99 (d, J=8.8 Hz, 2H), 4.18-4.06 (m, 1H), 3.79 (s, 3H), 3.77-3.67 (m, 1H). LC-MS (ESI): m/z 494 [M+H]⁺.

The procedure set forth above for General Procedure I (Route A, Y=N, Z=SMe) was used to synthesize the following compounds by using appropriate starting materials:

| Example | Structure | Characterization |
|---|---|---|
| 102 | 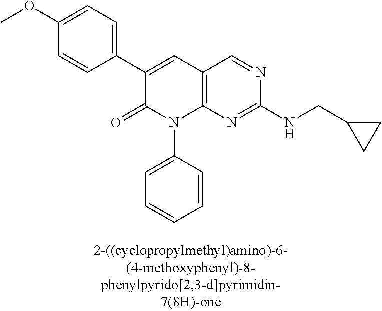 [SMILES: COc1ccc(-c2cc3cnc(NCC4CC4)nc3n(-c3ccccc3)c2=O)cc1] | LC-MS (ESI): m/z 399 [M + H]⁺. 1H NMR (400 MHz, DMSO- d6) δ: (the ratio of two tautomers: 3:2) 8.71 (s, 0.4H), 8.66 (s, 0.6H), 7.97 (s, 1H), 7.84-7.78 (m, 0.6H), 7.63 (d, J = 8.6 Hz, 2H), 7.55-7.40 (m, 3.4H), 7.29 (d, J = 7.4 Hz, 2H), 6.96 (d, J = 8.7 Hz, 2H), 3.78 (s, 3H), 3.17-3.12 (m, 0.8H), 2.78- 2.71 (m, 1.2H), 1.07-0.95 (m, 0.4H), 0.84-0.71 (m, 0.6H), 0.40-0.31 (m, 0.8H), 0.27- 0.11 (m, 2H), −0.06-0.19 (m, 1.2H) |
| 103 | 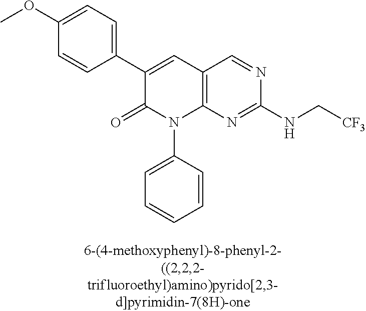 [SMILES: COc1ccc(-c2cc3cnc(NCC(F)(F)F)nc3n(-c3ccccc3)c2=O)cc1] | LC-MS (ESI): m/z 427 [M + H]⁺. 1H NMR (400 MHz, DMSO- d6) (the ratio of two tautomers: 3:2) δ: 8.77 (s, 1H), 8.25-8.19 (m, 0.6H), 8.02 (s, 1H), 7.98-7.92 (m, 0.4H), 7.63 (d, J = 8.8 Hz, 2H), 7.57-7.40 (m, 3H), 7.29 (s, 2H), 6.97 (d, J = 8.8 Hz, 2H), 4.14-4.02 (m, 0.8H), 3.78 (s, 3H), 3.74-3.65 (m, 1.2H). |
| 104 | 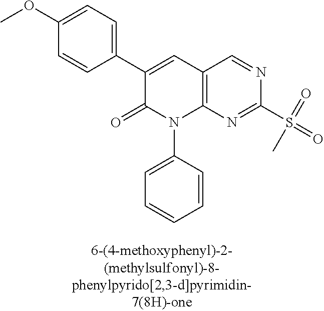 [SMILES: COc1ccc(-c2cc3cnc(S(C)(=O)=O)nc3n(-c3ccccc3)c2=O)cc1] | LC-MS (ESI): m/z 408 [M + H]+. 1H NMR (400 MHz, CDCl3) δ: 9.09 (s, 1H), 7.92 (s, 1H), 7.75 (d, J = 8.8 Hz, 2H), 7.62-7.49 (m, 3H), 7.28 (d, J = 7.5 Hz, 2H), 6.99 (d, J = 8.8 Hz, 2H), 3.87 (s, 3H), 3.03 (s, 3H). |
| 105 | 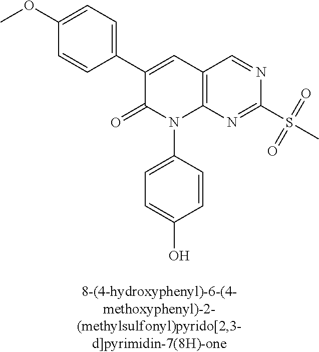 | LC-MS (ESI): m/z 424 [M + H]+. 1H NMR (400 MHz, DMSO- d6) δ: 9.77 (s, 1H), 9.32 (s, 1H), 8.34 (s, 1H), 7.75 (d, J = 9.2 Hz, 2H), 7.18 (d, J = 8.8 Hz, 2H), 7.05 (d, J = 8.8 Hz, 2H), 6.91 (d, J = 8.8 Hz, 2H), 3.83 (s, 3H), 3.18 (s, 3H). |
| 106 | 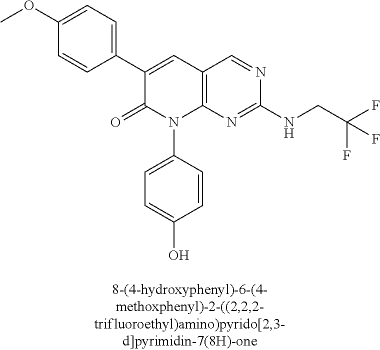 [SMILES: COc1ccc(-c2cc3cnc(NCC(F)(F)F)nc3n(-c3ccc(O)cc3)c2=O)cc1] | LC-MS (ESI): m/z 443 [M + H]+. 1H NMR (400 MHz, DMSO- d6) (the ratio of two tautomers: 3:2) δ: 9.62 (s, 1H), 8.76 (s, 1H), 8.31-7.87 (m, 2H), 7.63 (d, J = 8.8 Hz, 2H), 7.06 (d, J = 6.2 Hz, 2H), 6.98 (d, J = 8.8 Hz, 2H), 6.87 (d, J = 8.8 Hz, 2H), 4.21-4.10 (m, 1.2H), 3.83 (s, 3H), 3.85- 3.75 (m, 0.8H, overlapped). |
| 107 | 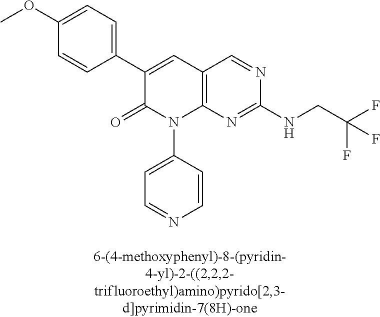 [SMILES: COc1ccc(-c2cc3cnc(NCC(F)(F)F)nc3n(-c3ccncc3)c2=O)cc1] | LC-MS (ESI): m/z 428 [M + H]+. 1H NMR (400 MHz, CDCl3) (the ratio of two tautomers: 7:3) δ: 8.82 (s, 2H), 8.61 (s, 1H), 7.73 (s, 1H), 7.64 (d, J = 7.2 Hz, 2H), 7.27 (s, 2H), 6.96 (d, J = 8.0 Hz, 2H), 5.78-5.52 (m, 1H), 4.32-4.05 (m, 0.6H), 3.85 (s, 3H), 3.88- 3.65 (m, 1.4H, overlapped). |
| 108 | 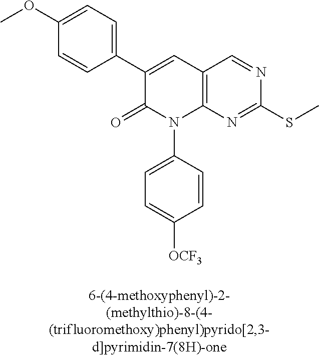 | LC-MS (ESI): m/z 460 [M + H]+. 1H NMR (400 MHz, DMSO- d6) δ: 8.97 (s, 1H), 8.20 (s, 1H), 7.71 (d, J = 8.8 Hz, 2H), 7.57 (s, 4H), 7.02 (d, J = 8.8 Hz, 2H), 3.81 (s, 3H), 2.19 (s, 3H). |
| 109 | 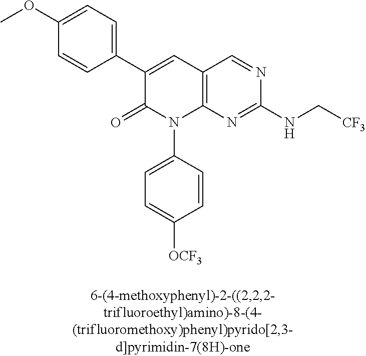 [SMILES: COc1ccc(-c2cc3cnc(NCC(F)(F)F)nc3n(-c3ccc(OC(F)(F)F)cc3)c2=O)cc1] | LC-MS (ESI): m/z 511 [M + H]+. 1H NMR (400 MHz, DMSO- d6) (the ratio of two tautomers: 7:3) δ: 8.84-8.77 (m, 1H), 8.32-8.26 (m, 0.7H), 8.06 (s, 1H), 7.99-7.93 (m, 0.3H), 7.65 (d, J = 8.8 Hz, 2H), 7.58-7.48 (m, 4H), 6.99 (d, J = 8.8 Hz, 2H), 4.18-4.09 (m, 0.6H), 3.80 (s, 3H), 3.74- 3.65 (m, 1.4H). |
| 110 | 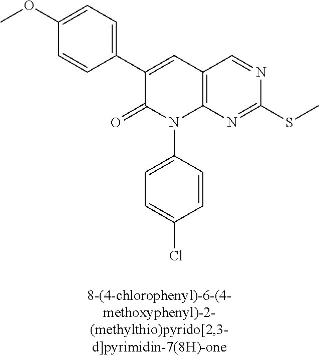 | LC-MS (ESI): m/z 410 [M + H]+. 1H NMR (400 MHz, CDCl3) δ: 8.70 (s, 1H), 7.78 (s, 1H), 7.68 (d, J = 9.2 Hz, 2H), 7.52 (d, J = 9.2 Hz, 2H), 7.24 (d, J = 8.8 Hz, 2H), 6.96 (d, J = 9.2 Hz, 2H), 3.85 (s, 3H), 2.24 (s, 3H). |
| 111 | 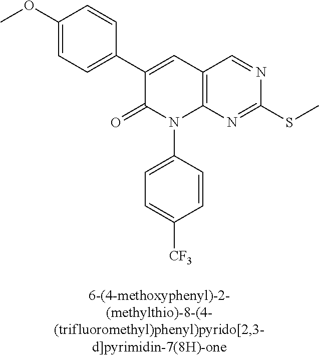 | LC-MS (ESI): m/z 444 [M + H]+. 1H NMR (400 MHz, DMSO- d6) δ: 8.98 (s, 1H), 8.21 (s, 1H), 7.95 (d, J = 8.4 Hz, 2H), 7.70 (t, J = 8.8 Hz, 4H), 7.02 (d, J = 8.8 Hz, 2H), 3.80 (s, 3H), 2.19 (s, 3H). |
| 112 | 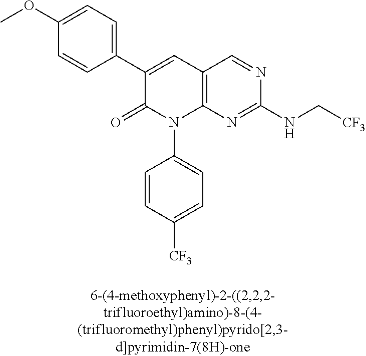 [SMILES: COc1ccc(-c2cc3cnc(NCC(F)(F)F)nc3n(-c3ccc(C(F)(F)F)cc3)c2=O)cc1] | LC-MS (ESI): m/z 495 [M + H]+. 1H NMR (400 MHz, CDCl3) (the ratio of two tautomers: 3:2) δ: 8.60 (s, 1H), 7.81 (d, J = 8.3 Hz, 2H), 7.73 (s, 1H), 7.65 (d, J = 8.9 Hz, 2H), 7.42 (d, J = 8.1 Hz, 2H), 6.95 (d, J = 8.9 Hz, 2H), 5.65-5.30 (m, 1H, two set of m peaks), 4.50-4.05 (m, 0.6H), 3.84 (s, 3H), 3.75-3.45 (m, 1.2H). |
| 113 | 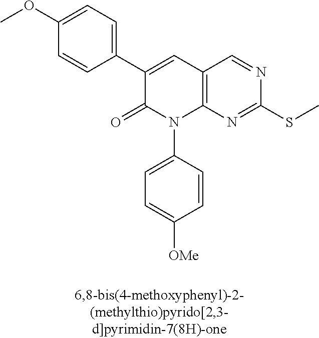 | LC-MS (ESI): m/z 406 [M + H]+. 1H NMR (400 MHz, DMSO- d6) δ: 8.94 (s, 1H), 8.15 (s, 1H), 7.69 (d, J = 8.8 Hz, 2H), 7.27 (d, J = 8.8 Hz, 2H), 7.08 (d, J = 8.8 Hz, 2H), 7.01 (d, J = 8.9 Hz, 2H), 3.83 (s, 3H), 3.80 (s, 3H), 2.23 (s, 3H). |
| 114 | 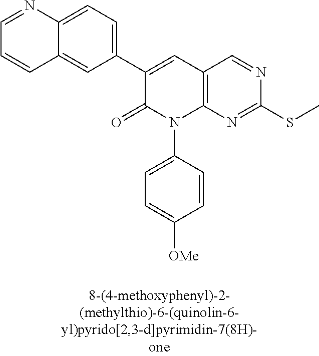 [SMILES: COc1ccc(-n2c(=O)c(-c3ccc4ncccc4c3)cc3cnc(SC)nc32)cc1] | LC-MS (ESI): m/z 427 [M + H]+. 1H NMR (400 MHz, DMSO- d6) δ: 9.07 (s, 1H), 8.99 (dd, J = 4.0 Hz, 1.6 Hz, 1H), 8.51- 8.46 (m, 3H), 8.15-8.14 (m, 2H), 7.64 (dd, J = 8.0, 4.0 Hz, 1H), 7.39 (d, J = 8.8 Hz, 2H), 7.17 (d, J = 8.8 Hz, 2H), 3.90 (s, 3H), 2.32 (s, 3H). |
| 115 | 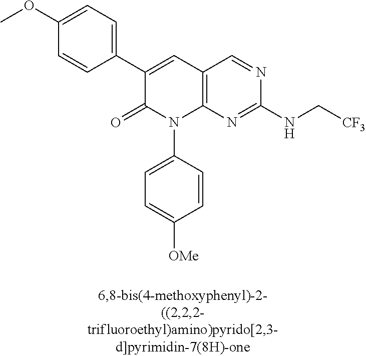 [SMILES: COc1ccc(-c2cc3cnc(NCC(F)(F)F)nc3n(-c3ccc(OC)cc3)c2=O)cc1] | LC-MS (ESI): m/z 457 [M + H]+. 1H NMR (400 MHz, DMSO- d6) δ: (two tautomers ratio 1:1) 8.77 (s, 1H), 8.28-8.21 (m, 0.5H), 8.01 (s, 1H), 7.98- 7.89 (m, 0.5H), 7.63 (d, J = 8.8 Hz, 2H), 7.26-7.16 (m, 2H), 7.06 (d, J = 8.7 Hz, 2H), 6.98 (d, J = 8.9 Hz, 2H), 4.21-4.01 (m, 1H), 3.82 (s, 3H), 3.79 (s, 3H), 3.80-3.69 (m, 1H, overlapped) |
| 116 | 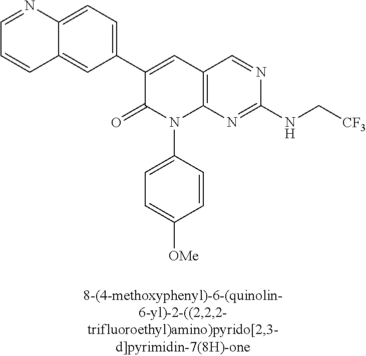 [SMILES: COc1ccc(-n2c(=O)c(-c3ccc4ncccc4c3)cc3cnc(NCC(F)(F)F)nc32)cc1] | LC-MS (ESI): m/z 478 [M + H]+. 1H NMR (400 MHz, DMSO- d6) (the ratio of two tautomers: 1:1) δ: 8.91 (dd, J = 4.0 Hz, 1.6 Hz, 1H), 8.88- 8.82 (m, 1H), 8.41 (d, J = 7.2 Hz, 1H), 8.37-8.34 (m, 1.5H), 8.29 (s, 1H), 8.10-7.99 (m, 2.5H), 7.56 (dd, J = 8.4, 4.0 Hz, 1H), 7.30-7.25 (m, 2H), 7.08 (d, J = 8.8 Hz, 2H), 4.19-4.10 (m, 1H), 3.84 (s, 3H), 3.80-3.74 (m, 1H). |
| 117 | 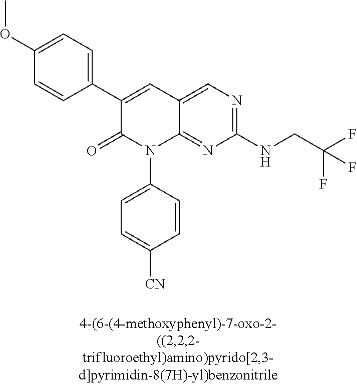 [SMILES: COc1ccc(-c2cc3cnc(NCC(F)(F)F)nc3n(-c3ccc(C#N)cc3)c2=O)cc1] | LC-MS (ESI): m/z 452 [M + H]+. 1H NMR (400 MHz, DMSO- d6) (the ratio of two tautomers: 1:1) δ: 8.81 (br, 1H), 8.35-8.02 (m, 4H), 7.68- 7.60 (m, 4H), 6.99 (d, J = 8.8 Hz, 2H), 4.13 (br, 1H), 3.80 (s, 3H), 3.70 (br, 1H). |
| 118 | 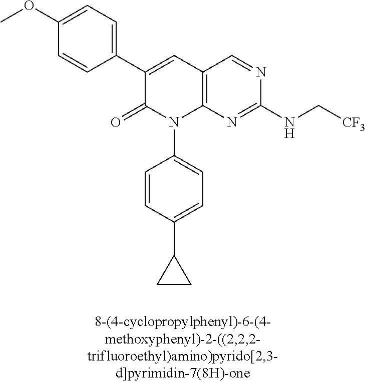 [SMILES: COc1ccc(-c2cc3cnc(NCC(F)(F)F)nc3n(-c3ccc(C4CC4)cc3)c2=O)cc1] | LC-MS (ESI): m/z 467 [M + H]+. 1H NMR (400 MHz, DMSO- d6) (the ratio of two tautomers: 1:1) δ: 8.82-8.73 (m, 1H), 8.29-8.20 (m, 0.5H), 8.02 (s, 1H), 8.00-7.92 (m, 0.5H), 7.63 (d, J = 8.4 Hz, 2H), 7.26-7.10 (m, 4H), 6.98 (d, J = 8.4 Hz, 2H), 4.18-4.04 (m, 1H), 3.78 (s, 3H), 3.75- 3.65 (m, 1H), 2.06-1.96 (m, 1H), 1.07-0.97 (m, 2H), 0.79- 0.67 (m, 2H). |

### Preparation of Example 119 via General Procedure I (Route B, Y=CH, Z=Cl

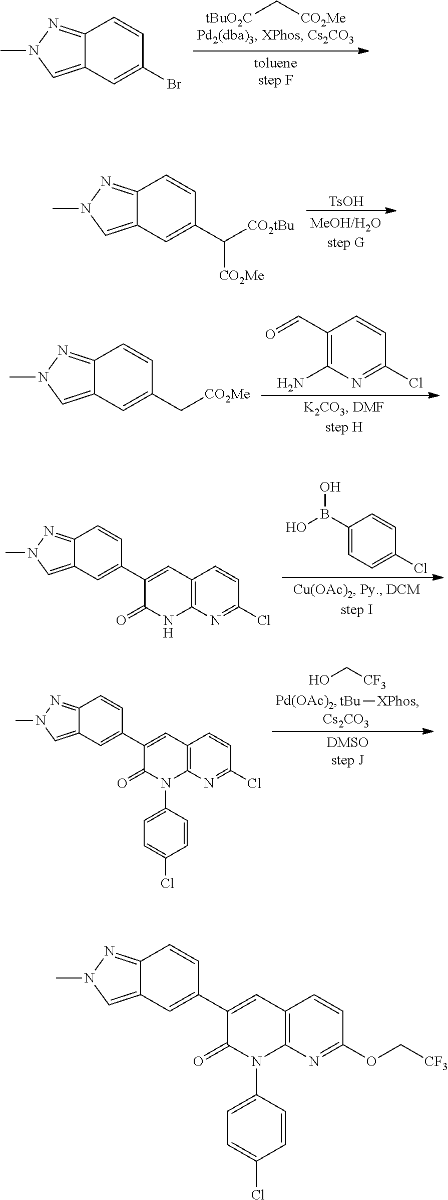

### Step F: 1-(tert-butyl) 3-methyl 2-(2-methyl-2H-indazol-5-yl)malonate

A mixture of 5-bromo-2-methyl-2H-indazole (20 g, 95.7 mmol, 1.0 eq.), tert-butyl methyl malonate (18.6 g, 105.3 mmol, 1.1 eq.), Pd₂(dba)₃ (4.4 g, 4.8 mmol, 0.05 eq.), X-Phos (4.6 g, 9.6 mmol, 0.1 eq.) and Cs₂CO₃ (62.4 g, 191.4 mmol, 2.0 eq.) in toluene (250 mL) was stirred at 100° C. for 2 hrs under N2 atmosphere. The mixture was filtered through a short pad of Celite®, and the filtrate was concentrated under reduced pressure and the residue was purified by flash column chromatography on silica gel to afford 1-(tert-butyl) 3-methyl 2-(2-methyl-2H-indazol-5-yl)malonate (26.4 g, 91% yield) as a yellow oil. LC-MS (ESI): m/z 305 [M+H]⁺.

### Step G: methyl 2-(2-methyl-2H-indazol-5-yl)acetate

To a solution of 1-(tert-butyl) 3-methyl 2-(2-methyl-2H-indazol-5-yl)malonate (24 g, 78.9 mmol, 1.0 eq.) in MeOH/H₂O mixture (200 mL, 1:1, v:v) was added TsOH monohydrate (18.6 g, 157.8 mmol, 2.0 eq.) at room temperature. The resulting mixture was stirred at 100° C. for 14 hrs. The reaction mixture was quenched with ice water (200 mL) and extracted with EtOAc (200 mL×3). The combined organic layers were washed with brine (200 mL), dried over Na₂SO₄ and concentrated under reduced pressure. The residue was purified by flash column chromatography on silica gel to afford methyl 2-(2-methyl-2H-indazol-5-yl)acetate (9 g, 56% yield) as a yellow oil. LC-MS (ESI): m/z 205 [M+H]⁺.

### Step H: 7-chloro-3-(2-methyl-2H-indazol-5-yl)-1,8-naphthyridin-2(1H)-one

To a solution of methyl 2-(2-methyl-2H-indazol-5-yl)acetate (1.4 g, 6.9 mmol, 1.0 eq.) in DMF (15 mL) was added K₂CO₃ (2.8 g, 20.6 mmol, 3.0 eq.) and 2-amino-6-chloronicotinaldehyde (1.0 g, 6.4 mmol, 0.95 eq.) at room temperature. The resulting mixture was stirred at 100° C. for 2 hrs. The reaction was quenched with ice water (20 mL), the resulting precipitate was filtered, the filter cake was collected and dried under reduced pressure to afford 7-chloro-3-(2-methyl-2H-indazol-5-yl)-1,8-naphthyridin-2(1H)-one (1.8 g, 91% yield) as a white solid. LC-MS (ESI): m/z 311 [M+H]⁺.

### Step I: 7-chloro-1-(4-chlorophenyl)-3-(2-methyl-2H-indazol-5-yl)-1,8-naphthyridin-2(1H)-one

A mixture of 7-chloro-3-(2-methyl-2H-indazol-5-yl)-1,8-naphthyridin-2(1H)-one (300 mg, 0.97 mmol, 1.0 eq.), (4-chlorophenyl)boronic acid (196 mg, 1.26 mmol, 1.3 eq.), Cu(OAc)₂ (211 mg, 1.16 mmol, 1.2 eq.) and pyridine (229 mg, 2.9 mmol, 3 eq.) in DCM (3 mL) was stirred at 40° C. under 02 atmosphere for 14 hrs. The reaction mixture was diluted with H₂O (20 mL) and extracted with EtOAc (20 mL×3), the combined organic layers were washed with brine (30 mL), dried over Na₂SO₄ and concentrated under reduced pressure. The residue was purified by flash column chromatography on silica gel to give 7-chloro-1-(4-chlorophenyl)-3-(2-methyl-2H-indazol-5-yl)-1,8-naphthyridin-2(1H)-one (240 mg, 59% yield) as a yellow solid. LC-MS (ESI): m/z 421 [M+H]⁺.

### Step J: 1-(4-chlorophenyl)-3-(2-methyl-2H-indazol-5-yl)-7-(2,2,2-trifluoroethoxy)-1,8-naphthyridin-2(1H)-one

A mixture of 7-chloro-1-(4-chlorophenyl)-3-(2-methyl-2H-indazol-5-yl)-1,8-naphthyridin-2(1H)-one (70 mg, 0.17 mmol, 1.0 eq.), 2,2,2-trifluoroethan-1-ol (340 mg, 3.4 mmol, 20 eq.), Cs₂CO₃ (111 mg, 0.34 mmol, 2.0 eq.), Pd(OAc)₂ (8 mg, 0.034 mmol, 0.2 eq.) and tBu-XPhos (14 mg, 0.034 mmol, 0.2 eq.) in DMSO (2 mL) was stirred at 100° C. in a sealed tube under N2 atmosphere overnight. The reaction mixture was diluted with H₂O (20 mL) and extracted with EtOAc (20 mL×3), the combined organic layers were washed with brine (20 mL), dried over Na₂SO₄ and concentrated under reduced pressure. The residue was purified by RP-prep-HPLC to afford 1-(4-chlorophenyl)-3-(2-methyl-2H-indazol-5-yl)-7-(2,2,2-trifluoroethoxy)-1,8-naphthyridin-2(1H)-one (Example 119).

¹H NMR (400 MHz, DMSO-d₆) δ: 8.41 (s, 1H), 8.28 (d, J=8.0 Hz, 1H), 8.27 (s, 1H), 8.13 (s, 1H), 7.65-7.60 (m, 3H), 7.57 (dd, J=9.1 Hz, 1.6 Hz, 1H), 7.43 (d, J=8.4 Hz, 2H), 6.92 (d, J=8.4 Hz, 1H), 4.61 (q, J=9.0 Hz, 2H), 4.18 (s, 3H). LC-MS (ESI): m/z=485 [M+H]⁺.

The procedure set forth above for General Procedure I (Route B, Y=CH, Z=Cl) was used to synthesize the following compounds by using appropriate starting materials:

| Example | Structure | Characterization |
|---|---|---|
| 120 | 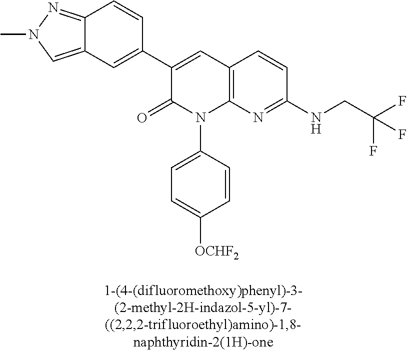 [SMILES: Cn1cc2cc(-c3cc4ccc(NCC(F)(F)F)nc4n(-c4ccc(OC(F)F)cc4)c3=O)ccc2n1] | LC-MS (ESI): m/z 516 [M + H]+. 1H NMR (400 MHz, DMSO- d6) δ: 8.36 (s, 1H), 8.07 (d, J = 3.1 Hz, 2H), 7.90 (d, J = 8.5 Hz, 1H), 7.85 (s, 1H), 7.61- 7.54 (m, 2H), 7.37-7.29 (m, 4H), 7.31 (t, J_{HF} = 74.0 Hz, 1H), 6.55 (d, J = 8.5 Hz, 1H), 4.17 (s, 3H), 3.84-3.70 (m, 2H). |
| 121 | 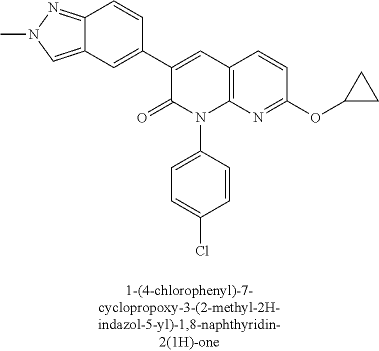 [SMILES: Cn1cc2cc(-c3cc4ccc(OC5CC5)nc4n(-c4ccc(Cl)cc4)c3=O)ccc2n1] | LC-MS (ESI): m/z 443 [M + H]+. 1H NMR (400 MHz, DMSO- d6) δ: 8.40 (s, 1H), 8.24 (s, 1H), 8.21 (d, J = 8.4 Hz, 1H), 8.13-8.12 (m, 1H), 7.65-7.56 (m, 4H), 7.46-7.40 (m, 2H), 6.84 (d, J = 8.4 Hz, 1H), 4.19 (s, 3H), 3.80-3.78 (m, 1H), 0.62-0.52 (m, 4H). |
| 122 | 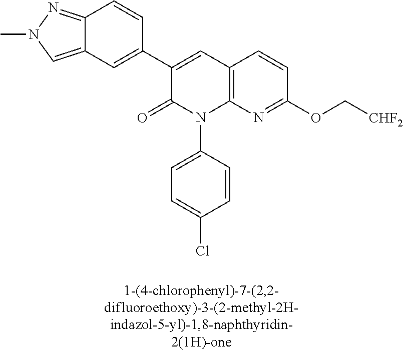 [SMILES: Cn1cc2cc(-c3cc4ccc(OCC(F)F)nc4n(-c4ccc(Cl)cc4)c3=O)ccc2n1] | LC-MS (ESI): m/z = 467 [M + H]+. 1H NMR (400 MHz, DMSO- d6) δ: 8.41 (s, 1H), 8.29-8.21 (m, 2H), 8.13 (s, 1H), 7.70- 7.54 (m, 4H), 7.45 (d, J = 7.6 Hz, 2H), 6.87 (d, J = 8.0 Hz, 1H), 6.12 (t, J_{HF} = 54.7 Hz, 1H), 4.28-4.10 (m, 5H). |
| 123 | 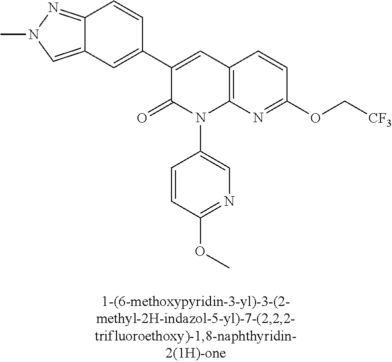 [SMILES: COc1ccc(-n2c(=O)c(-c3ccc4nn(C)cc4c3)cc3ccc(OCC(F)(F)F)nc32)cn1] | LC-MS (ESI): m/z 482 [M + H]+. 1H NMR (400 MHz, DMSO- d6) δ: 8.47 (s, 1H), 8.35 (d, J = 7.2 Hz, 1H), 8.34 (s, 1H), 8.26 (d, J = 2.8 Hz, 1H), 8.19 (s, 1H), 7.85 (dd, J = 8.8 Hz, 2.4 Hz, 1H), 7.69 (d, J = 8.8 Hz, 1H), 7.64 (dd, J = 9.2 Hz, 1.6 Hz, 1H), 7.08 (d, J = 8.8 Hz, 1H), 7.00 (d, J = 8.4 Hz, 1H), 4.71 (q, J_{HF} = 8.8 Hz, 2H), 4.25 (s, 3H), 4.00 (s, 3H). |
| 124 | 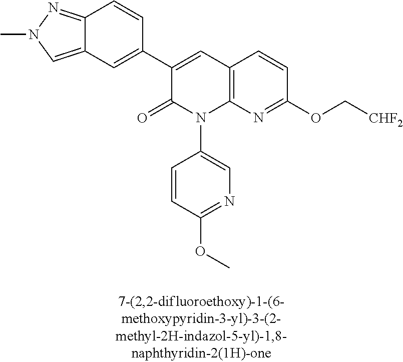 [SMILES: COc1ccc(-n2c(=O)c(-c3ccc4nn(C)cc4c3)cc3ccc(OCC(F)F)nc32)cn1] | LC-MS (ESI): m/z 464 [M + H]+. 1H NMR (400 MHz, DMSO- d6) δ: 8.45 (s, 1H), 8.34-8.21 (m, 3H), 8.17 (s, 1H), 7.84 (dd, J = 8.4 Hz, 2.4 Hz, 1H), 7.67 (d, J = 8.8 Hz, 1H), 7.62 (d, J = 9.2 Hz, 1H), 7.06 (d, J = 8.8 Hz, 1H), 6.92 (d, J = 8.4 Hz, 1H), 6.21 (tt, J_{HF} = 55.2 Hz, J = 3.2 Hz, 1H), 4.33- 4.24 (m, 2H), 4.23 (s, 3H), 3.98 (s, 3H). |
| 301 | 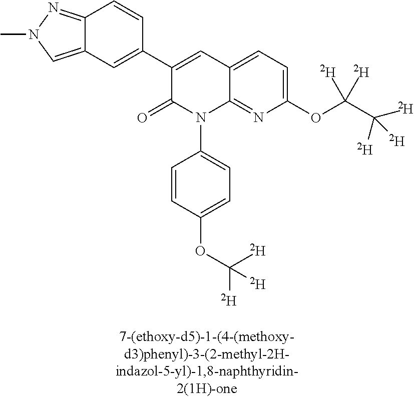 [SMILES: [2H]C([2H])([2H])Oc1ccc(-n2c(=O)c(-c3ccc4nn(C)cc4c3)cc3ccc(OC([2H])([2H])C([2H])([2H])[2H])nc32)cc1] | LC-MS: m/z 435 (M + H)+. 1H NMR (400 MHz, DMSO- d6) δ: 8.39 (s, 1H), 8.19 (s, 1H), 8.16-8.09 (m, 2H), 7.63- 7.55 (m, 2H), 7.25 (d, J = 8.8 Hz, 2H), 7.07 (d, J = 8.9 Hz, 2H), 6.70 (d, J = 8.4 Hz, 1H), 4.18 (s, 3H). |
| 302 | 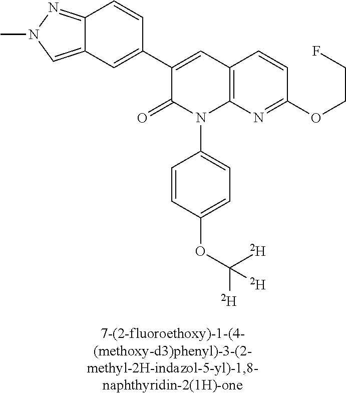 [SMILES: [2H]C([2H])([2H])Oc1ccc(-n2c(=O)c(-c3ccc4nn(C)cc4c3)cc3ccc(OCCF)nc32)cc1] | LC-MS: m/z 448 (M + H)+. 1H NMR (400 MHz, DMSO- d6) δ: 8.39 (s, 1H), 8.23-8.17 (m, 2H), 8.11 (s, 1H), 7.59 (d, J = 9.0 Hz, 2H), 7.26 (d, J = 8.8 Hz, 2H), 7.08 (d, J = 8.8 Hz, 2H), 6.79 (d, J = 8.4 Hz, 1H), 4.63-4.56 (m, 1H), 4.50- 4.44 (m, 1H), 4.18 (s, 3H), 4.17-4.08 (m, 2H). |
| 303 | 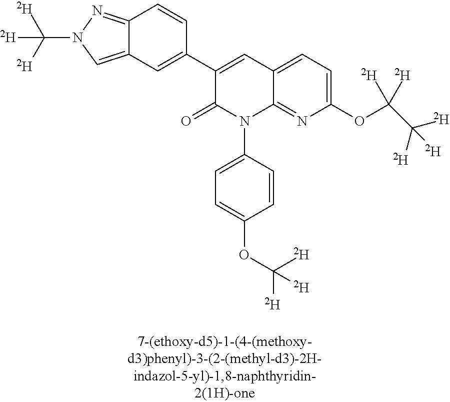 [SMILES: [2H]C([2H])([2H])Oc1ccc(-n2c(=O)c(-c3ccc4nn(C([2H])([2H])[2H])cc4c3)cc3ccc(OC([2H])([2H])C([2H])([2H])[2H])nc32)cc1] | LC-MS: m/z 438 (M + H)+. 1H NMR (400 MHz, DMSO- d6) δ: 8.38 (s, 1H), 8.19 (s, 1H), 8.16-8.09 (m, 2H), 7.58 (q, J = 9.0 Hz, 2H), 7.25 (d, J = 8.8 Hz, 2H), 7.07 (d, J = 8.8 Hz, 2H), 6.70 (d, J = 8.4 Hz, 1H). |
| 304 | 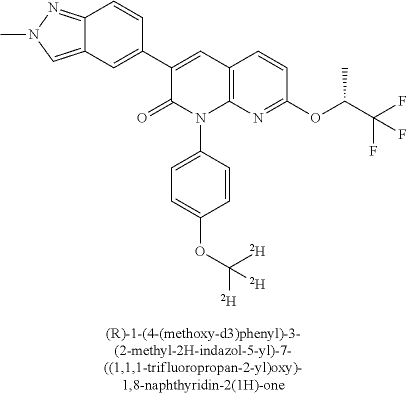 [SMILES: [2H]C([2H])([2H])Oc1ccc(-n2c(=O)c(-c3ccc4nn(C)cc4c3)cc3ccc(O[C@H](C)C(F)(F)F)nc32)cc1] | LC-MS: m/z 498 (M + H)+. 1H NMR (400 MHz, DMSO- d6) δ: 8.40 (s, 1H), 8.25 (d, J = 6.8 Hz, 2H), 8.12 (s, 1H), 7.59 (q, J = 9.0 Hz, 2H), 7.28 (t, J = 7.2 Hz, 2H), 7.09 (d, J = 8.8 Hz, 2H), 6.85 (d, J = 8.4 Hz, 1H), 5.12-5.05 (m, 1H), 4.18 (s, 3H), 1.22 (d, J = 6.5 Hz, 3H). |
| 305 | 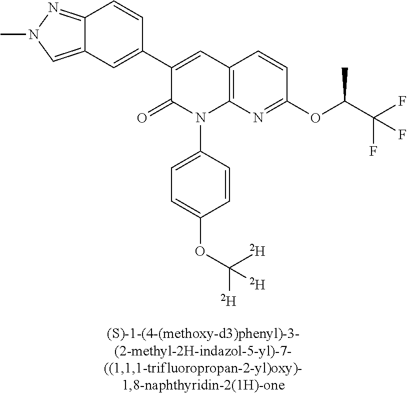 [SMILES: [2H]C([2H])([2H])Oc1ccc(-n2c(=O)c(-c3ccc4nn(C)cc4c3)cc3ccc(O[C@@H](C)C(F)(F)F)nc32)cc1] | LC-MS: m/z 498 (M + H)+. 1H NMR (400 MHz, DMSO- d6) δ: 8.40 (s, 1H), 8.25 (d, J = 7.0 Hz, 2H), 8.12 (s, 1H), 7.60 (q, J = 9.1 Hz, 2H), 7.28 (t, J = 7.2 Hz, 2H), 7.10 (d, J = 8.7 Hz, 2H), 6.85 (d, J = 8.4 Hz, 1H), 5.12-5.05 (m, 1H), 4.18 (s, 3H), 1.22 (d, J = 6.5 Hz, 3H). |

General Procedure II

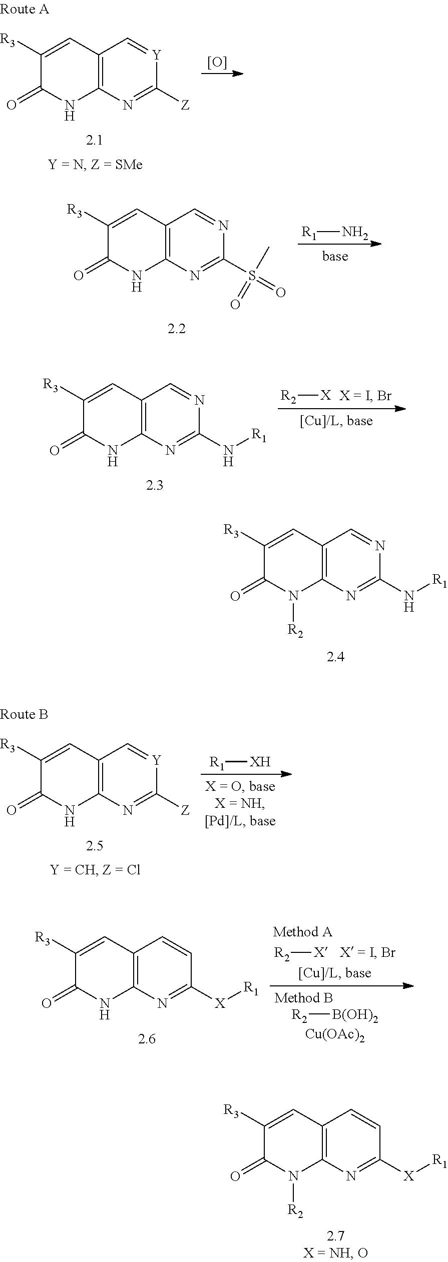

Compounds of structure 2.4 and 2.7 were obtained through the scheme depicted as General Procedure II. For Route A (where Y=N and Z=SMe), thioether 2.1 (compound 1.3 in General Procedure I) was oxidized to afford sulfone 2.2. The desired R₁ group was introduced through a nucleophilic aromatic substitution reaction to afford compound 2.3. A copper mediated Ullmann coupling was used to introduce the desired R2 group, yielding compounds of structure 2.4. For Route B (where Y=CH and Z=Cl) the desired R₁ group was introduced through a palladium mediated Buchwald-Hartwig coupling using heteroaryl-chloride 2.5 (compound 1.8 in General Procedure I) to generate heterocycle 2.6. The desired R2 group was then introduced either through a copper mediated Ullmann coupling (Method A) or copper mediated Chan-Lam coupling (Method B) to afford compounds of structure 2.7.

### Preparation of Example 125 via General Procedure II (Route A, Y=N, Z=SMe

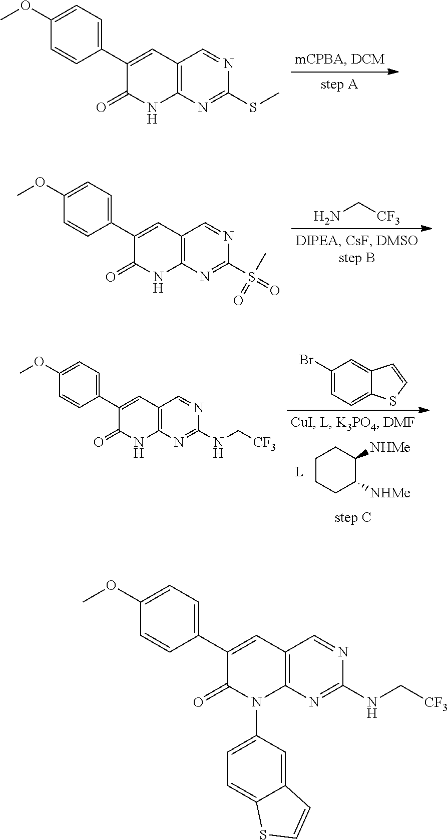

### Step A: 6-(4-methoxyphenyl)-2-(methylsulfonyl)pyrido[2,3-d]pyrimidin-7(8H)-one

To a solution of 6-(4-methoxyphenyl)-2-(methylthio)pyrido[2,3-d]pyrimidin-7(8H)-one (1.4 g, 4.7 mmol, 1.0 eq.) in DCM (100 mL) was added m-CPBA (2.4 g, 13.9 mmol, 2.96 eq.) at room temperature in several portions. The resulting mixture was stirred for additional 2 hrs. Then the reaction mixture was quenched with ice water (100 mL), the excess of mCPBA was quenched by washing with Na₂S203 (sat. aq.) and extracting with DCM (50 mL×3). The combined organic layers were washed with brine (50 mL), dried over Na₂SO₄ and concentrated under reduced pressure. The residue was purified by flash column chromatography on silica gel to afford 6-(4-methoxyphenyl)-2-(methylsulfonyl)pyrido[2,3-d]pyrimidin-7(8H)-one (1.5 g, 96% yield) as a yellow solid. LC-MS (ESI): m/z 332 [M+H]⁺.

### Step B: 6-(4-methoxyphenyl)-2-(2,2,2-trifluoroethylamino)pyrido[2,3-d]pyrimidin-7(8H)-one

To a solution of 6-(4-methoxyphenyl)-2-(methylsulfonyl)pyrido[2,3-d]pyrimidin-7(8H)-one (1.5 g, 4.5 mmol, 1.0 eq.) in DMSO (20 mL) was added CsF (70 mg, 0.46 mmol, 0.1 eq.), DIPEA (1.7 g, 13.2 mmol, 2.93 eq.) and 2,2,2-trifluoroethanamine (1.3 g, 13.2 mmol, 2.93 eq.) at room temperature in a sealed tube. The resulting mixture was stirred at 80° C. overnight. Then the reaction mixture was quenched with ice water (50 mL) and extracted with DCM (50 mL×3). The combined organic layers were washed with brine (50 mL), dried over Na₂SO₄ and concentrated under reduced pressure. The residue was purified by flash column chromatography on silica gel to afford 6-(4-methoxyphenyl)-2-(2,2,2-trifluoroethylamino)pyrido[2,3-d]pyrimidin-7(8H)-one (700 mg, 44% yield) as a yellow solid. LC-MS (ESI): m/z 351 [M+H]⁺.

### Step C: 8-(benzo[b]thiophen-5-yl)-6-(4-methoxyphenyl)-2-(2,2,2-trifluoroethylamino)pyrido[2,3-d]pyrimidin-7(8H)-one

To a solution of 6-(4-methoxyphenyl)-2-(2,2,2-trifluoroethylamino)pyrido[2,3-d]pyrimidin-7(8H)-one (60 mg, 0.17 mmol, 1.0 eq.) in DMF (3.0 mL) was added CuI (32 mg, 0.17 mmol. 1.0 eq.), (1R,2R)—N1,N2-dimethylcyclohexane-1,2-diamine (48 mg, 0.34 mmol, 2.0 eq.), K₃PO₄ (72 mg, 0.34 mmol, 2.0 eq.) and 5-bromobenzo[b]thiophene (72 mg, 0.34 mmol, 2.0 eq.) at room temperature. The resulting mixture was stirred at 100° C. under N2 atmosphere for 14 hrs. Then the reaction mixture was quenched with ice water (15 mL) and extracted with EtOAc (20 mL×3). The combined organic layers were washed with brine (20 mL), dried over Na₂SO₄ and concentrated under reduced pressure. The residue was purified by RP-prep-HPLC to afford 8-(benzo[b]thiophen-5-yl)-6-(4-methoxyphenyl)-2-(2,2,2-trifluoroethylamino)pyrido[2,3-d]pyrimidin-7(8H)-one (Example 125).

1H NMR (400 MHz, DMSO-d₆) δ: 8.85-8.77 (m, 1H), 8.28-8.22 (m, 0.5H), 8.14 (d, J=8.4 Hz, 1H), 8.07 (s, 1H), 7.98-7.92 (m, 0.5H), 7.86 (d, J=5.2 Hz, 2H), 7.67 (d, J=9.2 Hz, 2H), 7.55-7.47 (m, 1H), 7.34-7.27 (m, 1H), 6.99 (d, J=8.8 Hz, 2H), 4.15-4.04 (m, 1H), 3.80 (s, 3H), 3.70-3.58 (m, 1H). LC-MS (ESI): m/z 483 [M+H]⁺

The procedure set forth above for General Procedure II (Route A, Y=N, Z=SMe) was used to synthesize the following compounds by using appropriate starting materials:

| Example | Structure | Characterization |
|---|---|---|
| 126 | 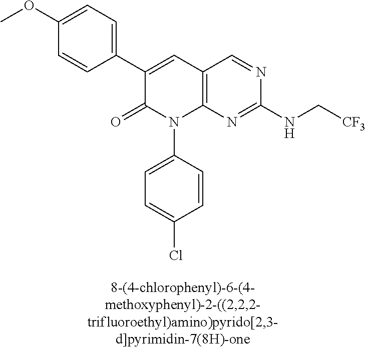 [SMILES: COc1ccc(-c2cc3cnc(NCC(F)(F)F)nc3n(-c3ccc(Cl)cc3)c2=O)cc1] | LC-MS (ESI): m/z 461 [M + H]+. 1H NMR (400 MHz, CDCl3) (the ratio of two tautomers: 1:1) δ: 8.58 (s, 1H), 7.71 (s, 1H), 7.65 (d, J = 8.8 Hz, 2H), 7.51 (d, J = 8.4 Hz, 2H), 7.22 (d, J = 8.8 Hz, 2H), 6.95 (d, J = 8.8 Hz, 2H), 5.62-5.32 (m, 1H), 4.23-4.09 (m, 1H), 3.84 (s, 3H), 3.88-3.72 (m, 1H). |
| 127 | 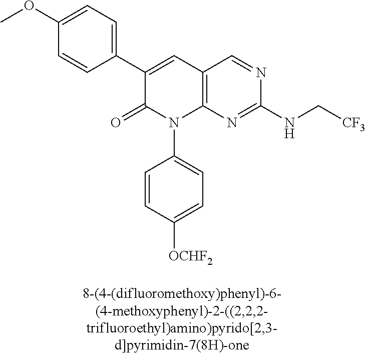 [SMILES: COc1ccc(-c2cc3cnc(NCC(F)(F)F)nc3n(-c3ccc(OC(F)F)cc3)c2=O)cc1] | LC-MS (ESI): m/z 493 [M + H]+. 1H NMR (400 MHz, CDCl3) (the ratio of two tautomers: 1:1) δ: 8.62-8.58 (m, 1H), 7.72 (s, 1H), 7.66 (d, J = 8.7 Hz, 2H), 7.26 (s, 4H), 6.95 (d, J = 8.7 Hz, 2H), 6.58 (t, J_{HF} = 73.6 Hz, 1H), 5.60-5.33 (m, 1H), 4.19-4.15 (m, 1H), 3.64 (s, 3H), 3.79-3.66 (m, 1H). |
| 128 | 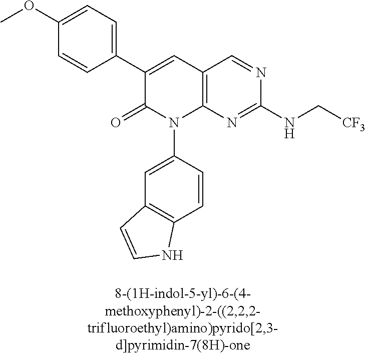 [SMILES: COc1ccc(-c2cc3cnc(NCC(F)(F)F)nc3n(-c3ccc4[nH]ccc4c3)c2=O)cc1] | LC-MS (ESI): m/z 466 [M + H]+. 1H NMR (400 MHz, CD3OD) (the ratio of two tautomers: 1:1) δ: 8.73 (s, 1H), 7.97 (s, 1H), 7.65 (d, J = 8.8 Hz, 2H), 7.56 (d, J = 8.8 Hz, 1H), 7.47 (d, J = 2.0 Hz, 1H), 7.35 (d, J = 3.2 Hz, 1H), 7.01-6.96 (m, 2H), 6.55 (d, J = 2.8 Hz, 1H), 4.60 (s, 1H), 4.21-4.06 (m, 1H), 3.84 (s, 3H), 3.74-3.61 (m, 1H). |
| 129 | 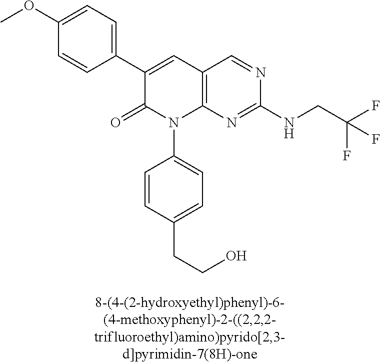 [SMILES: COc1ccc(-c2cc3cnc(NCC(F)(F)F)nc3n(-c3ccc(CCO)cc3)c2=O)cc1] | LC-MS (EIS): m/z 471 [M + H]+. 1H NMR (400 MHz, DMSO-d6) (the ratio of two tautomers: 1:1) δ: 8.83-8.74 (m, 1H), 8.28-8.20 (m, 0.5H), 8.03 (s, 1H), 8.01- 7.94 (m, 0.5H), 7.65 (d, J = 8.8 Hz, 2H), 7.36 (d, J = 7.6 Hz, 2H), 7.25-7.16 (m, 2H), 6.99 (d, J = 8.8 Hz, 2H), 4.73 (t, J = 4.4 Hz, 1H), 4.15-4.11 (m, 1H), 3.80 (s, 3H), 3.75-3.62 (m, 3H), 2.82 (t, J = 6.8 Hz, 2H). |

### Preparation of Example 130 via General Procedure II (Route B, Method A, Y=CH, Z=Cl, X=NH

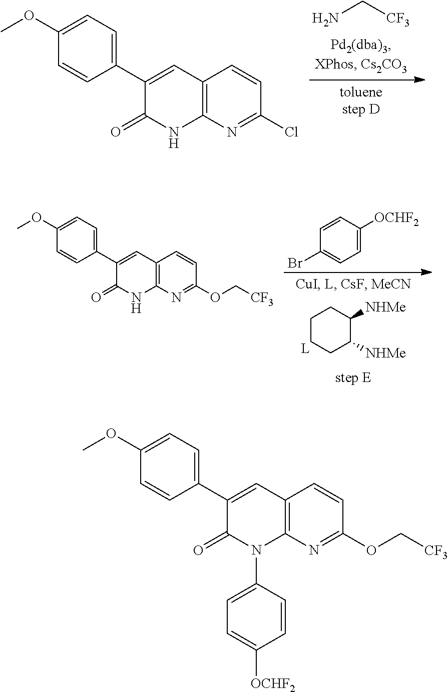

### Step D: 3-(4-methoxyphenyl)-7-((2,2,2-trifluoroethyl)amino)-1,8-naphthyridin-2(1H)-one

To a solution of 7-chloro-3-(4-methoxyphenyl)-1,8-naphthyridin-2(1H)-one (92 mg, 0.32 mmol, 1.0 eq.) in toluene (2 mL) was added 2,2,2-trifluoroethan-1-amine (160 mg, 1.6 mmol, 5.0 eq.) and Pd₂(dba)₃ (28 mg, 0.03 mmol, 0.1 eq.), XPhos (31 mg, 0.06 mmol, 0.2 eq.) and Cs₂CO₃ (208 mg, 0.64 mmol, 2.0 eq.) at room temperature. The resulting mixture was stirred at 100° C. under N2 atmosphere for 2 hrs. Then the reaction mixture was quenched with water (15 mL) and extracted with EtOAc (20 mL×3). The combined organic layers were washed with brine (20 mL), dried over Na₂SO₄ and concentrated under reduced pressure. The residue was purified by flash column chromatography on silica gel to afford 3-(4-methoxyphenyl)-7-((2,2,2-trifluoroethyl)amino)-1,8-naphthyridin-2(1H)-one (60 mg, 54% yield) as a white solid. LC-MS (ESI): m/z 350 [M+H]⁺.

### Step E: 1-(4-(difluoromethoxy)phenyl)-3-(4-methoxyphenyl)-7-(2,2,2-trifluoroethylamino)-1,8-naphthyridin-2(1H)-one

To a solution of 3-(4-methoxyphenyl)-7-(2,2,2-trifluoroethylamino)-1,8-naphthyridin-2(1H)-one (60 mg, 0.17 mmol, 1.0 eq.) in MeCN (3 mL) was added CuI (32 mg, 0.17 mmol, 1.0 eq.), (1R,2R)—N1,N2-dimethylcyclohexane-1,2-diamine (48 mg, 0.34 mmol, 2.0 eq.), CsF (78 mg, 0.51 mmol, 3.0 eq.) and 1-bromo-4-(difluoromethoxy)benzene (58 mg, 0.26 mmol, 1.5 eq.) at room temperature in a sealed tube. The resulting mixture was stirred at 90° C. under N2 atmosphere for 14 hrs. Then the reaction mixture was quenched with ice water (15 mL) and extracted with EtOAc (15 mL×3). The combined organic layers were washed with brine (20 mL), dried over Na₂SO₄ and concentrated under reduced pressure. The residue was purified by RP-prep-HPLC to afford 1-(4-(difluoromethoxy)phenyl)-3-(4-methoxyphenyl)-7-(2,2,2-trifluoroethylamino)-1,8-naphthyridin-2(1H)-one (Example 130).

¹H NMR (400 MHz, CDCl₃) δ: 7.66-7.62 (m, 4H), 7.27-7.16 (m, 4H), 6.86 (d, J=8.0 Hz, 2H), 6.49 (t, J_{HF}=72.0 Hz, 1H), 6.30 (s, 1H), 5.36-5.35 (m, 1H), 3.77 (s, 3H), 3.67-3.62 (m, 2H). LC-MS (ESI): m/z 492 [M+H]⁺.

The procedure set forth above for General Procedure II (Route B, Method A, Y=CH, Z=Cl, X=NH) was used to synthesize the following compounds by using appropriate starting materials:

| Example | Structure | Characterization |
|---|---|---|
| 131 | 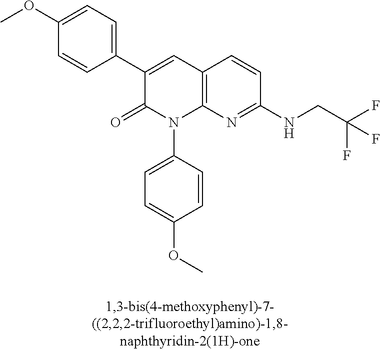 [SMILES: COc1ccc(-c2cc3ccc(NCC(F)(F)F)nc3n(-c3ccc(OC)cc3)c2=O)cc1] | LC-MS (ESI): m/z 456 [M + H]+. 1H NMR (400 MHz, CDCl3) δ: 7.68-7.57 (m, 4H), 7.13 (d, J = 8.4 Hz, 2H), 6.97 (d, J = 8.8 Hz, 2H), 6.86 (d, J = 8.8 Hz, 2H), 6.27 (d, J = 8.4 Hz, 1H), 4.92 (br, 1H), 3.80 (s, 3H), 3.76 (s, 3H), 3.74-3.67 (m, 2H). |
| 132 | 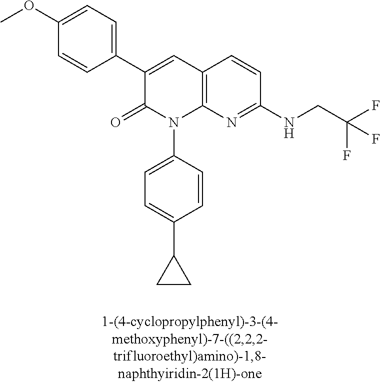 [SMILES: COc1ccc(-c2cc3ccc(NCC(F)(F)F)nc3n(-c3ccc(C4CC4)cc3)c2=O)cc1] | LC-MS (ESI): m/z 466 [M + H]+. 1H NMR (400 MHz, DMSO-d6) δ: 7.97 (s, 1H), 7.86 (d, J = 8.4 Hz, 1H), 7.83-7.75 (m, 1H), 7.67 (d, J = 8.4 Hz, 2H), 7.20 (d, J = 7.2 Hz, 2H), 7.09 (d, J = 7.2 Hz, 2H), 6.97 (d, J = 7.2 Hz, 2H), 6.52 (d, J = 8.0 Hz, 1H), 3.83-3.65 (m, 5H), 2.13-1.95 (m, 1H), 1.15-0.95 (m, 2H), 0.75-0.68 (m, 2H). |
| 133 | 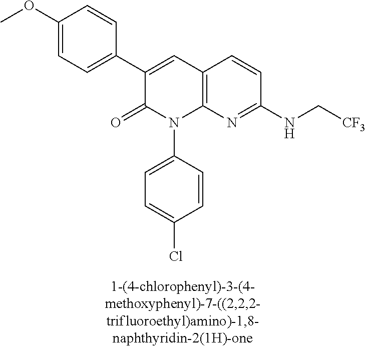 [SMILES: COc1ccc(-c2cc3ccc(NCC(F)(F)F)nc3n(-c3ccc(Cl)cc3)c2=O)cc1] | LC-MS (ESI): m/z 460 [M + H]+. 1H NMR (400 MHz, DMSO-d6) δ: 8.00 (s, 1H), 7.94-7.81 (m, 2H), 7.67 (d, J = 8.8 Hz, 2H), 7.57 (d, J = 8.4 Hz, 2H), 7.31 (d, J = 8.8 Hz, 2H), 6.97 (d, J = 8.8 Hz, 2H), 6.55 (d, J = 8.4 Hz, 1H), 3.79 (s, 3H), 3.97-3.68 (m, 2H). |
| 134 | 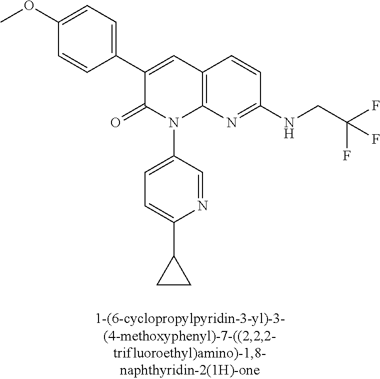 [SMILES: COc1ccc(-c2cc3ccc(NCC(F)(F)F)nc3n(-c3ccc(C4CC4)nc3)c2=O)cc1] | LC-MS (ESI): m/z 467 [M + H]+. 1H NMR (400 MHz, DMSO-d6, 0.8 HCOOH) δ: 8.40 (s, 0.8H), 8.26 (d, J = 2.4 Hz, 1H), 8.00 (s, 1H), 7.88 (d, J = 8.8 Hz, 1H), 7.89-7.85 (m, 1H), 7.66 (d, J = 8.8 Hz, 2H), 7.58 (dd, J = 8.4 Hz, 2.4 Hz, 1H), 7.44 (d, J = 8.4 Hz, 1H), 6.97 (d, J = 8.8 Hz, 2H), 6.55 (d, J = 8.8 Hz, 1H), 3.79 (s, 3H), 3.77- 3.65 (m, 2H), 2.26-2.07 (m, 1H), 1.08-0.95 (m, 4H). |
| 135 | 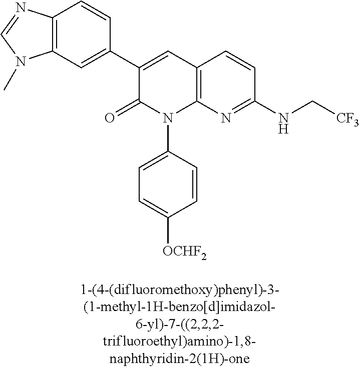 [SMILES: Cn1cnc2ccc(-c3cc4ccc(NCC(F)(F)F)nc4n(-c4ccc(OC(F)F)cc4)c3=O)cc21] | LC-MS (ESI): m/z 516 [M + H]+. 1H NMR (400 MHz, DMSO-d6) δ: 8.20 (s, 1H), 8.12 (s, 1H), 7.98 (s, 1H), 7.92 (d, J = 8.4 Hz, 1H), 7.95-7.85 (m, 1H), 7.66 (d, J = 8.4 Hz, 1H), 7.56 (dd, J = 8.4 Hz, 1.6 Hz, 1H), 7.40- 7.30 (m, 4H), 7.31 (t, J_{HF} = 72.0 Hz, 1H), 6.56 (d, J = 8.4 Hz, 1H), 3.85 (s, 3H), 3.83-3.69 (m, 2H). |
| 306 | 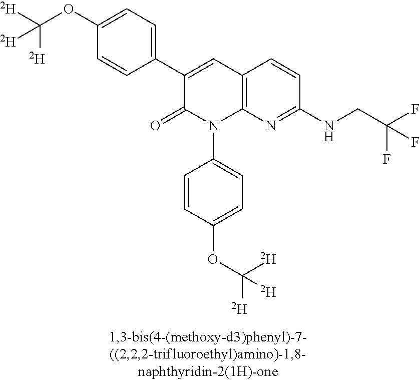 [SMILES: [2H]C([2H])([2H])Oc1ccc(-c2cc3ccc(NCC(F)(F)F)nc3n(-c3ccc(OC([2H])([2H])[2H])cc3)c2=O)cc1] | LC-MS: m/z 462 (M + H)+. 1H NMR (400 MHz, DMSO-d6) δ: 7.95 (s, 1H), 7.86 (d, J = 8.5 Hz, 1H), 7.79 (s, 1H), 7.70-7.60 (m, 2H), 7.15 (d, J = 8.0 Hz, 2H), 7.04 (d, J = 8.0 Hz, 2H), 6.96 (d, J = 8.0 Hz, 2H), 6.51 (d, J = 8.5 Hz, 1H), 3.82-3.75 (m, 2H). |
| 307 | 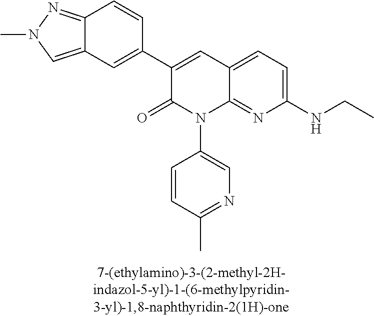 [SMILES: CCNc1ccc2cc(-c3ccc4nn(C)cc4c3)c(=O)n(-c3ccc(C)nc3)c2n1] | LC-MS: m/z 411 (M + H)+ 1H NMR (400 MHz, DMSO-d6) δ: 8.35 (s, 1H), 8.33 (s, 1H), 8.06 (d, J = 1.2 Hz, 1H), 8.02 (s, 1H), 7.78 (d, J = 8.4 Hz, 1H), 7.66 (dd, J = 8.0 Hz, 2.4 Hz, 1H), 7.61-7.51 (m, 2H), 7.41 (d, J = 8.0 Hz, 1H), 7.34 (s, 1H), 6.40 (d, J = 8.4 Hz, 1H), 4.17 (s, 3H), 2.93 (t, J = 6.8 Hz, 2H), 2.56 (s, 3H), 0.89 (t, J = 6.8 Hz, 3H). |
| 308 | 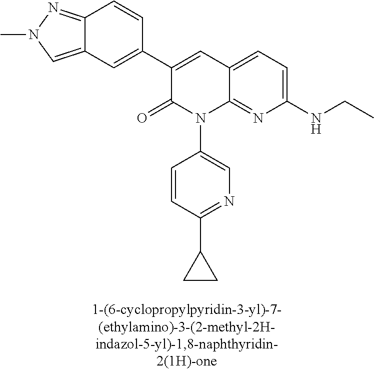 [SMILES: CCNc1ccc2cc(-c3ccc4nn(C)cc4c3)c(=O)n(-c3ccc(C4CC4)nc3)c2n1] | LC-MS: m/z 437 (M + H)+ 1H NMR (400 MHz, DMSO-d6) δ: 8.35 (s, 1H), 8.28 (d, J = 2.4 Hz, 1H), 8.06 (t, J = 1.2 Hz, 1H), 8.02 (s, 1H), 7.78 (d, J = 8.4 Hz, 1H), 7.61 (dd, J = 8.0 Hz, 2.4 Hz, 1H), 7.57-7.54 (m, 2H), 7.45 (d, J = 8.0 Hz, 1H), 7.34 (s, 1H), 6.39 (d, J = 8.4 Hz, 1H), 4.17 (s, 3H), 3.08-2.85 (m, 2H), 2.22-2.18 (m, 1H), 1.06-0.93 (m, 4H), 0.89 (t, J = 7.2 Hz, 3H). |
| 309 | 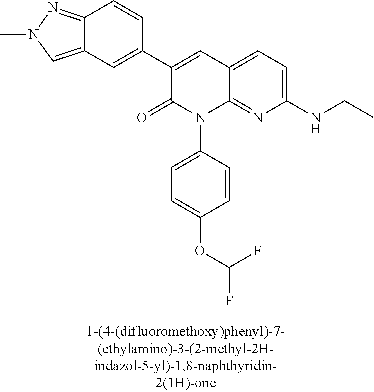 [SMILES: CCNc1ccc2cc(-c3ccc4nn(C)cc4c3)c(=O)n(-c3ccc(OC(F)F)cc3)c2n1] | LC-MS: m/z 462 (M + H)+ 1H NMR (400 MHz, DMSO-d6) δ: 8.34 (s, 1H), 8.06 (s, 1H), 8.00 (s, 1H), 7.76 (s, 1H), 7.56 (br, 2H), 7.32 (br, 6H), 6.52-6.23 (m, 1H), 4.16 (s, 3H), 2.92 (br, 2H), 0.87 (br, 3H). |
| 310 | 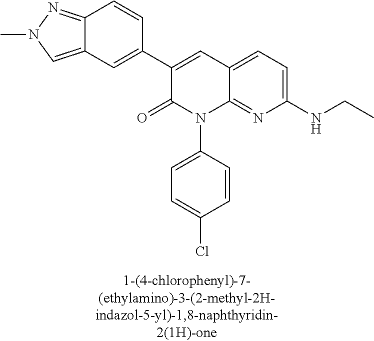 [SMILES: CCNc1ccc2cc(-c3ccc4nn(C)cc4c3)c(=O)n(-c3ccc(Cl)cc3)c2n1] | LC-MS: m/z 430 (M + H)+. 1H NMR (400 MHz, DMSO-d6) δ: 8.32 (s, 1H), 8.06 (d, J = 1.2 Hz, 1H), 8.01 (s, 1H), 7.77 (d, J = 8.4 Hz, 1H), 7.56 (d, J = 4.4, Hz, 4H), 7.36-7.29 (m, 3H), 6.38 (d, J = 8.4 Hz, 1 H), 4.17 (s, 3H), 2.93 (q, J = 7.2 Hz, 2H), 0.90 (t, J = 7.2 Hz, 3H). |
| 311 | 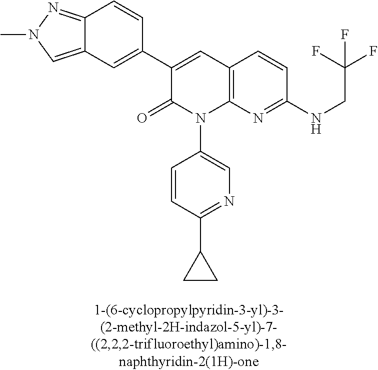 [SMILES: Cn1cc2cc(-c3cc4ccc(NCC(F)(F)F)nc4n(-c4ccc(C5CC5)nc4)c3=O)ccc2n1] | LC-MS: m/z 491 (M + H)+. 1H NMR (400 MHz, DMSO-d6) δ: 8.36 (s, 1H), 8.28 (d, J = 2.4 Hz, 1H), 8.08 (d, J = 2.0 Hz, 2H), 7.93-7.86 (m, 2H), 7.66-7.53 (m, 3H), 7.45 (d, J = 8.4 Hz, 1H), 6.56 (d, J = 8.4 Hz, 1H), 4.17 (s, 3H), 3.80- 3.74 (m, 2H), 2.22-2.18 (m, 1H), 1.10-0.96 (m, 4H). |
| 312 | 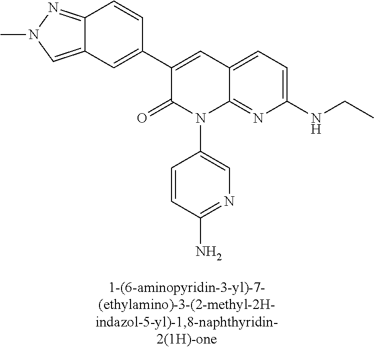 [SMILES: CCNc1ccc2cc(-c3ccc4nn(C)cc4c3)c(=O)n(-c3ccc(N)nc3)c2n1] | LC-MS: m/z 412 (M + H)+. 1H NMR (400 MHz, DMSO-d6) δ: 8.34 (s, 1H), 8.05 (s, 1H), 7.96 (s, 1H), 7.81-7.66 (m, 2H), 7.55 (s, 2H), 7.32-7.25 (m, 2H), 6.55 (d, J = 8.7 Hz, 1H), 6.38 (d, J = 8.5 Hz, 1H), 6.00 (s, 2H), 4.17 (s, 3H), 3.05-2.93 (m, 2H), 0.96 (t, J = 7.2 Hz, 3H). |

### Preparation of Example 136 via General Procedure II (Route B, Method A, Y=CH, Z=Cl, X=O

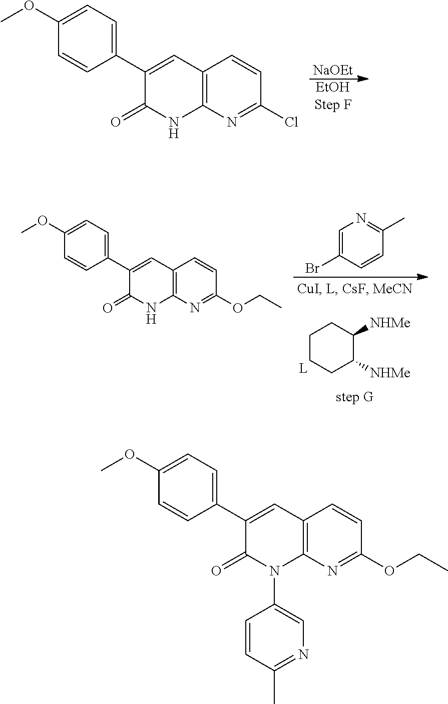

### Step F: 7-ethoxy-3-(4-methoxyphenyl)-1,8-naphthyridin-2(1H)-one

To a solution of 7-chloro-3-(4-methoxyphenyl)-1,8-naphthyridin-2(1H)-one (200 mg, 0.70 mmol, 1.0 eq.) in EtOH (10 mL) was added NaOEt (142 mg, 2.1 mmol, 3.0 eq.) at room temperature. The resulting mixture was stirred at 100° C. for 2 hrs. The mixture was quenched with ice water (30 mL) and extracted with EtOAc (30 mL×3). The combined organic layers were washed with brine (20 mL), dried over Na₂SO₄ and concentrated under reduced pressure. The residue was purified by flash column chromatography on silica gel to afford 7-ethoxy-3-(4-methoxyphenyl)-1,8-naphthyridin-2(1H)-one (200 mg, 97% yield) as a white solid. LC-MS (ESI): m/z 297 [M+H]⁺.

### Step G: 7-ethoxy-3-(4-methoxyphenyl)-1-(6-methylpyridin-3-yl)-1,8-naphthyridin-2(1H)-one

To a solution of 7-ethoxy-3-(4-methoxyphenyl)-1,8-naphthyridin-2(1H)-one (60 mg, 0.2 mmol, 1.0 eq.) in MeCN (3 mL) was added CuI (38 mg, 0.2 mmol, 1.0 eq.), (1R,2R)—N1,N2-dimethylcyclohexane-1,2-diamine (57 mg, 0.4 mmol, 2.0 eq.), CsF (91 mg, 0.6 mmol, 3.0 eq.) and 5-bromo-2-methylpyridine (51 mg, 0.3 mmol, 1.5 eq.) at room temperature in a sealed tube. The resulting mixture was stirred at 90° C. under N2 atmosphere overnight. Then the reaction mixture was diluted with water (15 mL) and extracted with EtOAc (15 mL×3). The combined organic layers were washed with brine (20 mL), dried over Na₂SO₄ and concentrated under reduced pressure. The residue was purified by RP-prep-HPLC to afford 7-ethoxy-3-(4-methoxyphenyl)-1-(6-methylpyridin-3-yl)-1,8-naphthyridin-2(1H)-one (Example 136).

¹H NMR (400 MHz, DMSO-d₆) δ: 8.62 (d, J=5.6 Hz, 1H), 8.16 (s, 1H), 8.15 (d, J=8.0 Hz, 1H), 7.69 (d, J=8.8 Hz, 2H), 7.33 (d, J=1.6 Hz, 1H), 7.26 (dd, J=5.6 Hz, 1.6 Hz, 1H), 6.99 (d, J=8.8 Hz, 2H), 6.73 (d, J=8.4 Hz, 1H), 3.93 (q, J=6.8 Hz, 2H), 3.80 (s, 3H), 2.55 (s, 3H), 1.10 (t, J=6.8 Hz, 3H). LC-MS (ESI): m/z 388 [M+H]⁺.

The procedure set forth above for General Procedure II (Route B, Method A, Y=CH, Z=Cl, X=O) was used to synthesize the following compounds by using appropriate starting materials:

| Example | Structure | Characterization |
|---|---|---|
| 137 | 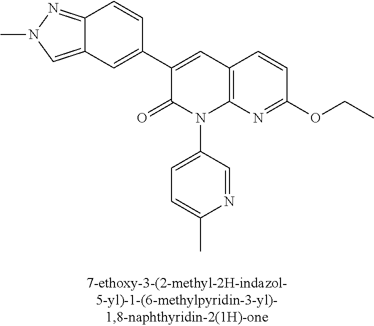 [SMILES: CCOc1ccc2cc(-c3ccc4nn(C)cc4c3)c(=O)n(-c3ccc(C)nc3)c2n1] | LC-MS (ESI): m/z 412 [M + H]+. 1H NMR (400 MHz, DMSO-d6) δ: 8.63 (d, J = 5.2 Hz, 1H), 8.40 (s, 1H), 8.24 (s, 1H), 8.17 (d, J = 8.4 Hz, 1H), 8.11 (s, 1H), 7.63- 7.55 (m, 2H), 7.37-7.34 (m, 1H), 7.29 (dd, J = 5.6, 2.0 Hz, 1H), 6.75 (d, J = 8.4 Hz, 1H), 4.18 (s, 3H), 3.95 (q, J = 7.2 Hz, 2H), 2.55 (s, 3H), 1.11 (t, J = 7.2 Hz, 3H). |
| 138 | 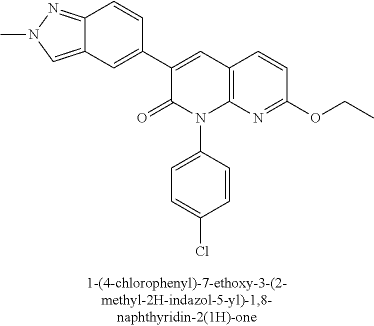 [SMILES: CCOc1ccc2cc(-c3ccc4nn(C)cc4c3)c(=O)n(-c3ccc(Cl)cc3)c2n1] | LC-MS (ESI): m/z 431 [M + H]+. 1H NMR (400 MHz, DMSO-d6) δ: 8.40 (s, 1H), 8.23 (s, 1H), 8.17 (d, J = 8.4 Hz, 1H), 8.12 (s, 1H), 7.64- 7.51 (m, 4H), 7.42 (d, J = 8.4 Hz, 2H), 6.73 (d, J = 8.3 Hz, 1H), 4.19 (s, 3H), 3.96 (q, J = 7.2 Hz, 2H), 1.11 (t, J = 7.2 Hz, 3H). |
| 139 | 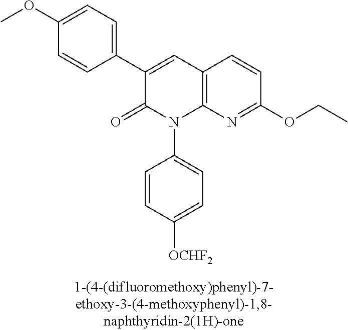 [SMILES: CCOc1ccc2cc(-c3ccc(OC)cc3)c(=O)n(-c3ccc(OC(F)F)cc3)c2n1] | LC-MS (ESI): m/z 439 [M + H]+. 1H NMR (400 MHz, DMSO-d6) δ: 8.14 (s, 1H), 8.13 (d, J = 8.4 Hz, 1H), 7.69 (d, J = 8.8 Hz, 2H), 7.41 (d, J = 8.8 Hz, 2H), 7.34 (t, J_{HF} = 74.0 Hz, 1H), 7.33 (d, J = 8.8 Hz, 2H), 6.99 (d, J = 8.8 Hz, 2H), 6.70 (d, J = 8.4 Hz, 1H), 3.93 (q, J = 6.8 Hz, 2H), 3.79 (s, 3H), 1.08 (t, J = 6.8 Hz, 3H). |
| 140 | 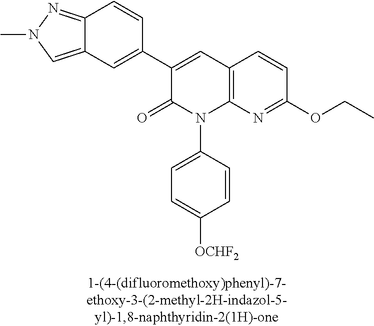 [SMILES: CCOc1ccc2cc(-c3ccc4nn(C)cc4c3)c(=O)n(-c3ccc(OC(F)F)cc3)c2n1] | LC-MS (ESI): m/z 463 [M + H]+. 1H NMR (400 MHz, DMSO-d6) δ: 8.39 (s, 1H), 8.22 (s, 1H), 8.16 (d, J = 8.4 Hz, 1H), 8.11 (s, 1H), 7.63- 7.55 (m, 2H), 7.42 (d, J = 8.8 Hz, 2H), 7.34 (d, J = 8.8 Hz, 2H), 7.33 (t, J_{HF} = 74.0 Hz, 1H), 6.72 (d, J = 8.4 Hz, 1H), 4.18 (s, 3H), 3.95 (q, J = 7.2 Hz, 2H), 1.09 (t, J = 7.2 Hz, 3H). |
| 141 | 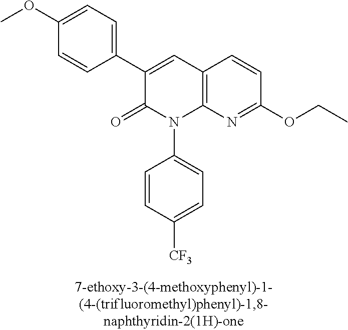 [SMILES: CCOc1ccc2cc(-c3ccc(OC)cc3)c(=O)n(-c3ccc(C(F)(F)F)cc3)c2n1] | LC-MS (ESI): m/z 441 [M + H]+. 1H NMR (400 MHz, DMSO-d6) δ: 8.17 (s, 1H), 8.15 (d, J = 8.4 Hz, 1H), 7.92 (d, J = 8.4 Hz, 2H), 7.70 (d, J = 8.8 Hz, 2H), 7.63 (d, J = 8.4 Hz, 2H), 6.99 (d, J = 8.8 Hz, 2H), 6.73 (d, J = 8.4 Hz, 1H), 3.90 (q, J = 7.2 Hz, 2H), 3.80 (s, 3H), 1.04 (t, J = 7.2 Hz, 3H). |
| 142 | 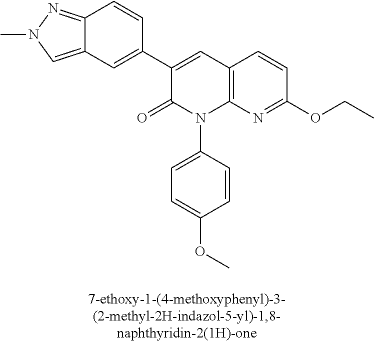 [SMILES: CCOc1ccc2cc(-c3ccc4nn(C)cc4c3)c(=O)n(-c3ccc(OC)cc3)c2n1] | LC-MS (ESI): m/z 427 [M + H]+. 1H NMR (400 MHz, DMSO-d6) δ: 8.39 (s, 1H), 8.19 (s, 1H), 8.17-8.07 (m, 2H), 7.59 (q, J = 9.0 Hz, 2H), 7.25 (d, J = 8.8 Hz, 2H), 7.08 (d, J = 8.8 Hz, 2H), 6.71 (d, J = 8.4 Hz, 1H), 4.18 (s, 3H), 3.96 (q, J = 7.0 Hz, 2H), 3.84 (s, 3H), 1.11 (t, J = 7.0 Hz, 3H). |
| 143 | 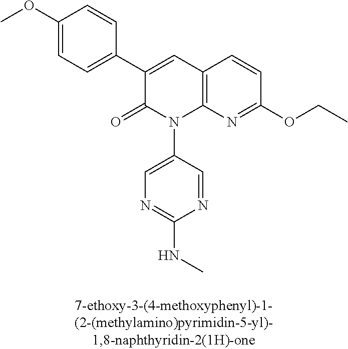 [SMILES: CCOc1ccc2cc(-c3ccc(OC)cc3)c(=O)n(-c3cnc(NC)nc3)c2n1] | LC-MS (ESI): m/z 404 [M + H]+. 1H NMR (400 MHz, DMSO-d6) δ: 8.29 (s, 2H), 8.14 (d, J = 8.4 Hz, 1H), 8.14 (s, 1H), 7.69 (d, J = 8.8 Hz, 2H), 7.37 (d, J = 4.8 Hz, 1H), 7.00 (d, J = 8.8 Hz, 2H), 6.74 (d, J = 8.4 Hz, 1H), 4.07 (q, J = 7.2 Hz, 2H), 3.80 (s, 3H), 2.86 (d, J = 4.4 Hz, 3H), 1.19 (t, J = 7.2 Hz, 3H). |
| 144 | 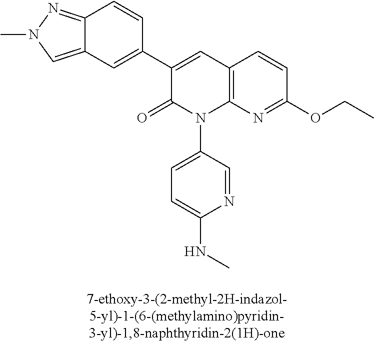 [SMILES: CCOc1ccc2cc(-c3ccc4nn(C)cc4c3)c(=O)n(-c3ccc(NC)nc3)c2n1] | LC-MS (ESI): m/z 427 [M + H]+. 1H NMR (400 MHz, DMSO-d6) δ: 8.40 (s, 1H), 8.18 (s, 1H), 8.14 (d, J = 8.4 Hz, 1H), 8.10 (s, 1H), 7.91 (d, J = 2.8 Hz, 1H), 7.61 (d, J = 9.2 Hz, 1H), 7.56 (dd, J = 9.2 Hz, 1.6 Hz, 1H), 7.37 (dd, J = 8.8 Hz, 2.4 Hz, 1H), 6.74-6.65 (m, 2H), 6.58 (d, J = 8.8 Hz, 1H), 4.18 (s, 3H), 4.03 (q, J = 7.2 Hz, 2H), 2.83 (d, J = 5.2 Hz, 3H), 1.16 (t, J = 6.8 Hz, 3H). |
| 145 | 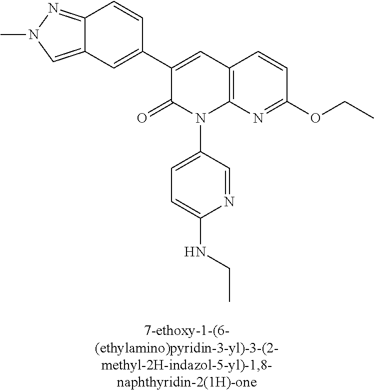 [SMILES: CCNc1ccc(-n2c(=O)c(-c3ccc4nn(C)cc4c3)cc3ccc(OCC)nc32)cn1] | LC-MS (ESI): m/z 441 [M + H]+. 1H NMR (400 MHz, DMSO-d6) δ: 8.40 (s, 1H), 8.19 (s, 1H), 8.14 (d, J = 8.4 Hz, 1H), 8.11 (s, 1H), 7.89 (d, J = 2.8 Hz, 1H), 7.61 (d, J = 9.1 Hz, 1H), 7.57 (dd, J = 9.2 Hz, 1.6 Hz, 1H), 7.36 (dd, J = 8.8 Hz, 2.8 Hz, 1H), 6.72 (d, J = 8.4 Hz, 1H), 6.66 (t, J = 5.6 Hz, 1H), 6.58 (d, J = 8.8 Hz, 1H), 4.19 (s, 3H), 4.05 (q, J = 7.2 Hz, 2H), 3.42-3.36 (m, 2H), 1.17 (t, J = 7.2 Hz, 6H). |
| 146 | 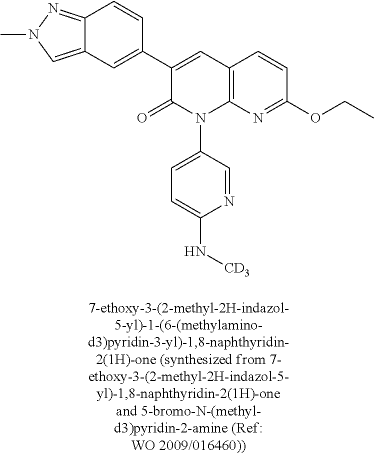 [SMILES: [2H]C([2H])([2H])Nc1ccc(-n2c(=O)c(-c3ccc4nn(C)cc4c3)cc3ccc(OCC)nc32)cn1] | LC-MS (ESI): m/z 430 [M + H]+. 1H NMR (400 MHz, DMSO-d6) δ: 8.40 (s, 1H), 8.19 (s, 1H), 8.14 (d, J = 8.4 Hz, 1H), 8.10 (s, 1H), 7.91 (d, J = 2.4 Hz, 1H), 7.59 (q, J = 9.2 Hz, 2H), 7.37 (dd, J = 8.8 Hz, 2.4 Hz, 1H), 6.72 (d, J = 8.4 Hz, 1H), 6.65 (s, 1H), 6.58 (d, J = 8.8 Hz, 1H), 4.18 (s, 3H), 4.04 (q, J = 7.2 Hz, 2H), 1.16 (t, J = 7.2 Hz, 3H). |
| 147 | 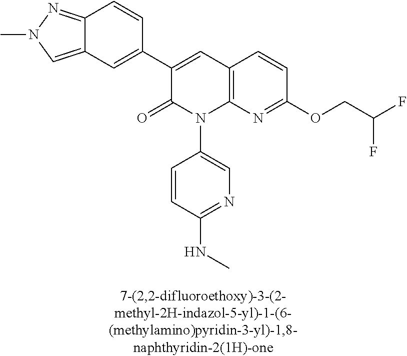 [SMILES: CNc1ccc(-n2c(=O)c(-c3ccc4nn(C)cc4c3)cc3ccc(OCC(F)F)nc32)cn1] | LC-MS (ESI): m/z 463 [M + H]+. 1H NMR (400 MHz, DMSO-d6) δ: 8.46 (s, 1H), 8.28 (t, J = 4.0 Hz, 2H), 8.17 (s, 1H), 8.00 (d, J = 2.4 Hz, 1H), 7.67 (d, J = 8.8 Hz, 1H), 7.62 (dd, J = 9.2 Hz, 1.2 Hz, 1H), 7.47 (dd, J = 8.8 Hz, 2.4 Hz, 1H), 6.91 (d, J = 8.4 Hz, 1H), 6.82 (br, 1H), 6.66 (d, J = 8.8 Hz, 1H), 6.25 (tt, J_{HF} = 55.2, J = 3.6 Hz, 1H), 4.32 (tt, J_{HF} = 14.8 Hz, J = 3.2 Hz, 2H), 4.24 (s, 3H), 2.89 (d, J = 4.8 Hz, 3H). |
| 148 | 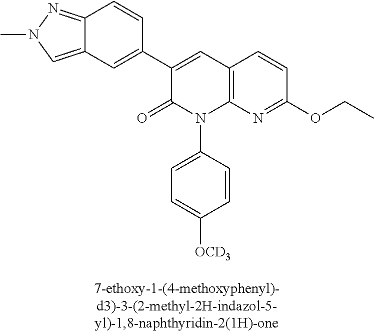 | LC-MS (ESI): m/z 430 [M + H]+. 1H NMR (400 MHz, DMSO-d6) δ: 8.38 (s, 1H), 8.19 (s, 1H), 8.16-8.06 (m, 2H), 7.58 (q, J = 9.2 Hz, 2H), 7.25 (d, J = 8.7 Hz, 2H), 7.07 (d, J = 8.7 Hz, 2H), 6.70 (d, J = 8.3 Hz, 1H), 4.18 (s, 3H), 3.96 (q, J = 7.0 Hz, 2H), 1.10 (t, J = 7.0 Hz, 3H). |
| 149 | 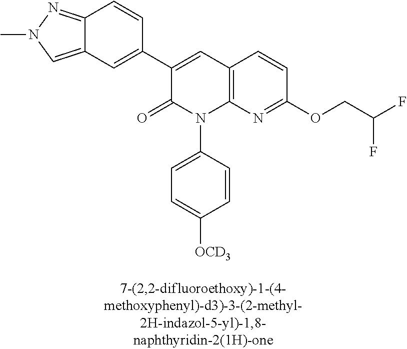 | LC-MS (ESI): m/z 466 [M + H]+. 1H NMR (400 MHz, DMSO-d6) δ: 8.40 (s, 1H), 8.23 (d, J = 8.1 Hz, 2H), 8.12 (s, 1H), 7.59 (q, J = 9.1 Hz, 2H), 7.28 (d, J = 8.3 Hz, 2H), 7.09 (d, J = 8.2 Hz, 2H), 6.84 (d, J = 8.3 Hz, 1H), 6.09 (t, J_{HF} = 55.2 Hz, 1H), 4.27-4.09 (m, 5H). |
| 150 | 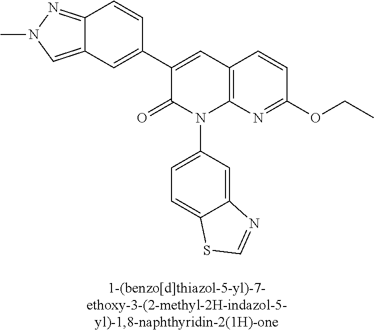 [SMILES: CCOc1ccc2cc(-c3ccc4nn(C)cc4c3)c(=O)n(-c3ccc4scnc4c3)c2n1] | LC-MS (ESI): m/z 454 [M + H]+. 1H NMR (400 MHz, DMSO-d6) δ: 9.47 (s, 1H), 8.38 (s, 1H), 8.32 (d, J = 8.4 Hz, 1H), 8.24 (s, 1H), 8.19 (d, J = 8.4 Hz, 1H), 8.12 (d, J = 4.4 Hz, 2H), 7.60 (s, 2H), 7.48 (d, J = 8.4 Hz, 1H), 6.72 (d, J = 8.4 Hz, 1H), 4.17 (s, 3H), 3.84 (q, J = 7.2 Hz, 2H), 0.99 (t, J = 7.2 Hz, 3H). |
| 313 | 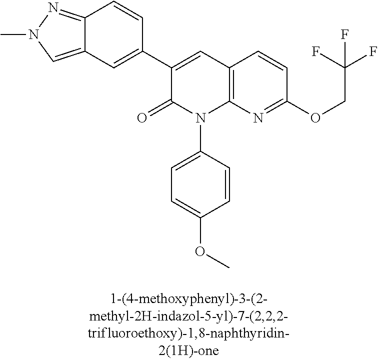 [SMILES: COc1ccc(-n2c(=O)c(-c3ccc4nn(C)cc4c3)cc3ccc(OCC(F)(F)F)nc32)cc1] | LC-MS: m/z 481 (M + H)+. 1H NMR (400 MHz, DMSO-d6) δ: 8.39 (s, 1H), 8.26 (d, J = 8.4 Hz, 1H), 8.23 (s, 1H), 8.11 (s, 1H), 7.61 (d, J = 9.1 Hz, 1H), 7.57 (dd, J = 9.1 Hz, 1.5 Hz, 1H), 7.26 (d, J = 8.8 Hz, 2H), 7.09 (d, J = 8.9 Hz, 2H), 6.89 (d, J = 8.3 Hz, 1H), 4.61 (q, JHF = 9.0 Hz, 2H), 4.18 (s, 3H), 3.83 (s, 3H). |
| 314 | 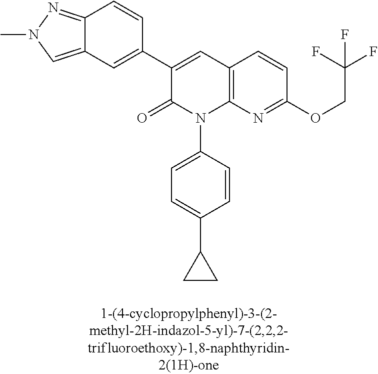 [SMILES: Cn1cc2cc(-c3cc4ccc(OCC(F)(F)F)nc4n(-c4ccc(C5CC5)cc4)c3=O)ccc2n1] | LC-MS: m/z 491 (M + H)+. 1H NMR (400 MHz, DMSO-d6) δ: 8.39 (s, 1H), 8.26 (s, 1H), 8.24 (s, 1H), 8.11 (s, 1H), 7.63-7.56 (m, 2H), 7.22 (d, J = 8.0 Hz, 4H), 6.88 (d, J = 7.8 Hz, 1H), 4.58 (q, JHF = 8.7 Hz, 2H), 4.17 (s, 3H), 2.06-1.98 (m, 1H), 1.04-1.01 (m, 2H), 0.74-0.69 (m, 2H). |
| 315 | 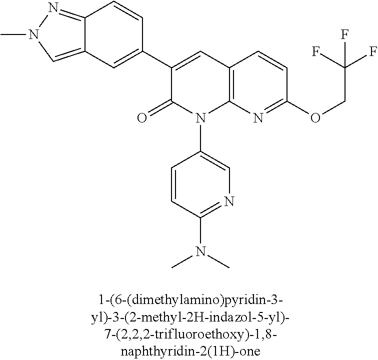 [SMILES: CN(C)c1ccc(-n2c(=O)c(-c3ccc4nn(C)cc4c3)cc3ccc(OCC(F)(F)F)nc32)cn1] | LC-MS: m/z 495 (M + H)+ 1H NMR (400 MHz, DMSO-d6) δ: 8.41 (s, 1H), 8.34-8.22 (m, 3H), 8.16-8.06 (m, 1H), 7.67-7.54 (m, 2H), 6.93 (d, J = 8.4 Hz, 1H), 6.69 (dd, J = 1.6 Hz, 0.8 Hz, 1H), 6.59 (dd, J = 5.2 Hz, 1.6 Hz, 1H), 4.74-4.61 (m, 2H), 4.18 (s, 3H), 3.04 (s, 6H). |
| 316 | 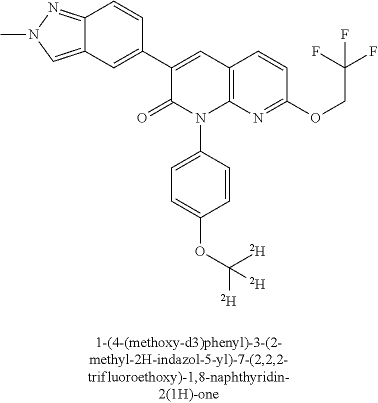 [SMILES: [2H]C([2H])([2H])Oc1ccc(-n2c(=O)c(-c3ccc4nn(C)cc4c3)cc3ccc(OCC(F)(F)F)nc32)cc1] | LC-MS: m/z 484 (M + H)+ 1H NMR (400 MHz, CDCl3) δ: 8.15 (s, 1H), 7.87-7.82 (m, 3H), 7.65 (d, J = 9.1 Hz, 1H), 7.54 (dd, J = 9.1 Hz, 1.5 Hz, 1H), 7.15 (d, J = 8.9 Hz, 2H), 6.99 (d, J = 8.9 Hz, 2H), 6.66 (d, J = 8.3 Hz, 1H), 4.31 (q, JHF = 8.5 Hz, 2H), 4.16 (s, 3H). |

### Preparation of Example 151 via General Procedure II (Route B, Method B, Y=CH, Z=Cl, X=O

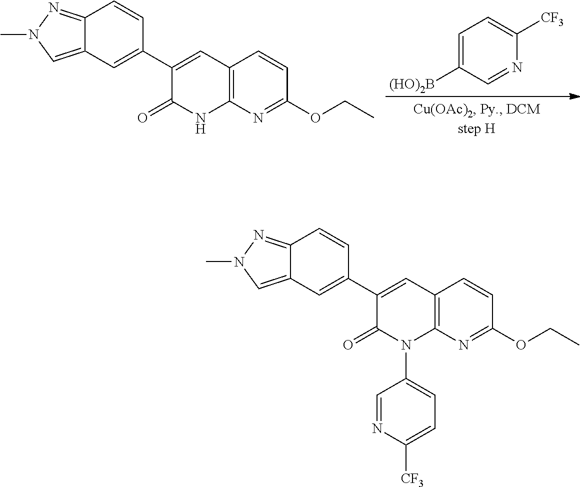

### Step H: 7-ethoxy-3-(2-methyl-2H-indazol-5-yl)-1-(6-(trifluoromethyl)pyridin-3-yl)-1,8-naphthyridin-2(1H)-one

To a solution of 7-ethoxy-3-(2-methyl-2H-indazol-5-yl)-1,8-naphthyridin-2(1H)-one (60 mg, 0.19 mmol, 1.0 eq.) in DCM (5 mL) was added Cu(OAc)₂ (40 mg, 0.21 mmol, 1.1 eq.), 6-(trifluoromethyl)pyridin-3-ylboronic acid (55 mg, 0.29 mmol, 1.5 eq.) and pyridine (31 μL, 0.38 mmol, 2.0 eq.) at room temperature. The resulting mixture was stirred at 40° C. for 14 hrs. Then the reaction mixture was diluted with water (15 mL) and extracted with DCM (15 mL×3). The combined organic layers were washed with brine (20 mL), dried over Na₂SO₄ and concentrated under reduced pressure. The residue was purified by RP-prep-HPLC to afford 7-ethoxy-3-(2-methyl-2H-indazol-5-yl)-1-(6-(trifluoromethyl)pyridin-3-yl)-1,8-naphthyridin-2(1H)-one (Example 151).

¹H NMR (400 MHz, DMSO-d₆) δ: 8.88 (d, J=2.0 Hz, 1H), 8.40 (s, 1H), 8.29 (s, 1H), 8.28 (dd, J=8.4 Hz, 2.0 Hz, 1H), 8.21 (d, J=8.4 Hz, 1H), 8.16 (d, J=8.4 Hz, 1H), 8.13 (s, 1H), 7.64-7.57 (m, 2H), 6.78 (d, J=8.4 Hz, 1H), 4.18 (s, 3H), 3.93 (q, J=7.2 Hz, 2H), 1.08 (t, J=7.2 Hz, 3H). LC-MS (ESI): m/z 466 [M+H]⁺.

The procedure set forth above for General Procedure II (Route B, Method B, Y=CH, Z=Cl, X=O) was used to synthesize the following compounds by using appropriate starting materials:

| Example | Structure | Characterization |
|---|---|---|
| 152 | 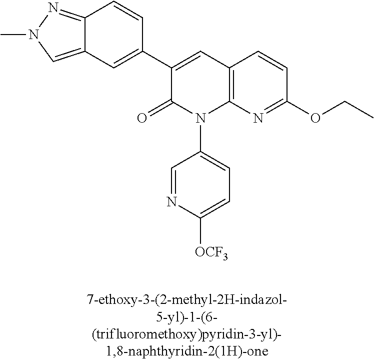 [SMILES: CCOc1ccc2cc(-c3ccc4nn(C)cc4c3)c(=O)n(-c3ccc(OC(F)(F)F)nc3)c2n1] | LC-MS (ESI): m/z 482 [M + H]+. 1H NMR (400 MHz, DMSO-d6) δ: 8.47 (s, 1H), 8.40 (s, 1H), 8.27 (s, 1H), 8.18 (d, J = 8.4 Hz, 2H), 8.13 (s, 1H), 7.64-7.57 (m, 2H), 7.53 (d, J = 8.8 Hz, 1H), 6.76 (d, J = 8.4 Hz, 1H), 4.18 (s, 3H), 3.96 (q, J = 7.2 Hz, 2H), 1.10 (t, J = 7.2 Hz, 3H). |
| 153 | 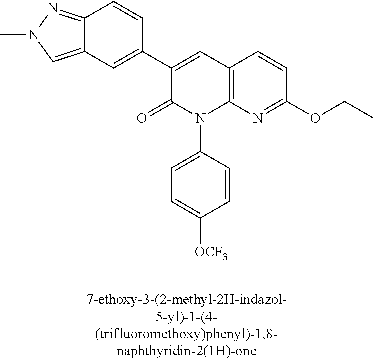 [SMILES: CCOc1ccc2cc(-c3ccc4nn(C)cc4c3)c(=O)n(-c3ccc(OC(F)(F)F)cc3)c2n1] | LC-MS (ESI): m/z 481 [M + H]+. 1H NMR (400 MHz, DMSO-d6) δ: 8.40 (s, 1H), 8.24 (s, 1H), 8.17 (d, J = 8.4 Hz, 1H), 8.13 (s, 1H), 7.62- 7.52 (m, 6H), 6.73 (d, J = 8.4 Hz, 1H), 4.19 (s, 3H), 3.94 (q, J = 6.8 Hz, 2H), 1.06 (t, J = 6.8 Hz, 3H). |
| 154 | 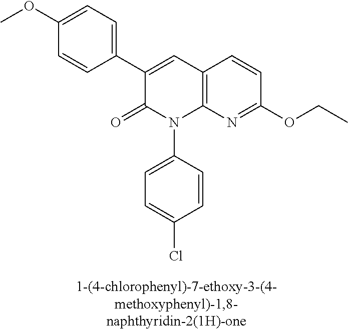 [SMILES: CCOc1ccc2cc(-c3ccc(OC)cc3)c(=O)n(-c3ccc(Cl)cc3)c2n1] | LC-MS (ESI): m/z 407 [M + H]+. 1H NMR (400 MHz, DMSO-d6) δ 8.14 (t, J = 4.2 Hz, 2H), 7.69 (d, J = 8.8 Hz, 2H), 7.60 (d, J = 8.8 Hz, 2H), 7.40 (d, J = 8.8 Hz, 2H), 7.99 (d, J = 8.8 Hz, 2H), 6.71 (d, J = 8.4 Hz, 1H), 3.94 (q, J = 7.0 Hz, 2H), 3.80 (s, 3H), 1.10 (t, J = 7.0 Hz, 3H). |
| 155 | 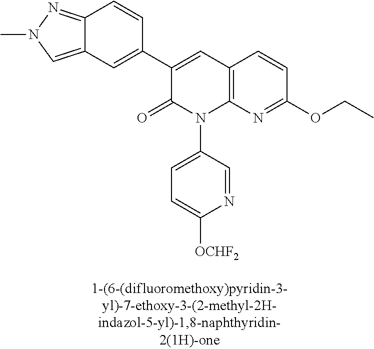 [SMILES: CCOc1ccc2cc(-c3ccc4nn(C)cc4c3)c(=O)n(-c3ccc(OC(F)F)nc3)c2n1] | LC-MS (ESI): m/z 464 [M + H]+. 1H NMR (400 MHz, DMSO-d6) δ: 8.40 (s, 1H), 8.34 (d, J = 2.4 Hz, 1H), 8.25 (s, 1H), 8.18 (d, J = 8.4 Hz, 1H), 8.12 (s, 1H), 8.05 (dd, J = 8.4 Hz, 2.4 Hz, 1H), 7.82 (t, J_{HF} = 72.8 Hz, 1H), 7.62 (d, J = 9.2 Hz, 1H), 7.57 (dd, J = 9.2 Hz, 1.6 Hz, 1H), 7.30 (d, J = 8.8 Hz, 1H), 6.76 (d, J = 8.4 Hz, 1H), 4.18 (s, 3H), 3.98 (q, J = 6.8 Hz, 2H), 1.13 (t, J = 7.2 Hz, 3H). |
| 156 | 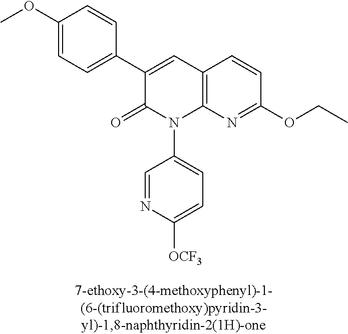 [SMILES: CCOc1ccc2cc(-c3ccc(OC)cc3)c(=O)n(-c3ccc(OC(F)(F)F)nc3)c2n1] | LC-MS (ESI): m/z 458 [M + H]+. 1H NMR (400 MHz, DMSO-d6) δ: 8.45 (s, 1H), 8.19 (s, 1H), 8.16 (d, J = 8.4 Hz, 2H), 7.70 (d, J = 8.4 Hz, 2H), 7.52 (d, J = 8.4 Hz, 1H), 7.0 (d, J = 8.4 Hz, 2H), 6.75 (d, J = 8.4 Hz, 1H), 3.95 (q, J = 6.8 Hz, 2H), 3.80 (s, 3H), 1.09 (t, J = 6.8 Hz, 3H). |
| 157 | 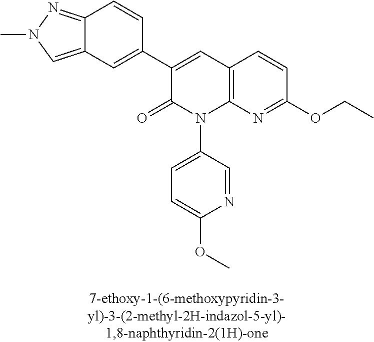 [SMILES: CCOc1ccc2cc(-c3ccc4nn(C)cc4c3)c(=O)n(-c3ccc(OC)nc3)c2n1] | LC-MS (ESI): m/z 428 [M + H]+. 1H NMR (400 MHz, DMSO-d6) δ: 8.47 (s, 1H), 8.30 (s, 1H), 8.28-8.20 (m, 2H), 8.18 (s, 1H), 7.84 (dd, J = 8.8 Hz, 2.4 Hz, 1H), 7.73- 7.60 (m, 2H), 7.07 (d, J = 8.4 Hz, 1H), 6.81 (d, J = 8.4 Hz, 1H), 4.25 (s, 3H), 4.04 (q, J = 7.2 Hz, 2H), 3.99 (s, 3H), 1.19 (t, J = 7.2 Hz, 3H). |
| 158 | 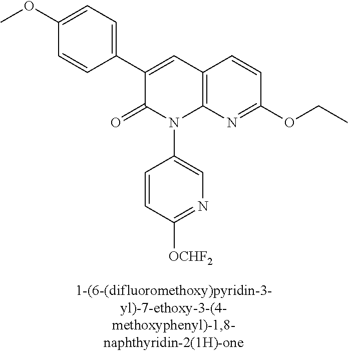 [SMILES: CCOc1ccc2cc(-c3ccc(OC)cc3)c(=O)n(-c3ccc(OC(F)F)nc3)c2n1] | LC-MS (ESI): m/z 440 [M + H]+. 1H NMR (400 MHz, DMSO-d6) δ: 8.32 (s, 1H), 8.16 (d, J = 9.3 Hz, 2H), 8.03 (d, J = 8.6 Hz, 1H), 7.81 (t, J_{HF} = 72.8 Hz, 1H), 7.70 (d, J = 8.3 Hz, 2H), 7.29 (d, J = 8.7 Hz, 1H), 7.00 (d, J = 8.3 Hz, 2H), 6.74 (d, J = 8.5 Hz, 1H), 3.97 (q, J = 6.9 Hz, 2H), 3.80 (s, 3H), 1.12 (t, J = 6.9 Hz, 3H). |
| 159 | 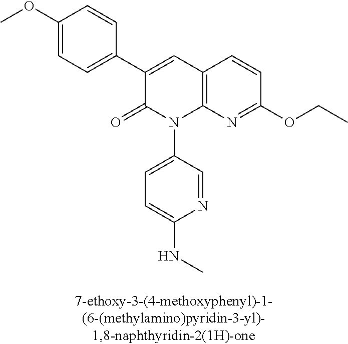 [SMILES: CCOc1ccc2cc(-c3ccc(OC)cc3)c(=O)n(-c3ccc(NC)nc3)c2n1] | LC-MS (ESI): m/z 403 [M + H]+. 1H NMR (400 MHz, DMSO-d6) δ: 8.12 (d, J = 8.4 Hz, 2H), 7.89 (s, 1H), 7.69 (d, J = 8.0 Hz, 2H), 7.35 (d, J = 8.0 Hz, 1H), 6.99 (d, J = 8.4 Hz, 2H), 6.71 (d, J = 8.4 Hz, 2H), 6.57 (d, J = 8.8 Hz, 1H), 4.02 (q, J = 6.8 Hz, 2H), 3.80 (s, 3H), 2.82 (d, J = 4.0 Hz, 3H), 1.15 (t, J = 6.8 Hz, 3H). |
| 160 | 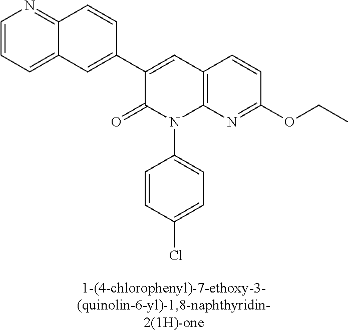 [SMILES: CCOc1ccc2cc(-c3ccc4ncccc4c3)c(=O)n(-c3ccc(Cl)cc3)c2n1] | LC-MS (ESI): m/z 428.1 [M + H]+. 1H NMR (400 MHz, DMSO-d6) δ: 8.92 (dd, J = 4.2 Hz, 1.7 Hz, 1H), 8.42 (s, 1H), 8.41 (s, 1H), 8.40 (s, 1H), 8.21 (d, J = 8.5 Hz, 1H), 8.13 (dd, J = 8.8 Hz, 2.0 Hz, 1H), 8.06 (d, J = 8.8 Hz, 1H), 7.62 (d, J = 8.4 Hz, 2H), 7.56 (dd, J = 8.3 Hz, 4.2 Hz, 1H), 7.45 (d, J = 8.8 Hz, 2H), 6.77 (d, J = 8.4 Hz, 1H), 3.97 (q, J = 7.0 Hz, 2H), 1.12 (t, J = 7.0 Hz, 3H). |
| 161 | 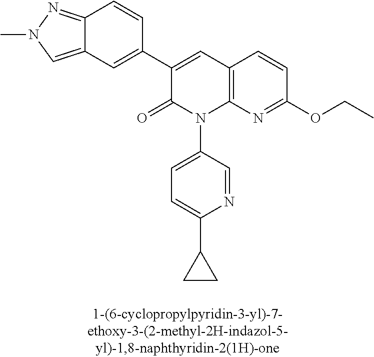 [SMILES: CCOc1ccc2cc(-c3ccc4nn(C)cc4c3)c(=O)n(-c3ccc(C4CC4)nc3)c2n1] | LC-MS (ESI): m/z 438 [M + H]+. 1H NMR (400 MHz, DMSO-d6) δ: 8.46 (s, 1H), 8.43 (d, J = 2.4 Hz, 1H), 8.30 (s, 1H), 8.23 (d, J = 8.4 Hz, 1H), 8.17 (s, 1H), 7.77 (dd, J = 8.4 Hz, 2.4 Hz, 1H), 7.68 (d, J = 8.8 Hz, 1H), 7.63 (dd, J = 9.2 Hz, 1.6 Hz, 1H), 7.55 (d, J = 8.0 Hz, 1H), 6.80 (d, J = 8.4 Hz, 1H), 4.24 (s, 3H), 4.01 (q, J = 6.8 Hz, 2H), 2.31-2.26 (m, 1H), 1.16 (t, J = 6.8 Hz, 3H), 1.09-1.05 (m, 4H). |
| 162 | 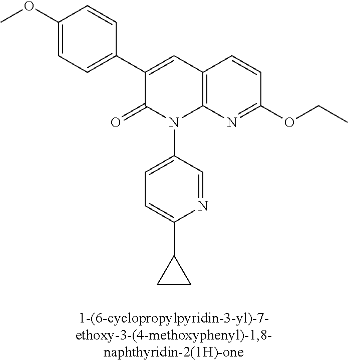 [SMILES: CCOc1ccc2cc(-c3ccc(OC)cc3)c(=O)n(-c3ccc(C4CC4)nc3)c2n1] | LC-MS (ESI): m/z 414 [M + H]+. 1H NMR (400 MHz, DMSO-d6) δ: 8.36 (d, J = 2.4 Hz, 1H), 8.16-8.14 (m, 2H), 7.71-7.66 (m, 3H), 7.49 (d, J = 8.0 Hz, 1H), 7.00 (d, J = 8.8 Hz, 2H), 6.73 (d, J = 8.4 Hz, 1H), 3.94 (q, J = 7.2 Hz, 2H), 3.80 (s, 3H), 2.27- 2.17 (m, 1H), 1.10 (t, J = 7.2 Hz, 3H), 1.05-0.92 (m, 4H). |
| 163 | 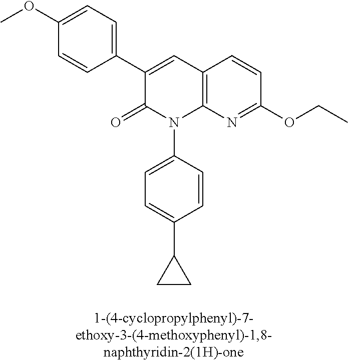 [SMILES: CCOc1ccc2cc(-c3ccc(OC)cc3)c(=O)n(-c3ccc(C4CC4)cc3)c2n1] | LC-MS (ESI): m/z 413 [M + H]+. 1H NMR (400 MHz, DMSO-d6) δ: 8.12 (d, J = 8.4 Hz, 1H), 8.11 (s, 1H), 7.68 (d, J = 8.8 Hz, 2H), 7.21 (d, J = 8.4 Hz, 2H), 7.16 (d, J = 8.4 Hz, 2H), 6.98 (d, J = 8.8 Hz, 1H), 6.68 (d, J = 8.4 Hz, 1H), 3.92 (q, J = 8.8 Hz, 2H), 3.79 (s, 3H), 2.10-1.99 (m, 1H), 1.07 (t, J = 8.8 Hz, 3H), 1.05-0.99 (m, 2H), 0.75-0.72 (m, 2H). |
| 164 | 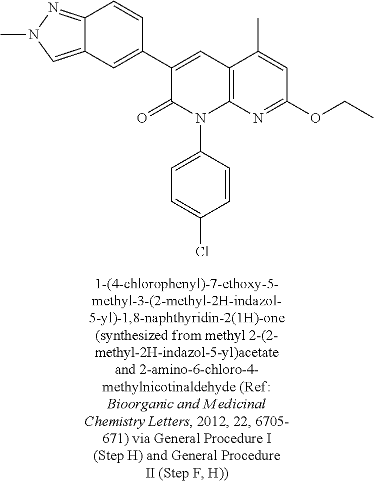 [SMILES: CCOc1cc(C)c2cc(-c3ccc4nn(C)cc4c3)c(=O)n(-c3ccc(Cl)cc3)c2n1] | LC-MS (ESI): m/z 445 [M + H]+. 1H NMR (400 MHz, DMSO-d6) δ: 8.40 (s, 1H), 8.17 (s, 1H), 8.13 (s, 1H), 7.62-7.57 (m, 4H), 7.38 (d, J = 8.4 Hz, 2H), 6.61 (s, 1H), 4.18 (s, 3H), 3.92 (q, J = 7.2 Hz, 2H), 2.61 (s, 3H), 1.36 (t, J = 7.2 Hz, 3H). |
| 165 | 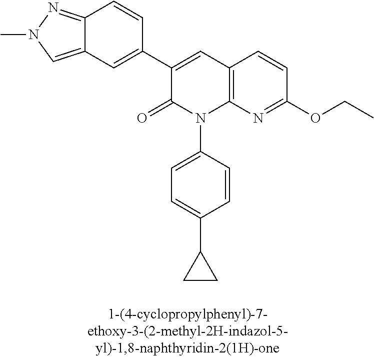 [SMILES: CCOc1ccc2cc(-c3ccc4nn(C)cc4c3)c(=O)n(-c3ccc(C4CC4)cc3)c2n1] | LC-MS (ESI): m/z 437 [M + H]+. 1H NMR (400 MHz, DMSO-d6) δ: 8.39 (s, 1H), 8.19 (s, 1H), 8.17-8.04 (m, 2H), 7.62-7.49 (m, 2H), 7.21 (q, J = 8.6 Hz, 4H), 6.70 (d, J = 8.4 Hz, 1H), 4.18 (s, 3H), 3.93 (q, J = 7.0 Hz, 2H), 2.07-1.95 (m, 1H), 1.07 (t, J = 7.0 Hz, 3H), 1.05-0.96 (m, 2H), 0.83-0.65 (m, 2H). |
| 166 | 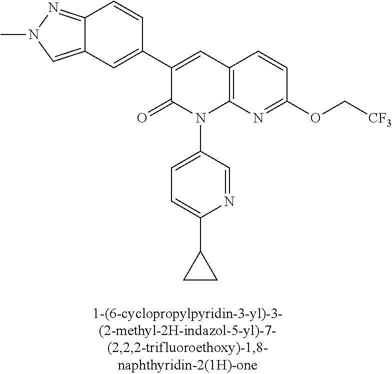 [SMILES: Cn1cc2cc(-c3cc4ccc(OCC(F)(F)F)nc4n(-c4ccc(C5CC5)nc4)c3=O)ccc2n1] | LC-MS (ESI): m/z 492 [M + H ]+. 1H NMR (400 MHz, DMSO-d6) δ: 8.40 (d, J = 8.0 Hz, 2H), 8.29 (d, J = 6.4 Hz, 2H), 8.13 (s, 1H), 7.72 (d, J = 7.2 Hz, 1H), 7.64- 7.56 (m, 2H), 7.51 (d, J = 8.0 Hz, 1H), 6.93 (d, J = 8.4 Hz, 1H), 4.61 (q, J = 8.8 Hz, 2H), 4.19 (s, 3H), 2.27-2.19 (m, 1H), 1.12-0.92 (m, 4H). |
| 167 | 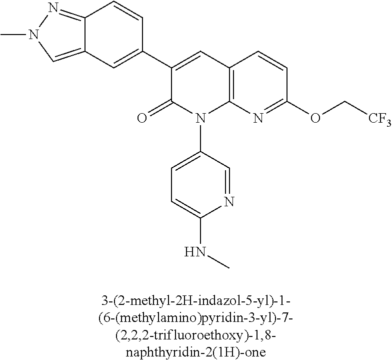 [SMILES: CNc1ccc(-n2c(=O)c(-c3ccc4nn(C)cc4c3)cc3ccc(OCC(F)(F)F)nc32)cn1] | LC-MS (ESI): m/z 481 [M + H]+. 1H NMR (400 MHz, DMSO-d6) δ: 8.41 (s, 1H), 8.27 (d, J = 8.4 Hz, 1H), 8.24 (s, 1H), 8.12 (s, 1H), 7.93 (d, J = 2.4 Hz, 1H), 7.63 (d, J = 8.8 Hz, 1H), 7.57 (dd, J = 8.8 Hz, 1.6 Hz, 1H), 7.38 (dd, J = 8.8 Hz, 2.4 Hz, 1H), 6.91 (d, J = 8.4 Hz, 1H), 6.71 (d, J = 4.8 Hz, 1H), 6.59 (d, J = 8.8 Hz, 1H), 4.78-4.66 (m, 2H), 4.19 (s, 3H), 2.84 (d, J = 4.8 Hz, 3H). |
| 168 | 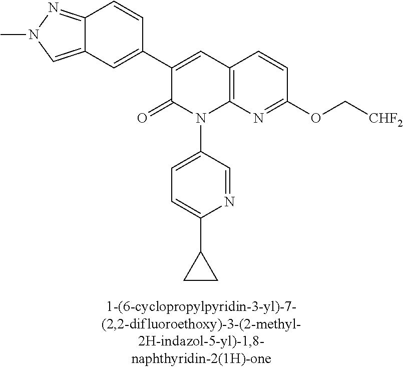 [SMILES: Cn1cc2cc(-c3cc4ccc(OCC(F)F)nc4n(-c4ccc(C5CC5)nc4)c3=O)ccc2n1] | LC-MS (ESI): m/z 474 [M + H]+. 1H NMR (400 MHz, DMSO-d6) δ: 8.41 (d, J = 4.0 Hz, 2H), 8.28 (s, 1H), 8.26 (d, J = 8.4 Hz, 1H), 8.13 (s, 1H), 7.73 (dd, J = 8.4 Hz, 2.4 Hz, 1H), 7.63 (d, J = 9.2 Hz, 1H), 7.58 (dd, J = 9.2 Hz, 1.6 Hz, 1H), 7.51 (d, J = 8.0 Hz, 1H), 6.88 (d, J = 8.4 Hz, 1H), 6.11 (tt, J_{HF} = 55.2 Hz, J = 4.0 Hz, 1H), 4.19 (s, 3H), 4.18 (dt, J_{HF} = 14.4 Hz, J = 3.6 Hz, 2H), 2.29-2.15 (m, 1H), 1.08-0.95 (m, 4H). |
| 169 | 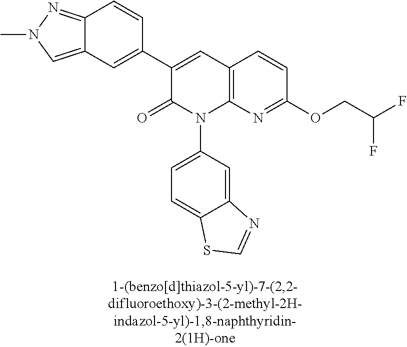 [SMILES: Cn1cc2cc(-c3cc4ccc(OCC(F)F)nc4n(-c4ccc5scnc5c4)c3=O)ccc2n1] | LC-MS (ESI): m/z 490 [M + H]+. 1H NMR (400 MHz, DMSO- d6) δ: 9.49 (s, 1H), 8.40 (s, 1H), 8.30 (s, 1H), 8.26 (t, J = 9.7 Hz, 3H), 8.15 (s, 1H), 7.65-7.54 (m, 3H), 6.87 (d, J = 8.4 Hz, 1H), 6.02 (t, J_{HF} = 55.2 Hz, 1H), 4.18 (s, 3H), 4.05 (t, J_{HF} = 13.2 Hz, 2H). |
| 317 | 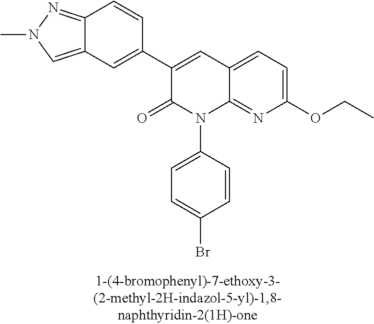 [SMILES: CCOc1ccc2cc(-c3ccc4nn(C)cc4c3)c(=O)n(-c3ccc(Br)cc3)c2n1] | LC-MS: m/z 475 (M + H)+. 1H NMR (400 MHz, DMSO-d6) δ: 8.39 (s, 1H), 8.22 (s, 1H), 8.16 (d, J = 8.4 Hz, 1H), 8.11 (s, 1H), 7.74 (d, J = 8.5 Hz, 2H), 7.63- 7.54 (m, 2H), 7.35 (d, J = 8.6 Hz, 2H), 6.73 (d, J = 8.4 Hz, 1H), 4.18 (s, 3H), 3.96 (q, J = 7.0 Hz, 2H), 1.11 (t, J = 7.0 Hz, 3H). |
| 318 | 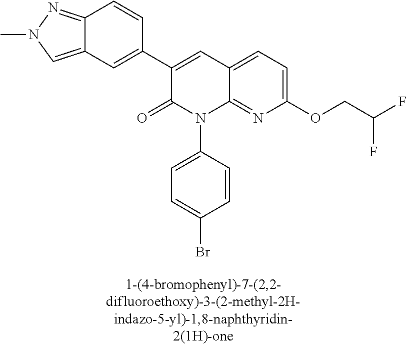 [SMILES: Cn1cc2cc(-c3cc4ccc(OCC(F)F)nc4n(-c4ccc(Br)cc4)c3=O)ccc2n1] | LC-MS: m/z 511 (M + H)+. 1H NMR (400 MHz, DMSO-d6) δ: 8.41 (s, 1H), 8.27 (s, 1H), 8.25 (s, 1H), 8.13 (s, 1H), 7.76 (d, J = 8.3 Hz, 2H), 7.63 (d, J = 8.9 Hz, 1H), 7.58 (d, J = 8.9 Hz, 1H), 7.39 (d, J = 8.3 Hz, 2H), 6.87 (d, J = 8.4 Hz, 1H), 6.12 (t, JHF = 55.2 Hz, 1H), 4.23-4.17 (m, 5H). |
| 319 | 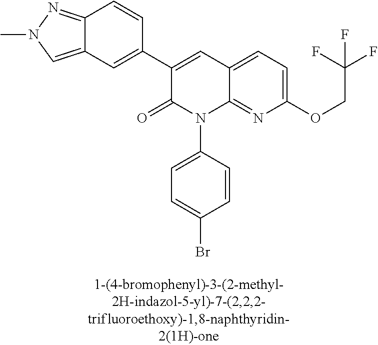 [SMILES: Cn1cc2cc(-c3cc4ccc(OCC(F)(F)F)nc4n(-c4ccc(Br)cc4)c3=O)ccc2n1] | LC-MS: m/z 529 (M + H)+. 1H NMR (400 MHz, DMSO-d6) δ: 8.40 (s, 1H), 8.27 (d, J = 9.7 Hz, 2H), 8.12 (s, 1H), 7.75 (d, J = 8.4 Hz, 2H), 7.66-7.55 (m, 2H), 7.36 (d, J = 8.5 Hz, 2H), 6.91 (d, J = 8.3 Hz, 1H), 4.60 (q, JHF = 8.9 Hz, 2H), 4.18 (s, 3H). |
| 320 | 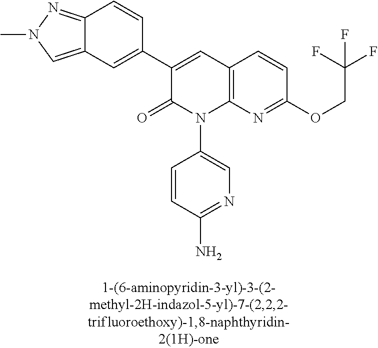 [SMILES: Cn1cc2cc(-c3cc4ccc(OCC(F)(F)F)nc4n(-c4ccc(N)nc4)c3=O)ccc2n1] | LC-MS: m/z 467 (M + H)+. 1H NMR (400 MHz, DMSO-d6) δ: 8.40 (s, 1H), 8.31-8.19 (m, 2H), 8.11 (d, J = 1.6 Hz, 1H), 7.85 (s, 1H), 7.72-7.51 (m, 2H), 7.37 (dd, J = 8.8 Hz, 2.4 Hz, 1H), 6.91 (d, J = 8.4 Hz, 1H), 6.59 (d, J = 8.8 Hz, 1H), 6.14 (s, 2H), 4.71 (q, JHF = 9.0 Hz, 2H), 4.18 (s, 3H). |

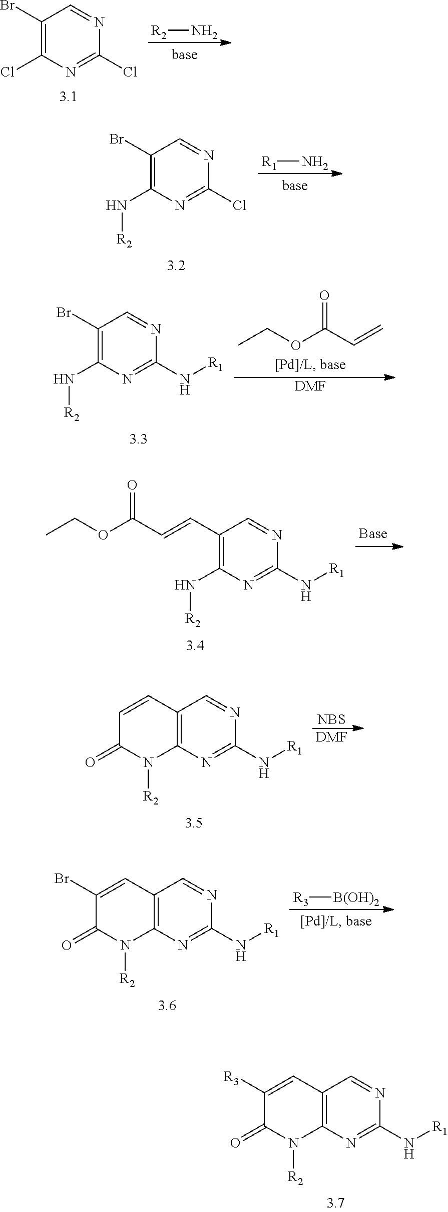

Compounds of structure 3.7 were obtained through the scheme depicted as General Procedure III. Beginning with heterocycle 3.1, a nucleophilic aromatic substitution reaction was used to introduce the desired R2 group to afford heterocycle 3.2. The desired R₁ group was also installed through a nucleophilic aromatic substitution reaction to afford diamino-pyrimidine 3.3. Compound 3.4 was generated through a palladium-mediated Heck coupling between heteroaryl-bromide 3.3 and ethyl acrylate. Treatment of vinyl-ester 3.4 with base led to ring-closure, which afforded compound 3.5. Halogenation of compound 3.5 with NBS gave heteroaryl-bromide 3.6, and the desired R3 group was introduced through a palladium mediated Suzuki coupling to afford compounds of structure 3.7.

### Preparation of Example 170 via General Procedure III

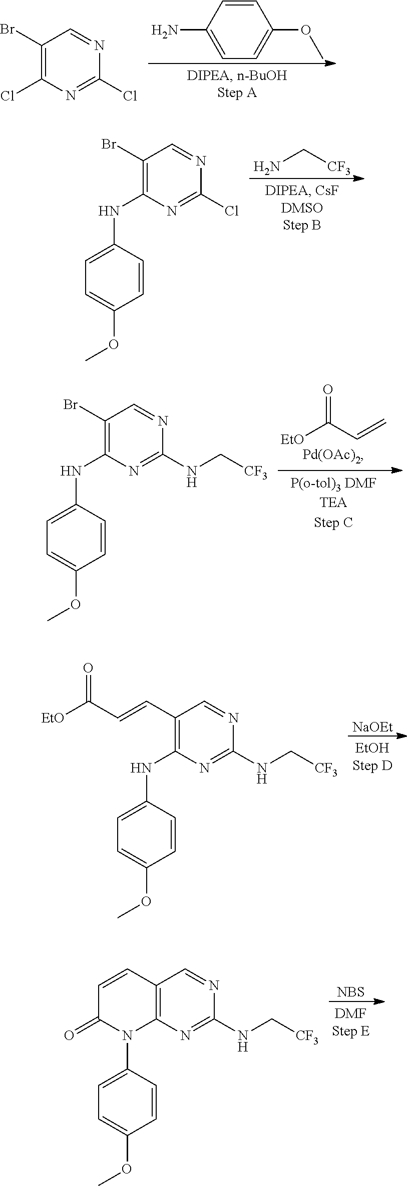

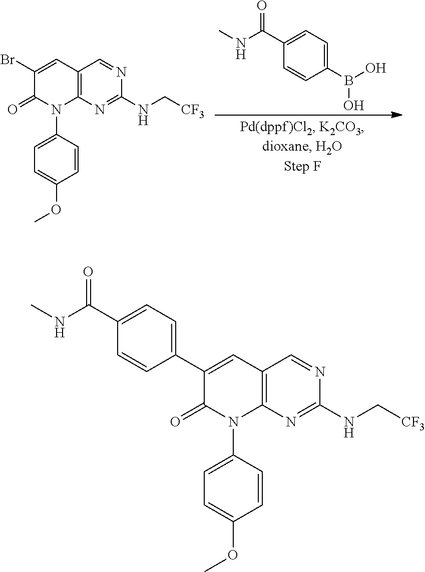 [SMILES: COc1ccc(-n2c(=O)c(Br)cc3cnc(NCC(F)(F)F)nc32)cc1 ; CNC(=O)c1ccc(B(O)O)cc1 ; CNC(=O)c1ccc(-c2cc3cnc(NCC(F)(F)F)nc3n(-c3ccc(OC)cc3)c2=O)cc1]

### Step A: 5-bromo-2-chloro-N-(4-methoxyphenyl)pyrimidin-4-amine

To a solution of 5-bromo-2,4-dichloropyrimidine (15 g, 66.4 mmol, 1.0 eq.) in n-BuOH (150 mL) was added 4-methoxyphenylamine (8.17 g, 66.4 mmol, 1.0 eq.) and DIPEA (12.85 g, 99.6 mmol, 1.5 eq.). Then the reaction mixture was stirred at 100° C. overnight. Then the mixture was diluted with (300 mL) and extracted with EtOAc (300 mL×3). The combined organic layers were washed with brine (200 mL), dried over Na₂SO₄ and concentrated under reduced pressure. The residue was purified by flash column chromatography on silica gel to afford 5-bromo-2-chloro-N-(4-methoxyphenyl)pyrimidin-4-amine (15 g, 72% yield) as a white solid. LC-MS (ESI): m/z 314, 316 [M+H]⁺.

### Step B: 5-bromo-N4-(4-methoxyphenyl)-N2-(2,2,2-trifluoroethyl)pyrimidine-2,4-diamine

A mixture of 5-bromo-2-chloro-N-(4-methoxyphenyl)pyrimidin-4-amine (5 g, 16 mmol, 1.0 eq.), DIPEA (20.6 g, 160 mmol, 10.0 eq.), CsF (243 mg, 1.6 mmol, 0.1 eq.), and 2,2,2-trifluoroethylamine (1.58 g, 16 mmol, 1.0 eq.) in DMSO (30 mL) was stirred in a sealed tube at 80° C. for 14 hrs. Then the reaction mixture was diluted with water (50 mL) and extracted with EtOAc (50 mL×3). The combined organic layers were washed with brine (50 mL), dried over Na₂SO₄ and concentrated under reduced pressure. The residue was purified by flash column chromatography on silica gel to afford 5-bromo-N4-(4-methoxyphenyl)-N2-(2,2,2-trifluoroethyl)pyrimidine-2,4-diamine (1.2 g, 20% yield) as an off-white solid. LC-MS (ESI): m/z 377, 379 [M+H]⁺.

### Step C: (E)-ethyl 3-(4-(4-methoxyphenylamino)-2-(2,2,2-trifluoroethylamino)pyrimidin-5-yl)acrylate

To a solution of 5-bromo-N4-(4-methoxyphenyl)-N2-(2,2,2-trifluoroethyl)pyrimidine-2,4-diamine (1.13 g, 3 mmol, 1.0 eq.) in DMF (20 mL) was added ethyl acrylate (1.2 g, 12 mmol, 4.0 eq.), tri-o-tolylphosphine (182 mg, 0.6 mmol, 0.2 eq.), TEA (2.1 g, 21 mmol, 7.0 eq.) and Pd(OAc)₂ (67 mg, 0.3 mmol, 0.1 eq.). The reaction mixture was stirred at 100° C. under N2 atmosphere for 14 hrs. Then the reaction mixture was diluted with water (30 mL) and extracted with EtOAc (30 mL×3). The combined organic layers were washed with brine (30 mL), dried over Na₂SO₄ and concentrated under reduced pressure. The residue was purified by flash column chromatography on silica gel to afford (E)-ethyl 3-(4-(4-methoxyphenylamino)-2-(2,2,2-trifluoroethylamino)pyrimidin-5-yl)acrylate (1.0 g, 84% yield) as an off-white solid. LC-MS (ESI): m/z 397 [M+H]⁺.

### Step D: 8-(4-methoxyphenyl)-2-(2,2,2-trifluoroethylamino)pyrido[2,3-d]pyrimidin-7(8H)-one

A mixture of (E)-ethyl 3-(4-(4-methoxyphenylamino)-2-(2,2,2-trifluoroethylamino)pyrimidin-5-yl)acrylate (1.0 g, 2.53 mmol, 1.0 eq.) and EtONa (860 mg, 12.65 mmol, 5.0 eq.) in EtOH (20 mL) was refluxed for 1 hr. Then the reaction mixture was cooled down to room temperature, diluted with water (30 mL) and extracted with EtOAc (30 mL×3). The combined organic layers were washed with brine (30 mL), dried over Na₂SO₄ and concentrated under reduced pressure. The residue was purified by flash column chromatography on silica gel to afford 8-(4-methoxyphenyl)-2-(2,2,2-trifluoroethylamino)pyrido[2,3-d]pyrimidin-7(8H)-one (0.8 g, 90% yield) as a yellow solid. LC-MS (ESI): m/z 351 [M+H]⁺.

### Step E: 6-bromo-8-(4-methoxyphenyl)-2-(2,2,2-trifluoroethylamino)pyrido[2,3-d]pyrimidin-7(8H)-one

To a solution of 8-(4-methoxyphenyl)-2-(2,2,2-trifluoroethylamino)pyrido[2,3-d]pyrimidin-7(8H)-one (0.8 g, 2.29 mmol, 1.0 eq.) in DMF (10 mL) was added NBS (620 mg, 3.5 mmol, 1.53 eq.) in several portions. The reaction mixture was stirred at room temperature for 14 hrs. Then the reaction mixture was diluted with water (30 mL) and extracted with EtOAc (30 mL×3). The combined organic layers were washed with brine (30 mL), dried over Na₂SO₄ and concentrated under reduced pressure. The residue was purified by flash column chromatography on silica gel to afford 6-bromo-8-(4-methoxyphenyl)-2-(2,2,2-trifluoroethylamino)pyrido[2,3-d]pyrimidin-7(8H)-one (750 mg, 77% yield) as a white solid. LC-MS (ESI): m/z 429, 431 [M+H]⁺.

### Step F: 4-(8-(4-methoxyphenyl)-7-oxo-2-(2,2,2-trifluoroethylamino)-7,8-dihydropyrido[2,3-d]pyrimidin-6-yl)-N-methylbenzamide

A mixture of 6-bromo-8-(4-methoxyphenyl)-2-(2,2,2-trifluoroethylamino)pyrido[2,3-d]pyrimidin-7(8H)-one (43 mg, 0.1 mmol, 1.0 eq.), 4-(methylcarbamoyl)phenylboronic acid (36 mg, 0.2 mmol, 2.0 eq.), K₂CO₃ (28 mg, 0.2 mmol, 2.0 eq.) and Pd(dppf)Cl₂ (8 mg, 0.01 mmol, 0.1 eq.) in dioxane/water mixture (5 mL, 4/1, v:v) was stirred at 100° C. under N2 atmosphere for 2 hrs. Then the reaction mixture was diluted with water (10 mL) and extracted with DCM (10 mL×3). The combined organic layers were washed with brine (20 mL), dried over Na₂SO₄ and concentrated under reduced pressure. The residue was purified by RP-prep-HPLC to afford 4-(8-(4-methoxyphenyl)-7-oxo-2-(2,2,2-trifluoroethylamino)-7,8-dihydropyrido[2,3-d]pyrimidin-6-yl)-N-methylbenzamide (Example 170).

¹ H NMR (400 MHz, DMSO-d₆) (the ratio of two tautomers: 1:1) δ: 8.83-8.80 (m, 1H), 8.49 (d, J=4.0 Hz, 1H), 8.39-8.32 (m, 0.5H), 8.18 (s, 1H), 8.09-8.02 (m, 0.5H), 7.88 (d, J=8.0 Hz, 2H), 7.78 (d, J=8.4 Hz, 2H), 7.28-7.19 (m, 2H), 7.07 (d, J=8.0 Hz, 2H), 4.19-4.07 (m, 1H), 3.82 (s, 3H), 3.80-3.69 (m, 1H), 2.80 (d, J=4.4 Hz, 3H). LC-MS (ESI): m/z 484 [M+H]⁺.

The procedure set forth above for General Procedure III was used to synthesize the following compounds by using appropriate starting materials:

| Example | Structure | Characterization |
|---|---|---|
| 171 | 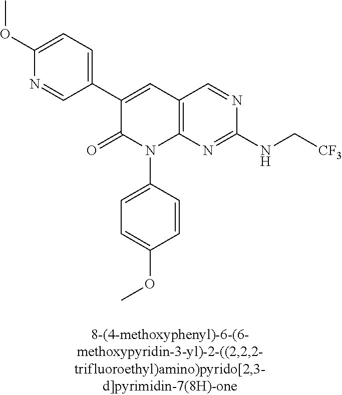 [SMILES: COc1ccc(-n2c(=O)c(-c3ccc(OC)nc3)cc3cnc(NCC(F)(F)F)nc32)cc1] | LC-MS (ESI): m/z 458 [M + H]+. 1H NMR (400 MHz, DMSO-d6) (the ratio of two tautomers: 1:1) δ: 8.82-8.74 (m, 1H), 8.48 (d, J = 2.4 Hz, 1H), 8.35-8.28 (m, 0.5H), 8.12 (s, 1H), 8.05-7.98 (m, 0.5H), 8.01 (dd, J = 8.8 Hz, 2.4 Hz, 1H), 7.28-7.18 (m, 2H), 7.06 (d, J = 8.4 Hz, 2H), 6.88 (d, J = 8.8 Hz, 1H), 4.18-4.07 (m, 1H), 3.89 (s, 3H), 3.83 (s, 3H), 3.79- 3.71 (m, 1H). |
| 172 | 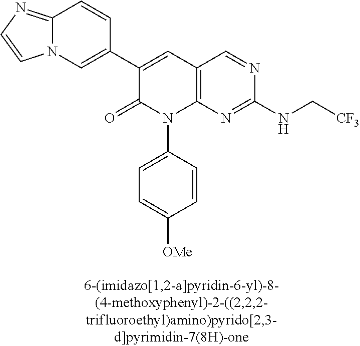 [SMILES: COc1ccc(-n2c(=O)c(-c3ccc4nccn4c3)cc3cnc(NCC(F)(F)F)nc32)cc1] | LC-MS (ESI): m/z 467 [M + H]+. 1H NMR (400 MHz, CDCl3) (the ratio of two tautomers: 1:1) δ: 9.05 (s, 1H), 8.64 (s, 1H), 7.91 (s, 1H), 7.66 (d, J = 6.4 Hz, 1H), 7.63 (s, 1H), 7.56 (s, 1H), 7.45 (d, J = 6.4 Hz, 1H), 7.18 (d, J = 5.2 Hz, 2H), 7.08 (d, J = 5.6 Hz, 2H), 5.69-5.51 (m, 1H), 4.25-4.13 (m, 1H), 3.89 (s, 3H), 3.84-3.77 (m, 1H). |
| 173 | 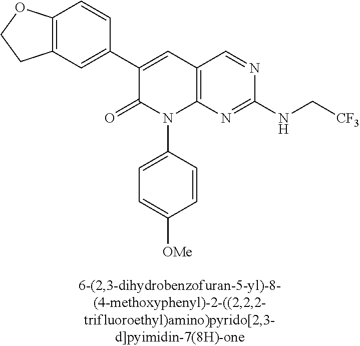 [SMILES: COc1ccc(-n2c(=O)c(-c3ccc4c(c3)CCO4)cc3cnc(NCC(F)(F)F)nc32)cc1] | LC-MS (ESI): m/z 469 [M + H]+. 1H NMR (400 MHz, DMSO-d6) (the ratio of two tautomers: 1:1) δ: 8.76 (s, 1H), 8.27-8.21 (m, 0.5H), 8.00 (s, 1H), 7.97-7.91 (m, 0.5H), 7.56 (s, 1H), 7.42 (d, J = 8.2 Hz, 1H), 7.22-7.20 (m, 2H), 7.06 (d, J = 8.8 Hz, 2H), 6.80 (d, J = 8.4 Hz, 1H), 4.57 (t, J = 8.8 Hz, 2H), 4.14-4.08 (m, 1H), 3.82 (s, 3H), 3.79-3.70 (m, 1H), 3.23 (t, J = 8.8 Hz, 2H). |
| 174 | 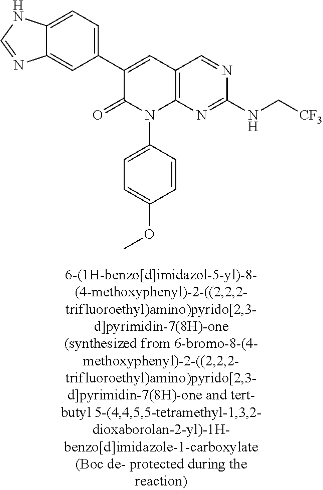 | LC-MS (ESI): m/z 476 [M + H]+. 1HNMR (400 MHz, DMSO- d6) (the ratio of two tautomers: 1:1) δ: 9.24 (s, 1H), 8.85-8.83 (m, 1H), 8.38-8.32 (m, 0.5H), 8.20 (s, 1H), 8.12 (s, 1H), 8.08-8.02 (m, 0.5H), 7.84 (d, J = 8.8 Hz, 1H), 7.78 (d, J = 8.8 Hz, 1H), 7.29-7.21 (m, 2H), 7.08 (d, J = 8.4 Hz, 2H), 4.19- 4.11 (m, 1H), 3.83 (s, 3H), 3.80-3.74 (m, 1H). |
| 175 | 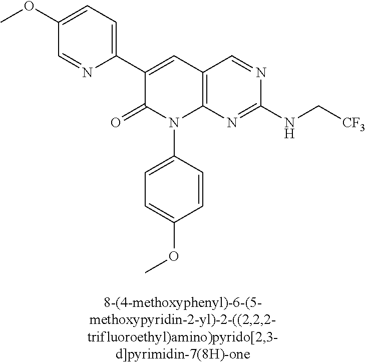 [SMILES: COc1ccc(-n2c(=O)c(-c3ccc(OC)cn3)cc3cnc(NCC(F)(F)F)nc32)cc1] | LC-MS (ESI): m/z 458 [M + H]+. 1H NMR (400 MHz, CDCl3) (the ratio of two tautomers: 1:1) δ: 8.67 (s, 1H), 8.61 (s, 1H), 8.44 (d, J = 8.8 Hz, 1H), 8.37 (s, 1H), 7.20-7.18 (m, 3H), 7.08 (d, J = 8.0 Hz, 2H), 5.64-5.43 (m, 1H), 3.89 (s, 6H), 3.14-3.10 (m, 2H). |
| 176 | 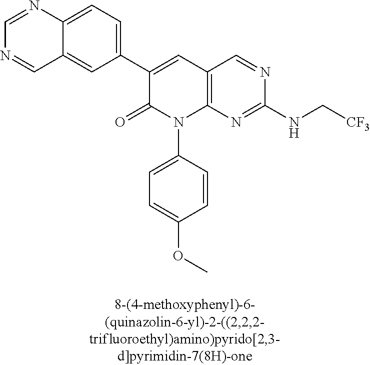 [SMILES: COc1ccc(-n2c(=O)c(-c3ccc4ncncc4c3)cc3cnc(NCC(F)(F)F)nc32)cc1] | LC-MS (ESI): m/z 479 [M + H]+. 1H NMR (400 MHz, DMSO-d6) (the ratio of two tautomers: 1:1) δ: 9.65 (s, 1H), 9.31 (s, 1H), 8.88-8.84 (m, 1H), 8.54 (d, J = 1.6 Hz, 1H), 8.43-8.39 (m, 0.5H), 8.36 (dd, J = 9.2 Hz, 2.4 Hz, 1H), 8.35 (s, 1H), 8.14-8.09 (m, 0.5H), 8.07 (d, J = 9.2 Hz, 1H), 7.30-7.25 (m, 2H), 7.08 (d, J = 8.4 Hz, 2H), 4.20-4.10 (m, 1H), 3.83 (s, 3H), 3.82-3.74 (m, 1H). |
| 177 | 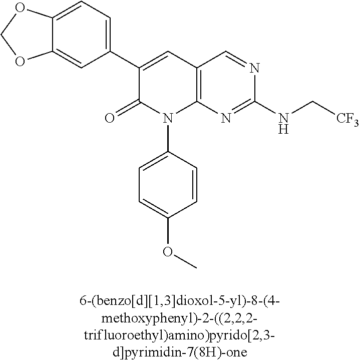 [SMILES: COc1ccc(-n2c(=O)c(-c3ccc4c(c3)OCO4)cc3cnc(NCC(F)(F)F)nc32)cc1] | LC-MS (ESI): m/z 471 [M + H]+. 1H NMR (400 MHz, DMSO-d6) (the ratio of two tautomers: 1:1) δ: 8.81-8.73 (m, 1H), 8.32-8.23 (m, 0.5H), 8.04 (s, 1H), 8.01- 7.92 (m, 0.5H), 7.27-7.17 (m, 4H), 7.06 (d, J = 8.8 Hz, 2H), 6.97 (d, J = 8.4 Hz, 1H), 6.05 (s, 2H), 4.17-4.07 (m, 1H), 3.82 (s, 3H), 3.79- 3.69 (m, 1H). |
| 178 | 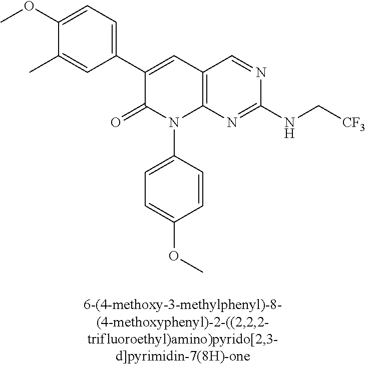 [SMILES: COc1ccc(-n2c(=O)c(-c3ccc(OC)c(C)c3)cc3cnc(NCC(F)(F)F)nc32)cc1] | LC-MS (ESI): m/z 471 [M + H]+. 1H NMR (400 MHz, DMSO-d6) (the ratio of two tautomers: 1:1) δ: 8.81-8.74 (m, 1H), 8.28-8.19 (m, 0.5H), 8.01 (s, 1H), 7.98- 7.90 (m, 0.5H), 7.53 (dd, J = 4.4 Hz, 2.0 Hz, 1H), 7.49 (d, J = 1.6 Hz, 1H), 7.25-7.16 (m, 2H), 7.06 (d, J = 8.8 Hz, 2H), 6.98 (d, J = 8.8 Hz, 1H), 4.18-4.06 (m, 1H), 3.82 (s, 6H), 3.79-3.69 (m, 1H), 2.19 (s, 3H). |
| 179 | 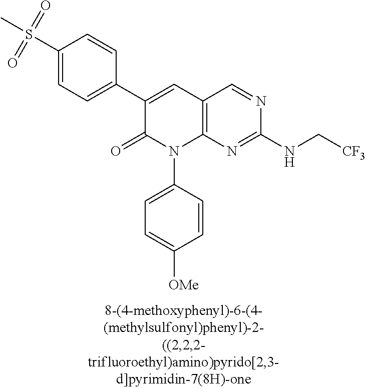 [SMILES: COc1ccc(-n2c(=O)c(-c3ccc(S(C)(=O)=O)cc3)cc3cnc(NCC(F)(F)F)nc32)cc1] | LC-MS (ESI): m/z 505 [M + H]+. 1H NMR (400 MHz, DMSO-d6) (the ratio of two tautomers: 1:1) δ: 8.87-8.79 (m, 1H), 8.44-8.39 (m, 0.5H), 8.24 (s, 1H), 8.14- 8.08 (m, 0.5H), 7.97 (s, 4H), 7.29-7.19 (m, 2H), 7.07 (d, J = 8.4 Hz, 2H), 4.16-4.14 (m, 1H), 3.83 (s, 3H), 3.78-3.75 (m, 1H), 3.26 (s, 3H). |
| 180 | 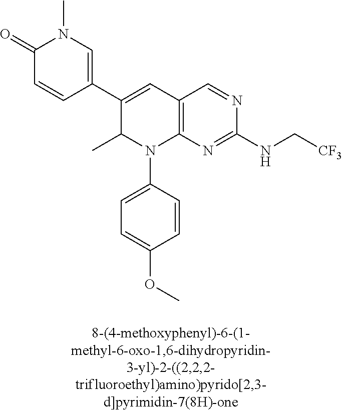 [SMILES: COc1ccc(N2c3nc(NCC(F)(F)F)ncc3C=C(c3ccc(=O)n(C)c3)C2C)cc1] | LC-MS (ESI): m/z 458 [M + H]+. 1H NMR (400 MHz, DMSO-d6) (the ratio of two tautomers: 1:1) δ: 8.75 (s, 1H), 8.30-8.26 (m, 0.5H), 8.26 (s, 1H), 8.08 (s, 1H), 8.04-7.98 (m, 0.5H), 7.81- 7.78 (m, 1H), 7.27-7.15 (m, 2H), 7.12-7.00 (m, 2H), 6.45 (d, J = 8.0 Hz, 1H), 4.18- 4.06 (m, 1H), 3.82 (s, 3H), 3.77-3.67 (m, 1H), 3.48 (s, 3H). |
| 181 | 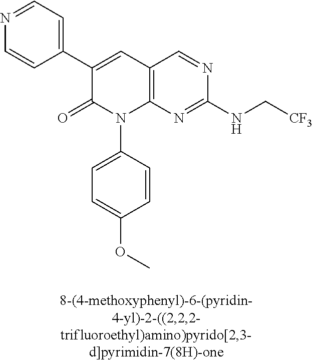 [SMILES: COc1ccc(-n2c(=O)c(-c3ccncc3)cc3cnc(NCC(F)(F)F)nc32)cc1] | LC-MS (ESI): m/z 428 [M + H]+. 1H NMR (400 MHz, DMSO-d6) (the ratio of two tautomers: 1:1) δ: 8.85 (d, J = 2.0 Hz, 1H), 8.82-8.77 (m, 1H), 8.54 (dd, J = 4.8 Hz, 2.0 Hz, 1H), 8.40-8.34 (m, 0.5H), 8.21 (s, 1H), 8.09- 8.07 (m, 1.5H), 7.45 (dd, J = 8.0 Hz, 4.8 Hz, 1H), 7.28- 7.19 (m, 2H), 7.06 (d, J = 8.8 Hz, 2H), 4.18-4.07 (m, 1H), 3.82 (s, 3H), 3.79-3.70 (m, 1H). |
| 182 | 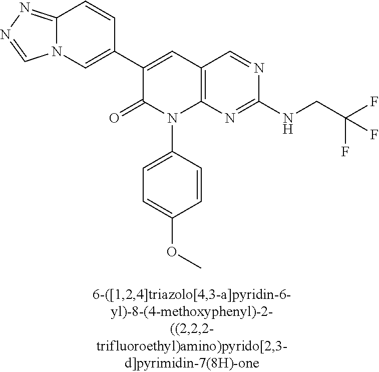 [SMILES: COc1ccc(-n2c(=O)c(-c3ccc4nncn4c3)cc3cnc(NCC(F)(F)F)nc32)cc1] | LC-MS (ESI): m/z 468 [M + H]+. 1HNMR (400 MHz, CDCl3) (the ratio of two tautomers: 1:1) δ: 8.70 (s, 1H), 8.44 (s, 1H), 8.18 (d, J = 7.2 Hz, 1H), 7.80 (d, J = 9.2 Hz, 1H), 7.30-7.28 (m, 1H), 7.22 (d, J = 8.8 Hz, 2H), 7.08 (d, J = 8.8 Hz, 2H), 6.80 (t, J = 6.8 Hz, 1H), 5.79-5.60 (m, 1H), 4.27-4.18 (m, 1H), 3.88 (s, 3H), 3.87-3.80 (m, 1H). |
| 183 | 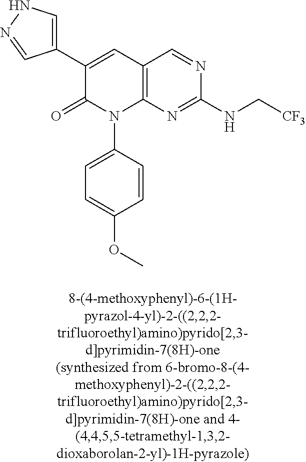 [SMILES: COc1ccc(-n2c(=O)c(-c3cn[nH]c3)cc3cnc(NCC(F)(F)F)nc32)cc1] | LC-MS (ESI): m/z 417 [M + H]+. 1H NMR (400 MHz, DMSO-d6) (the ratio of two tautomers: 1:1) δ: 12.93 (s, 1H), 8.72 (s, 1H), 8.47-8.23 (m, 2H), 8.22-7.86 (m, 2H), 7.25-7.15 (m, 2H), 7.06 (d, J = 8.8 Hz, 2H), 4.20-4.50 (m, 1H), 3.82 (s, 3H), 3.80-3.65 (m, 1H). |
| 184 | 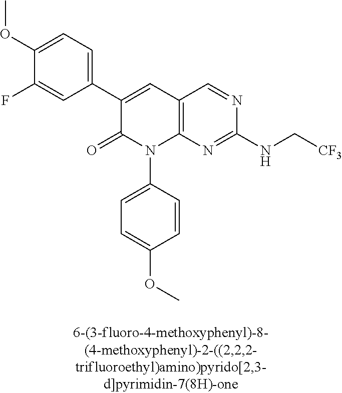 [SMILES: COc1ccc(-n2c(=O)c(-c3ccc(OC)c(F)c3)cc3cnc(NCC(F)(F)F)nc32)cc1] | LC-MS (ESI): m/z 475 [M + H]+. 1H NMR (400 MHz, DMSO-d6) (the ratio of two tautomers: 1:1) δ: 8.83-8.74 (m, 1H), 8.34-8.25 (m, 0.5H), 8.11 (s, 1H), 8.05- 7.96 (m, 0.5H), 7.60 (dd, J_{HF} = 13.2 Hz, J = 2.0 Hz, 1H), 7.52 (d, J = 8.4 Hz, 1H), 7.27-7.17 (m, 3H), 7.06 (d, J = 8.8 Hz, 2H), 4.19-4.06 (m, 1H), 3.88 (s, 3H), 3.82 (s, 3H), 3.79-3.68 (m, 1H). |
| 185 | 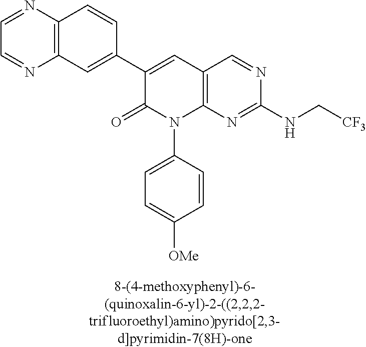 [SMILES: COc1ccc(-n2c(=O)c(-c3ccc4nccnc4c3)cc3cnc(NCC(F)(F)F)nc32)cc1] | LC-MS (ESI): m/z 479 [M + H]+. 1H NMR (400 MHz, DMSO-d6) (the ratio of two tautomers: 1:1) δ: 8.96 (dd, J = 10.0 Hz, 1.6 Hz, 2H), 8.85 (br, 1H), 8.46 (d, J = 1.9 Hz, 1H), 8.39 (s, 1.5H), 8.20 (dd, J = 8.8 Hz, 1.9 Hz, 1H), 8.12 (d, J = 8.8 Hz, 1H), 8.11- 8.07 (m, 0.5H), 7.27 (br, 2H), 7.08 (d, J = 8.4 Hz, 2H), 4.14 (br, 1H), 3.85-3.74 (m, 4H). |
| 186 | 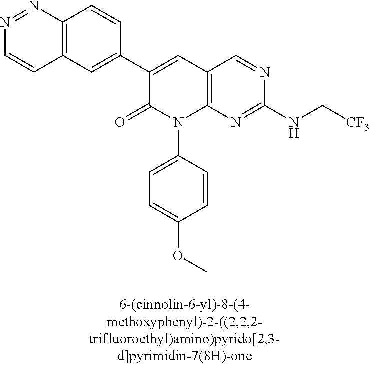 [SMILES: COc1ccc(-n2c(=O)c(-c3ccc4nnccc4c3)cc3cnc(NCC(F)(F)F)nc32)cc1] | LC-MS (ESI): m/z 479 [M + H]+. 1H NMR (400 MHz, DMSO-d6) (the ratio of two tautomers: 1:1) δ: 9.39 (d, J = 6.0 Hz, 1H), 8.89-8.86 (m, 1H), 8.50 (d, J = 8.8 Hz, 1H), 8.49-8.43 (m, 0.5H), 8.46 (d, J = 1.2 Hz, 1H), 8.41 (s, 1H), 8.29-8.25 (m, 2H), 8.19-8.13 (m, 0.5H), 7.30-7.24 (m, 2H), 7.09 (d, J = 8.4 Hz, 2H), 4.20-4.11 (m, 1H), 3.84 (s, 3H), 3.81-3.74 (m, 1H). |
| 187 | 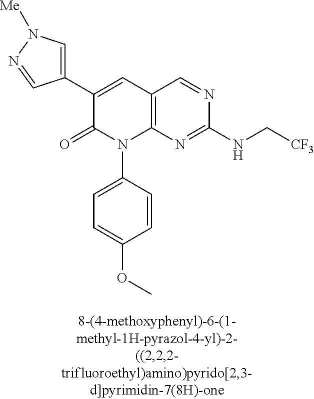 [SMILES: COc1ccc(-n2c(=O)c(-c3cnn(C)c3)cc3cnc(NCC(F)(F)F)nc32)cc1] | LC-MS (ESI): m/z 431 [M + H]+. 1H NMR (400 MHz, DMSO-d6) (the ratio of two tautomers: 1:1) δ: 8.72 (s, 1H), 8.26 (s, 1H), 8.24-8.16 (m, 1.5H), 8.00 (s, 1H), 7.96-7.87 (m, 0.5H), 7.25- 7.16 (m, 2H), 7.06 (d, J = 8.8 Hz, 2H), 4.16-4.05 (m, 1H), 3.86 (s, 3H), 3.82 (s, 3H), 3.79-3.67 (m, 1H). |
| 188 | 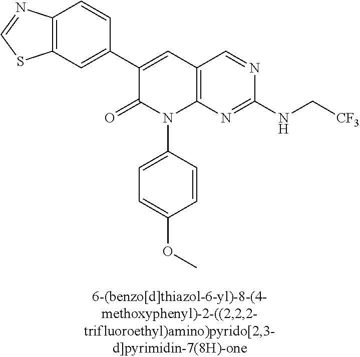 [SMILES: COc1ccc(-n2c(=O)c(-c3ccc4ncsc4c3)cc3cnc(NCC(F)(F)F)nc32)cc1] | LCMS (ESI): m/z 484 [M + H]+. 1H NMR (400 MHz, DMSO-d6) (the ratio of two tautomers: 1:1) δ: 9.43 (s, 1H), 8.84-8.81 (m, 1H), 8.53 (d, J = 1.2 Hz, 1H), 8.38- 8.32 (m, 0.5H), 8.22 (s, 1H), 8.13 (d, J = 8.8 Hz, 1H), 8.08-8.02 (m, 0.5H), 7.86 (dd, J = 8.8 Hz, 2.0 Hz, 1H), 7.28 (d, J = 7.6 Hz, 2H), 7.08 (d, J = 8.8 Hz, 2H), 4.18-4.09 (m, 1H), 3.83 (s, 3H), 3.79-3.72 (m, 1H). |
| 189 | 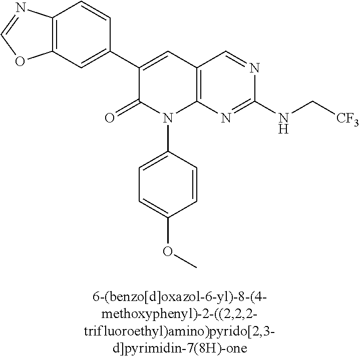 [SMILES: COc1ccc(-n2c(=O)c(-c3ccc4ncoc4c3)cc3cnc(NCC(F)(F)F)nc32)cc1] | LC-MS (ESI): m/z 468 [M + H]+. 1H NMR (400 MHz, CDCl3) (the ratio of two tautomers: 1:1) δ: 8.62 (s, 1H), 8.12 (s, 1H), 8.07 (s, 1H), 7.83 (s, 1H), 7.82 (d, J = 8.8 Hz, 1H), 7.67 (dd, J = 8.4, 1.6 Hz, 1H), 7.20 (d, J = 8.8 Hz, 2H), 7.07 (d, J = 8.8 Hz, 2H), 5.58-5.50 (m, 1H), 4.23-4.09 (m, 1H), 3.88 (s, 3H), 3.88-3.78 (m, 1H). |
| 190 | 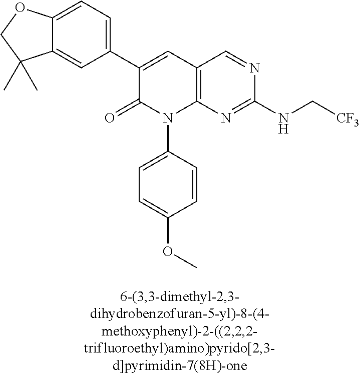 [SMILES: COc1ccc(-n2c(=O)c(-c3ccc4c(c3)C(C)(C)CO4)cc3cnc(NCC(F)(F)F)nc32)cc1] | LC-MS (ESI): m/z 497 [M + H]+. 1H NMR (400 MHz, DMSO-d6) (the ratio of two tautomers: 1:1) δ: 8.81-8.74 (m, 1H), 8.25-8.19 (m, 0.5H), 8.00 (s, 1H), 7.95- 7.89 (m, 0.5H), 7.53 (d, J = 1.2 Hz, 1H), 7.44 (dd, J = 8.4 Hz, 1.2 Hz, 1H), 7.22- 7.18 (m, 2H), 7.06 (d, J = 8.8 Hz, 2H), 6.81 (d, J = 8.4 Hz, 1H), 4.25 (s, 2H), 4.13-4.07 (m, 1H), 3.78 (s, 3H), 3.75- 3.70 (m, 1H), 1.31 (s, 6H). |
| 191 | 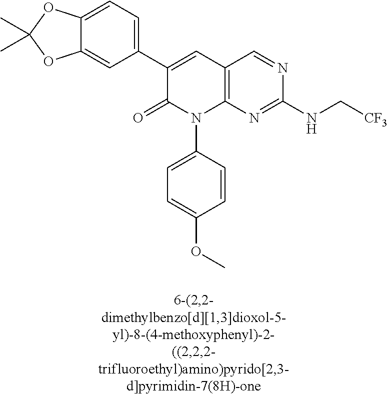 [SMILES: COc1ccc(-n2c(=O)c(-c3ccc4c(c3)OC(C)(C)O4)cc3cnc(NCC(F)(F)F)nc32)cc1] | LC-MS (ESI): m/z 499 [M + H]+. 1H NMR (400 MHz, DMSO-d6) (the ratio of two tautomers: 1:1) δ: 8.78-8.72 (m, 1H), 8.28-8.22 (m, 0.5H), 8.01 (s, 1H), 7.98- 7.92 (m, 0.5H), 7.21-7.18 (m, 2H), 7.15-7.12 (m, 2H), 7.06 (d, J = 8.8 Hz, 2H), 6.88 (d, J = 8.0 Hz, 1H), 4.15-4.08 (m, 1H), 3.82 (s, 3H), 3.79-3.70 (m, 1H), 1.67 (s, 6H). |
| 192 | 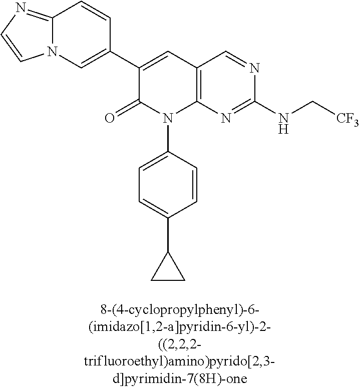 [SMILES: O=c1c(-c2ccc3nccn3c2)cc2cnc(NCC(F)(F)F)nc2n1-c1ccc(C2CC2)cc1] | LC-MS (ESI): m/z 477 [M + H]+. 1H NMR (400 MHz, DMSO-d6) (the ratio of two tautomers: 1:1) δ: 9.07 (s, 1H), 8.83-8.79 (m, 1H), 8.39-8.18 (m, 2H), 8.00 (s, 1H), 7.65-7.51 (m, 3H), 7.25-7.12 (m, 4H), 4.13 (s, 1H), 3.73 (s, 1H), 2.01 (s, 1H), 1.03-1.00 (m, 2H), 0.85-0.67 (m, 2H). |
| 193 | 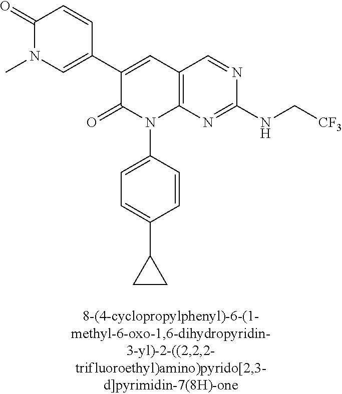 [SMILES: Cn1cc(-c2cc3cnc(NCC(F)(F)F)nc3n(-c3ccc(C4CC4)cc3)c2=O)ccc1=O] | LC-MS (ESI): m/z 468 [M + H]+. 1H NMR (400 MHz, DMSO-d6) (the ratio of two tautomers: 1:1) δ: 8.75 (br, 1H), 8.30-7.95 (m, 3H), 7.79 (dd, J = 9.5 Hz, 2.3 Hz, 1H), 7.23-7.10 (m, 4H), 6.46 (d, J = 9.5 Hz, 1H), 4.11 (br, 1H), 3.71 (br, 1H), 3.48 (s, 3H), 2.05 (br, 1H), 1.24-0.98 (m, 2H), 0.75-0.54 (m, 2H). |

General Procedure IV

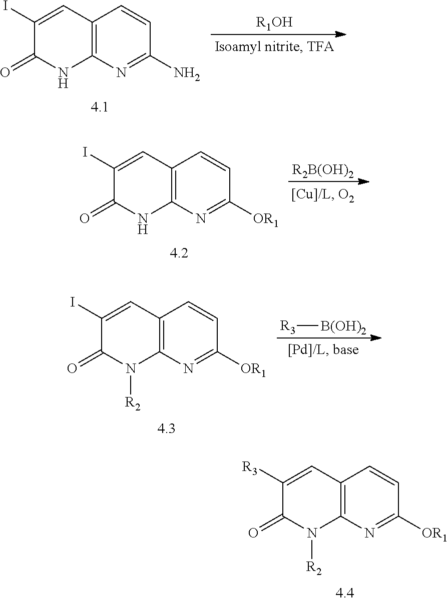

Compounds of structure 4.4 were obtained through the scheme depicted as General Procedure IV. Beginning with amino-heterocycle 4.1 (Ref: ACIE, 2005, 44, 596-598), the desired R₁ group was introduced with a Sandmeyer-type reaction to generate compound 4.2. The desired R2 group was introduced through a copper mediated Chan-Lam coupling to generate compound 4.3. Lastly, the desired R3 group was introduced through a palladium mediated Suzuki coupling to afford compounds of structure 4.4.

### Preparation of Example 194 via General Procedure IV

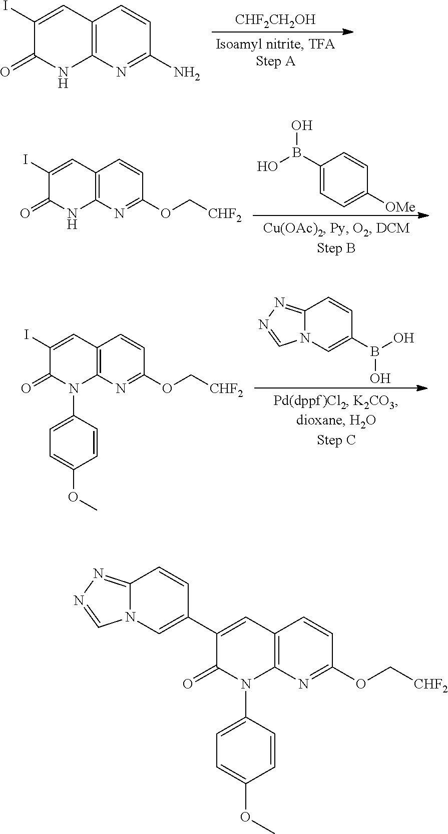

### Step A: 7-(2,2-difluoroethoxy)-3-iodo-1,8-naphthyridin-2(1H)-one

To a solution of 7-amino-3-iodo-1,8-naphthyridin-2(1H)-one (1.0 g, 3.5 mmol, 1.0 eq.) (Ref: ACIE, 2005, 44, 596-598) in CF₂CH₂OH (10 mL) was added isoamyl nitrite (2.0 g, 17.4 mmol, 5.0 eq.) and TFA (2.0 g, 17.4 mmol, 5.0 eq.), the reaction mixture was stirred at 40° C. under N2 atmosphere for 16 hours. Then the reaction mixture was filtered and the filtrate was concentrated under reduced pressure, the residue was purified by flash column chromatography on silica gel to give 7-(2,2-difluoroethoxy)-3-iodo-1,8-naphthyridin-2(1H)-one (400 mg, 33% yield) as a brown solid. LC-MS (ESI): m/z 353 [M+H]⁺.

### Step B: 7-(2,2-difluoroethoxy)-3-iodo-1-(4-methoxyphenyl)-1,8-naphthyridin-2(1H)-one

A mixture of 7-(2,2-difluoroethoxy)-3-iodo-1,8-naphthyridin-2(1H)-one (200 mg, 0.57 mmol, 1.0 eq.), 4-methoxyphenylboronic acid (130 mg, 0.85 mmol, 1.5 eq.), Cu(OAc)₂ (103 mg, 0.57 mmol, 1.0 eq.) and pyridine (180 mg, 2.3 mmol, 4.0 eq.) in DCM (10 mL) was stirred at 40° C. under 02 atmosphere for 14 hrs. The mixture was diluted with water (20 mL) and extracted with DCM (20 mL×3), The combined organic layers were washed with brine (20 mL), dried over Na₂SO₄ and concentrated under reduced pressure. The residue was purified by flash column chromatography on silica gel to afford 7-(2,2-difluoroethoxy)-3-iodo-1-(4-methoxyphenyl)-1,8-naphthyridin-2(1H)-one (100 mg, 38% yield) as a brown solid. LC-MS (ESI): m/z 459 [M+H]⁺.

### Step C: 3-([1,2,4]triazolo[4,3-a]pyridin-6-yl)-7-(2,2-difluoroethoxy)-1-(4-methoxyphenyl)-1,8-naphthyridin-2(1H)-one

A mixture of 7-(2,2-difluoroethoxy)-3-iodo-1-(4-methoxyphenyl)-1,8-naphthyridin-2(1H)-one (100 mg, 0.21 mmol, 1.0 eq.), [1,2,4]triazolo[4,3-a]pyridin-6-ylboronic acid (71 mg, 0.42 mmol, 2.0 eq.), Pd(dppf)Cl₂ (16 mg, 0.02 mmol, 0.1 eq.) and K₂CO₃ (90 mg, 0.63 mmol, 3.0 eq.) in dioxane/H₂O mixture (12.5 mL, 4:1, v:v) was degassed with N2, stirred at 100° C. under N2 atmosphere for 14 hrs. The reaction mixture was concentrated under reduced pressure, the residue was purified by flash column chromatography on silica gel column and RP-prep-HPLC to afford 3-([1,2,4]triazolo[4,3-a]pyridin-6-yl)-7-(2,2-difluoroethoxy)-1-(4-methoxyphenyl)-1,8-naphthyridin-2(1H)-one (Example 194).

¹H NMR (400 MHz, DMSO-d₆) δ: 9.34 (s, 1H), 9.22 (s, 1H), 8.48 (s, 1H), 8.25 (d, J 8.4 Hz, (H), 7.86 (d, J 9.6 Hz, H), 7.77 (dd, J=9.6 Hz, 1.6 Hz, 1H), 7.29 (d, J 8.8 Hz, 2H), 7.10 (d, J 8.8 Hz, 2H), 6.90 (d, J=8.4 Hz, 1H), 6.11 (tt, J_{HF}=55.2 Hz, J=3.6 Hz, 1H), 4.19 (td, J_{HF} 14.4, J=3.6 Hz, 2H), 3.84 (s, 3H). LC-MS (ESI): m/z 450 [M+H]⁺.

The procedure set forth above for General Procedure IV was used to synthesize the following compounds by using appropriate starting materials:

| Example | Structure | Characterization |
|---|---|---|
| 195 | 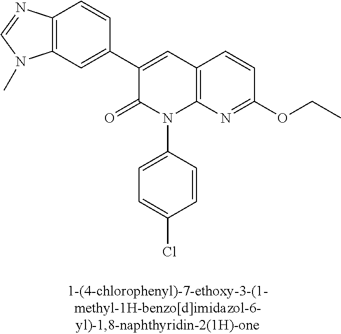 [SMILES: CCOc1ccc2cc(-c3ccc4ncn(C)c4c3)c(=O)n(-c3ccc(Cl)cc3)c2n1] | LC-MS (ESI): m/z 431 [M + H]+. 1H NMR (400 MHz, DMSO- d6) δ: 8.27 (s, 1H), 8.22 (s, 1H), 8.18 (d, J = 8.4 Hz, 1H), 7.99 (s, 1H), 7.69 (d, J = 8.4 Hz, 1H), 7.62 (d, J = 8.6 Hz, 2H), 7.57 (d, J = 8.5 Hz, 1.1 Hz, 1H), 7.43 (d, J = 8.6 Hz, 2H), 6.74 (d, J = 8.4 Hz, 1H), 3.96 (q, J = 7.0 Hz, 2H), 3.86 (s, 3H), 1.11 (t, J = 7.0 Hz, 3H). |
| 196 | 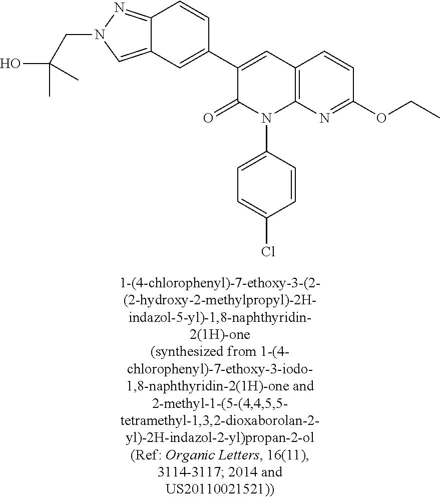 [SMILES: CCOc1ccc2cc(-c3ccc4nn(CC(C)(C)O)cc4c3)c(=O)n(-c3ccc(Cl)cc3)c2n1] | LC-MS (ESI): m/z 489 [M + H]+. 1H NMR (400 MHz, DMSO- d6) δ: 8.36 (s, 1H), 8.22 (s, 1H), 8.16 (d, J = 8.3 Hz, 1H), 8.12 (s, 1H), 7.68-7.52 (m, 4H), 7.42 (d, J = 7.8 Hz, 2H), 6.73 (d, J = 8.0 Hz, 1H), 4.86 (s, 1H), 4.35 (s, 2H), 3.95 (q, J = 7.6 Hz, 2H), 1.12 (s, 6H), 1.11 (t, J = 7.6 Hz, 3H). |
| 197 | 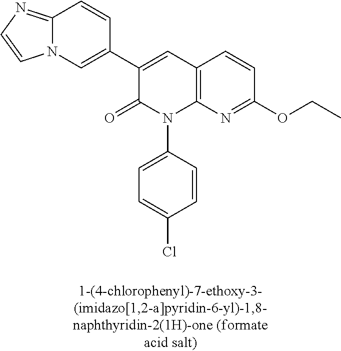 [SMILES: CCOc1ccc2cc(-c3ccc4nccn4c3)c(=O)n(-c3ccc(Cl)cc3)c2n1] | LC-MS (ESI): m/z 417 [M + H]+. 1H NMR (400 MHz, DMSO- d6, 0.4 HCOOH) δ: 9.16 (s, 1H), 8.40 (s, 1H), 8.18 (d, J = 8.4 Hz, 1H), 8.14 (s, 0.4H), 8.02 (s, 1H), 7.64-7.60 (m, 5H), 7.43 (d, J = 8.4 Hz, 2H), 6.77 (d, J = 8.4 Hz, 1H), 3.97 (q, J = 6.8 Hz, 2H), 1.11 (t, J = 6.8 Hz, 3H). |
| 198 | 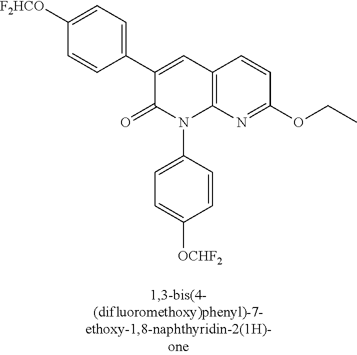 [SMILES: CCOc1ccc2cc(-c3ccc(OC(F)F)cc3)c(=O)n(-c3ccc(OC(F)F)cc3)c2n1] | LC-MS (ESI): m/z 475 [M + H]+. 1H NMR (400 MHz, DMSO- d6) δ: 8.22 (s, 1H), 8.15 (d, J = 8.4 Hz, 1H), 7.79 (d, J = 8.8 Hz, 2H), 7.42 (d, J = 9.2 Hz, 2H), 7.36 (t, J_{HF} = 74.0 Hz, 1H), 7.34 (d, J = 8.8 Hz, 2H), 7.31 (t, J_{HF} = 74.0 Hz, 7.24 (d, J = 8.8 Hz, 2H), 6.73 (d, J = 8.4 Hz, 1H), 3.94 (q, J = 7.2 Hz, 2H), 1.08 (t, J = 7.2 Hz, 3H). |
| 199 | 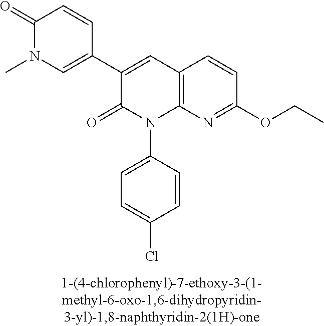 [SMILES: CCOc1ccc2cc(-c3ccc(=O)n(C)c3)c(=O)n(-c3ccc(Cl)cc3)c2n1] | LC-MS (ESI): m/z 408 [M + H]+. 1H NMR (400 MHz, DMSO- d6) δ: 8.34 (d, J = 2.5 Hz, 1H), 8.21 (s, 1H), 8.11 (d, J = 8.4 Hz, 1H), 7.85 (dd, J = 9.5 Hz, 2.6 Hz, 1H), 7.61 (d, J = 8.6 Hz, 2H), 7.39 (d, J = 8.6 Hz, 2H), 6.74 (d, J = 8.4 Hz, 1H), 6.47 (d, J = 9.5 Hz, 1H), 3.94 (q, J = 7.0 Hz, 2H), 3.50 (s, 3H), 1.10 (t, J = 7.0 Hz, 3H). |
| 200 | 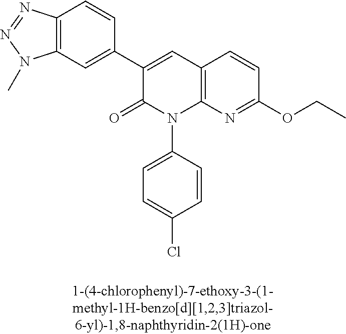 [SMILES: CCOc1ccc2cc(-c3ccc4nnn(C)c4c3)c(=O)n(-c3ccc(Cl)cc3)c2n1] | LC-MS (ESI): m/z 432 [M + H]+. 1H NMR (400 MHz, DMSO- d6) δ: 8.43 (s, 1H), 8.30 (s, 1H), 8.26 (d, J = 8.5 Hz, 1H), 8.13 (d, J = 8.7 Hz, 1H), 7.82 (dd, J = 8.8 Hz, 1.2 Hz, 1H), 7.69 (d, J = 8.6 Hz, 2H), 7.50 (d, J = 8.6 Hz, 2H), 6.83 (d, J = 8.4 Hz, 1H), 4.39 (s, 3H), 4.03 (q, J = 7.0 Hz, 2H), 1.18 (t, J = 7.0 Hz, 3H). |
| 201 | 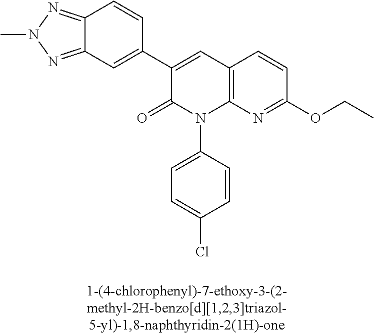 [SMILES: CCOc1ccc2cc(-c3ccc4nn(C)nc4c3)c(=O)n(-c3ccc(Cl)cc3)c2n1] | LC-MS (ESI): m/z 432 [M + H]+. 1H NMR (400 MHz, DMSO- d6) δ: 8.33 (s, 1H), 8.26 (s, 1H), 8.17 (d, J = 8.4 Hz, 1H), 7.93 (d, J = 8.9 Hz, 1H), 7.77 (dd, J = 9.0 Hz, 1.1 Hz, 1H), 7.62 (d, J = 8.6 Hz, 2H), 7.44 (d, J = 8.6 Hz, 2H), 6.74 (d, J = 8.4 Hz, 1H), 4.52 (s, 3H), 3.96 (q, J = 7.0 Hz, 2H), 1.11 (t, J = 7.0 Hz, 3H). |
| 202 | 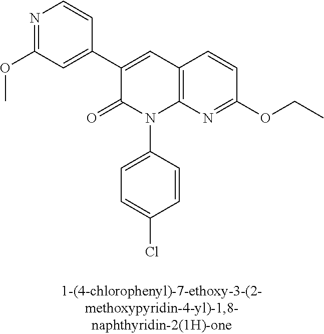 [SMILES: CCOc1ccc2cc(-c3ccnc(OC)c3)c(=O)n(-c3ccc(Cl)cc3)c2n1] | LC-MS (ESI): m/z 408 [M + H]+. 1H NMR (400 MHz, DMSO- d6) δ: 8.43 (s, 1H), 8.21 (d, J = 5.6 Hz, 1H), 8.17 (d, J = 8.4 Hz, 1H), 7.61 (d, J = 8.4 Hz, 2H), 7.41 (d, J = 8.4 Hz, 2H), 7.37 (d, J = 5.4 Hz, 1H), 7.25 (s, 1H), 6.76 (d, J = 8.4 Hz, 1H), 3.95 (q, J = 6.8 Hz, 2H), 3.88 (s, 3H), 1.10 (t, J = 6.8 Hz, 3H). |
| 203 | 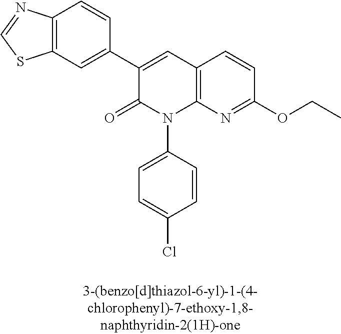 [SMILES: CCOc1ccc2cc(-c3ccc4ncsc4c3)c(=O)n(-c3ccc(Cl)cc3)c2n1] | LC-MS (ESI): m/z 434 [M + H]+. 1H NMR (400 MHz, DMSO- d6) δ: 9.43 (s, 1H), 8.55 (d, J = 1.2 Hz, 1H), 8.34 (s, 1H), 8.18 (d, J = 8.4 Hz, 1H), 8.14 (d, J = 8.4 Hz, 1H), 7.90 (dd, J = 8.4 Hz, 1.6 Hz, 1H), 7.62 (d, J = 8.4 Hz, 2H), 7.44 (d, J = 8.4 Hz, 2H), 6.76 (d, J = 8.4 Hz, 1H), 3.96 (d, J = 7.2 Hz, 2H), 1.12 (t, J = 7.2 Hz, 3H). |
| 204 | 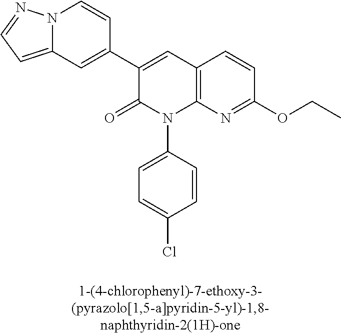 [SMILES: CCOc1ccc2cc(-c3ccn4nccc4c3)c(=O)n(-c3ccc(Cl)cc3)c2n1] | LC-MS (ESI): m/z 417 [M + H]+. 1H NMR (400 MHz, DMSO- d6) δ: 8.72 (d, J = 7.2 Hz, 1H), 8.44 (s, 1H), 8.22 (s, 1H), 8.18 (d, J = 8.4 Hz, 1H), 8.02 (d, J = 2.4 Hz, 1H), 7.62 (d, J = 8.4 Hz, 2H), 7.44 (d, J = 8.4 Hz, 2H), 7.27 (dd, J = 7.2 Hz, 2.0 Hz, 1H), 6.76 (d, J = 8.4 Hz, 1H), 6.68 (d, J = 2.0 Hz, 1H), 3.97 (q, J = 7.2 Hz, 2H), 1.11 (t, J = 7.2 Hz, 3H). |
| 205 | 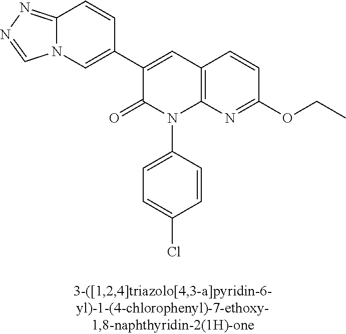 [SMILES: CCOc1ccc2cc(-c3ccc4nncn4c3)c(=O)n(-c3ccc(Cl)cc3)c2n1] | LC-MS (ESI): m/z 418 [M + H]+. 1H NMR (400 MHz, DMSO- d6) δ: 9.33 (s, 1H), 9.21 (s, 1H), 8.47 (s, 1H), 8.18 (d, J = 8.5 Hz, 1H), 7.91-7.71 (m, 2H), 7.62 (d, J = 8.8 Hz, 2H), 7.43 (d, J = 8.8 Hz, 2H), 6.78 (d, J = 8.4 Hz, 1H), 3.96 (q, J = 7.0 Hz, 2H), 1.11 (t, J = 7.0 Hz, 3H). |
| 206 | 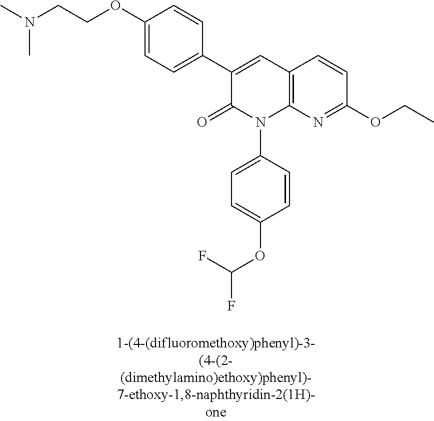 [SMILES: CCOc1ccc2cc(-c3ccc(OCCN(C)C)cc3)c(=O)n(-c3ccc(OC(F)F)cc3)c2n1] | LC-MS (ESI): m/z 496 [M + H]+. 1H NMR (400 MHz, DMSO- d6) δ: 8.13 (d, J = 8.0 Hz, 2H), 7.68 (d, J = 8.7 Hz, 2H), 7.35 (t, J_{HF} = 73.6 Hz, 1H), 7.57-7.13 (m, 4H), 7.00 (d, J = 8.8 Hz, 2H), 6.71 (d, J = 8.4 Hz, 1H), 4.09 (t, J = 5.7 Hz, 2H), 3.93 (q, J = 7.0 Hz, 2H), 2.66 (t, J = 5.7 Hz, 2H), 2.24 (s, 6H), 1.08 (t, J = 7.0 Hz, 3H). |
| 207 | 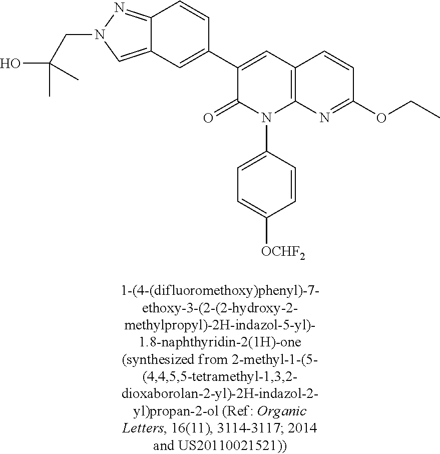 [SMILES: CCOc1ccc2cc(-c3ccc4nn(CC(C)(C)O)cc4c3)c(=O)n(-c3ccc(OC(F)F)cc3)c2n1] | LC-MS (ESI): m/z 521 [M + H]+. 1H NMR (400 MHz, DMSO- d6) δ: 8.36 (s, 1H), 8.21 (s, 1H), 8.16 (d, J = 8.4 Hz, 1H), 8.13 (s, 1H), 7.64 (d, J = 9.2 Hz, 1H), 7.57 (dd, J = 8.8 Hz, 1.6 Hz, 1H), 7.44 (d, J = 8.8 Hz, 2H), 7.35 (t, J_{HF} = 74.0 Hz, 1H), 7.34 (d, J = 8.8 Hz, 2H), 6.72 (d, J = 8.4 Hz, 1H), 4.88 (s, 1H), 4.35 (s, 2H), 3.95 (q, J = 7.2 Hz, 2H), 1.13 (s, 6H), 1.10 (t, J = 7.2 Hz, 3H). |
| 208 | 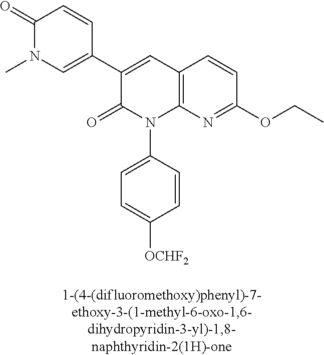 [SMILES: CCOc1ccc2cc(-c3ccc(=O)n(C)c3)c(=O)n(-c3ccc(OC(F)F)cc3)c2n1] | LC-MS (ESI): m/z 440 [M + H]+. 1H NMR (400 MHz, DMSO- d6) δ: 8.34 (d, J = 2.6 Hz, 1H), 8.21 (s, 1H), 8.10 (d, J = 8.5 Hz, 1H), 7.85 (dd, J = 9.5 Hz, 2.7 Hz, 1H), 7.37 (m, 4H), 7.34 (t, J_{HF} = 72.0 Hz, 1H), 6.73 (d, J = 8.4 Hz, 1H), 6.47 (d, J = 9.4 Hz, 1H), 3.94 (q, J = 7.0 Hz, 2H), 3.49 (s, 3H), 1.08 (t, J = 7.0 Hz, 3H). |
| 209 | 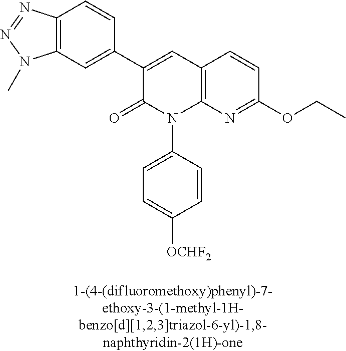 [SMILES: CCOc1ccc2cc(-c3ccc4nnn(C)c4c3)c(=O)n(-c3ccc(OC(F)F)cc3)c2n1] | LC-MS (ESI): m/z 464 [M + H]+. 1H NMR (400 MHz, DMSO- d6) δ: 8.37 (s, 1H), 8.24 (s, 1H), 8.19 (d, J = 8.4 Hz, 1H), 8.06 (d, J = 8.4 Hz, 1H), 7.76 (dd, J = 8.8 Hz, 1.4 Hz, 1H), 7.56- 7.14 (m, 4H), 7.35 (t, J_{HF} = 73.6 Hz, 1H), 6.76 (d, J = 8.4 Hz, 1H), 4.33 (s, 3H), 3.96 (q, J = 7.0 Hz, 2H), 1.10 (t, J = 7.0 Hz, 3H). |
| 210 | 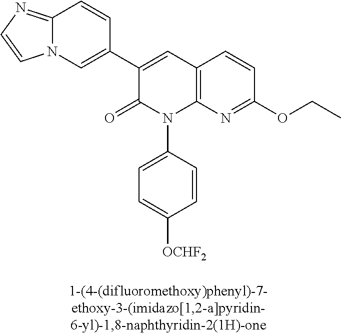 [SMILES: CCOc1ccc2cc(-c3ccc4nccn4c3)c(=O)n(-c3ccc(OC(F)F)cc3)c2n1] | LC-MS (ESI): m/z 449 [M + H]+. 1H NMR (400 MHz, DMSO- d6) δ: 9.16 (s, 1H), 8.40 (s, 1H), 8.15 (d, J = 9.0 Hz, 1H), 8.02 (s, 1H), 7.62 (s, 2H), 7.59 (s, 1H), 7.45 (d, J = 8.8 Hz, 2H), 7.39-7.32 (m, 2H), 7.36 (t, J_{HF} = 74 Hz, 1H), 6.76 (d, J = 8.4 Hz, 1H), 3.96 (q, J = 7.0 Hz, 2H), 1.10 (t, J = 7.0 Hz, 3H). |
| 211 | 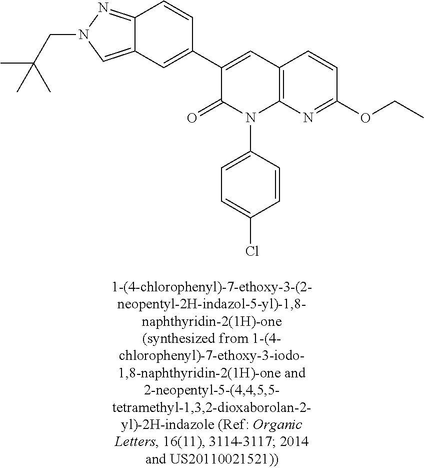 [SMILES: CCOc1ccc2cc(-c3ccc4nn(CC(C)(C)C)cc4c3)c(=O)n(-c3ccc(Cl)cc3)c2n1] | LC-MS (ESI): m/z 487 [M + H]+. 1H NMR (400 MHz, DMSO- d6) δ: 8.38 (s, 1H), 8.22 (s, 1H), 8.16 (d, J = 8.4 Hz, 1H), 8.11 (s, 1H), 7.64 (d, J = 9.2 Hz, 1H), 7.61 (d, J = 8.8 Hz, 2H), 7.56 (dd, J = 9.2 Hz, 1.2 Hz, 1H), 7.42 (d, J = 8.4 Hz, 2H), 6.73 (d, J = 8.4 Hz, 1H), 4.24 (s, 2H), 3.95 (q, J = 7.2 Hz, 2H), 1.11 (t, J = 6.8 Hz, 3H), 0.97 (s, 9H). |
| 212 | 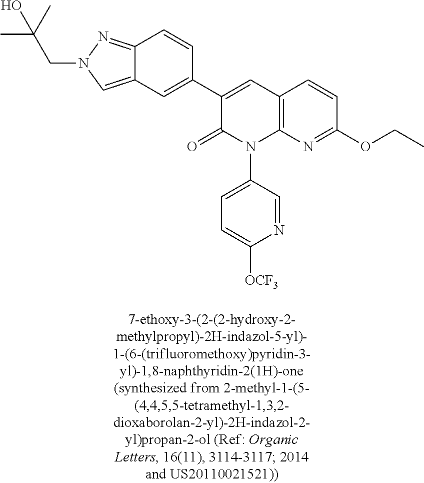 [SMILES: CCOc1ccc2cc(-c3ccc4nn(CC(C)(C)O)cc4c3)c(=O)n(-c3ccc(OC(F)(F)F)nc3)c2n1] | LC-MS (ESI): m/z 540 [M + H]+. 1H NMR (400 MHz, DMSO- d6) δ: 8.48 (d, J = 2.5 Hz, 1H), 8.37 (s, 1H), 8.27 (s, 1H), 8.19 (dd, J = 8.5 Hz, 2.0 Hz, 2H), 8.14 (s, 1H), 7.65 (d, J = 9.1 Hz, 1H), 7.57 (dd, J = 9.1 Hz, 1.5 Hz, 1H), 7.54 (d, J = 8.6 Hz, 1H), 6.77 (d, J = 8.6 Hz, 1H), 4.89 (s, 1H), 4.35 (s, 2H), 3.96 (q, J = 7.0 Hz, 2H), 1.12 (s, 6H), 1.10 (d, J = 7.0 Hz, 3H). |
| 213 | 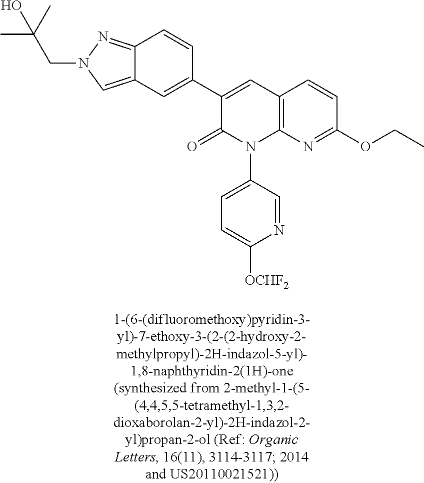 [SMILES: CCOc1ccc2cc(-c3ccc4nn(CC(C)(C)O)cc4c3)c(=O)n(-c3ccc(OC(F)F)nc3)c2n1] | LC-MS (ESI): m/z 522 [M + H]+. 1H NMR (400 MHz, DMSO- d6) δ: 8.36 (s, 1H), 8.34 (d, J = 2.2 Hz, 1H), 8.24 (s, 1H), 8.18 (d, J = 8.5 Hz, 1H), 8.13 (d, J = 1.4 Hz, 1H), 8.05 (dd, J = 8.7 Hz, 2.6 Hz, 1H), 7.82 (t, J_{HF} = 69.1 Hz, 1H), 7.63 (s, 1H), 7.57 (dd, J = 9.1 Hz, 1.6 Hz, 1H), 7.30 (dd, J = 8.6 Hz, 0.4 Hz, 1H), 6.76 (d, J = 8.4 Hz, 1H), 4.87 (s, 1H), 4.35 (s, 2H), 3.98 (q, J = 6.9 Hz, 2H), 1.13 (t, J = 6.9 Hz, 3H), 1.12 (s, 6H). |
| 214 | 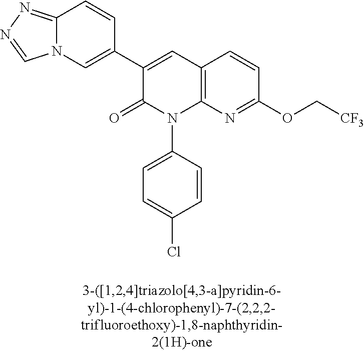 [SMILES: O=c1c(-c2ccc3nncn3c2)cc2ccc(OCC(F)(F)F)nc2n1-c1ccc(Cl)cc1] | LC-MS (ESI): m/z 472 [M + H]+. 1H NMR (400 MHz, DMSO- d6) δ: 9.35 (s, 1H), 9.21 (s, 1H), 8.52 (s, 1H), 8.31 (d, J = 8.5 Hz, 1H), 7.87 (d, J = 9.6 Hz, 1H), 7.77 (dd, J = 9.7, 1.5 Hz, 1H), 7.64 (d, J = 8.6 Hz, 2H), 7.45 (d, J = 8.6 Hz, 2H), 6.98 (d, J = 8.4 Hz, 1H), 4.63 (q, J = 9.0 Hz, 2H). |
| 215 | 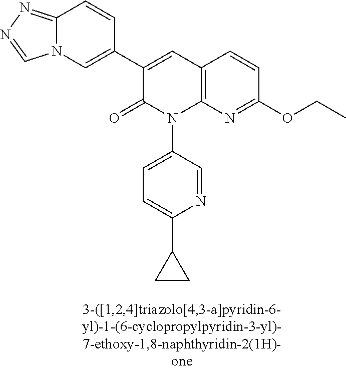 [SMILES: CCOc1ccc2cc(-c3ccc4nncn4c3)c(=O)n(-c3ccc(C4CC4)nc3)c2n1] | LC-MS (ESI): m/z 425 [M + H]+. 1H NMR (400 MHz, DMSO- d6) δ: 9.34 (s, 1H), 9.20 (s, 1H), 8.47 (s, 1H), 8.39 (d, J = 2.0 Hz, 1H), 8.19 (d, J = 8.4 Hz, 1H), 7.86 (d, J = 9.6 Hz, 1H), 7.77 (dd, J = 9.6 Hz, 1.6 Hz, 1H), 7.72 (dd, J = 8.0 Hz, 2.4 Hz, 1H), 7.50 (d, J = 8.4 Hz, 1H), 6.79 (d, J = 8.4 Hz, 1H), 3.97 (q, J = 6.8 Hz, 2H), 2.27- 2.16 (m, 1H), 1.11 (t, J = 6.8 Hz, 3H), 1.06-0.96 (m, 4H). |
| 216 | 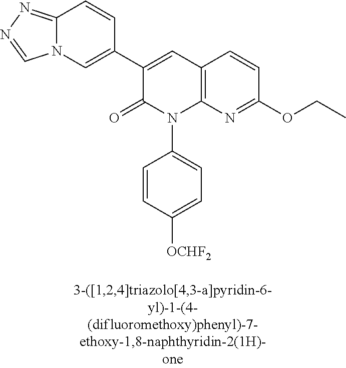 [SMILES: CCOc1ccc2cc(-c3ccc4nncn4c3)c(=O)n(-c3ccc(OC(F)F)cc3)c2n1] | LC-MS (ESI): m/z 450 [M + H]+. 1H NMR (400 MHz, DMSO- d6) δ: 9.38 (s, 1H), 9.26 (s, 1H), 8.51 (s, 1H), 8.22 (d, J = 8.4 Hz, 1H), 7.90 (d, J = 9.6 Hz, 1H), 7.82 (dd, J = 9.6 Hz, 1.6 Hz, 1H), 7.50 (d, J = 8.8 Hz, 2H), 7.41 (d, J = 8.8 Hz, 2H), 7.40 (t, J_{HF} = 73.6 Hz, 1H), 6.82 (d, J = 8.4 Hz, 1H), 4.01 (q, J = 7.2 Hz, 2H), 1.15 (t, J = 7.2 Hz, 3H). |
| 217 | 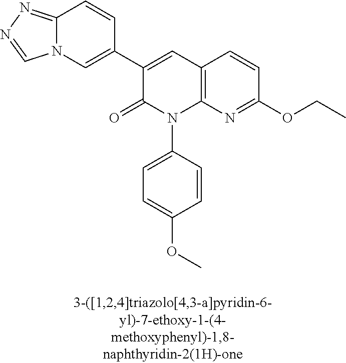 [SMILES: CCOc1ccc2cc(-c3ccc4nncn4c3)c(=O)n(-c3ccc(OC)cc3)c2n1] | LC-MS (ESI): m/z 414 [M + H]+. 1H NMR (400 MHz, DMSO- d6) δ: 9.33 (s, 1H), 9.21 (s, 1H), 8.44 (s, 1H), 8.17 (d, J = 8.5 Hz, 1H), 7.85 (d, J = 9.7 Hz, 1H), 7.77 (dd, J = 9.7, 1.6 Hz, 1H), 7.27 (d, J = 8.9 Hz, 2H), 7.09 (d, J = 8.9 Hz, 2H), 6.76 (d, J = 8.4 Hz, 1H), 3.98 (q, J = 7.0 Hz, 2H), 3.83 (s, 3H), 1.11 (t, J = 7.0 Hz, 3H). |
| 218 | 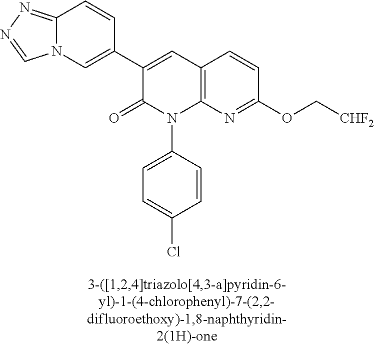 [SMILES: O=c1c(-c2ccc3nncn3c2)cc2ccc(OCC(F)F)nc2n1-c1ccc(Cl)cc1] | LC-MS (ESI): m/z 454 [M + H]+. 1H NMR (400 MHz, DMSO- d6, 0.4 HCO₂H salt) δ: 9.35 (s, 1H), 9.22 (s, 1H), 8.51 (s, 1H), 8.27 (d, J = 8.4 Hz, 1H), 8.17 (HCO₂H, s, 0.4H), 7.87 (d, J = 9.6 Hz, 1H), 7.77 (dd, J = 9.7, 1.4 Hz, 1H), 7.64 (d, J = 8.6 Hz, 2H), 7.46 (d, J = 8.6 Hz, 2H), 6.92 (d, J = 8.4 Hz, 1H), 6.13 (tt, J_{HF} = 55.2 Hz, J = 3.6 Hz, 1H), 4.19 (td, J_{HF} = 14.6 Hz, J = 3.6 Hz, 2H). |
| 219 | 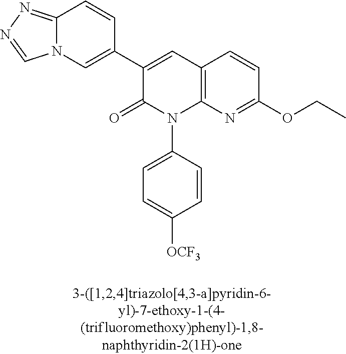 [SMILES: CCOc1ccc2cc(-c3ccc4nncn4c3)c(=O)n(-c3ccc(OC(F)(F)F)cc3)c2n1] | LC-MS (ESI): m/z 468 [M + H]+. 1H NMR (400 MHz, DMSO- d6) δ: 9.33 (s, 1H), 9.23 (s, 1H), 8.49 (s, 1H), 8.19 (d, J = 8.5 Hz, 1H), 7.87 (d, J = 9.6 Hz, 1H), 7.78 (dd, J = 9.7, 1.5 Hz, 1H), 7.60-7.52 (m, 4H), 6.78 (d, J = 8.4 Hz, 1H), 3.96 (q, J = 7.0 Hz, 2H), 1.07 (t, J = 7.0 Hz, 3H). |
| 220 | 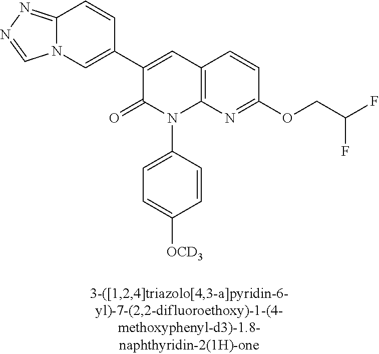 | LC-MS (ESI): m/z 453 [M + H]+. 1H NMR (400 MHz, DMSO- d6) δ: 9.33 (s, 1H), 9.21 (s, 1H), 8.47 (s, 1H), 8.25 (d, J = 8.4 Hz, 1H), 7.91-7.71 (m, 2H), 7.34-7.25 (m, 2H), 7.13-7.05 (m, 2H), 6.89 (d, J = 8.4 Hz, 1H), 6.10 (tt, J_{HF} = 55.2 Hz, J = 3.8 Hz, 1H), 4.18 (td, J_{HF} = 14.5, J = 3.8 Hz, 2H). |
| 221 | 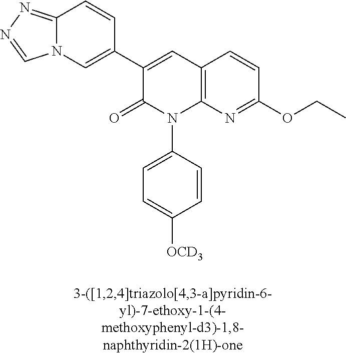 | LC-MS (ESI): m/z 417 [M + H]+. 1H NMR (400 MHz, DMSO- d6) δ: 9.32 (s, 1H), 9.21 (s, 1H), 8.43 (s, 1H), 8.16 (d, J = 8.5 Hz, 1H), 7.84 (d, J = 9.6 Hz, 1H), 7.76 (d, J = 10.8 Hz, 1H), 7.26 (d, J = 8.8 Hz, 2H), 7.08 (d, J = 8.8 Hz, 2H), 6.75 (d, J = 8.4 Hz, 1H), 3.97 (q, J = 7.2 Hz, 2H), 1.11 (t, J = 7.2 Hz, 3H). |
| 321 | 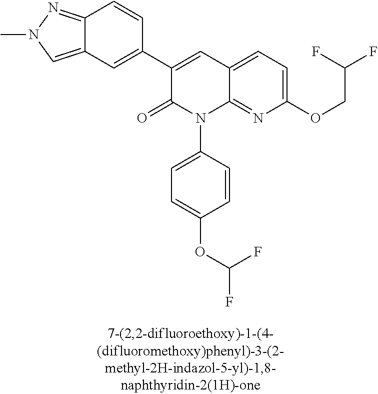 [SMILES: Cn1cc2cc(-c3cc4ccc(OCC(F)F)nc4n(-c4ccc(OC(F)F)cc4)c3=O)ccc2n1] | LC-MS: m/z 499 (M + H)+. 1H NMR (400 MHz, DMSO- d6) δ: 8.39 (s, 1H), 8.25 (s, 1H), 8.23 (s, 1H), 8.12 (s, 1H), 7.59 (d, J = 8.5 Hz, 2H), 7.45 (d, J = 8.0 Hz, 2H), 7.36 (d, J = 7.6 Hz, 2H), 7.32 (t, JHF = 73.8 Hz, 1H), 6.85 (d, J = 8.2 Hz, 1H), 6.08 (t, JHF = 55.0 Hz, 1H), 4.19-4.10 (m, 5H). |
| 322 | 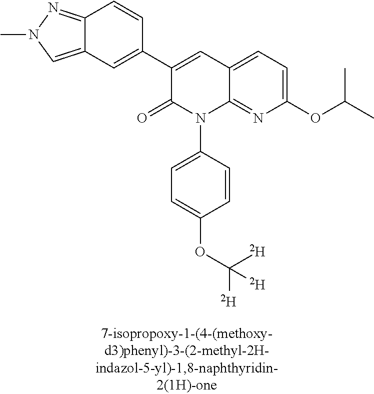 [SMILES: [2H]C([2H])([2H])Oc1ccc(-n2c(=O)c(-c3ccc4nn(C)cc4c3)cc3ccc(OC(C)C)nc32)cc1] | LC-MS: m/z 444 (M + H)+. 1H NMR (400 MHz, DMSO- d6) δ: 8.37 (s, 1H), 8.17 (s, 1H), 8.11 (d, J = 8.4 Hz, 2H), 7.63- 7.52 (m, 2H), 7.29-7.19 (m, 2H), 7.07 (d, J = 8.9 Hz, 2H), 6.64 (d, J = 8.4 Hz, 1H), 4.65- 4.59 (m, 1H), 4.17 (s, 3H), 1.09 (d, J = 6.2 Hz, 6H). |
| 323 | 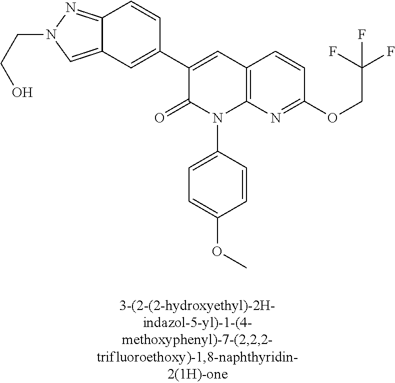 [SMILES: COc1ccc(-n2c(=O)c(-c3ccc4nn(CCO)cc4c3)cc3ccc(OCC(F)(F)F)nc32)cc1] | LC-MS: m/z 511 (M + H)+. 1H NMR (400 MHz, DMSO- d6) δ: 8.41 (s, 1H), 8.27 (d, J = 8.4 Hz, 1H), 8.24 (s, 1H), 8.13 (s, 1H), 7.65-7.60 (m, 1H), 7.57 (dd, J = 9.1 Hz, 1.6 Hz, 1H), 7.27 (d, J = 8.4 Hz, 2H), 7.09 (d, J = 8.4 Hz, 2H), 6.90 (d, J = 8.4 Hz, 1H), 4.98 (t, J = 5.2 Hz, 1H), 4.62 (q, JHF = 9.2 Hz, 2H), 4.47 (t, J = 5.2 Hz, 2H), 3.89 (q, J = 5.2 Hz, 2H), 3.83 (s, 3H) |

General Procedure V

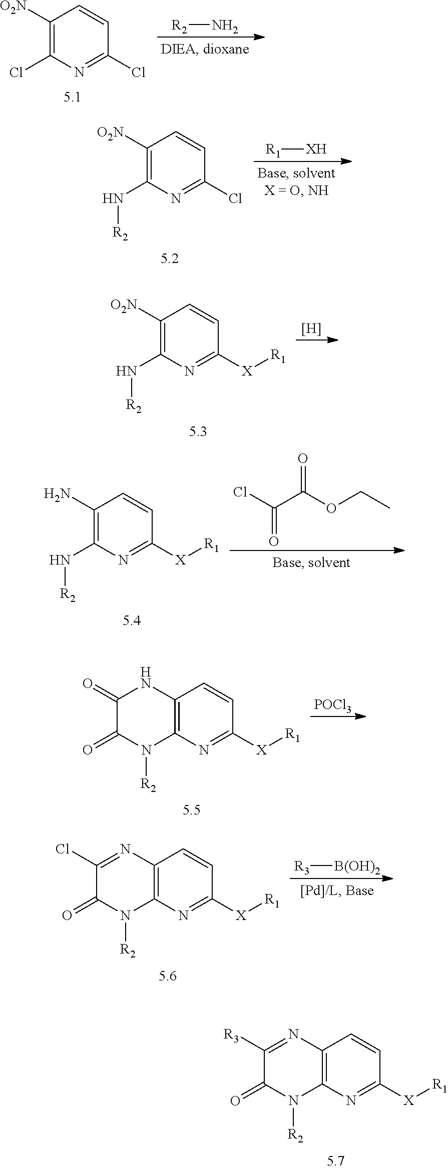

Compounds of structure 5.7 were obtained through the scheme depicted as General Procedure V. Beginning with substituted pyridine 5.1, the desired R2 group was introduced through nucleophilic aromatic substitution to generate amino-pyridine 5.2. The desired R₁ group was also introduced through nucleophilic aromatic substitution to generate substituted pyridine 5.3. Following reduction of nitro-pyridine 5.3 to diamino-pyridine 5.4, the bicyclic ring 5.5 was formed through a reaction with ethyl chlorooxoacetate and base. Heterocycle 5.5 was reacted with phosphoryl chloride to generate heteroaryl-chloride 5.6. The desired R3 group was introduced using a palladium mediated Suzuki coupling to afford compounds of structure 5.7.

### Preparation of Example 222 via General Procedure V

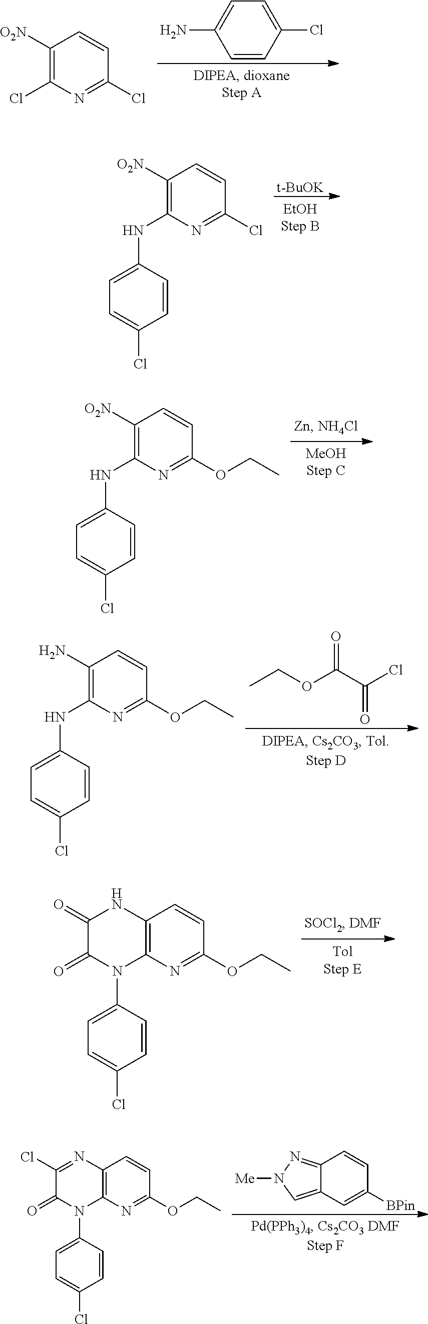

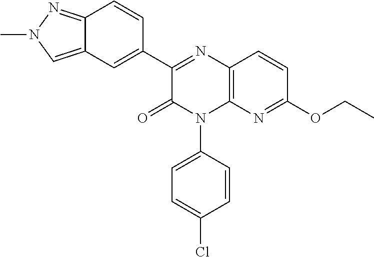 [SMILES: CCOc1ccc2nc(-c3ccc4nn(C)cc4c3)c(=O)n(-c3ccc(Cl)cc3)c2n1]

### Step A: 6-chloro-N-(4-chlorophenyl)-3-nitropyridin-2-amine

To a solution of 2,6-dichloro-3-nitropyridine (1.1 g, 5.73 mmol, 1.0 eq.) in dioxane (15 mL) was added 4-chloroaniline (0.8 g, 6.3 mmol, 1.1 eq.), DIPEA (2.2 g, 17.2 mmol, 3.0 eq.) at room temperature. The reaction mixture was stirred at 80° C. for 3 hrs. Then the reaction mixture was diluted with water (15 mL) and extracted with EtOAc (15 mL×3). The combined organic layers were washed with brine (20 mL), dried over Na₂SO₄ and concentrated under reduced pressure. The residue was purified by flash column chromatography on silica gel to afford 6-chloro-N-(4-chlorophenyl)-3-nitropyridin-2-amine (1.54 g, 95% yield) as a brown solid. LC-MS (ESI): m/z 284 [M+H]⁺.

### Step B: N-(4-chlorophenyl)-6-ethoxy-3-nitropyridin-2-amine

To a solution of 6-chloro-N-(4-chlorophenyl)-3-nitropyridin-2-amine (1.5 g, 5.3 mmol, 1.0 eq.) in EtOH (30 mL) was added t-BuOK (1.78 g, 15.9 mmol, 3.0 eq.) at 0° C. The reaction mixture was stirred at 80° C. for 3 hrs. The mixture was quenched with ice water (30 mL) and extracted with EtOAc (30 mL×3). The combined organic layers were washed with brine (20 mL), dried over Na₂SO₄ and concentrated under reduced pressure. The residue was purified by flash column chromatography on silica gel to afford N-(4-chlorophenyl)-6-ethoxy-3-nitropyridin-2-amine (1.1 g, 71% yield) as a brown solid. LC-MS (ESI): m/z 294 [M+H]⁺.

### Step C: N2-(4-chlorophenyl)-6-ethoxypyridine-2,3-diamine

A mixture of N-(4-chlorophenyl)-6-ethoxy-3-nitropyridin-2-amine (1.1 g, 3.75 mmol, 1.0 eq.), NH₄Cl (3.18 g, 60 mmol, 16.0 eq.) and zinc powder (1.9 g, 30 mmol, 8.0 eq.) in methanol (20 mL) was stirred at room temperature for 3 hrs. The reaction mixture was filtered and washed with methanol (30 mL), the filtrate was concentrated under reduced pressure to give N2-(4-chlorophenyl)-6-ethoxypyridine-2,3-diamine (950 mg, 96% yield) as a black solid. LC-MS (ESI): m/z 264 [M+H]⁺.

### Step D: 4-(4-chlorophenyl)-6-ethoxypyrido[2,3-b]pyrazine-2,3(1H,4H)-dione

To a solution of N2-(4-chlorophenyl)-6-ethoxypyridine-2,3-diamine (600 mg, 2.28 mmol, 1.0 eq.) and DIPEA (880 mg, 6.84 mmol, 3.0 eq.) in toluene/DCM mixture (15 mL, 5:1, v:v) was added ethyl 2-chloro-2-oxoacetate (621 mg, 4.56 mmol, 2.0 eq.) at 0° C. The reaction mixture was stirred at room temperature for 3 hrs. And then Cs₂CO₃ (2.23 g, 6.84 mmol, 3.0 eq.) was added to the above mixture. The reaction mixture was stirred at 80° C. for 14 hrs. The reaction mixture was cooled to room temperature, filtered and washed with DCM (30 mL), the filtrate was concentrated under reduced pressure. The residue was purified by flash column chromatography on silica gel to give 4-(4-chlorophenyl)-6-ethoxypyrido[2,3-b]pyrazine-2,3(1H,4H)-dione (300 mg, 42% yield) as a white solid. LC-MS (ESI): m/z 318 [M+H]⁺.

### Step E: 2-chloro-4-(4-chlorophenyl)-6-ethoxypyrido[2,3-b]pyrazin-3(4H)-one

To a solution of 4-(4-chlorophenyl)-6-ethoxypyrido[2,3-b]pyrazine-2,3(1H,4H)-dione (200 mg, 0.63 mmol, 1.0 eq.) in toluene (8 mL) was added SOCl2 (743 mg, 6.3 mmol, 10.0 eq.) and DMF (0.2 mL) at room temperature. The reaction mixture was stirred at 80° C. for 3 hrs. Then the reaction mixture was concentrated under reduced pressure, the residue was purified by flash column chromatography on silica gel to give 2-chloro-4-(4-chlorophenyl)-6-ethoxypyrido[2,3-b]pyrazin-3(4H)-one (200 mg, 95% yield) as a white solid. LC-MS (ESI): m/z 336 [M+H]⁺.

### Step F: 4-(4-chlorophenyl)-6-ethoxy-2-(2-methyl-2H-indazol-5-yl)pyrido[2,3-b]pyrazin-3(4H)-one

A mixture of 2-chloro-4-(4-chlorophenyl)-6-ethoxypyrido[2,3-b]pyrazin-3(4H)-one (100 mg, 0.3 mmol, 1.0 eq.), 2-methyl-5-(4,4,5,5-tetramethyl-1,3,2-dioxaborolan-2-yl)-2H-indazole (129 mg, 0.5 mmol, 1.67 eq.), K₂CO₃ (83 mg, 0.6 mmol, 2.0 eq.) and Pd(PPh₃)₄ (35 mg, 0.03 mmol, 0.1 eq.) in dioxane/H₂O mixture (5 mL, 10:1, v:v) was stirred at 100° C. for 4 hrs. Then the reaction mixture was diluted with water (15 mL) and extracted with EtOAc (15 mL×3). The combined organic layers were washed with brine (20 mL), dried over Na₂SO₄ and concentrated under reduced pressure. The residue was purified by RP-prep-HPLC to afford 4-(4-chlorophenyl)-6-ethoxy-2-(2-methyl-2H-indazol-5-yl)pyrido[2,3-b]pyrazin-3(4H)-one (Example 222).

¹H NMR (400 MHz, DMSO-d₆) δ: 8.94 (s, 1H), 8.51 (s, 1H), 8.23 (d, J=8.8 Hz, 1H), 8.14 (dd, J=9.2 Hz, 1.6 Hz, 1H), 7.70-7.62 (m, 3H), 7.55-7.48 (m, 2H), 6.83 (d, J=8.4 Hz, 1H), 4.19 (s, 3H), 3.99 (q, J=7.2 Hz, 2H), 1.14 (t, J=7.2 Hz, 3H). LC-MS (ESI): m/z 432 [M+H]⁺.

The procedure set forth above for General Procedure V was used to synthesize the following compounds by using appropriate starting materials:

| Example | Structure | Characterization |
|---|---|---|
| 223 | 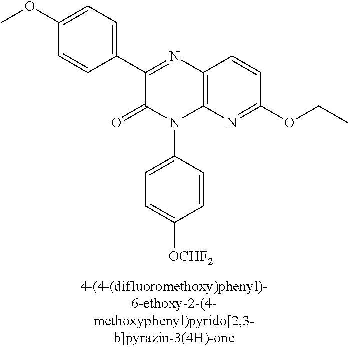 [SMILES: CCOc1ccc2nc(-c3ccc(OC)cc3)c(=O)n(-c3ccc(OC(F)F)cc3)c2n1] | LC-MS (ESI): m/z 440 [M + H]+. 1H NMR (400 MHz, DMSO- d6) δ: 8.31 (d, J = 9.0 Hz, 2H), 8.20 (d, J = 8.6 Hz, 1H), 7.51 (d, J = 8.8 Hz, 2H), 7.37 (d, J = 8.1 Hz, 2H), 7.36 (t, J_{HF} = 72.0 Hz, 1H), 7.05 (d, J = 9.0 Hz, 2H), 6.81 (d, J = 8.6 Hz, 1H), 3.98 (q, J = 7.0 Hz, 2H), 3.84 (s, 3H), 1.12 (t, J = 7.0 Hz, 3H). |
| 224 | 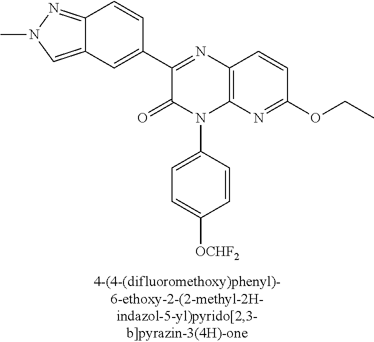 [SMILES: CCOc1ccc2nc(-c3ccc4nn(C)cc4c3)c(=O)n(-c3ccc(OC(F)F)cc3)c2n1] | LC-MS (ESI): m/z 464 [M + H]+. 1H NMR (400 MHz, DMSO- d6) δ: 8.93 (s, 1H), 8.50 (s, 1H), 8.23 (d, J = 8.4 Hz, 1H), 8.14 (dd, J = 9.2 Hz, 1.2 Hz, 1H), 7.66 (d, J = 9.2 Hz, 1H), 7.54-7.50 (m, 2H), 7.39 (t, J_{HF} = 69.2 Hz, 1H), 7.39-7.36 (m, 2H), 6.82 (d, J = 8.4 Hz, 1H), 4.19 (s, 3H), 3.99 (q, J = 7.2 Hz, 2H), 1.13 (t, J = 7.2 Hz, 3H). |
| 225 | 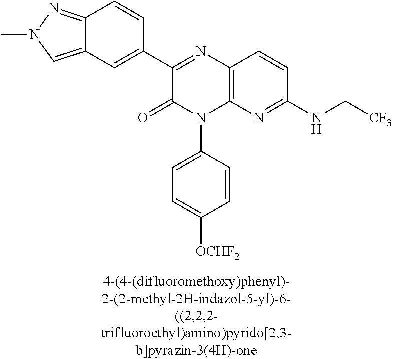 [SMILES: Cn1cc2cc(-c3nc4ccc(NCC(F)(F)F)nc4n(-c4ccc(OC(F)F)cc4)c3=O)ccc2n1] | LC-MS (ESI): m/z 517 [M + H]+. 1H NMR (400 MHz, DMSO- d6) δ: 8.89 (s, 1H), 8.47 (s, 1H), 8.12 (dd, J = 9.2 Hz, 1.6 Hz, 1H), 8.05-7.99 (m, 1H), 7.99 (d, J = 8.8 Hz, 1H), 7.63 (d, J = 9.6 Hz, 1H), 7.45 (d, J = 8.8 Hz, 2H), 7.35 (d, J = 8.8 Hz, 2H), 7.34 (t, J_{HF} = 72.0 Hz, 1H), 6.67 (d, J = 8.8 Hz, 1H), 4.18 (s, 3H), 3.84- 3.80 (m, 2H). |
| 226 | 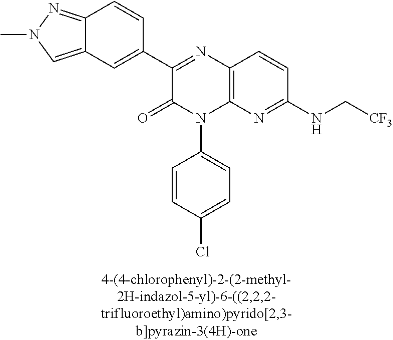 [SMILES: Cn1cc2cc(-c3nc4ccc(NCC(F)(F)F)nc4n(-c4ccc(Cl)cc4)c3=O)ccc2n1] | LC-MS (ESI): m/z 485 [M + H]+. 1H NMR (400 MHz, DMSO- d6) δ: 8.85 (s, 1H), 8.45 (s, 1H), 8.10 (d, J = 9.2 Hz, 1H), 7.99 (d, J = 8.4 Hz, 1H), 7.65-7.57 (m, 3H), 7.41 (dd, J = 8.4 Hz, 1.6 Hz, 2H), 6.67 (d, J = 8.8 Hz, 1H), 4.17 (s, 3H), 3.81 (q, J = 9.2 Hz, 2H). |
| 227 | 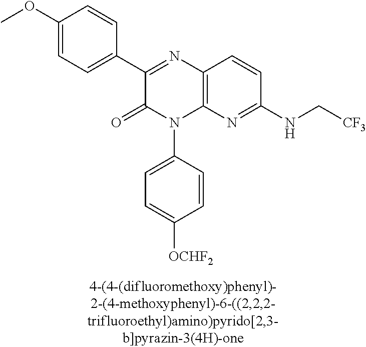 [SMILES: COc1ccc(-c2nc3ccc(NCC(F)(F)F)nc3n(-c3ccc(OC(F)F)cc3)c2=O)cc1] | LC-MS (ESI): m/z 493 [M + H]+ 1H NMR (400 MHz, DMSO- d6) δ: 8.27 (d, J = 9.0 Hz, 2H), 7.97 (t, J = 8.6 Hz, 2H), 7.43 (d, J = 8.8 Hz, 2H), 7.34 (d, J = 8.4 Hz, 2H), 7.35 (t, J_{HF} = 74 Hz, 1H), 7.02 (d, J = 9.0 Hz, 2H), 6.66 (d, J = 8.7 Hz, 1H), 3.83-3.78 (m, 5H). |
| 228 | 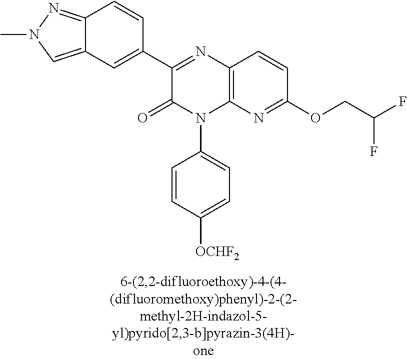 [SMILES: Cn1cc2cc(-c3nc4ccc(OCC(F)F)nc4n(-c4ccc(OC(F)F)cc4)c3=O)ccc2n1] | LC-MS (ESI): m/z 500 [M + H]+. 1H NMR (400 MHz, DMSO- d6) δ: 8.95 (s, 1H), 8.52 (s, 1H), 8.31 (d, J = 8.6 Hz, 1H), 8.15 (d, J = 9.2 Hz, 1H), 7.67 (d, J = 9.2 Hz, 1H), 7.55 (d, J = 8.0 Hz, 2H), 7.39 (d, J = 8.0 Hz, 2H), 7.36 (t, J_{HF} = 74.0 Hz, 1H), 6.96 (d, J = 8.4 Hz, 1H), 6.15 (t, J_{HF} = 55.1 Hz, 1H), 4.27-4.15 (m, 5H). |
| 324 | 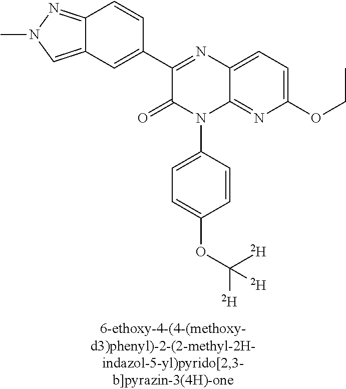 [SMILES: [2H]C([2H])([2H])Oc1ccc(-n2c(=O)c(-c3ccc4nn(C)cc4c3)nc3ccc(OCC)nc32)cc1] | LC-MS: m/z 431 (M + H)+. 1H NMR (400 MHz, CDCl3) δ: 9.10 (s, 1H), 8.30 (d, J = 9.3 Hz, 1H), 8.12 (d, J = 8.5 Hz, 1H), 7.96 (s, 1H), 7.75 (d, J = 9.0 Hz, 1H), 7.26 (s, 2H), 7.08 (d, J = 8.5 Hz, 2H), 6.71 (d, J = 8.5 Hz, 1H), 4.23 (s, 3H), 4.05 (q, J = 7.0 Hz, 2H), 1.21 (t, J = 7.0 Hz, 3H). |
| 325 | 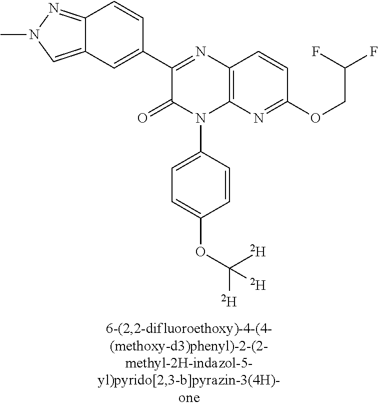 [SMILES: [2H]C([2H])([2H])Oc1ccc(-n2c(=O)c(-c3ccc4nn(C)cc4c3)nc3ccc(OCC(F)F)nc32)cc1] | LC-MS: m/z 467 (M + H)+. 1H NMR (400 MHz, DMSO- d6) δ: 8.95 (s, 1H), 8.52 (s, 1H), 8.30 (d, J = 8.6 Hz, 1H), 8.15 (dd, J = 9.2 Hz, 1.6 Hz, 1H), 7.67 (d, J = 9.2 Hz, 1H), 7.42-7.36 (m, 2H), 7.15-7.08 (m, 2H), 6.94 (d, J = 8.6 Hz, 1H), 6.16 (tt, JHF = 54.9 Hz, J = 3.6 Hz, 1H), 4.28-4.17 (m, 5H). |
| 326 | 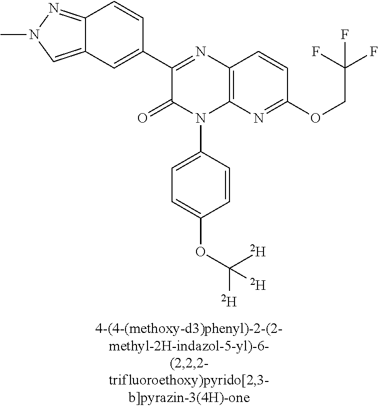 [SMILES: [2H]C([2H])([2H])Oc1ccc(-n2c(=O)c(-c3ccc4nn(C)cc4c3)nc3ccc(OCC(F)(F)F)nc32)cc1] | LC-MS: m/z 485 (M + H)+. 1H NMR (400 MHz, DMSO- d6) δ: 8.95 (s, 1H), 8.52 (s, 1H), 8.33 (d, J = 8.4 Hz, 1H), 8.14 (d, J = 9.0 Hz, 1H), 7.67 (d, J = 9.1 Hz, 1H), 7.36 (d, J = 8.5 Hz, 2H), 7.12 (d, J = 8.5 Hz, 2H), 6.99 (d, J = 8.5 Hz, 1H), 4.66 (q, J = 9.0 Hz, 2H), 4.19 (s, 3H). |
| 327 | 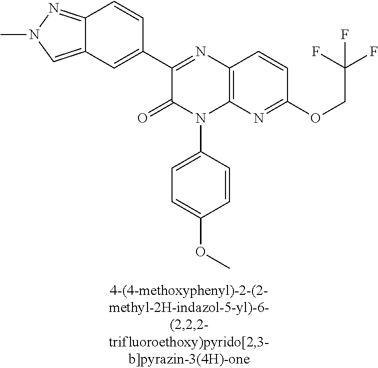 [SMILES: COc1ccc(-n2c(=O)c(-c3ccc4nn(C)cc4c3)nc3ccc(OCC(F)(F)F)nc32)cc1] | LC-MS: m/z 482 (M + H)+. 1H NMR (400 MHz, DMSO- d6) δ: 8.95 (s, 1H), 8.52 (s, 1H), 8.33 (d, J = 8.5 Hz, 1H), 8.15 (d, J = 9.2 Hz, 1H), 7.67 (d, J = 9.2 Hz, 1H), 7.36 (d, J = 8.8 Hz, 2H), 7.12 (d, J = 8.8 Hz, 2H), 7.00 (d, J = 8.5 Hz, 1H), 4.66 (q, JHF = 9.0 Hz, 2H), 4.19 (s, 3H), 3.85 (s, 3H). |
| 328 | 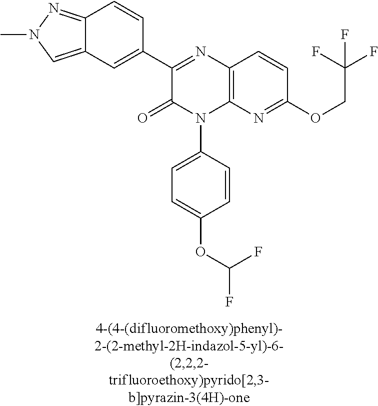 [SMILES: Cn1cc2cc(-c3nc4ccc(OCC(F)(F)F)nc4n(-c4ccc(OC(F)F)cc4)c3=O)ccc2n1] | LC-MS: m/z 518 (M + H)+. 1H NMR (400 MHz, DMSO- d6) δ: 8.94 (s, 1H), 8.52 (s, 1H), 8.34 (d, J = 8.6 Hz, 1H), 8.14 (dd, J = 9.2 Hz, 1.2 Hz, 1H), 7.67 (d, J = 9.2 Hz, 1H), 7.53 (d, J = 9.0 Hz, 2H), 7.39 (d, J = 8.7 Hz, 2H), 7.36 (t, JHF = 7.4 Hz, 1H), 7.00 (d, J = 8.6 Hz, 1H), 4.64 (q, JHF = 9.0 Hz, 2H), 4.19 (s, 3H). |
| 329 | 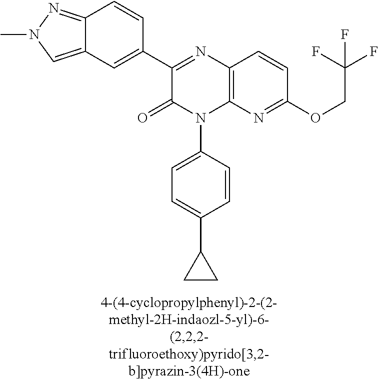 [SMILES: Cn1cc2cc(-c3nc4ccc(OCC(F)(F)F)nc4n(-c4ccc(C5CC5)cc4)c3=O)ccc2n1] | LC-MS: m/z 492 (M + H)+. 1H NMR (400 MHz, DMSO- d6) δ: 8.94 (s, 1H), 8.52 (s, 1H), 8.32 (d, J = 8.5 Hz, 1H), 8.14 (dd, J = 9.2 Hz, 1.5 Hz, 1H), 7.66 (d, J = 9.2 Hz, 1H), 7.28 (q, J = 8.6 Hz, 4H), 6.99 (d, J = 8.5 Hz, 1H), 4.63 (q, JHF = 9.0 Hz, 2H), 4.19 (s, 3H), 2.11-1.99 (m, 1H), 1.09- 0.97 (m, 2H), 0.81-0.69 (m, 2H). |
| 330 | 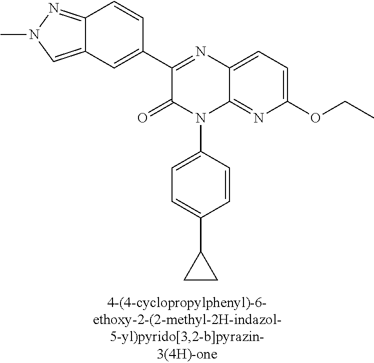 [SMILES: CCOc1ccc2nc(-c3ccc4nn(C)cc4c3)c(=O)n(-c3ccc(C4CC4)cc3)c2n1] | LC-MS: m/z 438 (M + H)+. 1H NMR (400 MHz, DMSO- d6) δ: 8.92 (s, 1H), 8.50 (s, 1H), 8.21 (d, J = 8.6 Hz, 1H), 8.14 (dd, J = 9.2 Hz, 1.5 Hz, 1H), 7.65 (d, J = 9.2 Hz, 1H), 7.27 (q, J = 8.6 Hz, 4H), 6.81 (d, J = 8.6 Hz, 1H), 4.18 (s, 3H), 3.98 (q, J = 7.0 Hz, 2H), 2.09-2.00 (m, 1H), 1.12 (t, J = 7.0 Hz, 3H), 1.06-1.00 (m, 2H), 0.79-0.73 (m, 2H). |
| 331 | 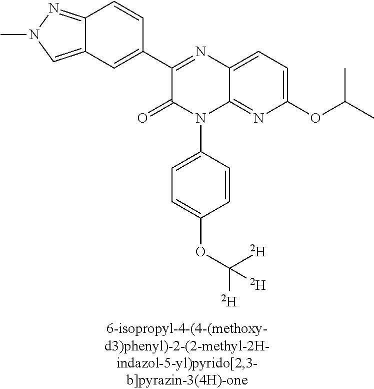 [SMILES: [2H]C([2H])([2H])Oc1ccc(-n2c(=O)c(-c3ccc4nn(C)cc4c3)nc3ccc(OC(C)C)nc32)cc1] | LC-MS: m/z 445 (M + H)+. 1H NMR (400 MHz, DMSO- d6): δ: 8.93 (s, 1H), 8.50 (s, 1H), 8.19 (d, J = 8.6 Hz, 1H), 8.14 (d, J = 9.2 Hz, 1H), 7.65 (d, J = 9.2 Hz, 1H), 7.34 (d, J = 8.8 Hz, 2H), 7.10 (d, J = 8.8 Hz, 2H), 6.76 (d, J = 8.6 Hz, 1H), 4.73-4.60 (m, 1H), 4.19 (s, 3H), 1.13 (d, J = 6.1 Hz, 6H). |
| 332 | 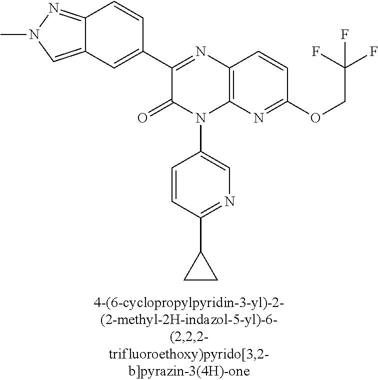 [SMILES: Cn1cc2cc(-c3nc4ccc(OCC(F)(F)F)nc4n(-c4ccc(C5CC5)nc4)c3=O)ccc2n1] | LC-MS: m/z 493 (M + H)+. 1H NMR (400 MHz, DMSO- d6) δ: 8.94 (s, 1H), 8.53 (s, 1H), 8.47 (d, J = 2.2 Hz, 1H), 8.35 (d, J = 8.6 Hz, 1H), 8.14 (dd, J = 9.2 Hz, 1.3 Hz, 1H), 7.79 (dd, J = 8.3 Hz, 2.4 Hz, 1H), 7.67 (d, J = 9.2 Hz, 1H), 7.53 (d, J = 8.3 Hz, 1H), 7.02 (d, J = 8.6 Hz, 1H), 4.66 (q, JHF = 9.0 Hz, 2H), 4.19 (s, 3H), 2.30-2.20 (m, 1H), 1.07- 0.97 (m, 4H). |
| 333 | 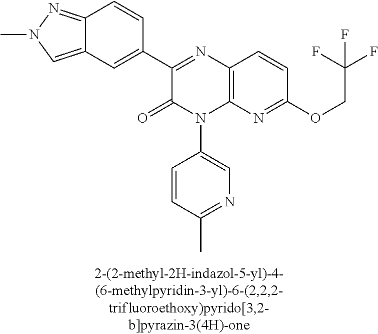 [SMILES: Cc1ccc(-n2c(=O)c(-c3ccc4nn(C)cc4c3)nc3ccc(OCC(F)(F)F)nc32)cn1] | LC-MS: m/z 467 (M + H)+. 1H NMR (400 MHz, DMSO- d6) δ: 8.88 (s, 1H), 8.66 (d, J = 2.0 Hz, 1H), 8.48 (s, 1H), 8.34 (d, J = 8.6 Hz, 1H), 8.11 (dd, J = 9.2 Hz, 1.5 Hz, 1H), 8.05 (dd, J = 8.3 Hz, 2.3 Hz, 1H), 7.67 (dd, J = 8.6 Hz, 6.4 Hz, 2H), 7.01 (d, J = 8.6 Hz, 1H), 4.63 (q, JHF = 8.9 Hz, 2H), 4.16 (s, 3H), 2.65 (s, 3H). |
| 334 | 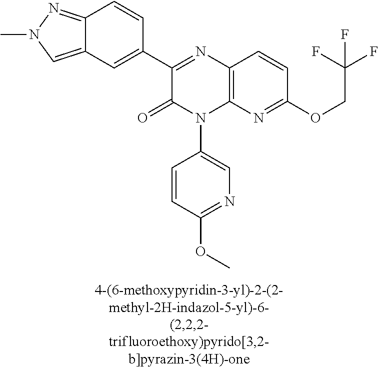 [SMILES: COc1ccc(-n2c(=O)c(-c3ccc4nn(C)cc4c3)nc3ccc(OCC(F)(F)F)nc32)cn1] | LC-MS: m/z 483 (M + H)+. 1H NMR (400 MHz, DMSO- d6) δ: 8.95 (s, 1H), 8.53 (s, 1H), 8.35 (d, J = 8.6 Hz, 1H), 8.28 (d, J = 2.3 Hz, 1H), 8.15 (dd, J = 9.2 Hz, 1.6 Hz, 1H), 7.85 (dd, J = 8.8 Hz, 2.6 Hz, 1H), 7.68 (d, J = 9.2 Hz, 1H), 7.04 (t, J = 8.5 Hz, 2H), 4.69 (q, JHF = 9.0 Hz, 2H), 4.19 (s, 3H), 3.95 (s, 3H). |
| 335 | 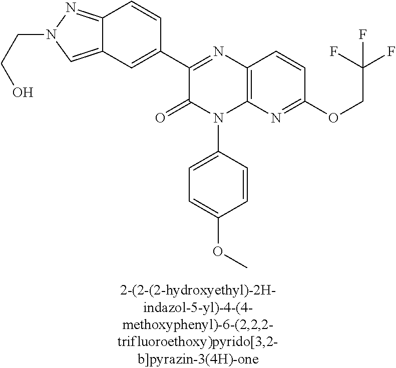 [SMILES: COc1ccc(-n2c(=O)c(-c3ccc4nn(CCO)cc4c3)nc3ccc(OCC(F)(F)F)nc32)cc1] | LC-MS: m/z 512 (M + H)+. 1H NMR (400 MHz, DMSO- d6) δ: 8.96 (s, 1H), 8.53 (s, 1H), 8.32 (d, J = 8.5 Hz, 1H), 8.15 (d, J = 9.2 Hz, 1H), 7.68 (d, J = 9.2 Hz, 1H), 7.36 (d, J = 8.6 Hz, 2H), 7.12 (d, J = 8.6 Hz, 2H), 6.99 (d, J = 8.5 Hz, 1H), 5.01 (t, J = 5.2 Hz, 1H), 4.66 (q, JHF = 8.9 Hz, 2H), 4.47 (t, J = 5.0 Hz, 2H), 3.94-3.87 (m, 2H), 3.83 (s, 3H). |
| 336 | 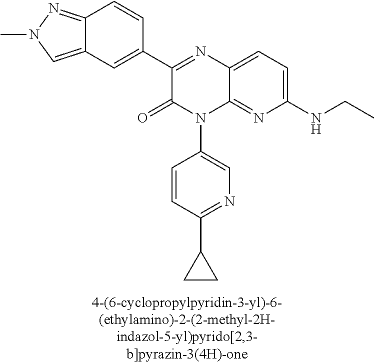 [SMILES: CCNc1ccc2nc(-c3ccc4nn(C)cc4c3)c(=O)n(-c3ccc(C4CC4)nc3)c2n1] | LC-MS: m/z 438 (M + H)+ 1H NMR (400 MHz, DMSO- d6) δ: 8.86 (s, 1H), 8.45 (s, 1H), 8.38 (d, J = 2.4 Hz, 1H), 8.12 (d, J = 9.2 Hz, 1H), 7.87 (d, J = 8.8 Hz, 1H), 7.72 (dd, J = 8.0 Hz, 2.4 Hz, 1H), 7.63 (d, J = 9.2 Hz, 1H), 7.55 (s, 1H), 7.49 (d, J = 8.2 Hz, 1H), 6.53 (d, J = 8.8 Hz, 1H), 4.18 (s, 3H), 2.99 (q, J = 6.4 Hz, 2H), 2.29-2.16 (m, 1H), 1.14- 0.76 (m, 7H). |
| 337 | 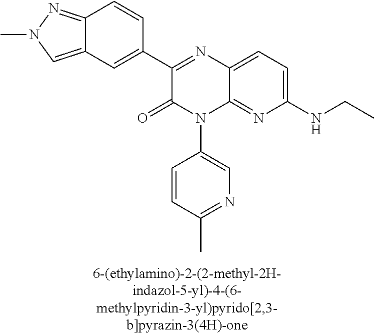 [SMILES: CCNc1ccc2nc(-c3ccc4nn(C)cc4c3)c(=O)n(-c3ccc(C)nc3)c2n1] | LC-MS: m/z 412 (M + H)+ 1H NMR (400 MHz, DMSO- d6) δ: 8.87 (s, 1H), 8.45 (s, 1H), 8.44 (s, 1H), 8.13 (dd, J = 9.2 Hz, 1.6 Hz, 1H), 7.87 (d, J = 8.8 Hz, 1H), 7.67 (dd, J = 8.2 Hz, 2.4 Hz, 1H), 7.62 (dd, J = 9.2 Hz, 0.8 Hz, 1H), 7.58 (s, 1H), 7.45 (d, J = 8.2 Hz, 1H), 6.54 (d, J = 8.8 Hz, 1H), 4.17 (s, 3H), 2.96 (q, J = 6.8 Hz, 2H), 2.58 (s, 3H), 0.93 (t, J = 7.2 Hz, 3H). |
| 338 | 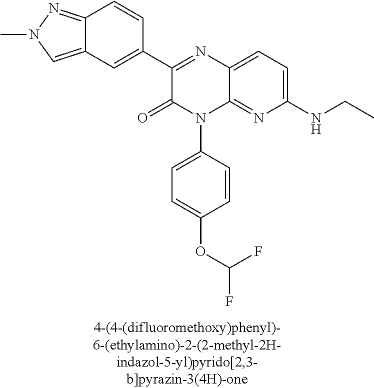 [SMILES: CCNc1ccc2nc(-c3ccc4nn(C)cc4c3)c(=O)n(-c3ccc(OC(F)F)cc3)c2n1] | LC-MS: m/z 463 (M + H)+ 1H NMR (400 MHz, DMSO- d6) δ: 8.80 (t, J = 1.2 Hz, 1H), 8.37 (s, 1H), 8.06 (dd, J = 9.2 Hz, 1.6 Hz, 1H), 7.79 (d, J = 8.8 Hz, 1H), 7.56 (d, J = 9.2 Hz, 1H), 7.41-7.35 (m, 3H), 7.28 (t, J_{HF} = 74 Hz, 1H), 7.32-7.26 (m, 2H), 6.45 (d, J = 8.8 Hz, 1H), 4.11 (s, 3H), 2.90 (t, J = 7.2 Hz, 2H), 0.85 (t, J = 7.2 Hz, 3H). |
| 339 | 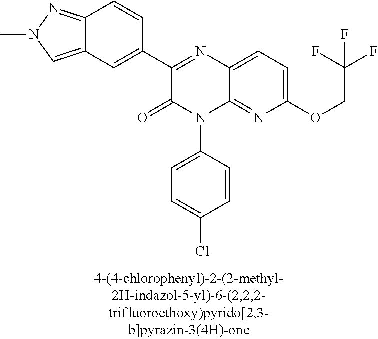 [SMILES: Cn1cc2cc(-c3nc4ccc(OCC(F)(F)F)nc4n(-c4ccc(Cl)cc4)c3=O)ccc2n1] | LC-MS: m/z 486 (M + H)+. 1H NMR (400 MHz, DMSO- d6) δ: 8.95 (s, 1H), 8.53 (s, 1H), 8.35 (d, J = 8.4 Hz, 1H), 8.15 (dd, J = 9.2 Hz, 1.6 Hz, 1H), 7.68 (d, J = 8.0 Hz, 3H), 7.52 (d, J = 8.0 Hz, 2H), 7.02 (d, J = 8.4 Hz, 1H), 4.65 (q, J_{HF} = 9.2 Hz, 2H), 4.19 (s, 3H). |
| 340 | 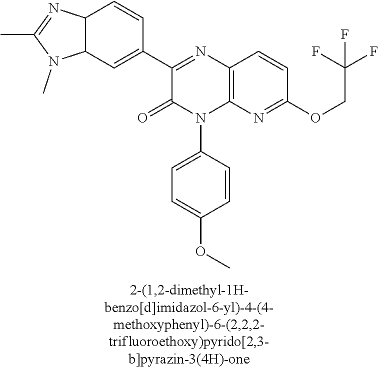 [SMILES: COc1ccc(-n2c(=O)c(C3=CC4C(C=C3)N=C(C)N4C)nc3ccc(OCC(F)(F)F)nc32)cc1] | LC-MS: m/z 496 (M + H)+. 1H NMR (400 MHz, DMSO- d6) δ: 8.51 (s, 1H), 8.34 (d, J = 8.4 Hz, 1H), 8.14 (d, J = 7.6 Hz, 1H), 7.60 (d, J = 8.4 Hz, 1H), 7.37 (d, J = 8.4 Hz, 2H), 7.13 (d, J = 8.4 Hz, 2H), 7.00 (d, J = 8.4 Hz, 1H), 4.67 (q, J_{HF} = 9.2 Hz, 2H), 3.85 (s, 3H), 3.77 (s, 3H), 2.57 (s, 3H). |
| 341 | 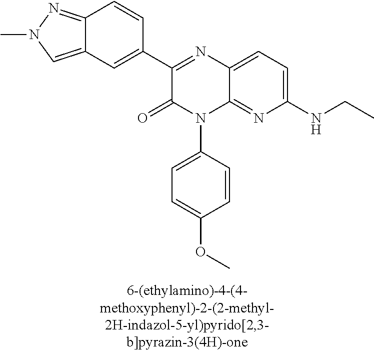 [SMILES: CCNc1ccc2nc(-c3ccc4nn(C)cc4c3)c(=O)n(-c3ccc(OC)cc3)c2n1] | LC-MS: m/z 427 (M + H)+. 1H NMR (400 MHz, DMSO- d6) δ: 8.87 (s, 1H), 8.44 (s, 1H), 8.12 (dd, J = 9.2 Hz, 1.6 Hz, 1H), 7.84 (d, J = 8.8 Hz, 1H), 7.61 (d, J = 9.2 Hz, 1H), 7.48 (s, 1H), 7.27 (d, J = 8.9 Hz, 2H), 7.07 (d, J = 8.9 Hz, 2H), 6.50 (d, J = 8.8 Hz, 1H), 4.17 (s, 3H), 3.83 (s, 3H), 3.05-2.88 (m, 2H), 0.94 (t, J = 7.1 Hz, 3H). |

General Procedure VI

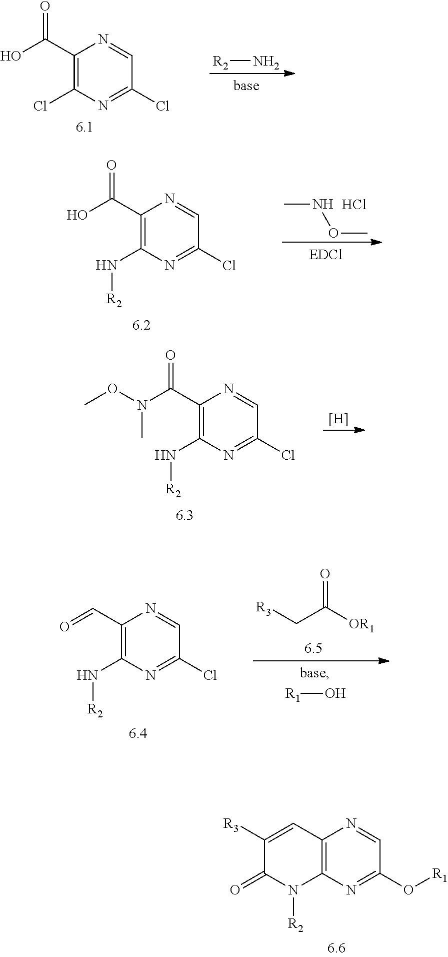

Compounds of structure 6.6 were obtained through the scheme depicted as General Procedure VI. Beginning with substituted pyrazine 6.1, the desired R2 group was introduced through nucleophilic aromatic substitution to generate amino-pyrazine 6.2. Weinreb amide 6.3 was then formed allowing for selective reduction to aldehyde 6.4. Substituted heterocycle 6.4 was then reacted with ester 6.5 and base to simultaneously introduce the desired R₃ and R₁ groups, close the bicyclic ring system, and afford compounds of structure 6.6.

### Preparation of Example 229 via General Procedure VI

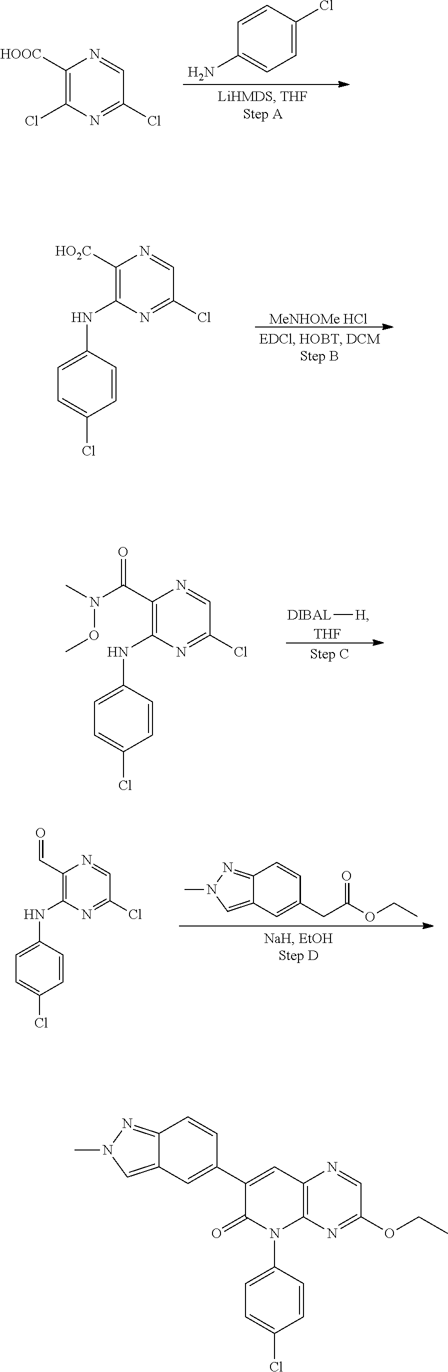

### Step A: 5-chloro-3-(4-chlorophenylamino)pyrazine-2-carboxylic acid

To a solution of 4-chloroaniline (397 mg, 3.13 mmol, 2.0 eq.) in dry THE (2 ml) was added LiHMDS (5 mL, 5 mmol, 1 M in THF, 3.2 eq.) drop-wise at −78° C. under N2 atmosphere. After stirring for 30 min, a solution of 3,5-dichloropyrazine-2-carboxylic acid (300 mg, 1.56 mol, 1.0 eq.) in dry THE (2 ml) was added drop-wise. The mixture was stirred at −78° C. for 30 min, and then allowed to warm to room temperature and stirred for 16 hrs. The mixture was quenched with H₂O (10 mL), the aqueous layer was adjusted pH=2 with dilute HCl (2 N, aq.), extracted with EtOAc (15 mL×3), The combined organic layers were washed with brine (20 mL), dried over Na₂SO₄ and concentrated under reduced pressure. The residue was purified by flash column chromatography on silica gel to afford (420 mg, 95% yield) as a yellow solid. LC-MS (ESI): m/z 284 [M+H]⁺.

### Step B: 5-chloro-3-(4-chlorophenylamino)-N-methoxy-N-methylpyrazine-2-carboxamide

A mixture of 5-chloro-3-(4-chlorophenylamino)pyrazine-2-carboxylic acid (410 mg, 1.45 mmol, 1.0 eq.), methoxy(methyl)amine hydrochloride (281 mg, 2.9 mmol, 2.0 eq.), DIPEA (748 mg, 5.8 mmol, 4.0 eq.) and HATU (2.2 g, 5.8 mmol, 4.0 eq.) in DCM (10 mL) was stirred at room temperature overnight. Then the reaction mixture was diluted with NH₄Cl (sat., aq.) (20 mL), then extracted with DCM (20 mL×3). The combined organic layers were washed with brine (20 mL), dried over Na₂SO₄ and concentrated under reduced pressure. The residue was purified by flash column chromatography on silica gel to afford 5-chloro-3-(4-chlorophenylamino)-N-methoxy-N-methylpyrazine-2-carboxamide (310 mg, 66% yield) as a yellow solid. LC-MS (ESI): m/z 327 [M+H]⁺.

### Step C: 5-chloro-3-(4-chlorophenylamino)pyrazine-2-carbaldehyde

To a solution of 5-chloro-3-(4-chlorophenylamino)-N-methoxy-N-methylpyrazine-2-carboxamide (50 mg, 0.15 mmol, 1.0 eq.) in dry THE (3 ml) was added DIBAL-H (0.15 mL, 0.23 mmol, 1.5 M in toluene, 1.5 eq.) drop-wise at −78° C. under N2 atmosphere. The mixture was stirred at −78° C. for 0.5 hr. The cold mixture was quenched directly with NH₄Cl (sat., aq.) (10 mL) and extracted with EtOAc (10 mL×3). The combined organic layers were washed with brine (20 mL), dried over Na₂SO₄ and concentrated under reduced pressure. The residue was rapidly purified by flash column chromatography on silica gel to give 5-chloro-3-[(4-chlorophenyl)amino]pyrazine-2-carbaldehyde as a semi-crude pale-yellow oil, which should be used immediately in the next step without further purification. LC-MS (ESI): m/z 268 [M+H]⁺.

### Step D: 5-(4-chlorophenyl)-3-ethoxy-7-(2-methyl-2H-indazol-5-yl)pyrido[2,3-b]pyrazin-6(5H)-one

A mixture of 5-chloro-3-[(4-chlorophenyl)amino]pyrazine-2-carbaldehyde (160 mg semi-crude, 0.6 mmol, 1.0 eq), ethyl 2-(2-methyl-2H-indazol-5-yl)acetate (131 mg, 0.6 mmol, 1 eq) and NaH (120 mg, 3.0 mmol, 5.0 eq, 60% in mineral oil) in EtOH (3 mL) was stirred at 60° C. overnight. The mixture was quenched with ice NH₄Cl (sat, aq.) (20 mL) and extracted with DCM (20 mL×3). The combined organic layers were washed with brine (20 mL), dried over Na₂SO₄ and concentrated under reduced pressure. The residue was purified by RP-prep-HPLC to afford 5-(4-chlorophenyl)-3-ethoxy-7-(2-methyl-2H-indazol-5-yl)pyrido[2,3-b]pyrazin-6(5H)-one (Example 229).

¹H NMR (400 MHz, DMSO-d₆) δ: 8.42 (s, 1H), 8.24 (s, 1H), 8.21 (s, 1H), 8.19 (s, 1H), 7.64 (d, J=8.4 Hz, 2H), 7.63-7.61 (m, 2H), 7.50 (d, J=8.4 Hz, 2H), 4.19 (s, 3H), 4.05 (q, J=7.2 Hz, 2H), 1.19 (t, J=7.2 Hz, 3H). LC-MS (ESI): m/z 432 [M+H]⁺.

The procedure set forth above for General Procedure VI was used to synthesize the following compounds by using appropriate starting materials.

| Example | Structure | Characterization |
|---|---|---|
| 230 | 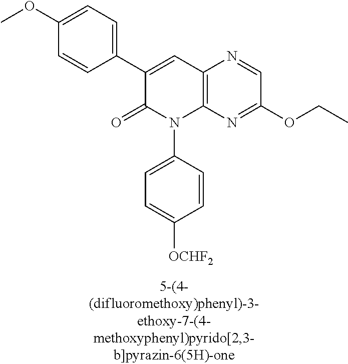 [SMILES: CCOc1cnc2cc(-c3ccc(OC)cc3)c(=O)n(-c3ccc(OC(F)F)cc3)c2n1] | LC-MS (ESI): m/z 440 [M + H]+. 1H NMR (400 MHz, DMSO- d6) δ: 8.21 (s, 1H), 8.13 (s, 1H), 7.75 (d, J = 8.8 Hz, 2H), 7.47 (d, J = 8.9 Hz, 2H), 7.39-7.32 (m, 2H), 7.35 (t, J_{HF} = 74.0 Hz, 1H), 7.01 (d, J = 8.9 Hz, 2H), 4.03 (q, J = 7.0 Hz, 2H), 3.81 (s, 3H), 1.15 (t, J = 7.0 Hz, 3H). |
| 231 | 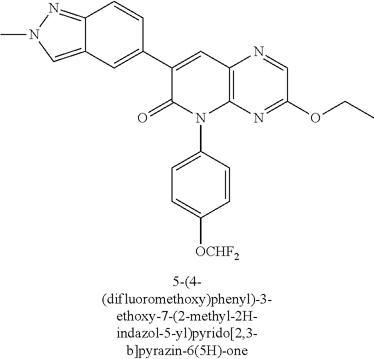 [SMILES: CCOc1cnc2cc(-c3ccc4nn(C)cc4c3)c(=O)n(-c3ccc(OC(F)F)cc3)c2n1] | LC-MS (ESI): m/z 464 [M + H]+. 1H NMR (400 MHz, DMSO- d6) δ: 8.41 (s, 1H), 8.23 (s, 1H), 8.19 (d, J = 7.6 Hz, 2H), 7.62 (s, 2H), 7.49 (d, J = 8.0 Hz, 2H), 7.37 (s, 2H), 7.35 (t, J_{HF} = 69.2 Hz, 1H), 4.19 (s, 3H), 4.04 (q, J = 7.2 Hz, 2H), 1.18-1.13 (m, 3H). |
| 342 | 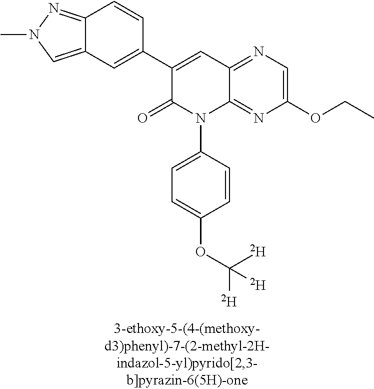 [SMILES: [2H]C([2H])([2H])Oc1ccc(-n2c(=O)c(-c3ccc4nn(C)cc4c3)cc3ncc(OCC)nc32)cc1] | LC-MS: m/z 431 (M + H)+. 1H NMR (400 MHz, DMSO- d6) δ: 8.40 (s, 1H), 8.20 (s, 1H), 8.17 (s, 2H), 7.65-7.55 (m, 2H), 7.30 (d, J = 8.7 Hz, 2H), 7.09 (d, J = 8.7 Hz, 2H), 4.18 (s, 3H), 4.04 (q, J = 7.0 Hz, 2H), 1.18 (t, J = 7.0 Hz, 3H). |

General Procedure VII

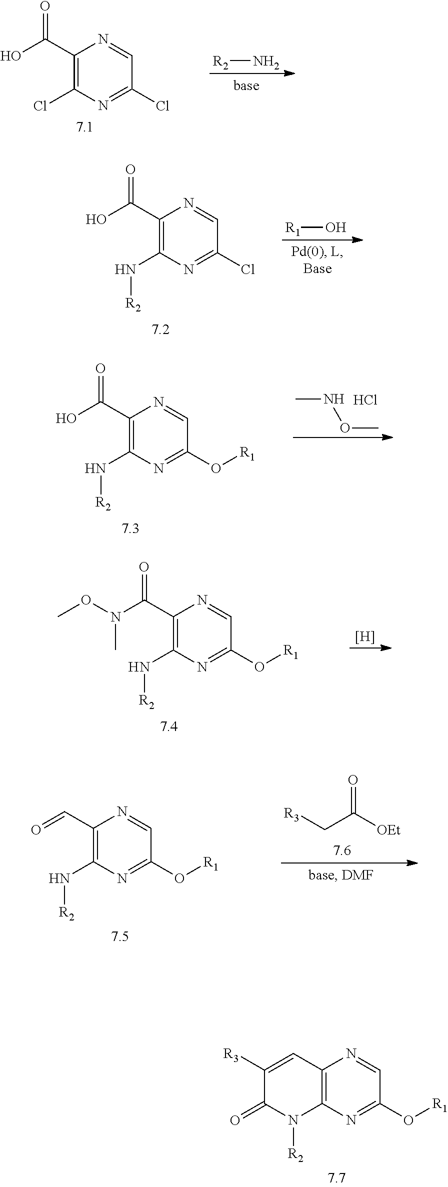

Compounds of structure 7.7 were obtained through the scheme depicted as General Procedure VII, which is a modification of General Procedure VI. Beginning with functionalized pyrazine 7.1, the desired R2 group was introduced through a nucleophilic aromatic substitution to generate amino-pyrazine 7.2. The desired R1 group was then introduced through a palladium mediated Buchwald-Hartwig coupling to generate substituted pyrazine 7.3. Weinreb amide 7.4 was then formed, which allowed for reduction to aldehyde 7.5. This substituted heterocycle 7.5 was then reacted with ester 7.6 and base to concurrently introduce the desired R3 and close the bicyclic ring system to afford compounds of structure 7.7.

### Preparation of Example 343 via General Procedure VII

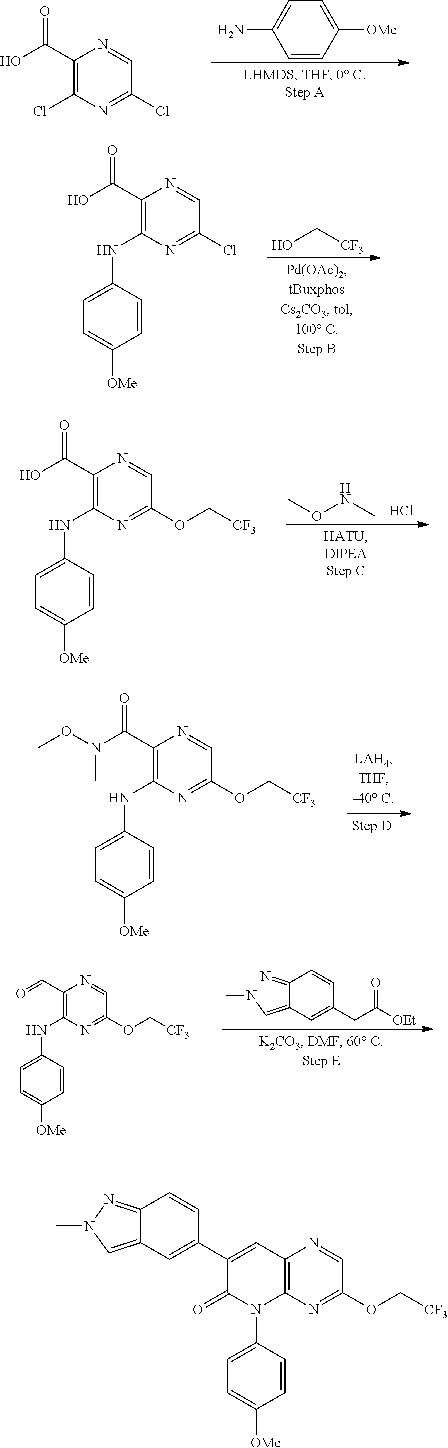

### Step A: 5-chloro-3-((4-methoxyphenyl)amino)pyrazine-2-carboxylic acid

To a solution of 4-methoxyaniline (510 mg, 4.2 mmol, 2.0 eq.) in dry THE (5 ml) was added LiHMDS (1 M in THF) (6.7 mL, 6.7 mmol, 3.2 eq.) dropwise at −78° C. under N2 atmosphere, the reaction mixture was stirred at this temperature for 30 min, then a solution of 3,5-dichloropyrazine-2-carboxylic acid (400 mg, 2.1 mmol, 1.0 eq.) in dry THE (3 mL) was added dropwise. The resulting mixture was stirred at −78° C. for additional 30 min, then allowed warm to room temperature and stirred for 16 hrs. After the completion, the reaction was quenched with H₂O (10 mL), the aqueous layer was adjusted pH=2 with HCl (2N, aq.), extracted with EtOAc (20 mL×3), the combined organic layers were washed with brine (20 mL), dried over Na₂SO₄ and concentrated under reduced pressure. The residue was purified by flash column chromatography on silica gel to give 5-chloro-3-((4-methoxyphenyl)amino)pyrazine-2-carboxylic acid (500 mg, 86%) as a yellow solid. LC-MS (ESI): m/z 280 [M+H]⁺.

### Step B: 3-((4-methoxyphenyl)amino)-5-(2,2,2-trifluoroethoxy)pyrazine-2-carboxylic acid

A mixture of 5-chloro-3-((4-methoxyphenyl)amino)pyrazine-2-carboxylic acid (500 mg, 1.79 mmol, 1.0 eq.), Pd(OAc)₂ (40 mg, 0.18 mmol, 0.1 eq.), t-BuXPhos (152 mg, 0.36 mmol, 0.2 eq.) and Cs₂CO₃ (1.75 g, 5.38 mmol, 3.0 eq.) in toluene (5 mL) and 2,2,2-trifluoroethan-1-ol (0.5 mL) was stirred at 100° C. under N2 atmosphere overnight. The reaction mixture was concentrated under reduced pressure, the residue was purified by flash column chromatography on silica gel to give 8-(4-chlorophenyl)-N-ethyl-7-[(4-methoxyphenyl)methoxy]pyrido[3,4-b]pyrazin-2-amine (500 mg, 82%) as a yellow solid. LC-MS (ESI): m/z 344 [M+H]⁺.

### Step C: N-methoxy-3-((4-methoxyphenyl)amino)-N-methyl-5-(2,2,2-trifluoroethoxy)pyrazine-2-carboxamide

A mixture of 3-((4-methoxyphenyl)amino)-5-(2,2,2-trifluoroethoxy)pyrazine-2-carboxylic acid (500 mg, 1.46 mmol, 1.0 eq.), methoxy(methyl)amine hydrochloride (284 mg, 2.91 mmol, 2.0 eq.), DIPEA (753 mg, 5.84 mmol, 4.0 eq.) and HATU (2.2 g, 5.84 mmol, 4.0 eq.) in DCM (10 mL) was stirred at room temperature overnight. Then the reaction mixture was diluted with H₂O (20 mL), extracted with DCM (20 mL×3). The combined organic layers were washed with brine (20 mL), dried over Na₂SO₄ and concentrated under reduced pressure, the residue was purified by flash column chromatography on silica gel to give N-methoxy-3-((4-methoxyphenyl)amino)-N-methyl-5-(2,2,2-trifluoroethoxy)pyrazine-2-carboxamide (400 mg, 71%) as a yellow solid. LC-MS (ESI): m/z 387 [M+H]⁺.

### Step D: 3-((4-methoxyphenyl)amino)-5-(2,2,2-trifluoroethoxy)pyrazine-2-carbaldehyde

To a solution of N-methoxy-3-((4-methoxyphenyl)amino)-N-methyl-5-(2,2,2-trifluoroethoxy)pyrazine-2-carboxamide (400 mg, 0.1 mmol, 1.0 eq.) in dry THE (7 ml) was added LiAlH₄ (12 mg, 0.3 mmol, 3 eq.) at −78° C. under N2 atmosphere, the reaction mixture was stirred at −78° C. for 30 min. After the completion, the reaction was quenched with 20 mL of NH₄Cl (sat. aq.) at −78° C., the resulting mixture was allowed warm to room temperature and extracted with EtOAc (15 mL×3). The combined organic layers were washed with brine (20 mL), dried over Na₂SO₄ and concentrated under reduced pressure, the residue was purified by flash column chromatography on silica gel to give 3-((4-methoxyphenyl)amino)-5-(2,2,2-trifluoroethoxy)pyrazine-2-carbaldehyde (300 mg, 88%) as a yellow solid. LC-MS (ESI): m/z 328 [M+H]⁺.

### Step E: 5-(4-methoxyphenyl)-7-(2-methyl-2H-indazol-5-yl)-3-(2,2,2-trifluoroethoxy)pyrido[2,3-b]pyrazin-6(5H)-one

To a solution of 3-((4-methoxyphenyl)amino)-5-(2,2,2-trifluoroethoxy)pyrazine-2-carbaldehyde (100 mg, 0.31 mmol, 1.0 eq.) and ethyl 2-(2-methyl-2H-indazol-5-yl)acetate (133 mg, 0.62 mmol, 2.0 eq.) in DMF (5 mL) was added K₂CO₃ (211 mg, 1.55 mmol, 5.0 eq.), the reaction mixture was stirred at 60° C. for 14 hrs. The reaction mixture was poured into water (20 mL) and extracted with EtOAc (15 mL×3). The combined organic layers were dried over Na₂SO₄, concentrated under reduced pressure, the residue was purified by flash column on silica gel to give 5-(4-methoxyphenyl)-7-(2-methyl-2H-indazol-5-yl)-3-(2,2,2-trifluoroethoxy)pyrido[2,3-b]pyrazin-6(5H)-one (Example 343).

¹H NMR (400 MHz, DMSO-d₆) δ (ppm): 8.42 (s, 1H), 8.40 (s, 1H), 8.21 (s, 1H), 8.18 (s, 1H), 7.67-7.51 (i, 2H), 7.32 (d, J=8.9 Hz, 2H), 7.10 (d, J=8.9 Hz, 2H), 4.70 (q, J_{HF}=8.9 Hz, 2H), 4.18 (s, 3H), 3.83 (s, 3H). LC-MS (ES): m/z 482 [M+H]⁺.

The procedure set forth above for General Procedure VII was used to synthesize the following compounds by using appropriate starting materials.

| Example | Structure | Characterization |
|---|---|---|
| 344 | 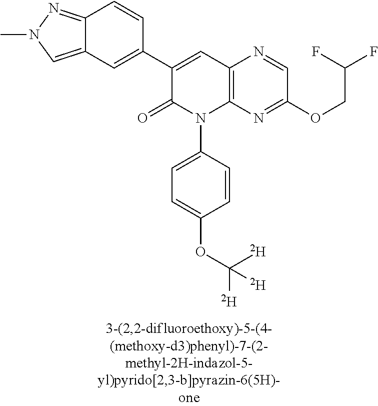 [SMILES: [2H]C([2H])([2H])Oc1ccc(-n2c(=O)c(-c3ccc4nn(C)cc4c3)cc3ncc(OCC(F)F)nc32)cc1] | LC-MS: m/z 467 (M + H)+. 1H NMR (400 MHz, DMSO- d6) δ: 8.42 (s, 1H), 8.34 (s, 1H), 8.20 (d, J = 9.1 Hz, 2H), 7.66- 7.58 (m, 2H), 7.33 (d, J = 8.8 Hz, 2H), 7.11 (d, J = 8.9 Hz, 2H), 6.21 (tt, JHF = 54.7 Hz, J = 3.4 Hz, 1H), 4.28 (td, JHF = 14.7 Hz, J = 3.4 Hz, 2H), 4.19 (s, 3H). |
| 345 | 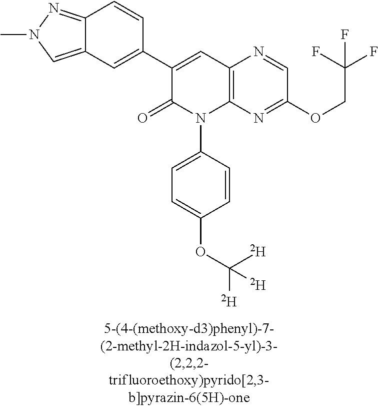 [SMILES: [2H]C([2H])([2H])Oc1ccc(-n2c(=O)c(-c3ccc4nn(C)cc4c3)cc3ncc(OCC(F)(F)F)nc32)cc1] | LC-MS: m/z 485 (M + H)+. 1H NMR (400 MHz, DMSO- d6) δ: 8.42 (d, J = 7.8 Hz, 2H), 8.22 (s, 1H), 8.19 (s, 1H), 7.62 (d, J = 1.6 Hz, 2H), 7.37-7.28 (m, 2H), 7.15-7.05 (m, 2H), 4.70 (q, JHF = 8.9 Hz, 2H), 4.19 (s, 3H). |

### Synthesis of 8-(4-bromophenyl)-6-(4-methoxyphenyl)-2-(2,2,2-trifluoroethylamino)pyrido[2,3-d]pyrimidin-7(8H)-one (Example 232)

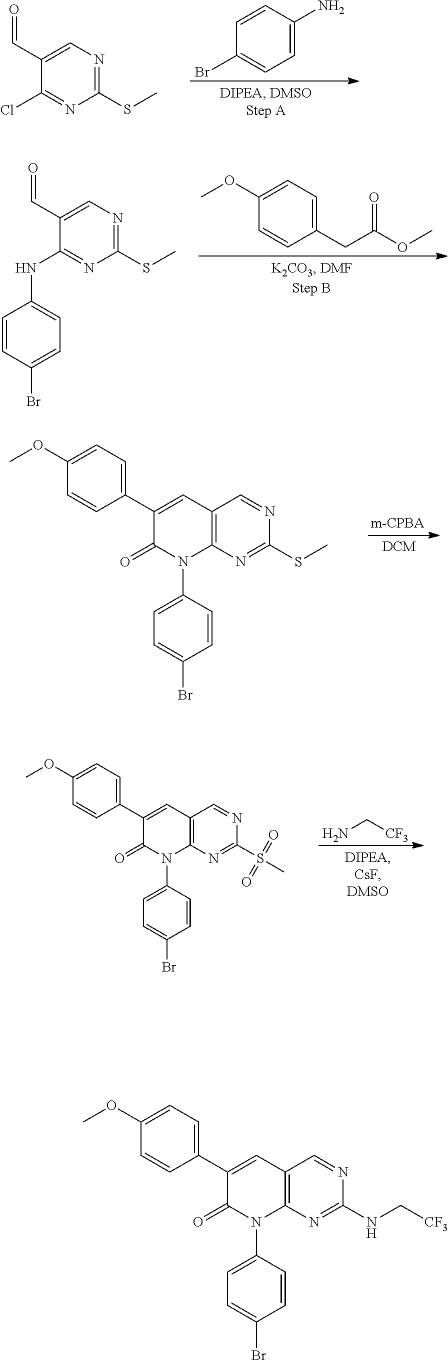

### Step A: 4-(4-bromophenylamino)-2-(methylthio)pyrimidine-5-carbaldehyde

A mixture of 4-chloro-2-(methylthio)pyrimidine-5-carbaldehyde (500 mg, 2.65 mmol, 1.0 eq.), 4-bromoaniline (502 mg, 2.92 mmol, 1.1 eq.) and DIPEA (685 mg, 5.3 mmol, 2.0 eq.) in DMSO (10 mL) was stirred at 100° C. for 1 hr. The reaction mixture was diluted with H₂O (30 mL) and extracted with EtOAc (20 mL×3). The combined organic layers were washed with brine (20 mL), dried over Na₂SO₄ and concentrated under reduced pressure. The residue was purified by flash column chromatography on silica gel to give 4-(4-bromophenylamino)-2-(methylthio)pyrimidine-5-carbaldehyde (700 mg, 81% yield) as a white solid. LC-MS (ESI): m/z 324, 326 [M+H]⁺.

### Step B: 8-(4-bromophenyl)-6-(4-methoxyphenyl)-2-(methylthio)pyrido[2,3-d]pyrimidin-7(8H)-one

A mixture of 4-(4-bromophenylamino)-2-(methylthio)pyrimidine-5-carbaldehyde (486 mg, 1.5 mmol, 1.0 eq.), methyl 2-(4-methoxyphenyl)acetate (301 mg, 1.7 mmol, 1.13 eq.) and K₂CO₃ (415 mg, 3.0 mmol, 2.0 eq.) in DMF (5 mL) was stirred at 110° C. for 2 hrs. Then the reaction mixture was diluted with H₂O (20 mL) and extracted with EtOAc (20 mL×3), The combined organic layers were washed with brine (20 mL), dried over Na₂SO₄ and concentrated under reduced pressure. The residue was purified by flash column chromatography on silica gel to give 8-(4-bromophenyl)-6-(4-methoxyphenyl)-2-(methylthio)pyrido[2,3-d]pyrimidin-7(8H)-one (510 mg, 75% yield) as a yellow solid. LC-MS (ESI): m/z 454, 456 [M+H]⁺.

8-(4-bromophenyl)-6-(4-methoxyphenyl)-2-(2,2,2-trifluoroethylamino)pyrido[2,3-d]pyrimidin-7(8H)-one (Example 232) was synthesized from 8-(4-bromophenyl)-6-(4-methoxyphenyl)-2-(methylthio)pyrido[2,3-d]pyrimidin-7(8H)-one and 2,2,2-trifluoroethanamine via General Procedure I (Route A, Steps C and D).

¹H NMR (400 MHz, DMSO-d₆) (the ratio of two tautomers: 1:1) δ: 8.79 (br, 1H), 8.31-7.96 (m, 2H), 7.73 (d, J=8.1 Hz, 2H), 7.65 (d, J=8.8 Hz, 2H), 7.32 (br, 2H), 6.99 (d, J=8.8 Hz, 2H), 4.17-3.67 (m, 5H). LC-MS (ESI): m/z 505, 507 [M+H]⁺.

### Synthesis of 8-(4-(1H-pyrazol-5-yl)phenyl)-6-(4-methoxyphenyl)-2-(2,2,2-trifluoroethylamino)pyrido[2,3-d]pyrimidin-7(8H)-one (Example 233)

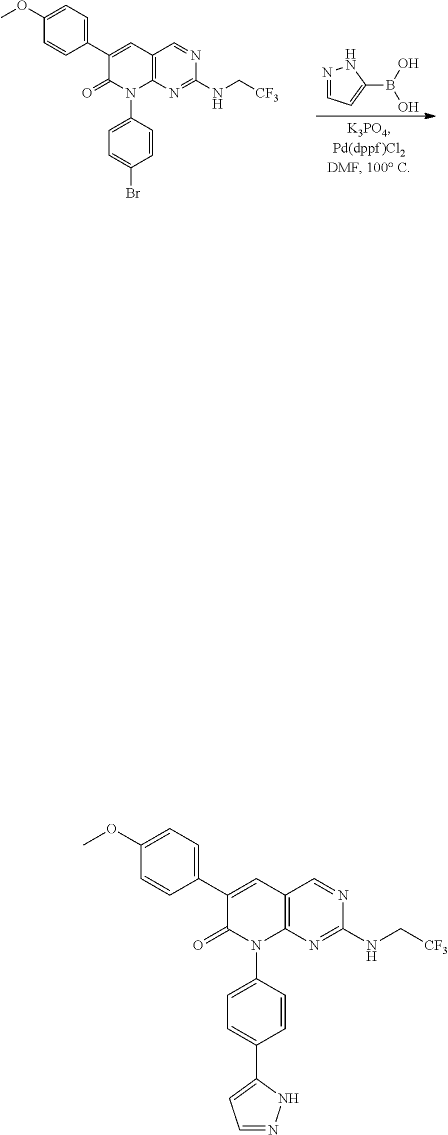 [SMILES: COc1ccc(-c2cc3cnc(NCC(F)(F)F)nc3n(-c3ccc(Br)cc3)c2=O)cc1 ; OB(O)c1ccn[nH]1 ; COc1ccc(-c2cc3cnc(NCC(F)(F)F)nc3n(-c3ccc(-c4ccn[nH]4)cc3)c2=O)cc1]

A mixture of 8-(4-bromophenyl)-6-(4-methoxyphenyl)-2-(2,2,2-trifluoroethylamino)pyrido[2,3-d]pyrimidin-7(8H)-one (Example 232, 50 mg, 0.1 mmol, 1.0 eq.), 1H-pyrazol-5-ylboronic acid (33 mg, 0.3 mmol, 3.0 eq.), Pd(dppf)₂Cl₂ (8 mg, 0.01 mmol, 0.1 eq.) and K₃PO₄ (85 mg, 0.4 mmol, 4.0 eq.) in DMF (3 mL) was stirred at 100° C. overnight under N2 atmosphere. Then the reaction mixture was diluted with H₂O (10 mL) and extracted with EtOAc (10 mL×3). The combined organic layers were washed with brine (10 mL), dried over Na₂SO₄ and concentrated under reduced pressure. The residue was purified by RP-prep-HPLC to give 8-(4-(1H-pyrazol-5-yl)phenyl)-6-(4-methoxyphenyl)-2-(2,2,2-trifluoroethylamino)pyrido[2,3-d]pyrimidin-7(8H)-one (Example 233).

¹H NMR (400 MHz, DMSO-d₆) (the ratio of two tautomers: 7:3) δ: 13.42 (s, 0.3H), 12.97 (s, 0.7H), 8.80 (d, J=7.6 Hz, 1H), 8.36-8.24 (m, 0.6H), 8.05 (s, 1H), 8.03-7.80 (m, 3.4H), 7.66 (d, J=8.8 Hz, 2H), 7.46-7.28 (m, 2H), 6.99 (d, J=8.4 Hz, 2H), 6.80 (s, 1H), 4.21-4.04 (m, 0.6H), 3.79 (s, 3H), 3.80-3.66 (m, 1.4H). LC-MS (ESI): m/z 493 [M+H]⁺.

### Synthesis of 8-(4-(1H-pyrazol-5-yl)phenyl)-6-(1-methyl-6-oxo-1,6-dihydropyridin-3-yl)-2-(2,2,2-trifluoroethylamino)pyrido[2,3-d]pyrimidin-7(8H)-one (Example 234)

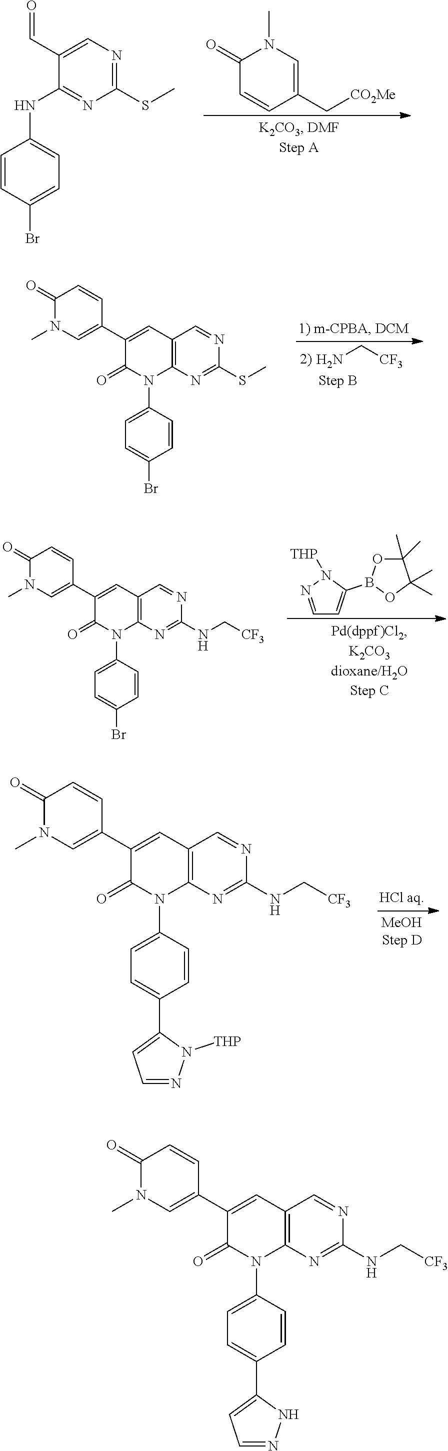

### Step A: 8-(4-bromophenyl)-6-(1-methyl-6-oxo-1,6-dihydropyridin-3-yl)-2-(methylthio)pyrido[2,3-d]pyrimidin-7(8H)-one

8-(4-bromophenyl)-6-(1-methyl-6-oxo-1,6-dihydropyridin-3-yl)-2-(methylthio)pyrido[2,3-d]pyrimidin-7(8H)-one was synthesized from methyl 2-(1-methyl-6-oxo-1,6-dihydropyridin-3-yl)acetate (Ref: WO 2005/5378, 2005, A2 Page 37-38) and 4-(4-bromophenylamino)-2-(methylthio)pyrimidine-5-carbaldehyde via the method for synthesis of Example 232 (step B). LC-MS (ESI): m/z 455, 457 [M+H]⁺.

### Step B: 8-(4-bromophenyl)-6-(1-methyl-6-oxo-1,6-dihydropyridin-3-yl)-2-(2,2,2-trifluoroethylamino)pyrido[2,3-d]pyrimidin-7(8H)-one

8-(4-bromophenyl)-6-(1-methyl-6-oxo-1,6-dihydropyridin-3-yl)-2-(2,2,2-trifluoroethylamino)pyrido[2,3-d]pyrimidin-7(8H)-one was synthesized from 8-(4-bromophenyl)-6-(1-methyl-6-oxo-1,6-dihydropyridin-3-yl)-2-(methylthio)pyrido[2,3-d]pyrimidin-7(8H)-one and 2,2,2-trifluoroethanamine via general procedure I (Route A, Steps C and D). LC-MS (ESI): m/z 506, 508 [M+H]⁺.

### Step C: 6-(1-methyl-6-oxo-1,6-dihydropyridin-3-yl)-8-(4-(1-(tetrahydro-2H-pyran-2-yl)-1H-pyrazol-5-yl)phenyl)-2-(2,2,2-trifluoroethylamino)pyrido[2,3-d]pyrimidin-7(8H)-one

A mixture of 8-(4-bromophenyl)-6-(1-methyl-6-oxo-1,6-dihydropyridin-3-yl)-2-(2,2,2-trifluoroethylamino)pyrido[2,3-d]pyrimidin-7(8H)-one (50 mg, 0.1 mmol, 1.0 eq.), 1-(tetrahydro-2H-pyran-2-yl)-5-(4,4,5,5-tetramethyl-1,3,2-dioxaborolan-2-yl)-1H-pyrazole (55 mg, 0.2 mmol, 2.0 eq.), Pd(dppf)Cl₂ (8 mg, 0.01 mmol, 0.01 eq.) and K₂CO₃ (41 mg, 0.3 mmol, 3.0 eq.) in dioxane-H₂O (10 mL, 9:1, v:v) was stirred at 100° C. under N2 atmosphere overnight. Then the reaction mixture was diluted with H₂O (10 mL) and extracted with EtOAc (10 mL×3). The combined organic layers were washed with brine (20 mL), dried over Na₂SO₄ and concentrated under reduced pressure. The residue was purified by flash column chromatography on silica gel to give 6-(1-methyl-6-oxo-1,6-dihydropyridin-3-yl)-8-(4-(1-(tetrahydro-2H-pyran-2-yl)-1H-pyrazol-5-yl)phenyl)-2-(2,2,2-trifluoroethylamino)pyrido[2,3-d]pyrimidin-7(8H)-one (30 mg, 52% yield). LC-MS (ESI): m/z 578 [M+H]⁺.

### Step D: 8-(4-(1H-pyrazol-5-yl)phenyl)-6-(1-methyl-6-oxo-1,6-dihydropyridin-3-yl)-2-(2,2,2-trifluoroethylamino)pyrido[2,3-d]pyrimidin-7(8H)-one

To a solution of 6-(1-methyl-6-oxo-1,6-dihydropyridin-3-yl)-8-(4-(1-(tetrahydro-2H-pyran-2-yl)-1H-pyrazol-5-yl)phenyl)-2-(2,2,2-trifluoroethylamino)pyrido[2,3-d]pyrimidin-7(8H)-one (30 mg, 0.05 mmol, 1.0 eq.) in methanol (5 mL) was added 1 M HCl (1 mL). The resulting mixture was stirred at room temperature for 3 hrs. Then the mixture was concentrated under reduced pressure and the residue was purified by RP-prep-HPLC to give 8-(4-(1H-pyrazol-5-yl)phenyl)-6-(1-methyl-6-oxo-1,6-dihydropyridin-3-yl)-2-((2,2,2-trifluoroethyl)amino)pyrido[2,3-d]pyrimidin-7(8H)-one (Example 234).

¹H NMR (400 MHz, DMSO-d₆) (the ratio of two tautomers: 1:1) δ: 12.96 (s, 1H), 8.77 (s, 1H), 8.30-7.78 (m, 7H), 7.33 (s, 2H), 6.79 (s, 1H), 6.46 (d, J=9.5 Hz, 1H), 4.12 (s, 2H), 3.48 (s, 3H). LC-MS (ESI): m/z 494 [M+H]⁺.

### Synthesis of 8-(4-methoxyphenyl)-6-(6-oxo-1,6-dihydropyridin-3-yl)-2-(2,2,2-trifluoroethylamino)pyrido[2,3-d]pyrimidin-7(8H)-one (Example 235

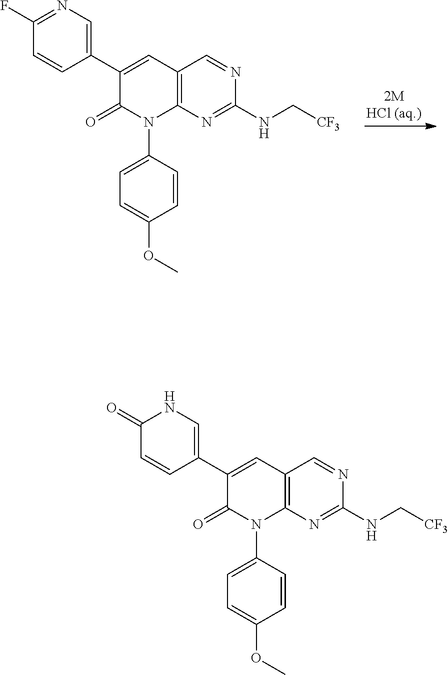 [SMILES: COc1ccc(-n2c(=O)c(-c3ccc(F)nc3)cc3cnc(NCC(F)(F)F)nc32)cc1 ; COc1ccc(-n2c(=O)c(-c3ccc(=O)[nH]c3)cc3cnc(NCC(F)(F)F)nc32)cc1]

A solution of 6-(6-fluoropyridin-3-yl)-8-(4-methoxyphenyl)-2-(2,2,2-trifluoroethylamino)pyrido[2,3-d]pyrimidin-7(8H)-one (synthesized from 6-bromo-8-(4-methoxyphenyl)-2-(2,2,2-trifluoroethylamino)pyrido[2,3-d]pyrimidin-7(8H)-one and 6-fluoropyridin-3-ylboronic acid via General Procedure III (Step F)) (46 mg, 0.1 mmol) in 2 M HCl (aq., 3 mL) was stirred at 100° C. for 2 hrs. Then the solution was concentrated under reduced pressure and the residue was purified by RP-prep-HPLC to give 8-(4-methoxyphenyl)-6-(6-oxo-1,6-dihydropyridin-3-yl)-2-(2,2,2-trifluoroethylamino)pyrido[2,3-d]pyrimidin-7(8H)-one (Example 235).

¹H NMR (400 MHz, DMSO-d₆) (the ratio of two tautomers: 1:1) δ: 11.74 (br, 1H), 8.74 (s, 1H), 8.30-8.26 (m, 0.5H), 8.10 (s, 1H), 8.09-7.97 (m, 0.5H), 7.96 (s, 1H), 7.83 (dd, J=9.6 Hz, 2.0 Hz, 1H), 7.21-7.19 (m, 2H), 7.06 (d, J=8.4 Hz, 2H), 6.41 (d, J=9.6 Hz, 1H), 4.17-4.06 (m, 1H), 3.82 (s, 3H), 3.78-3.70 (m, 1H). LC-MS (ESI): m/z 444 [M+H]⁺.

### Synthesis of 1-(4-chlorophenyl)-3-(2-methyl-2H-indazol-5-yl)-7-propyl-1,8-naphthyridin-2(1H)-one (Example 236)

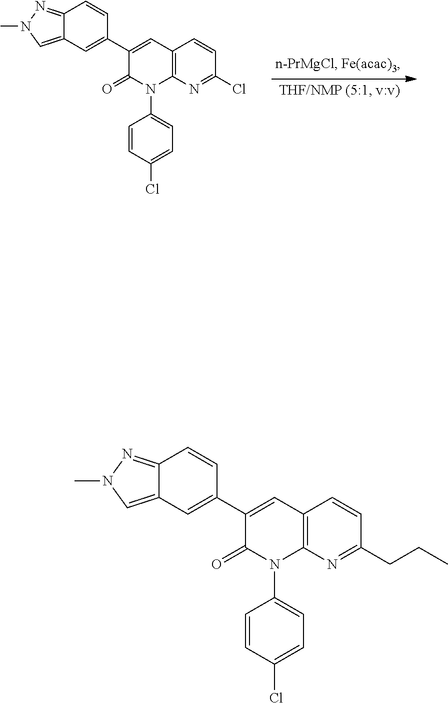 [SMILES: Cn1cc2cc(-c3cc4ccc(Cl)nc4n(-c4ccc(Cl)cc4)c3=O)ccc2n1 ; CCCc1ccc2cc(-c3ccc4nn(C)cc4c3)c(=O)n(-c3ccc(Cl)cc3)c2n1]

To a mixture of 7-chloro-1-(4-chlorophenyl)-3-(2-methyl-2H-indazol-5-yl)-1,8-naphthyridin-2(1H)-one (109 mg, 0.26 mmol, 1.0 eq., Intermediate from Example 119 synthesis (General Procedure I, Step I)) and Fe(acac)₃ (93 mg, 0.26 mmol, 1.0 eq.) in THE (5 mL) and NMP (1 mL) was added n-propylmagnesium chloride (1 M in diethyl ether, 4 mL, 4 mmol, 15.4 eq.) drop-wise at 0° C. under N2 atmosphere. The mixture was stirred at room temperature overnight and quenched carefully with ice water (10 mL). The resulting mixture was extracted with EtOAc (10 mL×3). The combined organic layers were dried over Na₂SO₄, concentrated under reduced pressure, and the crude residue was purified by RP-prep-HPLC to give 1-(4-chlorophenyl)-3-(2-methyl-2H-indazol-5-yl)-7-propyl-1,8-naphthyridin-2(1H)-one (Example 236).

¹H NMR (400 MHz, DMSO-d₆) δ: 8.41 (s, 1H), 8.25 (s, 1H), 8.16 (d, J=7.9 Hz, 1H), 8.13 (s, 1H), 7.65-7.54 (m, 4H), 7.41-7.33 (m, 2H), 7.20 (d, J=7.9 Hz, 1H), 4.18 (s, 3H), 2.60 (t, J=7.4 Hz, 2H), 1.63-1.43 (m, 2H), 0.80 (t, J=7.4 Hz, 3H). LC-MS (ESI): m/z 429 [M+H]⁺.

### Synthesis of 5-(4-(difluoromethoxy)phenyl)-7-(4-methoxyphenyl)-3-((2,2,2-trifluoroethyl)amino)pyrido[2,3-b]pyrazin-6(5H)-one (Example 237)

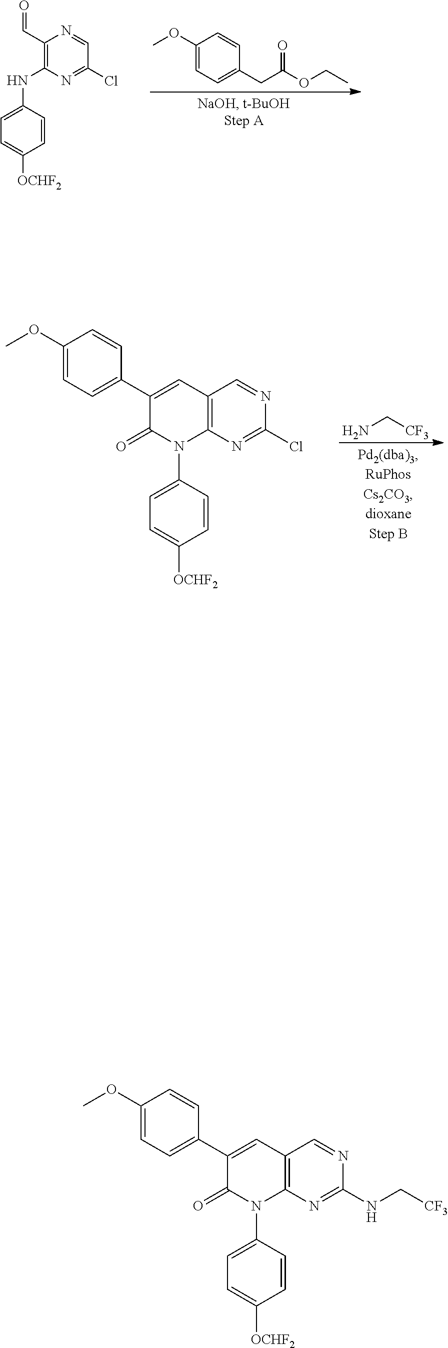 [SMILES: O=Cc1ncc(Cl)nc1Nc1ccc(OC(F)F)cc1 ; CCOC(=O)Cc1ccc(OC)cc1 ; COc1ccc(-c2cc3cnc(Cl)nc3n(-c3ccc(OC(F)F)cc3)c2=O)cc1 ; NCC(F)(F)F ; COc1ccc(-c2cc3cnc(NCC(F)(F)F)nc3n(-c3ccc(OC(F)F)cc3)c2=O)cc1]

### Step A: 3-chloro-5-(4-(difluoromethoxy)phenyl)-7-(4-methoxyphenyl)pyrido[2,3-b]pyrazin-6(5H)-one

To a solution of 5-chloro-3-((4-(difluoromethoxy)phenyl)amino)pyrazine-2-carbaldehyde (120 mg, 0.4 mmol, 1.0 eq.) (synthesized from 3,5-dichloropyrazine-2-carboxylic acid and 4-(difluoromethoxy)aniline via General Procedure VI (Steps A-C)) and ethyl 2-(4-methoxyphenyl)acetate (86 mg, 0.44 mmol, 1.1 eq.) in t-BuOH (5 mL) was added NaOH (32 mg, 0.8 mmol, 2.0 eq.), the reaction mixture was stirred at 80° C. for 3 hrs. the reaction mixture was diluted with H₂O (20 ml) and extracted with EtOAc (20 mL×3), the combined organic layers were washed with brine (10 ml), dried over Na₂SO₄ and concentrated under reduced pressure, the residue was purified by flash column chromatography on silica gel to afford 3-chloro-5-(4-(difluoromethoxy)phenyl)-7-(4-methoxyphenyl)pyrido[2,3-b]pyrazin-6(5H)-one (30 mg, 17% yield) as a brown solid. LC-MS (ESI): m/z 430 [M+H]⁺.

### Step B: 5-(4-(difluoromethoxy)phenyl)-7-(4-methoxyphenyl)-3-((2,2,2-trifluoroethyl)amino)pyrido[2,3-b]pyrazin-6(5H)-one

A mixture of 3-chloro-5-(4-(difluoromethoxy)phenyl)-7-(4-methoxyphenyl)pyrido[2,3-b]pyrazin-6(5H)-one (30 mg, 70 μmol, 1.0 eq.), Pd₂(dba)₃ (6 mg, 7 μmol, 0.1 eq.), RuPhos (7 mg, 14 μmol, 0.2 eq.) and Cs₂CO₃ (68 mg, 210 μmol, 3 eq.) in dioxane (2 mL) was degassed with N2 for 10 min, then 2,2,2-trifluoroethan-1-amine (69 mg, 700 μmol, 10 eq.) was added and the mixture was sealed in a tube. The resulting mixture was stirred at 100° C. for 15 hrs before being diluted with H₂O (20 ml) and extracted with EtOAc (20 mL×3). The combined organic layers were washed with brine (10 ml), dried over Na₂SO₄ and concentrated under reduced pressure. The resulting residue was purified by Prep-TLC to afford 5-(4-(difluoromethoxy)phenyl)-7-(4-methoxyphenyl)-3-((2,2,2-trifluoroethyl)amino)pyrido[2,3-b]pyrazin-6(5H)-one (Example 237).

¹H NMR (400 MHz, DMSO-d₆) δ: 8.39 (s, 1H), 8.01 (d, J=7.6 Hz, 2H), 7.71 (d, J=8.4 Hz, 2H), 7.42-7.30 (m, 4H), 7.32 (t, J_{HF}=73.8 Hz, 1H), 6.98 (d, J=8.8 Hz, 2H), 3.89-3.80 (m, 2H), 3.79 (s, 3H). LC-MS (ESI): m/z 493 [M+H]⁺.

### Synthesis of 4-(4-bromophenyl)-2-(2-methyl-2H-indazol-5-yl)-6-(2,2,2-trifluoroethoxy)pyrido[2,3-b]pyrazin-3(4H)-one (Example 346)

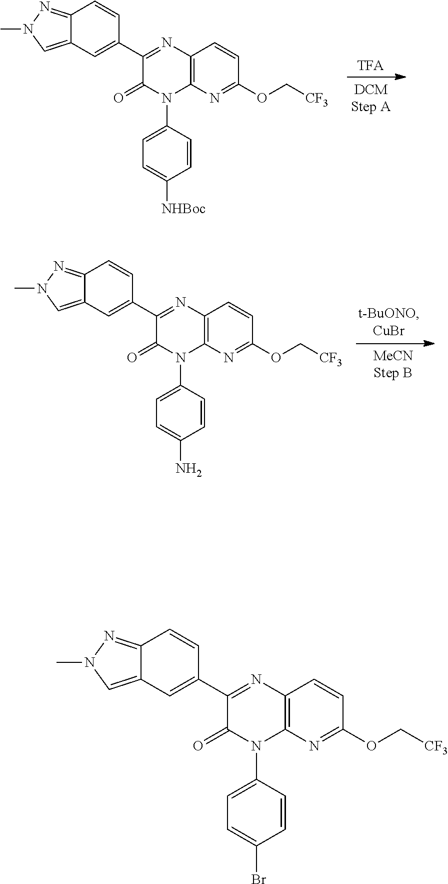 [SMILES: Cn1cc2cc(-c3nc4ccc(OCC(F)(F)F)nc4n(-c4ccc(NC(=O)OC(C)(C)C)cc4)c3=O)ccc2n1 ; Cn1cc2cc(-c3nc4ccc(OCC(F)(F)F)nc4n(-c4ccc(N)cc4)c3=O)ccc2n1 ; Cn1cc2cc(-c3nc4ccc(OCC(F)(F)F)nc4n(-c4ccc(Br)cc4)c3=O)ccc2n1]

### Step A: 4-(4-aminophenyl)-2-(2-methyl-2H-indazol-5-yl)-6-(2,2,2-trifluoroethoxy)pyrido[2,3-b]pyrazin-3(4H)-one

To a solution of tert-butyl (4-(2-(2-methyl-2H-indazol-5-yl)-3-oxo-6-(2,2,2-trifluoroethoxy)pyrido[2,3-b]pyrazin-4(3H)-yl)phenyl)carbamate (synthesized from 2,6-dichloro-3-nitropyridine and tert-butyl (4-aminophenyl)carbamate via General Procedure V (Step A-F)) (100 mg, 0.18 mmol, 1.0 eq.) in DCM (3 mL) was added TFA (1 mL), the reaction mixture was stirred at room temperature for 3 hours. TLC showed the reaction was complete. The reaction mixture was concentrated under reduced pressure, the residue was purified by flash column chromatography on silica gel to give 4-(4-aminophenyl)-2-(2-methyl-2H-indazol-5-yl)-6-(2,2,2-trifluoroethoxy)pyrido[2,3-b]pyrazin-3(4H)-one (70 mg, 85% yield) as a pale yellow oil. LC-MS (ESI): m/z 467 [M+H]⁺.

### Step B: 4-(4-bromophenyl)-2-(2-methyl-2H-indazol-5-yl)-6-(2,2,2-trifluoroethoxy)pyrido[2,3-b]pyrazin-3(4H)-one

To a suspension of 4-(4-aminophenyl)-2-(2-methyl-2H-indazol-5-yl)-6-(2,2,2-trifluoroethoxy)pyrido[2,3-b]pyrazin-3(4H)-one (50 mg, 0.11 mmol, 1.0 eq.) and CuBr (31 mg, 0.22 mmol, 2.0 eq.) in MeCN (8 mL) was added a solution of tert-butyl nitrite (22 mg, 0.22 mmol, 2.0 eq.) in MeCN (1 mL) dropwise at 0° C., the resulting mixture was stirred at room temperature for 2 hours. The reaction was quenched by adding Na₂SO₃ (sat. aq.) (10 mL), extracted with EtOAc (15 mL×3). The combined organic layers were concentrated under reduced pressure, the residue was purified by RP-prep-HPLC to give 4-(4-bromophenyl)-2-(2-methyl-2H-indazol-5-yl)-6-(2,2,2-trifluoroethoxy)pyrido[2,3-b]pyrazin-3(4H)-one (Example 346).

¹H NMR (400 MHz, DMSO-d₆) δ (ppm): 8.95 (s, 1H), 8.53 (s, 1H), 8.35 (d, J=8.5 Hz, 1H), 8.19-8.11 (m, 1H), 7.80 (d, J=8.5 Hz, 2H), 7.67 (d, J=9.2 Hz, 1H), 7.45 (d, J=8.6 Hz, 2H), 7.02 (d, J=8.5 Hz, 1H), 4.66 (q, J_{HF}=9.0 Hz, 2H), 4.19 (s, 3H). LC-MS (ESI): m/z 530.0[M+H]⁺.

### Synthesis of 2-(2-(2-aminoethyl)-2H-indazol-5-yl)-4-(4-methoxyphenyl)-6-(2,2,2-trifluoroethoxy)pyrido[2,3-b]pyrazin-3(4H)-one (Example 347)

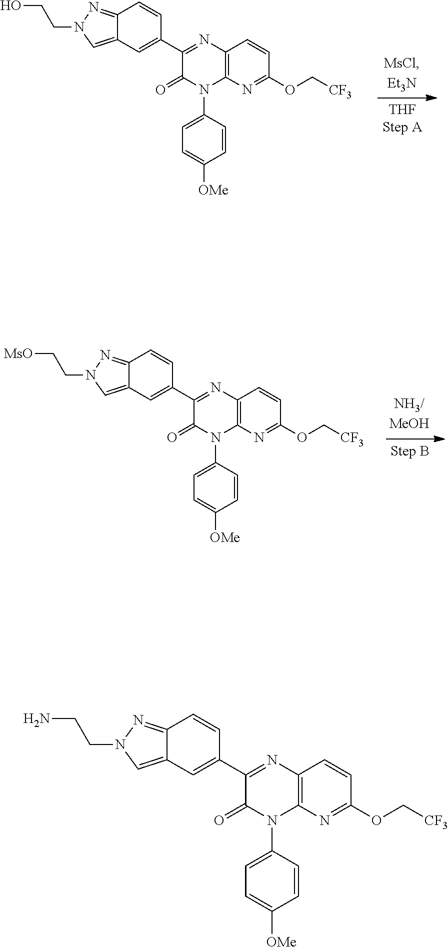 [SMILES: COc1ccc(-n2c(=O)c(-c3ccc4nn(CCO)cc4c3)nc3ccc(OCC(F)(F)F)nc32)cc1 ; COc1ccc(-n2c(=O)c(-c3ccc4nn(CCOS(C)(=O)=O)cc4c3)nc3ccc(OCC(F)(F)F)nc32)cc1 ; COc1ccc(-n2c(=O)c(-c3ccc4nn(CCN)cc4c3)nc3ccc(OCC(F)(F)F)nc32)cc1]

### Step A: 2-(5-(4-(4-methoxyphenyl)-3-oxo-6-(2,2,2-trifluoroethoxy)-3,4-dihydropyrido[2,3-b]pyrazin-2-yl)-2H-indazol-2-yl)ethyl methanesulfonate

To a solution of 2-(2-(2-hydroxyethyl)-2H-indazol-5-yl)-4-(4-methoxyphenyl)-6-(2,2,2-trifluoroethoxy)pyrido[2,3-b]pyrazin-3(4H)-one (Example 335) (70 mg, 0.137 mmol, 1.0 eq.) in DCM (5 mL) was added Et₃N (42 mg, 0.411 mmol, 3.0 eq.) and MsCl (47 mg, 0.411 mmol, 3.0 eq.), The resulting mixture was stirred at room temperature for 2 h. After completion of the reaction, the reaction mixture was diluted with H₂O (10 mL), extracted with EtOAc (5 mL×3). The combined organic layers were washed with brine (10 mL) and dried over Na₂SO₄, concentrated under reduced pressure, the residue was purified by flash chromatography to afford 2-(5-(4-(4-methoxyphenyl)-3-oxo-6-(2,2,2-trifluoroethoxy)-3,4-dihydropyrido[2,3-b]pyrazin-2-yl)-2H-indazol-2-yl)ethyl methanesulfonate (100 mg, crude) as a brown solid, which used in the next step directly without further purification. LC-MS (ESI): m/z 590.0 [M+H]⁺.

### Step B: 2-(2-(2-aminoethyl)-2H-indazol-5-yl)-4-(4-methoxyphenyl)-6-(2,2,2-trifluoroethoxy)pyrido[2,3-b]pyrazin-3(4H)-one (Example 347)

To a solution of 2-(5-(4-(4-methoxyphenyl)-3-oxo-6-(2,2,2-trifluoroethoxy)-3,4-dihydropyrido[2,3-b]pyrazin-2-yl)-2H-indazol-2-yl)ethyl methanesulfonate (100 mg, 0.170 mmol, 1.0 eq.) was added NH₃/MeOH (7N in MeOH) (1.0 mL), the resulting mixture was sealed in a pressure resistant tube, and stirred at 80° C. for 14 hrs. After completion of the reaction, the mixture was concentrated under reduced pressure. The residue was purified by RP-prep-HPLC to give 2-(2-(2-aminoethyl)-2H-indazol-5-yl)-4-(4-methoxyphenyl)-6-(2,2,2-trifluoroethoxy)pyrido[2,3-b]pyrazin-3(4H)-one (Example 347).

¹H NMR (400 MHz, DMSO-d₆) δ (ppm): 8.96 (s, 1H), 8.56 (s, 1H), 8.33 (d, J=8.5 Hz, 1H), 8.27 (s, 1H), 8.15 (dd, J=9.2 Hz, 1.6 Hz, 1H), 7.69 (d, J=9.2 Hz, 1H), 7.39-7.33 (m, 2H), 7.15-7.10 (m, 2H), 7.00 (d, J=8.5 Hz, 1H), 4.66 (q, J_{HF}=9.0 Hz, 2H), 4.48 (t, J=5.8 Hz, 2H), 3.84 (s, 3H), 3.17 (t, J=6.1 Hz, 2H). LC-MS (ESI): m/z 511 [M+H]⁺.

### Synthesis of 2-(2-methyl-2H-indazol-5-yl)-4-(6-(methylamino)pyridin-3-yl)-6-(2,2,2-trifluoroethoxy)pyrido[2,3-b]pyrazin-3(4H)-one (Example 348)

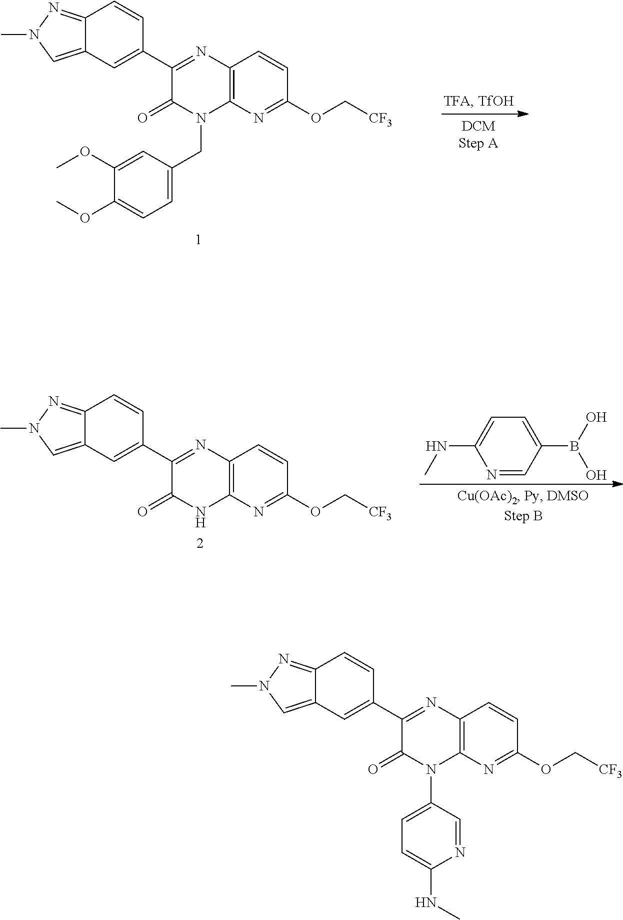 [SMILES: COc1ccc(Cn2c(=O)c(-c3ccc4nn(C)cc4c3)nc3ccc(OCC(F)(F)F)nc32)cc1OC ; Cn1cc2cc(-c3nc4ccc(OCC(F)(F)F)nc4[nH]c3=O)ccc2n1 ; CNc1ccc(B(O)O)cn1 ; CNc1ccc(-n2c(=O)c(-c3ccc4nn(C)cc4c3)nc3ccc(OCC(F)(F)F)nc32)cn1]

### Step A: 2-(2-methyl-2H-indazol-5-yl)-6-(2,2,2-trifluoroethoxy)pyrido[2,3-b]pyrazin-3(4H)-one

To a solution of 4-[(3,4-dimethoxyphenyl)methyl]-2-(2-methyl-2H-indazol-6-yl)-6-(2,2,2-trifluoroethoxy)-3H,4H-pyrido[2,3-b]pyrazin-3-one (422 mg, 0.80 mmol, 1.0 eq.) (synthesized from 2,6-dichloro-3-nitropyridine and (3,4-dimethoxyphenyl)methanamine via General Procedure V (Step A-F)) in DCM (2 mL) was added TFA (2 mL) and trifluoromethanesulfonic acid (0.2 mL) at 0° C., the reaction mixture was stirred at room temperature overnight. The reaction mixture was poured into ice-water (10 mL) slowly, then a precipitation was formed, the solid was collected and purified by flash column chromatography on silica gel to give 2-(2-methyl-2H-indazol-6-yl)-6-(2,2,2-trifluoroethoxy)-3H,4H-pyrido[2,3-b]pyrazin-3-one (190 mg, 63%) as a white solid. LC-MS (ESI): m/z 376 [M+H]⁺.

### Step B: 2-(2-methyl-2H-indazol-5-yl)-4-(6-(methylamino)pyridin-3-yl)-6-(2,2,2-trifluoroethoxy)pyrido[2,3-b]pyrazin-3(4H)-one

To a solution of 2-(2-methyl-2H-indazol-5-yl)-6-(2,2,2-trifluoroethoxy)pyrido[2,3-b]pyrazin-3(4H)-one (70 mg, 0.18 mmol, 1.0 eq.) in DMSO (4 mL) was added (6-(methylamino)pyridin-3-yl)boronic acid (56 mg, 0.37 mmol, 2.0 eq.), Cu(OAc)₂ (33 mg, 0.18 mmol, 1.0 eq.) and pyridine (30 μL, 0.37 mmol, 2.0 eq.), the reaction mixture was stirred at 80° C. under air atmosphere for 15 hrs. The reaction mixture was poured into ice water (10 mL), extracted with EtOAc (15 mL×3), the combined organic layers were washed with brine (30 mL), dried over Na₂SO₄ and concentrated under reduced pressure, the residue was purified by RP-prep-HPLC to give 2-(2-methyl-2H-indazol-5-yl)-4-(6-(methylamino)pyridin-3-yl)-6-(2,2,2-trifluoroethoxy)pyrido[2,3-b]pyrazin-3(4H)-one (Example 348).

¹H NMR (400 MHz, DMSO-d₆) δ (ppm): 8.94 (t, J=1.2 Hz, 1H), 8.53 (s, 1H), 8.33 (d, J=8.4 Hz, 1H), 8.14 (dd, J=9.2 Hz, 1.6 Hz, 1H), 8.02 (d, J=2.4 Hz, 1H), 7.67 (d, J=9.2 Hz, 1H), 7.46 (dd, J=8.8 Hz, 2.4 Hz, 1H), 7.01 (d, J=8.4 Hz, 1H), 6.80 (d, J=4.8 Hz, 1H), 6.61 (d, J=8.8 Hz, 1H), 4.75 (q, J_{HF}=9.2 Hz, 2H), 4.19 (s, 3H), 2.84 (d, J=4.8 Hz, 3H). LC-MS (ESI): m/z 482 [M+H]⁺.

### Synthesis of 4-(6-(dimethylamino)pyridin-3-yl)-2-(2-methyl-2H-indazol-5-yl)-6-(2,2,2-trifluoroethoxy)pyrido[2,3-b]pyrazin-3(4H)-one (Example 349)

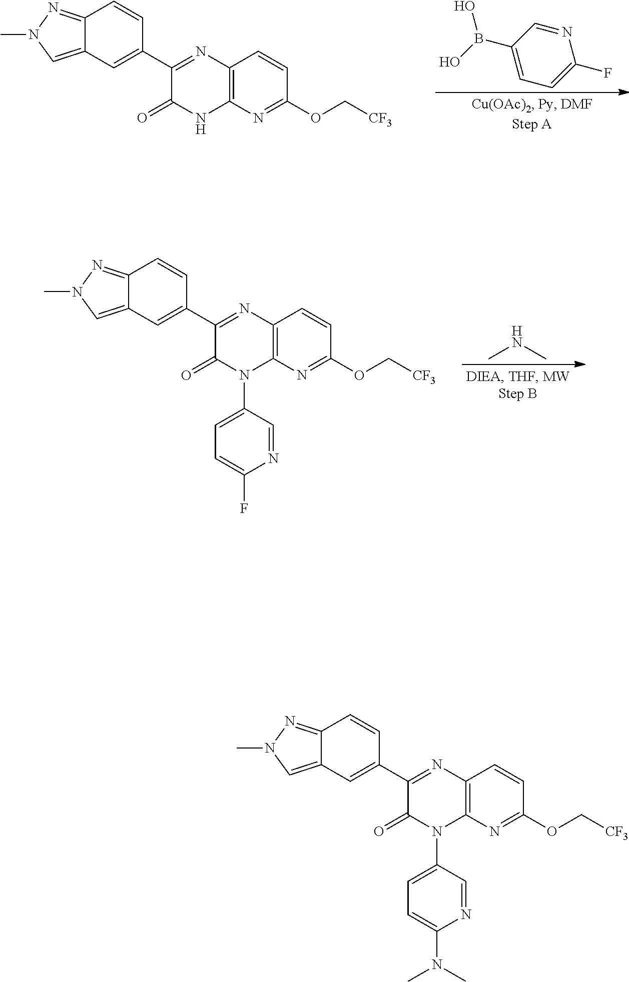

### Step A: 4-(6-fluoropyridin-3-yl)-2-(2-methyl-2H-indazol-5-yl)-6-(2,2,2-trifluoroethoxy)pyrido[2,3-b]pyrazin-3(4H)-one

To a solution of 2-(2-methyl-2H-indazol-5-yl)-6-(2,2,2-trifluoroethoxy)pyrido[2,3-b]pyrazin-3(4H)-one (200 mg, 0.53 mmol, 1.0 eq.) in dry DMF (5 mL) was added (6-fluoropyridin-3-yl)boronic acid (150.18 mg, 1.06 mmol, 2.0 eq.) Cu(OAc)₂ (193.58 mg, 1.06 mmol, 2.0 eq.) and pyridine (172 μL, 2.12 mmol, 4.0 eq.), the resulting mixture was stirred at 80° C. under 02 atmosphere (1 atm) for 14 hrs. After completion of the reaction, the reaction was quenched by adding H₂O (5 mL), extracted with EtOAc (10 mL×3). The combined organic layers were washed with brine (10 mL) and dried over Na₂SO₄, concentrated under reduced pressure, the residue was purified by flash chromatography to afford 4-(6-fluoropyridin-3-yl)-2-(2-methyl-2H-indazol-5-yl)-6-(2,2,2-trifluoroethoxy)pyrido[2,3-b]pyrazin-3(4H)-one (150 mg, 47%) as a yellow solid. LC-MS (ESI): m/z 471 [M+H]⁺.

### Step B: 4-(6-(dimethylamino)pyridin-3-yl)-2-(2-methyl-2H-indazol-5-yl)-6-(2,2,2-trifluoroethoxy)pyrido[2,3-b]pyrazin-3(4H)-one (Example 349)

4-(6-fluoropyridin-3-yl)-2-(2-methyl-2H-indazol-5-yl)-6-(2,2,2-trifluoroethoxy)pyrido[2,3-b]pyrazin-3(4H)-one (50 mg, 0.106 mmol, 1.0 eq.), dimethylamine solution (2M in THF) (0.22 mL, 0.44 mmol, 4.0 eq.) and DIEA (76 μL, 0.44 mmol, 4.0 eq.) added in a pressure resistant tube, the resulting mixture was irradiated under microwave (150 W) at 80° C. for 2 hrs. After the completion, the reaction mixture was concentrated under reduced pressure, the residue was purified by RP-prep-HPLC to afford 4-[6-(dimethylamino)pyridin-3-yl]-2-(2-methyl-2H-indazol-5-yl)-6-(2,2,2-trifluoroethoxy)-3H,4H-pyrido[2,3-b]pyrazin-3-one (Example 349).

¹H NMR (400 MHz, DMSO-d₆) δ (ppm): 8.95 (d, J=1.2 Hz, 1H), 8.53 (s, 1H), 8.35 (d, J=8.4 Hz, 1H), 8.29 (d, J=5.2 Hz, 1H), 8.14 (dd, J=9.2 Hz, 1.6 Hz, 1H), 7.68 (d, J=9.2 Hz, 1H), 7.02 (d, J=8.4 Hz, 1H), 6.80 (d, J=1.6 Hz, 1H), 6.67 (dd, J=5.2 Hz, 1.6 Hz, 1H), 4.72 (q, J_{HF}=8.8 Hz, 2H), 4.19 (s, 3H), 3.05 (s, 6H). LC-MS (ESI): m/z 496 [M+H]⁺

### Synthesis of 4-(6-aminopyridin-3-yl)-2-(2-methyl-2H-indazol-5-yl)-6-(2,2,2-trifluoroethoxy)pyrido[2,3-b]pyrazin-3(4H)-one (Example 350)

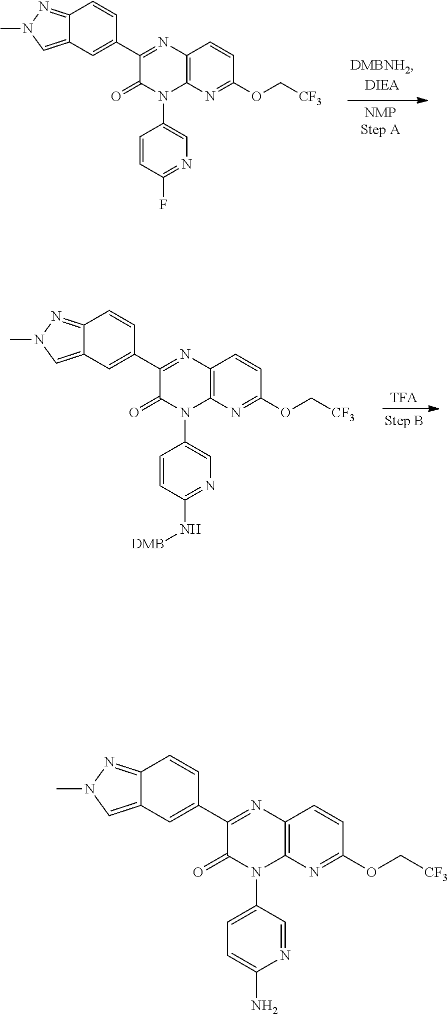

### Step A: 4-(6-((3,4-dimethylbenzyl)amino)pyridin-3-yl)-2-(2-methyl-2H-indazol-5-yl)-6-(2,2,2-trifluoroethoxy)pyrido[2,3-b]pyrazin-3(4H)-one

To a solution of 4-(6-fluoropyridin-3-yl)-2-(2-methyl-2H-indazol-5-yl)-6-(2,2,2-trifluoroethoxy)pyrido[2,3-b]pyrazin-3(4H)-one (100 mg, 0.213 mmol, 1.0 eq) and (2,4-dimethoxyphenyl)methanamine (48 μL, 0.319 mmol, 1.5 eq.) in dry NMP (5 mL) was added DIEA (105 μL, 0.638 mmol, 3.0 eq.), the resulting mixture was irradiated under microwave (150 W) at 130° C. for 1 hrs. After completion of the reaction, the reaction mixture was concentrated under reduced pressure, the residue was purified by flash column chromatography on silica gel to afford 4-(6-((3,4-dimethylbenzyl)amino)pyridin-3-yl)-2-(2-methyl-2H-indazol-5-yl)-6-(2,2,2-trifluoroethoxy)pyrido[2,3-b]pyrazin-3(4H)-one (50 mg, 35%) as a yellow solid. LC-MS (ESI): m/z 586 [M+H]⁺.

### Step B: 4-(6-aminopyridin-3-yl)-2-(2-methyl-2H-indazol-5-yl)-6-(2,2,2-trifluoroethoxy)pyrido[2,3-b]pyrazin-3(4H)-one (Example 350)

To a solution of 4-(6-((3,4-dimethylbenzyl)amino)pyridin-3-yl)-2-(2-methyl-2H-indazol-5-yl)-6-(2,2,2-difluoroethoxy)pyrido[2,3-b]pyrazin-3(4H)-one (50 mg, 0.081 mmol, 1.0 eq.) in dry DCM (3 mL) was added TFA (3 mL), the resulting mixture was stirred at 40° C. for 40 h, after the completion, the reaction mixture was concentrated under reduced pressure, the residue was purified by RP-prep-HPLC to afford 4-(6-aminopyridin-3-yl)-2-(2-methyl-2H-indazol-5-yl)-6-(2,2,2-trifluoroethoxy)pyrido[2,3-b]pyrazin-3(4H)-one (Example 350).

¹H NMR (400 MHz, DMSO-d₆) δ (ppm): 8.93 (d, J=2.4 Hz, 1H), 8.54 (s, 1H), 8.38 (d, J=8.8 Hz, 1H), 8.01-8.16 (m, 4H), 7.67 (d, J=1.6 Hz, 1H), 7.12 (s, 1H), 7.06 (d, J=8.4 Hz, 1H), 6.97-7.00 (m, 1H), 4.83 (q, J_{HF}=9.2 Hz, 2H), 4.20 (s, 3H). LC-MS (ESI): m/z 468 [M+H]⁺.

### Synthesis of 2-(2-amino-1-methyl-1H-benzo[d]imidazol-6-yl)-4-(4-methoxyphenyl)-6-(2,2,2-trifluoroethoxy)pyrido[2,3-b]pyrazin-3(4H)-one (Example 351)

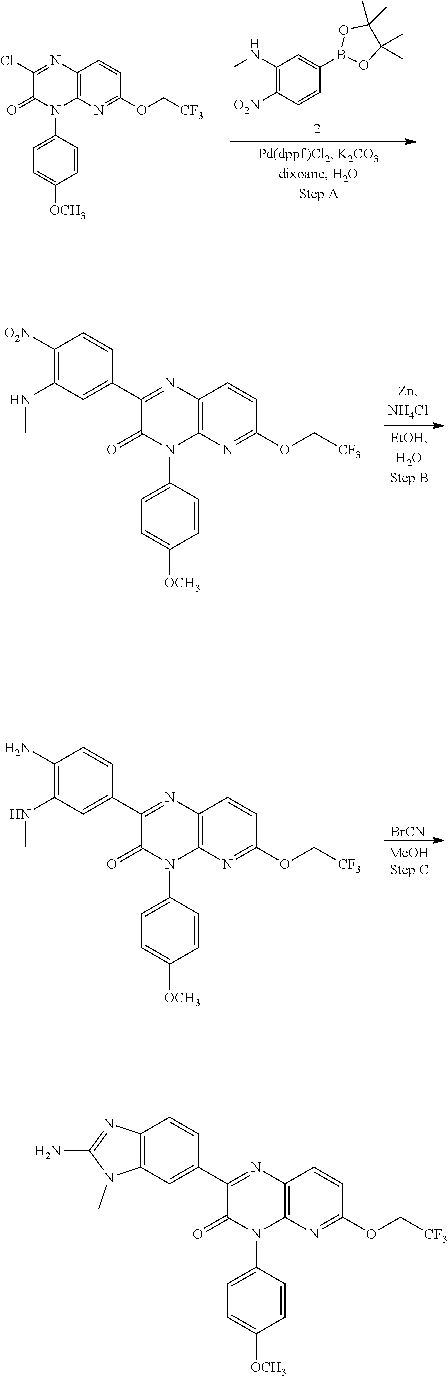

### Step A: 4-(4-methoxyphenyl)-2-(3-(methylamino)-4-nitrophenyl)-6-(2,2,2-trifluoroethoxy)pyrido[2,3-b]pyrazin-3(4H)-one

To a solution of 2-chloro-4-(4-methoxyphenyl)-6-(2,2,2-trifluoroethoxy)pyrido[2,3-b]pyrazin-3(4H)-one (100 mg, 0.25 mmol, 1.0 eq.), N-methyl-2-nitro-5-(4,4,5,5-tetramethyl-1,3,2-dioxaborolan-2-yl)aniline (86 mg, 0.31 mmol, 1.2 eq.) in dioxane (4 mL) and H₂O (0.4 mL) was added K₂CO₃ (89 mg, 0.64 mmol, 2.5 eq.) and Pd(dppf)Cl₂ (18 mg, 0.026 mmol, 0.1 eq), the reaction mixture was stirred at 100° C. under N2 atmosphere for 2 hrs. After completion, the reaction mixture was concentrated under reduced pressure, the residue was purified by column chromatography on silica gel to give 4-(4-methoxyphenyl)-2-(3-(methylamino)-4-nitrophenyl)-6-(2,2,2-trifluoroethoxy)pyrido[2,3-b]pyrazin-3(4H)-one (105 mg, 77%) as a brown solid. LC-MS (ESI): m/z 502 [M+H]⁺

### Step B: 2-(4-amino-3-(methylamino)phenyl)-4-(4-methoxyphenyl)-6-(2,2,2-trifluoroethoxy) pyrido[2,3-b]pyrazin-3(4H)-one

To a solution of 4-(4-methoxyphenyl)-2-(3-(methylamino)-4-nitrophenyl)-6-(2,2,2-trifluoroethoxy)pyrido[2,3-b]pyrazin-3(4H)-one (105 mg, 0.20 mmol, 1.0 eq.) in EtOH (6 mL) and H₂O (6 mL) was added NH₄Cl (106 mg, 2.0 mmol, 10.0 eq.) and Fe powder (115 mg, 2.0 mmol, 10.0 eq.), the reaction mixture was stirred at 100° C. for 2 hrs. After completion, the reaction mixture was filtered through a short pad of Celite®, the filtrate was diluted with H₂O (10 mL), extracted with EtOAc (15 mL×3), the combined organic layers were dried over Na₂SO₄, concentrated under reduced pressure, the residue was purified by column chromatography on silica gel to afford 2-(4-amino-3-(methylamino)phenyl)-4-(4-methoxyphenyl)-6-(2,2,2-trifluoroethoxy)pyrido[2,3-b]pyrazin-3(4H)-one (60 mg, 64%) as a brown solid. LC-MS (ESI): m/z 472 [M+H]⁺.

### Step C: 2-(2-amino-1-methyl-1H-benzo[d]imidazol-6-yl)-4-(4-methoxyphenyl)-6-(2,2,2-trifluoroethoxy)pyrido[2,3-b]pyrazin-3(4H)-one

To a solution of 2-(4-amino-3-(methylamino)phenyl)-4-(4-methoxyphenyl)-6-(2,2,2-trifluoroethoxy)pyrido[2,3-b]pyrazin-3(4H)-one (60 mg, 0.13 mmol, 1.0 eq.) in MeOH (4 mL) was added BrCN (20 mg, 0.19 mmol, 1.5 eq.), the reaction mixture was stirred at 80° C. for 2 hrs. The reaction mixture was concentrated under reduced pressure, the residue was purified by RP-prep-HPLC to give 2-(2-amino-1-methyl-1H-benzo[d]imidazol-6-yl)-4-(4-methoxyphenyl)-6-(2,2,2-trifluoroethoxy)pyrido[2,3-b]pyrazin-3(4H)-one (Example 351).

¹H NMR (400 MHz, DMSO-d₆) δ (ppm): 8.29 (d, J=8.4 Hz, 1H), 8.21 (d, J=1.6 Hz, 1H), 8.09 (dd, J=8.4 Hz, 1.7 Hz, 1H), 7.45-7.28 (m, 2H), 7.19 (d, J=8.4 Hz, 1H), 7.15-7.08 (m, 2H), 6.97 (d, J=8.4 Hz, 1H), 6.71 (s, 2H), 4.65 (q, J_{HF}=8.4 Hz, 2H), 3.85 (s, 3H), 3.53 (s, 3H). LC-MS (ESI): m/z 497.00 [M+H]⁺

### Synthesis of 8-amino-4-(4-methoxyphenyl)-2-(2-methyl-2H-indazol-5-yl)-6-(2,2,2-trifluoroethoxy)pyrido[2,3-b]pyrazin-3(4H)-one (Example 352)

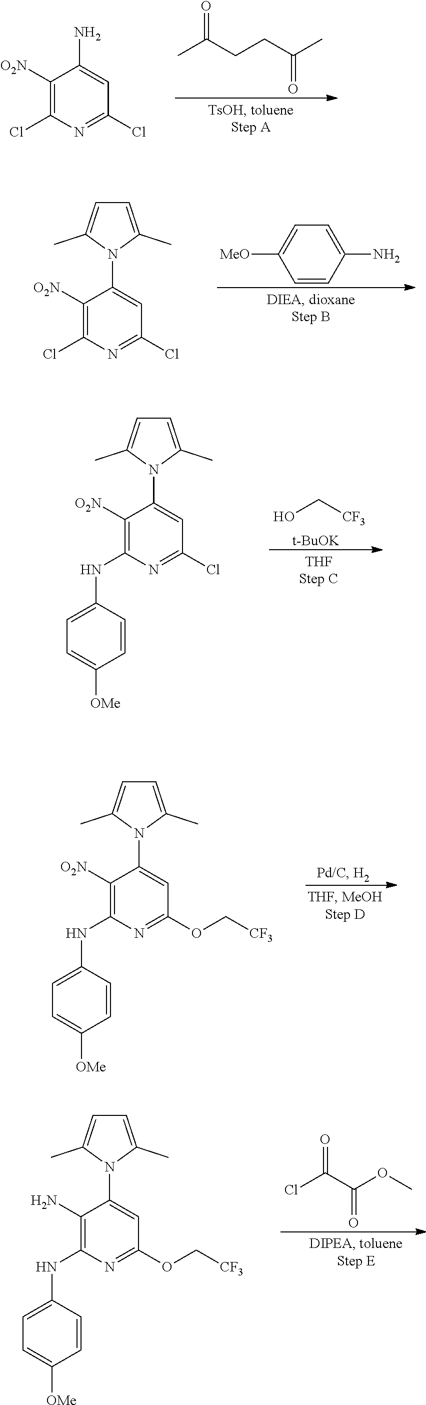

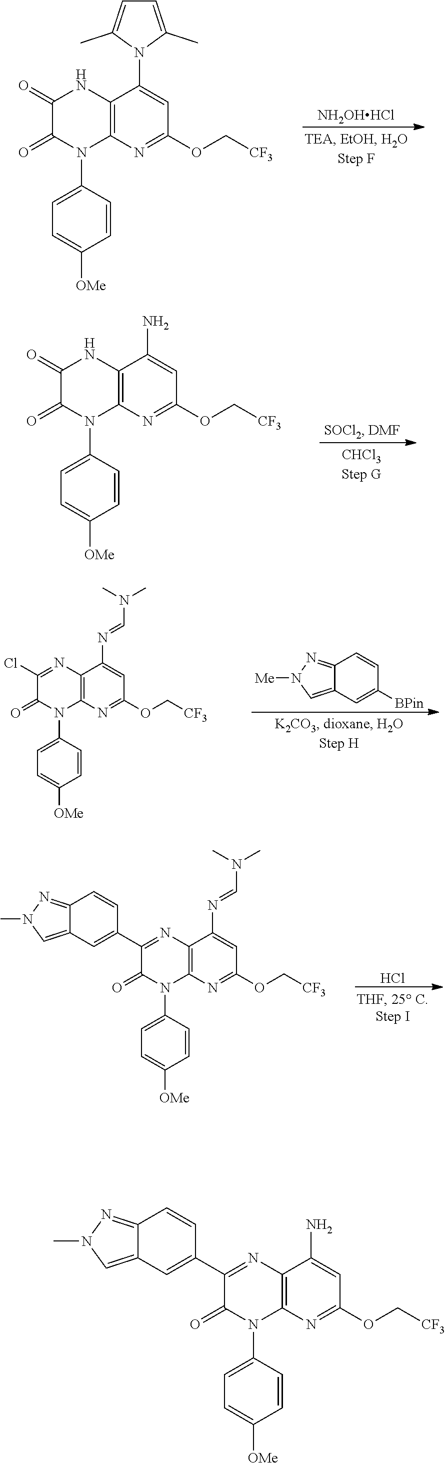

### Step A: 2,6-dichloro-4-(2,5-dimethyl-1H-pyrrol-1-yl)-3-nitropyridine

To a solution of 2,6-dichloro-3-nitropyridin-4-amine (3.4 g, 16.34 mmol, 1.0 eq.) in toluene (30 mL) was added TsOH (0.56 g, 3.26 mmol, 0.2 eq.) and hexane-2,5-dione (2.3 mL, 19.61 mmol, 1.2 eq.), the reaction mixture was stirred at 110° C. for 12 hrs. The reaction was complete as indicated by LCMS. The reaction mixture was quenched with water (100 mL) and extracted with EtOAc (150 mL×3), the combined organic layers were dried over anhydrous Na₂SO₄, filtered and concentrated under reduced pressure, the residue was purified by flash column chromatography on silica gel to afford 2,6-dichloro-4-(2,5-dimethyl-1H-pyrrol-1-yl)-3-nitropyridine (840 mg, 2.93 mmol, 18%) as a colorless oil. LC-MS (ESI): m/z 286 [M+H]⁺.

### Step B: 6-chloro-4-(2,5-dimethyl-1H-pyrrol-1-yl)-N-(4-methoxyphenyl)-3-nitropyridin-2-amine

To a solution of 2,6-dichloro-4-(2,5-dimethyl-1H-pyrrol-1-yl)-3-nitropyridine (840 mg, 2.93 mmol, 1.0 eq.) in 1,4-dioxane (8 mL) was added DIEA (1.55 mL, 8.80 mmol, 3.0 eq.) and 4-methoxyaniline (360 mg, 2.93 mmol, 1.0 eq.), the reaction mixture was stirred at 80° C. for 12 hrs. After completion, the reaction mixture was quenched with water (25 mL) and extracted with EtOAc (35 mL×3), the combined organic layers were dried over anhydrous Na₂SO₄, filtered and concentrated under reduced pressure, the residue was purified by flash column chromatography on silica gel to afford 6-chloro-4-(2,5-dimethyl-1H-pyrrol-1-yl)-N-(4-methoxyphenyl)-3-nitropyridin-2-amine (600 mg, 55%) as a colorless oil. LC-MS (ESI): m/z 373 [M+H]⁺.

### Step C: 4-(2,5-dimethyl-1H-pyrrol-1-yl)-N-(4-methoxyphenyl)-3-nitro-6-(2,2,2-trifluoroethoxy)pyridin-2-amine

To a solution of 6-chloro-4-(2,5-dimethyl-1H-pyrrol-1-yl)-N-(4-methoxyphenyl)-3-nitropyridin-2-amine (190 mg, 0.50 mmol, 1.0 eq.) in THE (5 mL) was added t-BuOK (171 mg, 1.52 mmol, 3.0 eq.) and 2,2,2-trifluoroethan-1-ol (0.04 mL, 0.61 mmol, 1.2 eq.) at 25° C. Then the mixture was stirred at 60° C. for 4 hrs. The reaction mixture was quenched poured into ice water (25 mL) and extracted with EtOAc (25 mL×3). The combined organic layers were dried over anhydrous Na₂SO₄, filtered and concentrated under reduced pressure, the residue was purified by flash column chromatography on silica gel to give 4-(2,5-dimethyl-1H-pyrrol-1-yl)-N-(4-methoxyphenyl)-3-nitro-6-(2,2,2-trifluoroethoxy)pyridin-2-amine (180 mg, 81%) as a colorless oil. LC-MS (ESI): m/z 437[M+H]⁺.

### Step D: 4-(2,5-dimethyl-1H-pyrrol-1-yl)-N2-(4-methoxyphenyl)-6-(2,2,2-trifluoroethoxy)pyridine-2,3-diamine

To a solution of 4-(2,5-dimethyl-1H-pyrrol-1-yl)-N-(4-methoxyphenyl)-3-nitro-6-(2,2,2-trifluoroethoxy)pyridin-2-amine (110 mg, 0.25 mmol, 1.0 eq.) in MeOH (4.5 mL) and THE (1.5 mL) was added 10% Pd/C (40 mg), the reaction mixture was degassed with H₂ and stirred at 25° C. under H₂ atmosphere (1 atm) for 3 hrs. LCMS showed the reaction was completed. The reaction mixture was filtered through a short pad of Celite®, the filtrate was concentrated under reduced pressure, the residue was purified by flash column chromatography on silica gel to afford 4-(2,5-dimethyl-1H-pyrrol-1-yl)-N2-(4-methoxyphenyl)-6-(2,2,2-trifluoroethoxy)pyridine-2,3-diamine (100 mg, 98%) was obtained as a brown oil. LC-MS (ESI): m/z 407 [M+H]⁺.

### Step E: 8-(2,5-dimethyl-1H-pyrrol-1-yl)-4-(4-methoxyphenyl)-6-(2,2,2-trifluoroethoxy)-1H,2H,3H,4H-pyrido[2,3-b]pyrazine-2,3-dione

To a solution of 4-(2,5-dimethyl-1H-pyrrol-1-yl)-N2-(4-methoxyphenyl)-6-(2,2,2-trifluoroethoxy)pyridine-2,3-diamine (200 mg, 0.49 mmol, 1.0 eq.) in toluene (4 mL) was added DIEA (276 μL, 1.54 mmol, 3.0 eq.) and methyl 2-chloro-2-oxoacetate (59 μL, 0.64 mmol, 1.3 eq.) at 0° C. Then the reaction mixture was stirred at 25° C. for 4 hrs. After completion, the reaction mixture was quenched with water (30 mL) and extracted with EtOAc (30 mL×3), the combined organic phase was dried over Na₂SO₄, filtered and concentrated under reduced pressure. The residue was purified by flash column chromatography on silica gel to afford 8-(2,5-dimethyl-1H-pyrrol-1-yl)-4-(4-methoxyphenyl)-6-(2,2,2-trifluoroethoxy)-1H,2H,3H,4H-pyrido[2,3-b]pyrazine-2,3-dione (40 mg, 18%) was obtained as a yellow oil. LC-MS (ESI): m/z 461[M+H]⁺.

### Step F: 8-amino-4-(4-methoxyphenyl)-6-(2,2,2-trifluoroethoxy)-1,4-dihydropyrido[2,3-b]pyrazine-2,3-dione

To a solution of 8-(2,5-dimethyl-1H-pyrrol-1-yl)-4-(4-methoxyphenyl)-6-(2,2,2-trifluoroethoxy)-1H,2H,3H,4H-pyridopyrazine-2,3-dione (80 mg, 0.17 mmol, 1.0 eq.) and NH₂OH HCl (604 mg, 8.69 mmol, 1.0 eq.) in EtOH (3 mL) and H₂O (1.5 mL) was added Et₃N (878 mg, 8.69 mmol, 1.0 eq.), the reaction mixture was stirred at 100° C. for 12 hrs. The reaction was complete as indicated by LCMS. The reaction mixture was concentrated under reduced pressure, the residue was purified by flash column chromatography on silica gel to afford 8-amino-4-(4-methoxyphenyl)-6-(2,2,2-trifluoroethoxy)-1,4-dihydropyrido[2,3-b]pyrazine-2,3-dione (40 mg, 60%) as a yellow solid. LC-MS (ESI): m/z=383 [M+H]⁺

### Step G: (Z)—N′-(2-chloro-4-(4-methoxyphenyl)-3-oxo-6-(2,2,2-trifluoroethoxy)-3,4-dihydropyrido[2,3-b]pyrazin-8-yl)-N,N-dimethylformimidamide

To a solution of 8-amino-4-(4-methoxyphenyl)-6-(2,2,2-trifluoroethoxy)-1,4-dihydropyrido[2,3-b]pyrazine-2,3-dione (40 mg, 0.10 mmol, 1.0 eq.) and DMF (9 mg, 0.12 mmol, 1.2 eq.) in CHC13 (3 mL) was added SOCl₂ (100 mg, 0.80 mmol, 8.0 eq.), the reaction mixture was stirred at 70° C. for 3 hrs. After completion, the reaction was quenched with sat. aqueous NaHCO₃ (10 mL), extracted with DCM (10 mL 3×). The combined organic layers were washed with brine (5 mL), dried over anhydrous Na₂SO₄, filtered and concentrated under reduced pressure to give crude (Z)—N′-(2-chloro-4-(4-methoxyphenyl)-3-oxo-6-(2,2,2-trifluoroethoxy)-3,4-dihydropyrido[2,3-b]pyrazin-8-yl)-N,N-dimethylformimidamide (40 mg) as a yellow solid, which was used for next step without further purification. LC-MS (ESI): m/z=456 [M+H]⁺.

### Step H: (E)-4-(4-methoxyphenyl)-2-(2-methyl-2H-indazol-5-yl)-8-((2-methylpropylidene)amino)-6-(2,2,2-trifluoroethoxy)pyrido[2,3-b]pyrazin-3(4H)-one

To a solution of (Z)—N′-(2-chloro-4-(4-methoxyphenyl)-3-oxo-6-(2,2,2-trifluoroethoxy)-3,4-dihydropyrido[2,3-b]pyrazin-8-yl)-N,N-dimethylformimidamide (40 mg, 0.088 mmol, 1.0 eq.), and (2-methyl-2H-indazol-5-yl)boronic acid (19 mg, 0.11 mmol, 1.2 eq.) in 1,4-dioxane (10 mL) and H₂O (1 mL) was added K₂CO₃ (24 mg, 0.18 mmol, 2.0 eq.) and Pd(dppf)Cl₂ (5 mg, 4.4 μmol, 0.1 eq.), the reaction mixture was stirred at 100° C. under N2 atmosphere for 3 hrs. The reaction mixture was concentrated under reduced pressure, the residue was purified by flash column to give (E)-4-(4-methoxyphenyl)-2-(2-methyl-2H-indazol-5-yl)-8-((2-methylpropylidene)amino)-6-(2,2,2-trifluoroethoxy)pyrido[2,3-b]pyrazin-3(4H)-one (25 mg) as a yellow solid. LC-MS (ESI): m/z=552 [M+H]⁺.

### Step I: 8-amino-4-(4-methoxyphenyl)-2-(2-methyl-2H-indazol-5-yl)-6-(2,2,2-trifluoroethoxy)pyrido[2,3-b]pyrazin-3(4H)-one

To a solution of (E)-4-(4-methoxyphenyl)-2-(2-methyl-2H-indazol-5-yl)-8-((2-methylpropylidene)amino)-6-(2,2,2-trifluoroethoxy)pyrido[2,3-b]pyrazin-3(4H)-one (25 mg, 0.0453 mmol, 1.0 eq.) in THE (4 mL) was added aqueous 2N HCl (1 mL), the reaction mixture was stirred at room temperature for 5 hrs. After completion, the reaction was quenched with NaHCO₃ (sat. aq.) (10 mL), extracted with EtOAc (10 mL×3). The combined organic layers were washed with brine (5 mL), dried over anhydrous Na₂SO₄, filtered and concentrated under reduced pressure, the residue was purified by RP-prep-HPLC to give 8-amino-4-(4-methoxyphenyl)-2-(2-methyl-2H-indazol-5-yl)-6-(2,2,2-trifluoroethoxy)pyrido[2,3-b]pyrazin-3(4H)-one (Example 352).

¹H NMR (400 MHz, DMSO-d₆) δ (ppm): 9.00 (s, 1H), 8.40 (s, 1H), 8.32 (dd, J=9.2 Hz, 1.7 Hz, 1H), 7.55 (d, J=9.2 Hz, 1H), 7.36-7.16 (m, 2H), 7.13-6.93 (m, 4H), 5.93 (s, 1H), 4.48 (q, J_{HF}=9.2 Hz, 2H), 4.11 (s, 3H), 3.77 (s, 3H). LC-MS (ESI): m/z=497 [M+H]⁺.

### Synthesis of 3-(2-amino-1-methyl-1H-benzo[d]imidazol-6-yl)-1-(4-methoxyphenyl)-7-(2,2,2-trifluoroethoxy)-1,8-naphthyridin-2(1H)-one (Example 353)

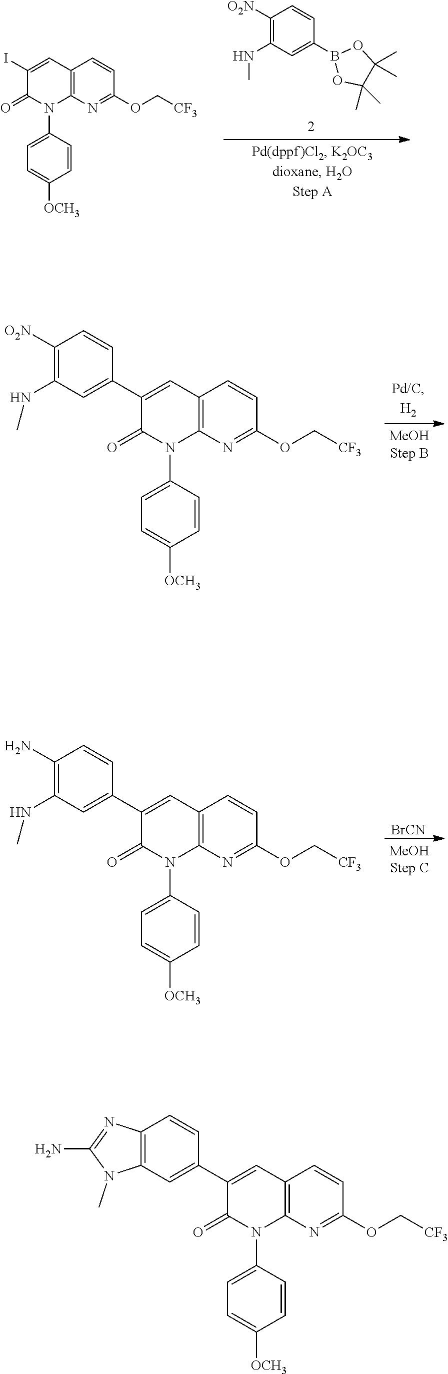

### Step A: 1-(4-methoxyphenyl)-3-(3-(methylamino)-4-nitrophenyl)-7-(2,2,2-trifluoroethoxy)-1,8-naphthyridin-2(1H)-one

A mixture of 3-iodo-1-(4-methoxyphenyl)-7-(2,2,2-trifluoroethoxy)-1,2-dihydro-1,8-naphthyridin-2-one (synthesized using 2,2,2-trifluoroethanol via General Procedure IV (Steps A-B)) (300 mg, 0.630 mmol, 1.0 eq), N-methyl-2-nitro-5-(tetramethyl-1,3,2-dioxaborolan-2-yl)aniline (210 mg, 0.75 mmol, 1.2 eq), K₂CO₃ (174 mg, 1.2 mmol, 2.0 eq) in dioxane (10 mL) and H₂O (2.5 mL) was added Pd(dppf)Cl₂ (46 mg, 0.06 mmol, 0.1 eq) and degassed with N2 and stirred under N2 at 100° C. for 14 hrs. After completion, the mixture was quenched by adding H₂O (10 mL), and extracted with EtOAc (10 mL×3). The combined organic layers were washed with brine (20 mL), dried over Na₂SO₄ and concentrated under reduced pressure, the residue was purified by flash column chromatography on silica gel to give 1-(4-methoxyphenyl)-3-[3-(methylamino)-4-nitrophenyl]-7-(2,2,2-trifluoroethoxy)-1,2-dihydro-1,8-naphthyridin-2-one (275 mg, 87%). LC-MS (ESI): m/z 501 [M+H]+.

### Step B: 3-(4-amino-3-(methylamino)phenyl)-1-(4-methoxyphenyl)-7-(2,2,2-trifluoroethoxy)-1,8-naphthyridin-2(1H)-one

To a solution of 1-(4-methoxyphenyl)-3-[3-(methylamino)-4-nitrophenyl]-7-(2,2,2-trifluoroethoxy)-1,2-dihydro-1,8-naphthyridin-2-one (86 mg, 0.17 mmol) in MeOH (10 mL) was added 10% Pd/C (20 mg), the resulting mixture was stirred at room temperature under H₂ atmosphere (1 atm) for 2 hrs. The suspension was filtered through a pad of Celite®, The filtrate was concentrated under reduced pressure to give crude 3-(4-amino-3-(methylamino)phenyl)-1-(4-methoxyphenyl)-7-(2,2,2-trifluoroethoxy)-1,8-naphthyridin-2(1H)-one (70 mg) as a black oil, which used directly for the next step without further purification. LC-MS (ESI): m/z 471 [M+H]⁺.

### Step C: 3-(2-amino-1-methyl-1H-benzo[d]imidazol-6-yl)-1-(4-methoxyphenyl)-7-(2,2,2-trifluoroethoxy)-1,8-naphthyridin-2(1H)-one

To a solution of 3-[4-amino-3-(methylamino)phenyl]-1-(4-methoxyphenyl)-7-(2,2,2-trifluoroethoxy)-1,2-dihydro-1,8-naphthyridin-2-one (70 mg, 0.15 mmol, 1.0 eq.) in MeOH (5 mL) was added BrCN (17 mg, 0.15 mmol, 1.0 eq.) and stirred at 80° C. for 1 hr. After completion, the reaction was quenched by adding H₂O (10 mL) and extracted with EtOAc (10 mL×3). The combined organic layers were washed with brine (20 mL), dried over Na₂SO₄ and concentrated under reduced pressure, the residue was purified by RP-prep-HPLC to give 3-(2-amino-1-methyl-1H-1,3-benzodiazol-6-yl)-1-(4-methoxyphenyl)-7-(2,2,2-trifluoroethoxy)-1,2-dihydro-1,8-naphthyridin-2-one (Example 353).

¹H NMR (400 MHz, DMSO-d₆) δ: 8.24 (d, J=8.4 Hz, 1H), 8.16 (s, 1H), 7.58 (d, J=1.5 Hz, 1H), 7.35 (dd, J=8.2 Hz, 1.7 Hz, 1H), 7.25 (d, J=8.9 Hz, 2H), 7.16 (d, J=8.2 Hz, 1H), 7.09 (d, J=9.0 Hz, 2H), 6.88 (d, J=8.3 Hz, 1H), 6.53 (s, 2H), 4.61 (q, J_{HF}=9.1 Hz, 2H), 3.83 (s, 3H), 3.51 (s, 3H). LC-MS (ESI): m/z 496 [M+H]⁺.

Biochemical Assay

Mat2A protein was expressed by recombinant baculovirus in SF9 infected cells using the Bac to Bac system cloned into the pFASTBAC1 vector (Invitrogen, Carlsbad, Calif.). Recombinant MAT2A was isolated from the cell lysate of 150 g of infected cells using HP Ni sepharose column chromatography. Recombinant MAT2A homodimer was eluted with 250 and 500 mM imidazole, and fractions containing MAT2A were identified by sodium dodecyl sulfate polyacrylamide gel electrophoresis and pooled.

For determination of the inhibitory potency of compounds against the MAT2A homodimer, protein was diluted to 4 μg/mL in assay buffer (50 mM Tris, pH 8.0, 50 mM KCl, 15 mM MgCl₂, 0.3 mM EDTA, 0.005% [w/v] bovine serum albumin [BSA]). Test compound was prepared in 100% dimethyl sulfoxide (DMSO) at 50× the desired final concentration. A 1 μL volume of compound dilution was added to 40 μL of enzyme dilution and the mixture was allowed to equilibrate for 60 minutes at 25° C. The enzymatic assay was initiated by the addition of 10 μL of substrate mix (500 μM ATP, pH 7.0, 40 μM L-methionine in 1× assay buffer), and the mixture was incubated for a further 60 minutes at 25° C. The reaction was halted and the liberated phosphate released by the enzyme in stoichiometric amounts by the production of S-adenosyl methionine (SAM) was measured using the PiColorLock Gold kit (Innova Biosciences, UK). Absolute product amounts were determined by comparison to a standard curve of potassium phosphate buffer, pH 8.0.

Specific compounds disclosed herein were tested in the foregoing assay and they were determined to inhibit MAT2A with an IC₅₀ according to the following scores: (A) less than 100 nM (>40% maximum inhibition), (B) between 100 nM and 1 μM (>38% maximum inhibition), (C) between 1 μM and 10 μM (>40% maximum inhibition), and (D) greater than 10 μM as shown in Table 7 below.

Cellular Assay of Target Engagement (SAM)

Measurement of MAT2A activity in cells was made by direct quantitation of the abundance of the product of its enzymatic activity, SAM. Cancer cells were treated with candidate MAT2A inhibitors for a suitable incubation period, and the cells were then lysed using a reagent which quenched any further enzyme activity. Soluble metabolites including SAM were collected and SAM itself was directly measured from the lysate using quantitative LC-MS/MS.

A typical assay was performed using an HCT116 human colon carcinoma cell line which was genetically engineered to delete the MTAP gene (commercially available from Horizon Discovery). This cell line was utilized because it was determined that loss of the MTAP gene predicts sensitivity to MAT2A inhibitors. Cells were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% CO₂ for 72 hrs.

To quantitate SAM levels following compound treatment, cells were gently washed once in ammonium carbonate buffer (75 mM at pH 7.4), placed on dry ice, and lysed with metabolite extraction buffer (80% cold methanol and 20% water (v/v) with acetic acid at 1M final concentration with 200 ng/mL deuterated d3-SAM as internal control). Following centrifugation at 4° C. at 3,200 rpm for 30 minutes, the supernatant was collected and stored at −80° C. until analysis by Liquid Chromatography with tandem Mass Spectrometry (LC-MS/MS). LC-MS/MS analysis was performed using an API6500 Mass Spectrometer (Sciex, Framingham, Mass., USA) operating in positive ion spray mode and equipped with a Waters UPLC Acquity (Waters, Milford, Mass., USA) BEH Amide column. Multiple Reaction Monitoring data was acquired for SAM and the d3-SAM standard, using a mass transition pair at m/z 399.2→250.1 and 402.2→250.1, respectively. In a typical LC-MS/MS analysis, the initial flow rate was 0.5 ml/min of 25% mobile phase A (acetonitrile and water at 5:95 (v/v) with 1% formic acid and 10 mM ammonium acetate) and 75% mobile phase B (acetonitrile and water at 95:5 (v/v) with 1% formic acid and 10 mM ammonium acetate), 0.2-0.5 minutes with 75%-35% mobile phase B, 25%-65% mobile phase A, at 0.5 min 65% mobile phase A and 35% mobile phase B, 1.0-1.1 minutes with 35%-75% mobile phase B, 65%-25% mobile phase A, at 1.1 min 25% mobile phase A and 75% mobile phase B with a total run time of 1.5 minutes.

Specific compounds disclosed herein were tested in the foregoing assay and they were determined to inhibit SAM with an IC₅₀ according to the following scores: (A) less than 100 nM (>60% maximum inhibition), (B) between 100 nM and 1 μM (>60% maximum inhibition), (C) greater than or equal to 1 μM (>60% maximum inhibition), and (NT) not tested, as shown in Table 5 below.

Assay for Inhibition of Cellular Proliferation

Test compound impact on cancer cell growth was assessed by treating cancer cells with compound for 4 days and then measuring proliferation using an ATP-based cell proliferation readout (Cell Titer Glo, Promega Corporation).

In a typical assay an isogenic pair of HCT116 human colon carcinoma cell lines which vary only in MTAP deletion status (HCT116 MTAP +/+ and HCT116 MTAP−/−) were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% C₀₂ for 4 days.

To measure inhibition of cellular proliferation, cells were allowed to equilibrate to room temperature for 30 minutes, and were then treated with 125 μL of Cell Titer Glo reagent. The plate was then covered with aluminum foil and shaken for 15 minutes to ensure complete mixing and full cell lysis. Luminescent signal was then measured using a plate-based luminometer Veritas version 1.9.2 using ATP standard curve to confirm assay reproducibility from run to run. This luminescence measure was converted to a proliferation index by subtracting from each data point the ATP luminescence signal measured from a bank (no cells) well and dividing by the ATP luminescence signal measured in 0.2% DMSO control well adjusted for signal in blank well. Compound activity was then represented as a percentage change in proliferation relative to a within-plate DMSO control against log 10 of compound concentration in molar (M) units.

Specific compounds disclosed herein were tested in the foregoing assay and they were determined to inhibit cellular proliferation with an IC₅₀ according to the following scores: (A) less than 100 nM (>30% maximum inhibition for MTAP −/−; >10% maximum inhibition for MTAP +/+), (B) between 100 nM and 1 μM (>30% maximum inhibition for MTAP −/−; >10% maximum inhibition for MTAP +/+), (C) greater than or equal to 1 μM, and (NT) not tested, as shown in Table 5 below.

TABLE 7

| | | | 4 Day | 4 Day |
|---|---|---|---|---|
| | | Cell 72 h | Relative | Relative |
| | | SAM | Growth | Growth |
| | Enzyme | Inhibition | Inhibition | Inhibition |
| Example | Inhibition | (MTAP −/−) | (MTAP −/−) | (MTAP +/+) |
| 101 | A | A | B | C |
| 102 | B | NT | NT | NT |
| 103 | B | NT | NT | NT |
| 104 | C | NT | NT | NT |
| 105 | C | NT | NT | NT |
| 106 | B | B | B | C |
| 107 | B | NT | NT | NT |
| 108 | C | NT | NT | NT |
| 109 | B | NT | NT | NT |
| 110 | B | NT | NT | NT |
| 111 | C | NT | NT | NT |
| 112 | A | NT | NT | NT |
| 113 | B | NT | NT | NT |
| 114 | B | NT | NT | NT |
| 115 | A | A | B | C |
| 116 | A | A | A | C |
| 117 | B | NT | NT | NT |
| 118 | A | A | A | C |
| 119 | A | A | A | C |
| 120 | A | A | A | C |
| 121 | A | A | A | C |
| 122 | A | A | A | B |
| 123 | A | A | A | C |
| 124 | A | A | A | B |
| 125 | A | NT | NT | NT |
| 126 | A | NT | NT | NT |
| 127 | A | A | B | C |
| 128 | B | NT | NT | NT |
| 129 | A | A | A | C |
| 130 | A | A | B | C |
| 131 | A | A | A | C |
| 132 | A | NT | NT | NT |
| 133 | A | NT | NT | NT |
| 134 | A | NT | NT | NT |
| 135 | A | A | A | C |
| 136 | A | NT | NT | NT |
| 137 | A | NT | NT | NT |
| 138 | A | A | A | B |
| 139 | A | A | B | C |
| 140 | A | A | A | B |
| 141 | A | NT | NT | NT |
| 142 | A | A | A | C |
| 143 | A | NT | NT | NT |
| 144 | A | A | A | B |
| 145 | A | A | A | C |
| 146 | A | NT | NT | NT |
| 147 | A | A | A | B |
| 148 | A | A | A | B |
| 149 | A | A | A | B |
| 150 | A | A | A | C |
| 151 | A | A | A | C |
| 152 | A | A | A | C |
| 153 | A | A | A | C |
| 154 | A | NT | NT | NT |
| 155 | A | A | A | C |
| 156 | B | NT | NT | NT |
| 157 | A | A | A | B |
| 158 | A | B | B | C |
| 159 | A | A | B | C |
| 160 | A | A | B | C |
| 161 | A | A | A | B |
| 162 | A | B | B | C |
| 163 | A | B | B | C |
| 164 | A | A | A | C |
| 165 | A | A | A | C |
| 166 | A | A | A | C |
| 167 | A | A | A | C |
| 168 | A | A | A | C |
| 169 | A | A | B | C |
| 170 | B | NT | NT | NT |
| 171 | A | B | C | C |
| 172 | A | A | B | C |
| 173 | A | A | A | C |
| 174 | A | A | C | C |
| 175 | B | NT | NT | NT |
| 176 | A | A | A | B |
| 177 | A | A | C | C |
| 178 | B | NT | NT | NT |
| 179 | B | NT | NT | NT |
| 180 | A | B | B | C |
| 181 | B | NT | NT | NT |
| 182 | C | NT | NT | NT |
| 183 | A | B | C | C |
| 184 | B | NT | NT | NT |
| 185 | A | A | B | C |
| 186 | A | A | B | C |
| 187 | A | B | B | C |
| 188 | A | A | B | C |
| 189 | A | B | B | C |
| 190 | A | NT | NT | NT |
| 191 | B | NT | NT | NT |
| 192 | A | NT | NT | NT |
| 193 | A | NT | NT | NT |
| 194 | A | A | A | B |
| 195 | A | A | A | B |
| 196 | A | A | B | B |
| 197 | A | A | A | C |
| 198 | A | NT | NT | NT |
| 199 | A | A | B | C |
| 200 | A | A | A | B |
| 201 | A | A | A | C |
| 202 | A | B | B | C |
| 203 | A | A | A | C |
| 204 | A | A | A | C |
| 205 | A | A | A | C |
| 206 | A | NT | NT | NT |
| 207 | A | A | B | B |
| 208 | A | A | A | B |
| 209 | A | A | A | B |
| 210 | A | A | A | B |
| 211 | A | NT | NT | NT |
| 212 | A | NT | NT | NT |
| 213 | A | B | B | C |
| 214 | A | A | B | C |
| 215 | A | A | A | NT |
| 216 | A | A | A | C |
| 217 | A | A | A | C |
| 218 | A | A | A | C |
| 219 | A | A | B | C |
| 220 | A | A | A | C |
| 221 | A | A | A | C |
| 222 | A | A | A | C |
| 223 | A | B | B | C |
| 224 | A | A | A | B |
| 225 | A | A | A | C |
| 226 | A | A | A | C |
| 227 | A | A | A | C |
| 228 | A | A | A | C |
| 229 | A | A | A | C |
| 230 | A | A | B | B |
| 231 | A | A | A | B |
| 232 | A | A | B | C |
| 233 | A | A | A | C |
| 234 | A | NT | NT | NT |
| 235 | A | B | B | C |
| 236 | A | A | A | C |
| 237 | A | A | A | C |
| 301 | A | A | A | B |
| 302 | A | A | A | B |
| 303 | A | A | A | B |
| 304 | A | A | NT | NT |
| 305 | A | A | A | C |
| 306 | A | NT | NT | NT |
| 307 | A | A | A | C |
| 308 | A | A | A | C |
| 309 | A | A | A | A |
| 310 | A | A | A | B |
| 311 | A | NT | NT | NT |
| 312 | A | NT | NT | NT |
| 313 | A | A | A | C |
| 314 | A | A | A | C |
| 315 | A | NT | NT | NT |
| 316 | A | A | A | C |
| 317 | A | A | B | B |
| 318 | A | A | A | B |
| 319 | A | A | A | C |
| 320 | A | A | A | B |
| 321 | A | A | A | C |
| 322 | A | A | A | C |
| 323 | A | NT | NT | NT |
| 324 | A | A | A | B |
| 325 | A | A | A | C |
| 326 | A | A | A | C |
| 327 | A | A | A | C |
| 328 | A | A | A | C |
| 329 | A | A | A | C |
| 330 | A | A | A | C |
| 331 | A | A | A | C |
| 332 | A | A | A | C |
| 333 | A | A | B | C |
| 334 | A | A | A | C |
| 335 | A | A | A | C |
| 336 | A | NT | NT | NT |
| 337 | A | NT | NT | NT |
| 338 | A | A | A | A |
| 339 | A | NT | NT | NT |
| 340 | A | A | A | C |
| 341 | A | A | A | C |
| 342 | A | A | A | B |
| 343 | A | A | A | C |
| 344 | A | A | A | C |
| 345 | A | A | B | C |
| 346 | A | A | A | C |
| 347 | A | A | B | C |
| 348 | A | A | A | C |
| 349 | A | NT | NT | NT |
| 350 | B | NT | NT | NT |
| 351 | A | A | A | C |
| 352 | A | A | A | B |
| 353 | A | A | A | C |

1. A compound according to Formula I: 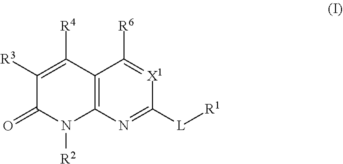
wherein:
X¹ is N or CR⁵;
L is O or NR;
R is H or C₁-C₆-alkyl;
R¹ is selected from the group consisting of C₁-C₆-alkyl, C₂-C₆-alkenyl, C₃-C₅-carbocyclyl, —(C₁-C₆-alkyl)(C₃-C₆-carbocyclyl), and —(C₁-C₆-alkyl)(C₃-C₆-cycloalkenyl), wherein:
any alkyl in R¹ is straight or branched;
R¹ is optionally substituted by 1-6 halo or 1-6 deuterium; and
when X¹ is N, L is NR, R is H, and R¹ is C₁-C₆-alkyl, then R¹ is substituted by 1-6 halo;
or when L is NR, then R and R¹ can be taken together in combination with L to form a 3- to 6-membered heterocycloalkyl (wherein 1-4 ring members are, independently, N, O, or S) optionally substituted by one or more R^{A};
R² and R³ are independently selected from the group consisting of C₆-C₁₀-aryl and 5- to 10-membered heteroaryl (wherein 1-4 heteroaryl members are, independently, N, O, or S),
wherein R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D};
R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D};
R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, or S);
wherein each alkyl, alkoxy, alkenyl, alkynyl, aryl, carbocyclyl, heterocycloalkyl, and heteroaryl moiety of R^{A} and R^{B} is, independently, optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S), and —(C₁-C₆-alkyl)-(3- to 14-membered heterocycle) (wherein 1-4 heterocycle members are, independently, N, O, or S), and —O(C₆-C₁₄-aryl),
wherein each alkyl, alkenyl, aryl, and heterocycloalkyl is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo;
R^{C} and R^{D} are, independently, H or C₁-C₆-alkyl; and
wherein the compound is not:
6-(2-chlorophenyl)-8-(4-fluorophenyl)-2-(methylthio)pyrido[2,3-d]pyrimidin-7(8H)-one;
2-(methylthio)-8-phenyl-6-(o-tolyl)pyrido[2,3-d]pyrimidin-7(8H)-one;
6-(4-hydroxyphenyl)-2-(methylthio)-8-phenylpyrido[2,3-d]pyrimidin-7(8H)-one;
6-(4-methoxyphenyl)-2-(methylthio)-8-phenylpyrido[2,3-d]pyrimidin-7(8H)-one;
6-(2,5-dimethoxyphenyl)-2-(methylthio)-8-phenylpyrido[2,3-d]pyrimidin-7(8H)-one;
6-(4-(tert-butyl)phenyl)-2-(methylthio)-8-phenylpyrido[2,3-d]pyrimidin-7(8H)-one; or
6-(2,6-dichlorophenyl)-2-(methylthio)-8-phenylpyrido[2,3-d]pyrimidin-7(8H)-one;
or a pharmaceutically acceptable salt thereof.

2. A compound according to Formula II: 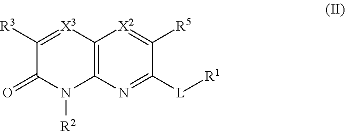
wherein:
X² is CR⁶ and X³ is N, or X² is N and X³ is CR⁴;
L is O, S, NR, or a bond;
R is H or C₁-C₆-alkyl;
R¹ is selected from the group consisting of C₁-C₆-alkyl or C₃-C₆-carbocyclyl, wherein:
any alkyl in R¹ is straight or branched;
R¹ is optionally substituted by 1-6 halo;
or when L is NR, then R and R¹ can be taken together in combination with L to form a 3- to 6-membered heterocycloalkyl (wherein 1-4 ring members are, independently, N, O, or S) optionally substituted by one or more R^{A};
R² and R³ are independently selected from the group consisting of C₆-C₁₀-aryl and 5- to 10-membered heteroaryl (wherein 1-4 heteroaryl members are, independently, N, O, or S),
wherein R² and R³ are independently and optionally substituted by one or more substituents that are selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halo, oxo, —CN, and NR^{C}R^{D};
R⁵ is selected from the group consisting of H, C₁-C₆-alkyl, C₁-C₆-alkoxy, C₂-C₆-alkenyl, C₂-C₆-alkynyl, halo, —CN, and NR^{C}R^{D};
R⁶ is selected from the group consisting of H, C₁-C₆-alkyl optionally substituted by one or more halo, —O(C₁-C₆-alkyl) optionally substituted by one or more halo, —OH, halo, —CN, —(C₁-C₆-alkyl)NR^{A}R^{B}, and NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, or S);
wherein each alkyl, alkoxy, alkenyl, alkynyl, aryl, carbocyclyl, heterocycloalkyl, and heteroaryl moiety of R^{A} and R^{B} is optionally and independently substituted with one or more substituents selected from the group consisting of hydroxy, deuterium, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl (wherein 1-4 ring members are, independently, N, O, or S), —(C₁-C₆-alkyl)-(3- to 14-membered heterocycle) (wherein 1-4 heterocycle members are, independently, N, O, or S), and —O(C₆-C₁₄-aryl),
wherein each alkyl, alkenyl, aryl, and heterocycloalkyl is optionally and independently substituted with one or more substituents selected from the group consisting of hydroxy, —OC₁-C₆-alkyl, halo, —NH₂, —(C₁-C₆-alkyl)NH₂, —C(O)OH, CN, and oxo;
R^{C} and R^{D} are, independently, H or C₁-C₆-alkyl;
or a pharmaceutically acceptable salt thereof.

3. The compound or pharmaceutically acceptable salt thereof according to claim 1, wherein X¹ is N.

4. The compound or pharmaceutically acceptable salt thereof according to claim 1, wherein X¹ is CR⁵.

5. The compound or pharmaceutically acceptable salt thereof according to claim 2, wherein (i) X² is CR⁶ and X³ is N or (ii) X² is N and X³ is CR⁴.

6. (canceled)

7. The compound or pharmaceutically acceptable salt thereof according to claim 1, wherein:
each of R⁴ and R⁵ is, independently, H or C₁-C₆-alkyl; and
R⁶ is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are, independently, H or C₁-C₆-alkyl).

8. The compound or pharmaceutically acceptable salt thereof according to claim 1, wherein at least one of R⁴, R⁵, and R⁶ is H.

9-12. (canceled)

13. The compound or pharmaceutically acceptable salt thereof according to claim 1, wherein R² is C₆-C₁₀-aryl.

14. (canceled)

15. The compound or pharmaceutically acceptable salt thereof according to claim 1, wherein R² is 5- to 10-membered heteroaryl, and wherein 1 ring member is N.

16. (canceled)

17. The compound or pharmaceutically acceptable salt thereof according to claim 1, wherein R³ is 5- to 10-membered heteroaryl.

18. (canceled)

19. The compound or pharmaceutically acceptable salt thereof according to claim 1, wherein R³ is C₆-C₁₀-aryl.

20-23. (canceled)

24. The compound or pharmaceutically acceptable salt thereof according to claim 1, wherein L is NR.

25. The compound or pharmaceutically acceptable salt thereof according to claim 1, wherein R¹ is C₁-C₃-alkyl that is optionally substituted by 1-3 fluoro or C₃-C₅-carbocyclyl.

26. The compound or pharmaceutically acceptable salt thereof according to claim 1, wherein:
R is H;
R¹ is C₁-C₃-alkyl that is optionally substituted by 1-3 fluoro;
R² is 5- to 10-membered heteroaryl (wherein 1 heteroaryl member is N) or C₆-C₁₀-aryl;
R³ is 5- to 10-membered heteroaryl (wherein 1 to 3 heteroaryl members are, independently, N, O, or S) or C₆-C₁₀-aryl; and
each of R⁴, R⁵, and R6 is H.

27-31. (canceled)

32. The compound or pharmaceutically acceptable salt thereof according to claim 1, wherein:
R is H;
X¹ is CR⁵;
R¹ is C₁-C₃-alkyl that is optionally substituted by 1-3 fluoro;
R² is substituted phenyl or substituted pyridyl;
R³ is selected from the group consisting of substituted phenyl, substituted benzimidazolyl, and triazolopyridinyl; and
each of R⁴, R⁵, and R⁶ is H.

33. The compound or pharmaceutically acceptable salt thereof according to claim 1, wherein the compound is

| Ex | |
|---|---|
| No | Structure |
| 101 | 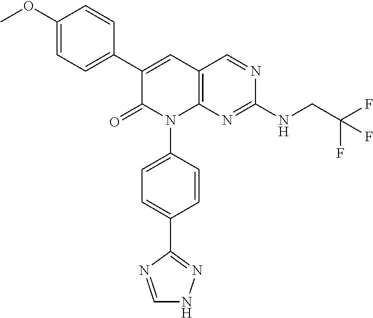 [SMILES: COc1ccc(-c2cc3cnc(NCC(F)(F)F)nc3n(-c3ccc(-c4nc[nH]n4)cc3)c2=O)cc1] |
| 102 | 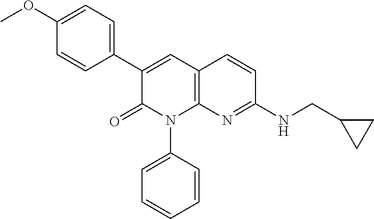 [SMILES: COc1ccc(-c2cc3ccc(NCC4CC4)nc3n(-c3ccccc3)c2=O)cc1] |
| 103 | 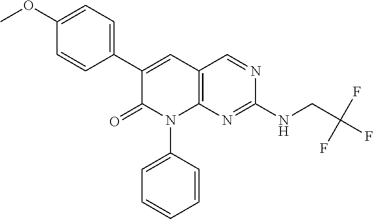 [SMILES: COc1ccc(-c2cc3cnc(NCC(F)(F)F)nc3n(-c3ccccc3)c2=O)cc1] |
| 106 | 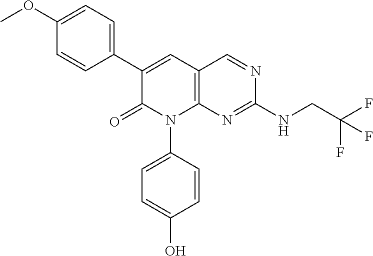 [SMILES: COc1ccc(-c2cc3cnc(NCC(F)(F)F)nc3n(-c3ccc(O)cc3)c2=O)cc1] |
| 107 | 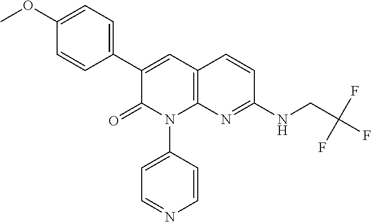 [SMILES: COc1ccc(-c2cc3ccc(NCC(F)(F)F)nc3n(-c3ccncc3)c2=O)cc1] |
| 109 | 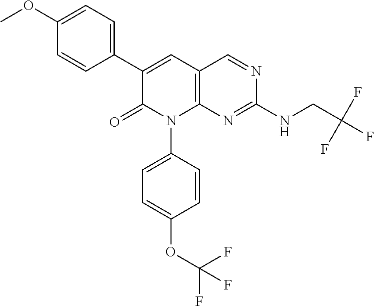 [SMILES: COc1ccc(-c2cc3cnc(NCC(F)(F)F)nc3n(-c3ccc(OC(F)(F)F)cc3)c2=O)cc1] |
| 112 | 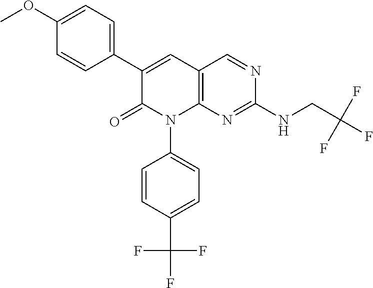 [SMILES: COc1ccc(-c2cc3cnc(NCC(F)(F)F)nc3n(-c3ccc(C(F)(F)F)cc3)c2=O)cc1] |
| 115 | 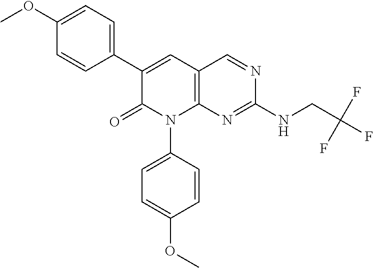 [SMILES: COc1ccc(-c2cc3cnc(NCC(F)(F)F)nc3n(-c3ccc(OC)cc3)c2=O)cc1] |
| 116 | 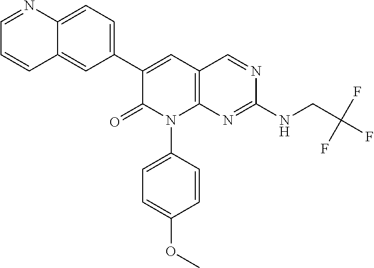 [SMILES: COc1ccc(-n2c(=O)c(-c3ccc4ncccc4c3)cc3cnc(NCC(F)(F)F)nc32)cc1] |
| 117 | 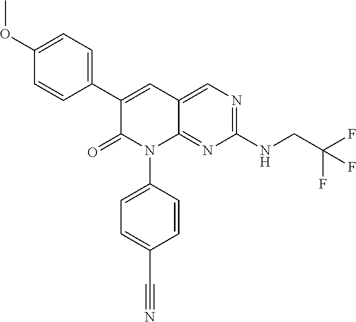 [SMILES: COc1ccc(-c2cc3cnc(NCC(F)(F)F)nc3n(-c3ccc(C#N)cc3)c2=O)cc1] |
| 118 | 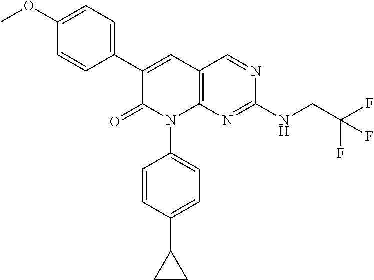 [SMILES: COc1ccc(-c2cc3cnc(NCC(F)(F)F)nc3n(-c3ccc(C4CC4)cc3)c2=O)cc1] |
| 119 | 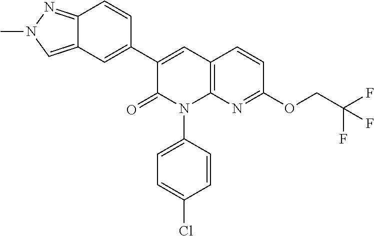 [SMILES: Cn1cc2cc(-c3cc4ccc(OCC(F)(F)F)nc4n(-c4ccc(Cl)cc4)c3=O)ccc2n1] |
| 120 | 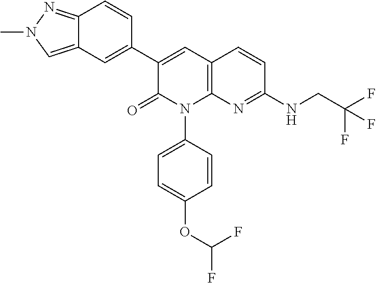 [SMILES: Cn1cc2cc(-c3cc4ccc(NCC(F)(F)F)nc4n(-c4ccc(OC(F)F)cc4)c3=O)ccc2n1] |
| 121 | 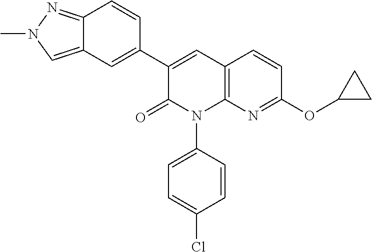 [SMILES: Cn1cc2cc(-c3cc4ccc(OC5CC5)nc4n(-c4ccc(Cl)cc4)c3=O)ccc2n1] |
| 122 | 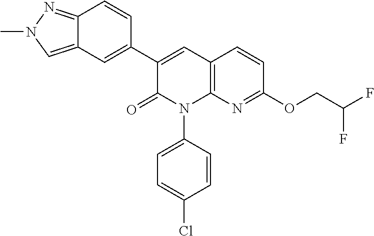 [SMILES: Cn1cc2cc(-c3cc4ccc(OCC(F)F)nc4n(-c4ccc(Cl)cc4)c3=O)ccc2n1] |
| 123 | 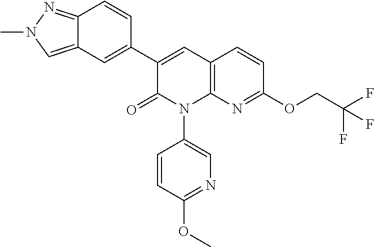 [SMILES: COc1ccc(-n2c(=O)c(-c3ccc4nn(C)cc4c3)cc3ccc(OCC(F)(F)F)nc32)cn1] |
| 124 | 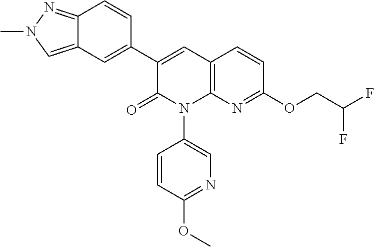 [SMILES: COc1ccc(-n2c(=O)c(-c3ccc4nn(C)cc4c3)cc3ccc(OCC(F)F)nc32)cn1] |
| 125 | 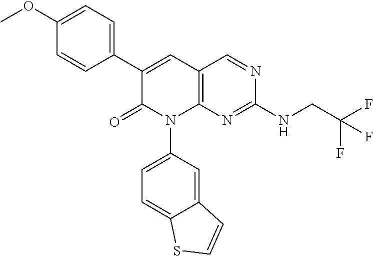 [SMILES: COc1ccc(-c2cc3cnc(NCC(F)(F)F)nc3n(-c3ccc4sccc4c3)c2=O)cc1] |
| 126 | 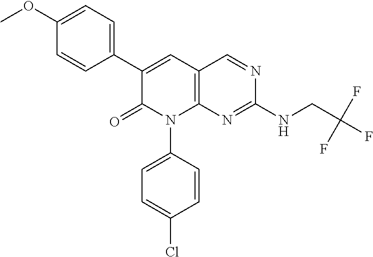 [SMILES: COc1ccc(-c2cc3cnc(NCC(F)(F)F)nc3n(-c3ccc(Cl)cc3)c2=O)cc1] |
| 127 | 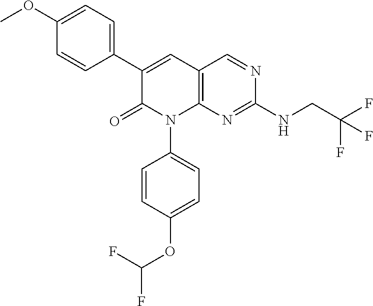 [SMILES: COc1ccc(-c2cc3cnc(NCC(F)(F)F)nc3n(-c3ccc(OC(F)F)cc3)c2=O)cc1] |
| 128 | 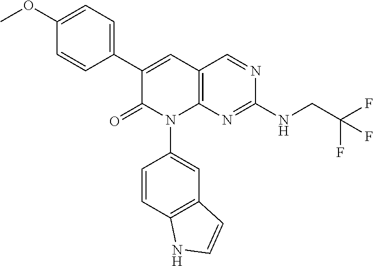 [SMILES: COc1ccc(-c2cc3cnc(NCC(F)(F)F)nc3n(-c3ccc4[nH]ccc4c3)c2=O)cc1] |
| 129 | 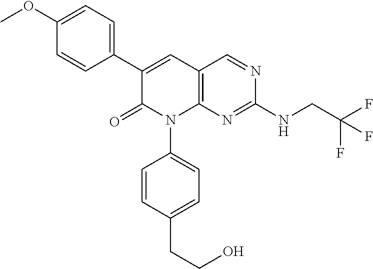 [SMILES: COc1ccc(-c2cc3cnc(NCC(F)(F)F)nc3n(-c3ccc(CCO)cc3)c2=O)cc1] |
| 130 | 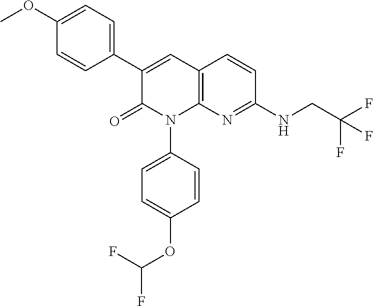 [SMILES: COc1ccc(-c2cc3ccc(NCC(F)(F)F)nc3n(-c3ccc(OC(F)F)cc3)c2=O)cc1] |
| 131 | 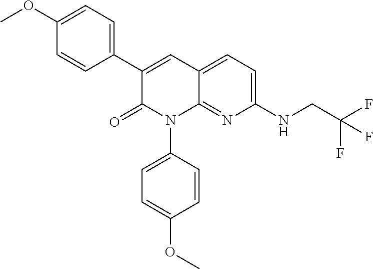 [SMILES: COc1ccc(-c2cc3ccc(NCC(F)(F)F)nc3n(-c3ccc(OC)cc3)c2=O)cc1] |
| 132 | 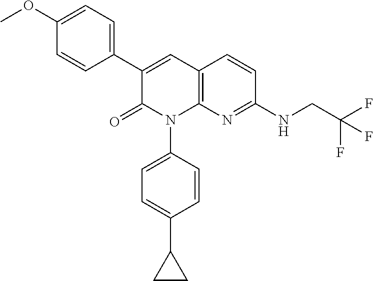 [SMILES: COc1ccc(-c2cc3ccc(NCC(F)(F)F)nc3n(-c3ccc(C4CC4)cc3)c2=O)cc1] |
| 133 | 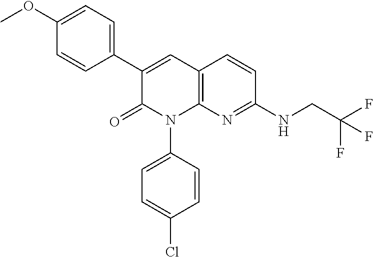 [SMILES: COc1ccc(-c2cc3ccc(NCC(F)(F)F)nc3n(-c3ccc(Cl)cc3)c2=O)cc1] |
| 134 | 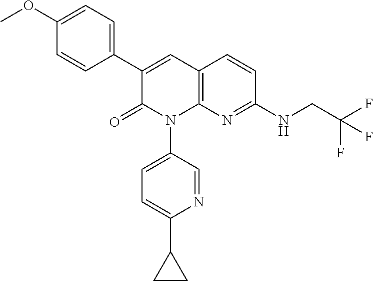 [SMILES: COc1ccc(-c2cc3ccc(NCC(F)(F)F)nc3n(-c3ccc(C4CC4)nc3)c2=O)cc1] |
| 135 | 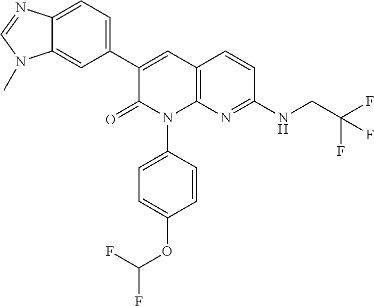 [SMILES: Cn1cnc2ccc(-c3cc4ccc(NCC(F)(F)F)nc4n(-c4ccc(OC(F)F)cc4)c3=O)cc21] |
| 136 | 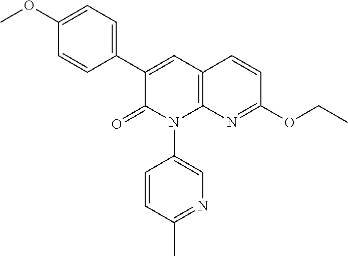 [SMILES: CCOc1ccc2cc(-c3ccc(OC)cc3)c(=O)n(-c3ccc(C)nc3)c2n1] |
| 137 | 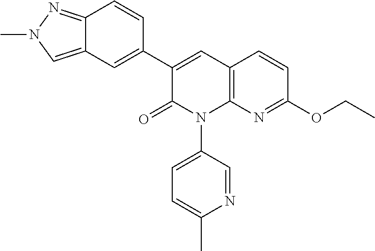 [SMILES: CCOc1ccc2cc(-c3ccc4nn(C)cc4c3)c(=O)n(-c3ccc(C)nc3)c2n1] |
| 138 | 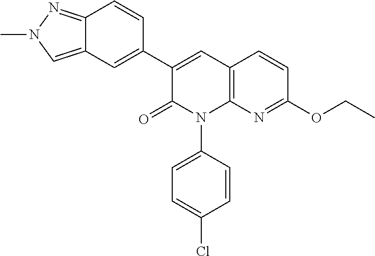 [SMILES: CCOc1ccc2cc(-c3ccc4nn(C)cc4c3)c(=O)n(-c3ccc(Cl)cc3)c2n1] |
| 139 | 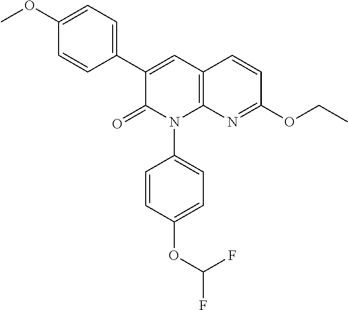 [SMILES: CCOc1ccc2cc(-c3ccc(OC)cc3)c(=O)n(-c3ccc(OC(F)F)cc3)c2n1] |
| 140 | 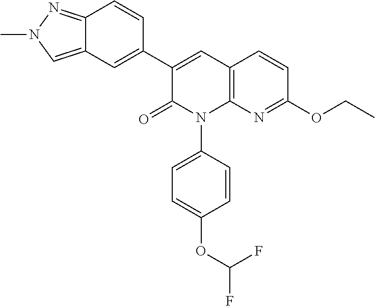 [SMILES: CCOc1ccc2cc(-c3ccc4nn(C)cc4c3)c(=O)n(-c3ccc(OC(F)F)cc3)c2n1] |
| 141 | 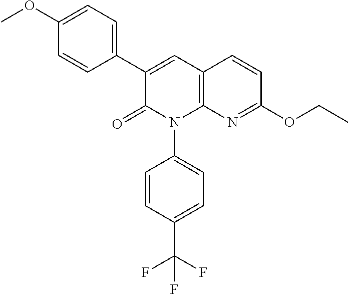 [SMILES: CCOc1ccc2cc(-c3ccc(OC)cc3)c(=O)n(-c3ccc(C(F)(F)F)cc3)c2n1] |
| 142 | 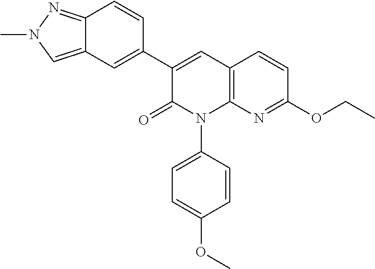 [SMILES: CCOc1ccc2cc(-c3ccc4nn(C)cc4c3)c(=O)n(-c3ccc(OC)cc3)c2n1] |
| 143 | 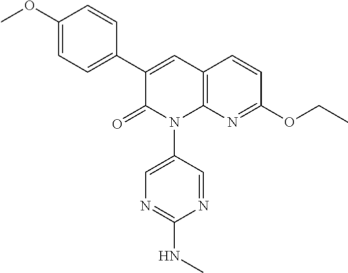 [SMILES: CCOc1ccc2cc(-c3ccc(OC)cc3)c(=O)n(-c3cnc(NC)nc3)c2n1] |
| 144 | 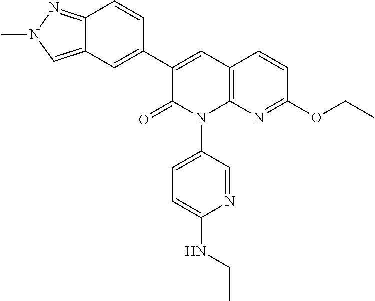 [SMILES: CCNc1ccc(-n2c(=O)c(-c3ccc4nn(C)cc4c3)cc3ccc(OCC)nc32)cn1] |
| 145 | 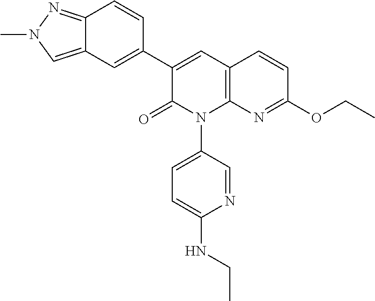 [SMILES: CCNc1ccc(-n2c(=O)c(-c3ccc4nn(C)cc4c3)cc3ccc(OCC)nc32)cn1] |
| 146 | 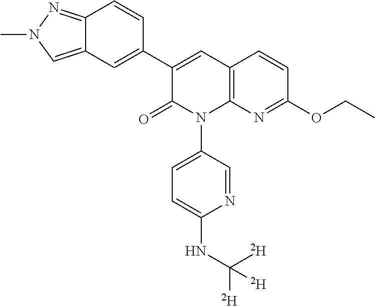 [SMILES: [2H]C([2H])([2H])Nc1ccc(-n2c(=O)c(-c3ccc4nn(C)cc4c3)cc3ccc(OCC)nc32)cn1] |
| 147 | 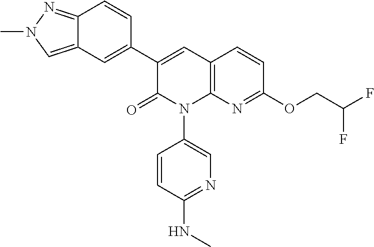 [SMILES: CNc1ccc(-n2c(=O)c(-c3ccc4nn(C)cc4c3)cc3ccc(OCC(F)F)nc32)cn1] |
| 148 | 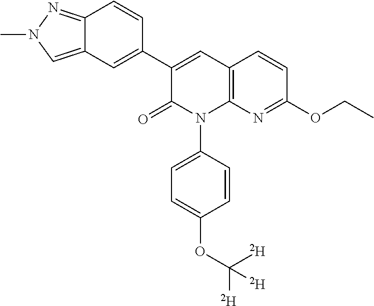 [SMILES: [2H]C([2H])([2H])Oc1ccc(-n2c(=O)c(-c3ccc4nn(C)cc4c3)cc3ccc(OCC)nc32)cc1] |
| 149 | 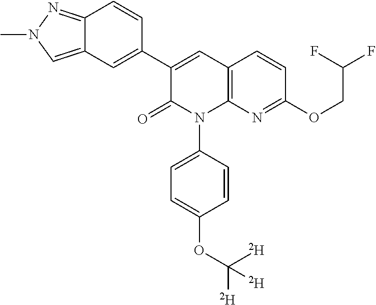 [SMILES: [2H]C([2H])([2H])Oc1ccc(-n2c(=O)c(-c3ccc4nn(C)cc4c3)cc3ccc(OCC(F)F)nc32)cc1] |
| 150 | 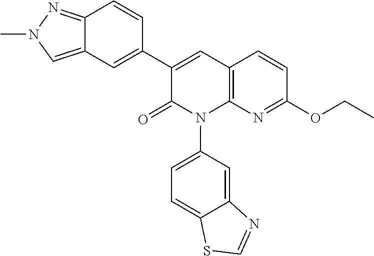 [SMILES: CCOc1ccc2cc(-c3ccc4nn(C)cc4c3)c(=O)n(-c3ccc4scnc4c3)c2n1] |
| 151 | 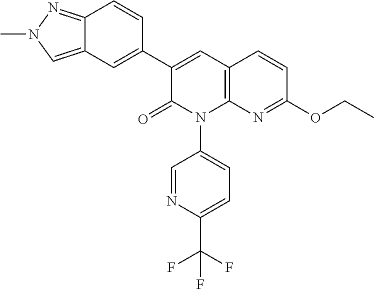 [SMILES: CCOc1ccc2cc(-c3ccc4nn(C)cc4c3)c(=O)n(-c3ccc(C(F)(F)F)nc3)c2n1] |
| 152 | 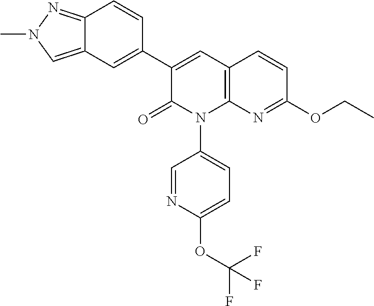 [SMILES: CCOc1ccc2cc(-c3ccc4nn(C)cc4c3)c(=O)n(-c3ccc(OC(F)(F)F)nc3)c2n1] |
| 153 | 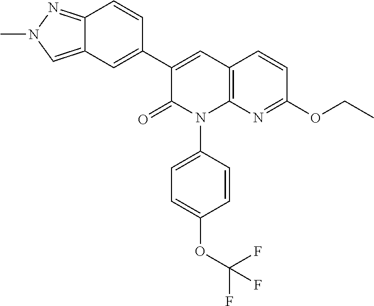 [SMILES: CCOc1ccc2cc(-c3ccc4nn(C)cc4c3)c(=O)n(-c3ccc(OC(F)(F)F)cc3)c2n1] |
| 154 | 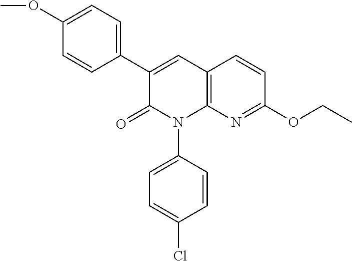 [SMILES: CCOc1ccc2cc(-c3ccc(OC)cc3)c(=O)n(-c3ccc(Cl)cc3)c2n1] |
| 155 | 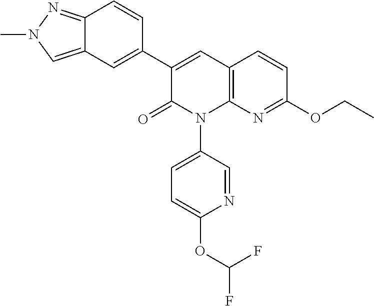 [SMILES: CCOc1ccc2cc(-c3ccc4nn(C)cc4c3)c(=O)n(-c3ccc(OC(F)F)nc3)c2n1] |
| 156 | 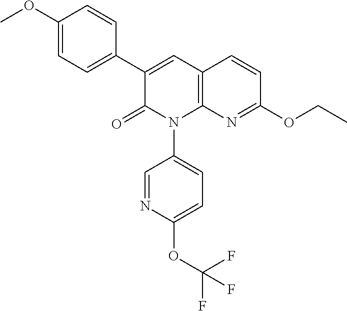 [SMILES: CCOc1ccc2cc(-c3ccc(OC)cc3)c(=O)n(-c3ccc(OC(F)(F)F)nc3)c2n1] |
| 157 | 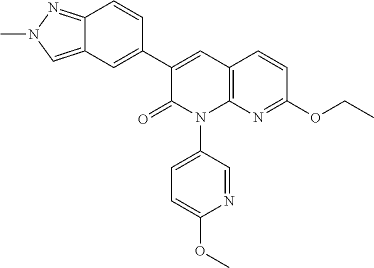 [SMILES: CCOc1ccc2cc(-c3ccc4nn(C)cc4c3)c(=O)n(-c3ccc(OC)nc3)c2n1] |
| 158 | 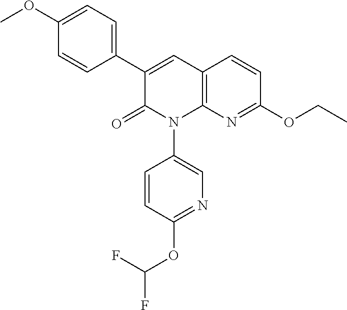 [SMILES: CCOc1ccc2cc(-c3ccc(OC)cc3)c(=O)n(-c3ccc(OC(F)F)nc3)c2n1] |
| 159 | 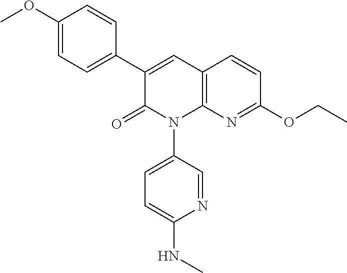 [SMILES: CCOc1ccc2cc(-c3ccc(OC)cc3)c(=O)n(-c3ccc(NC)nc3)c2n1] |
| 160 | 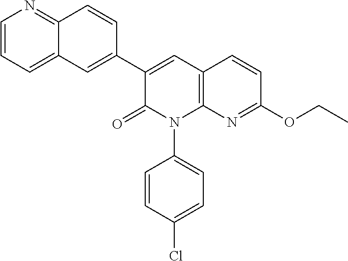 [SMILES: CCOc1ccc2cc(-c3ccc4ncccc4c3)c(=O)n(-c3ccc(Cl)cc3)c2n1] |
| 161 | 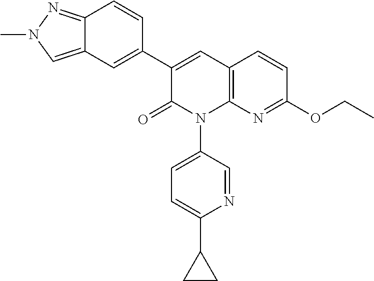 [SMILES: CCOc1ccc2cc(-c3ccc4nn(C)cc4c3)c(=O)n(-c3ccc(C4CC4)nc3)c2n1] |
| 162 | 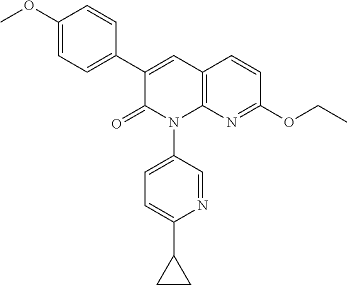 [SMILES: CCOc1ccc2cc(-c3ccc(OC)cc3)c(=O)n(-c3ccc(C4CC4)nc3)c2n1] |
| 163 | 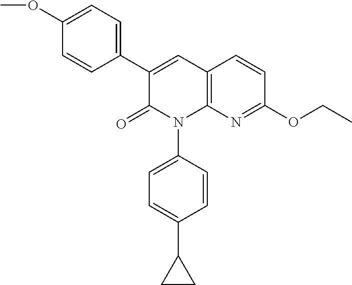 [SMILES: CCOc1ccc2cc(-c3ccc(OC)cc3)c(=O)n(-c3ccc(C4CC4)cc3)c2n1] |
| 124 | 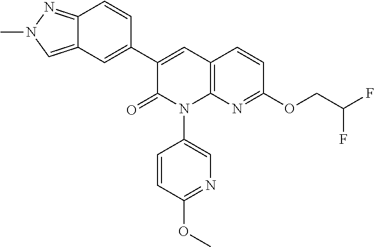 [SMILES: COc1ccc(-n2c(=O)c(-c3ccc4nn(C)cc4c3)cc3ccc(OCC(F)F)nc32)cn1] |
| 166 | 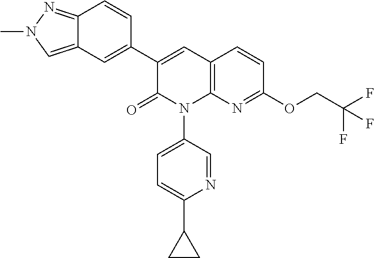 [SMILES: Cn1cc2cc(-c3cc4ccc(OCC(F)(F)F)nc4n(-c4ccc(C5CC5)nc4)c3=O)ccc2n1] |
| 122 | 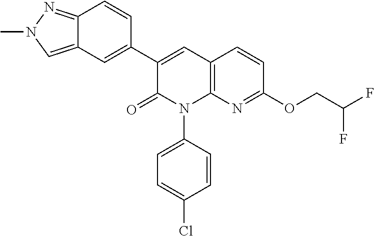 [SMILES: Cn1cc2cc(-c3cc4ccc(OCC(F)F)nc4n(-c4ccc(Cl)cc4)c3=O)ccc2n1] |
| 161 | 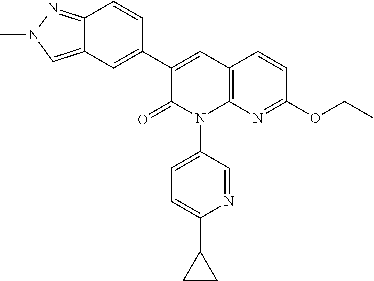 [SMILES: CCOc1ccc2cc(-c3ccc4nn(C)cc4c3)c(=O)n(-c3ccc(C4CC4)nc3)c2n1] |
| 165 | 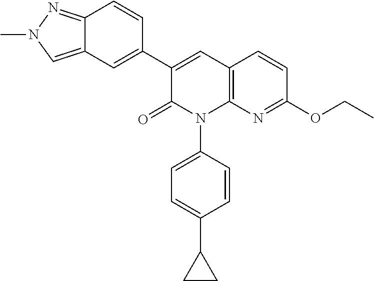 [SMILES: CCOc1ccc2cc(-c3ccc4nn(C)cc4c3)c(=O)n(-c3ccc(C4CC4)cc3)c2n1] |
| 138 | 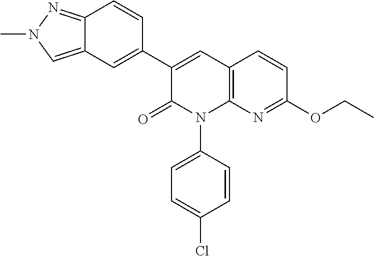 [SMILES: CCOc1ccc2cc(-c3ccc4nn(C)cc4c3)c(=O)n(-c3ccc(Cl)cc3)c2n1] |
| 196 | 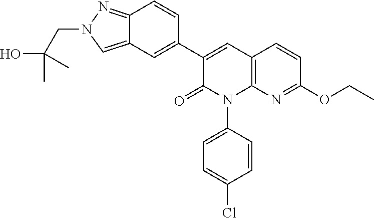 [SMILES: CCOc1ccc2cc(-c3ccc4nn(CC(C)(C)O)cc4c3)c(=O)n(-c3ccc(Cl)cc3)c2n1] |
| 149 | 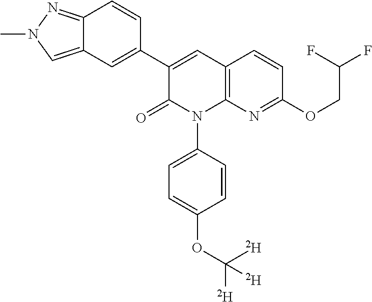 [SMILES: [2H]C([2H])([2H])Oc1ccc(-n2c(=O)c(-c3ccc4nn(C)cc4c3)cc3ccc(OCC(F)F)nc32)cc1] |
| 322 | 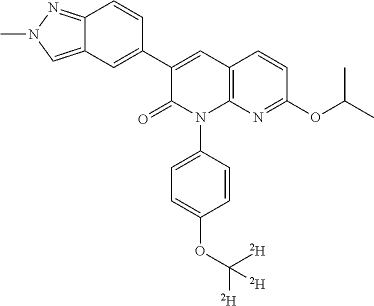 [SMILES: [2H]C([2H])([2H])Oc1ccc(-n2c(=O)c(-c3ccc4nn(C)cc4c3)cc3ccc(OC(C)C)nc32)cc1] |
| 303 | 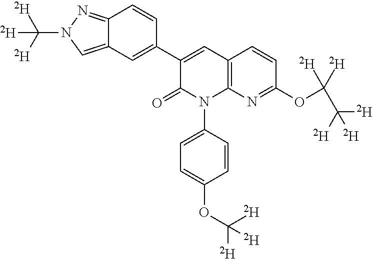 [SMILES: [2H]C([2H])([2H])Oc1ccc(-n2c(=O)c(-c3ccc4nn(C([2H])([2H])[2H])cc4c3)cc3ccc(OC([2H])([2H])C([2H])([2H])[2H])nc32)cc1] |
| 313 | 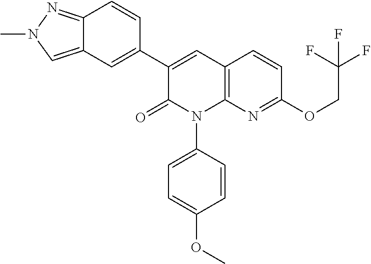 [SMILES: COc1ccc(-n2c(=O)c(-c3ccc4nn(C)cc4c3)cc3ccc(OCC(F)(F)F)nc32)cc1] |
| 142 | 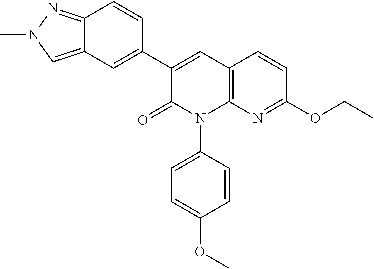 [SMILES: CCOc1ccc2cc(-c3ccc4nn(C)cc4c3)c(=O)n(-c3ccc(OC)cc3)c2n1] |
| 302 | 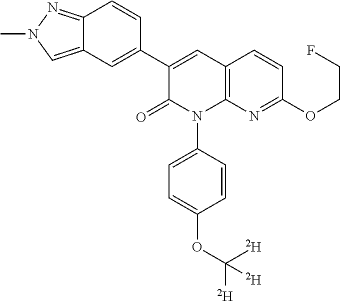 [SMILES: [2H]C([2H])([2H])Oc1ccc(-n2c(=O)c(-c3ccc4nn(C)cc4c3)cc3ccc(OCCF)nc32)cc1] |
| 195 | 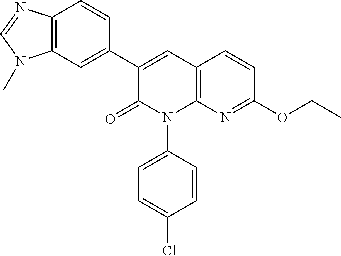 [SMILES: CCOc1ccc2cc(-c3ccc4ncn(C)c4c3)c(=O)n(-c3ccc(Cl)cc3)c2n1] |
| 314 | 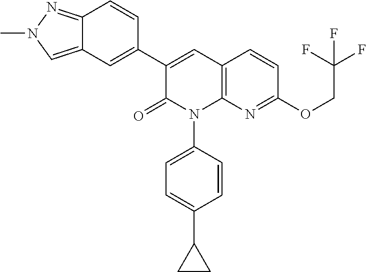 [SMILES: Cn1cc2cc(-c3cc4ccc(OCC(F)(F)F)nc4n(-c4ccc(C5CC5)cc4)c3=O)ccc2n1] |
| 210 | 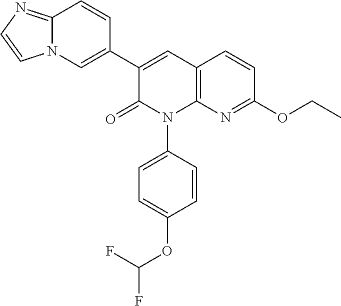 [SMILES: CCOc1ccc2cc(-c3ccc4nccn4c3)c(=O)n(-c3ccc(OC(F)F)cc3)c2n1] |
| 216 | 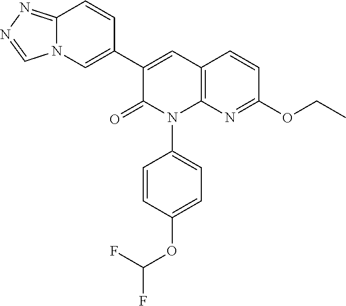 [SMILES: CCOc1ccc2cc(-c3ccc4nncn4c3)c(=O)n(-c3ccc(OC(F)F)cc3)c2n1] |
| 305 | 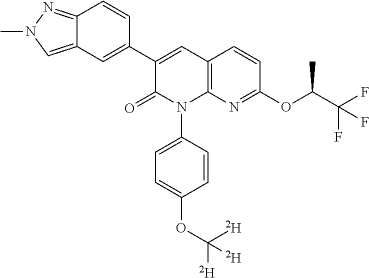 [SMILES: [2H]C([2H])([2H])Oc1ccc(-n2c(=O)c(-c3ccc4nn(C)cc4c3)cc3ccc(O[C@@H](C)C(F)(F)F)nc32)cc1] |
| 304 | 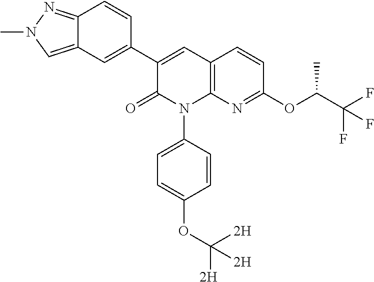 |
| 305 | 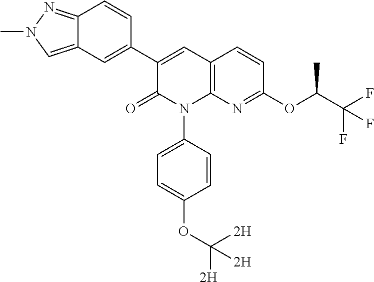 |
| 307 | 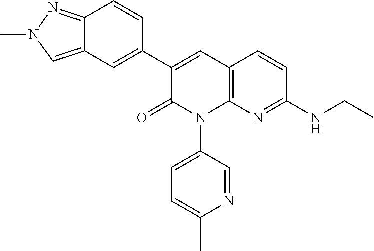 [SMILES: CCNc1ccc2cc(-c3ccc4nn(C)cc4c3)c(=O)n(-c3ccc(C)nc3)c2n1] |
| 308 | 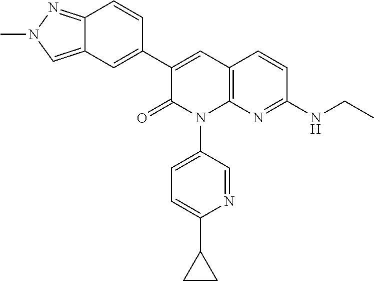 [SMILES: CCNc1ccc2cc(-c3ccc4nn(C)cc4c3)c(=O)n(-c3ccc(C4CC4)nc3)c2n1] |
| 309 | 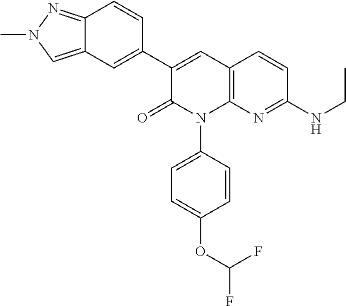 [SMILES: CCNc1ccc2cc(-c3ccc4nn(C)cc4c3)c(=O)n(-c3ccc(OC(F)F)cc3)c2n1] |
| 310 | 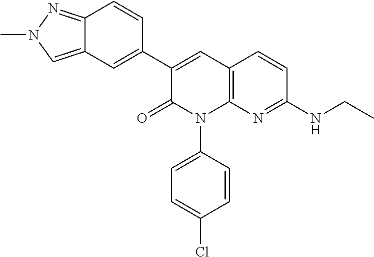 [SMILES: CCNc1ccc2cc(-c3ccc4nn(C)cc4c3)c(=O)n(-c3ccc(Cl)cc3)c2n1] |
| 311 | 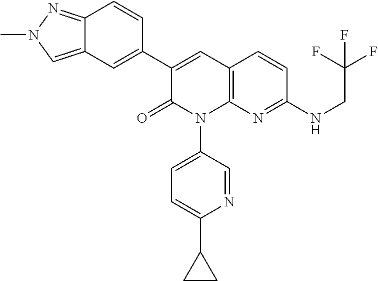 [SMILES: Cn1cc2cc(-c3cc4ccc(NCC(F)(F)F)nc4n(-c4ccc(C5CC5)nc4)c3=O)ccc2n1] |
| 164 | 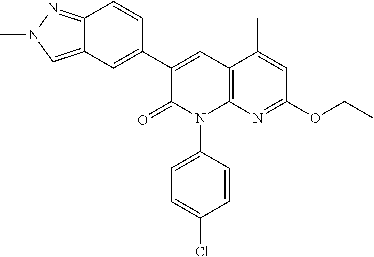 [SMILES: CCOc1cc(C)c2cc(-c3ccc4nn(C)cc4c3)c(=O)n(-c3ccc(Cl)cc3)c2n1] |
| 165 | 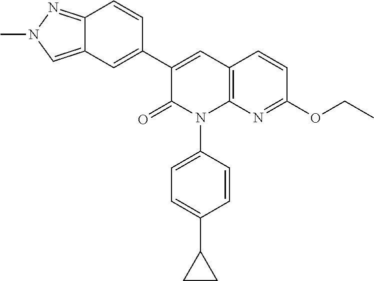 [SMILES: CCOc1ccc2cc(-c3ccc4nn(C)cc4c3)c(=O)n(-c3ccc(C4CC4)cc3)c2n1] |
| 166 | 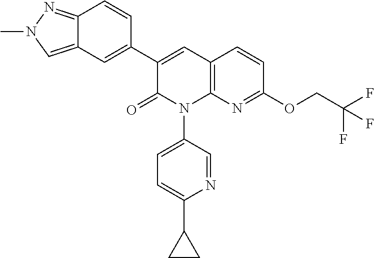 [SMILES: Cn1cc2cc(-c3cc4ccc(OCC(F)(F)F)nc4n(-c4ccc(C5CC5)nc4)c3=O)ccc2n1] |
| 167 | 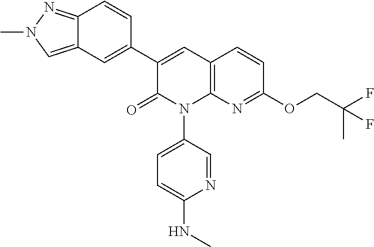 [SMILES: CNc1ccc(-n2c(=O)c(-c3ccc4nn(C)cc4c3)cc3ccc(OCC(C)(F)F)nc32)cn1] |
| 168 | 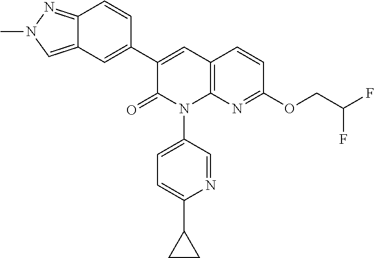 [SMILES: Cn1cc2cc(-c3cc4ccc(OCC(F)F)nc4n(-c4ccc(C5CC5)nc4)c3=O)ccc2n1] |
| 169 | 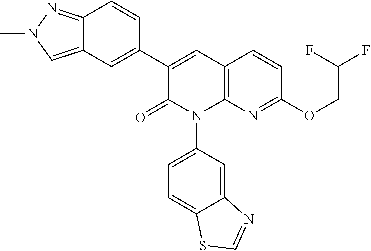 [SMILES: Cn1cc2cc(-c3cc4ccc(OCC(F)F)nc4n(-c4ccc5scnc5c4)c3=O)ccc2n1] |
| 170 | 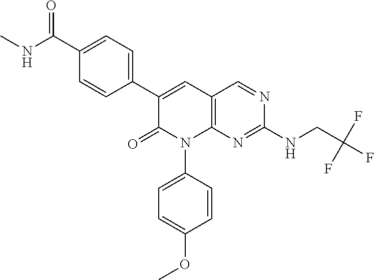 [SMILES: CNC(=O)c1ccc(-c2cc3cnc(NCC(F)(F)F)nc3n(-c3ccc(OC)cc3)c2=O)cc1] |
| 171 | 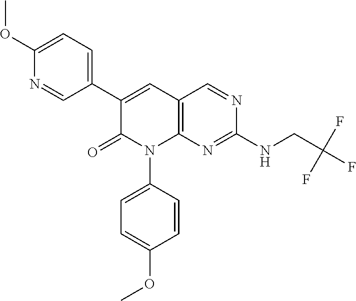 [SMILES: COc1ccc(-n2c(=O)c(-c3ccc(OC)nc3)cc3cnc(NCC(F)(F)F)nc32)cc1] |
| 172 | 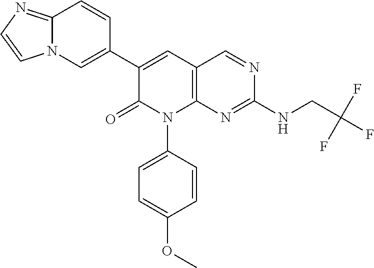 [SMILES: COc1ccc(-n2c(=O)c(-c3ccc4nccn4c3)cc3cnc(NCC(F)(F)F)nc32)cc1] |
| 173 | 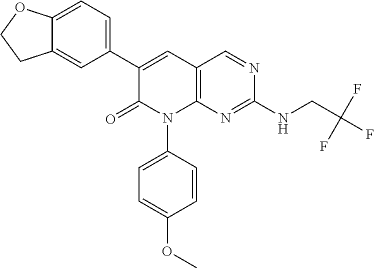 [SMILES: COc1ccc(-n2c(=O)c(-c3ccc4c(c3)CCO4)cc3cnc(NCC(F)(F)F)nc32)cc1] |
| 174 | 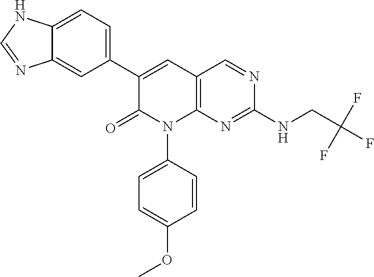 [SMILES: COc1ccc(-n2c(=O)c(-c3ccc4[nH]cnc4c3)cc3cnc(NCC(F)(F)F)nc32)cc1] |
| 175 | 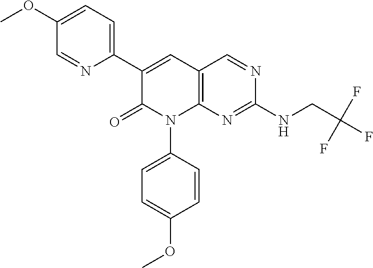 [SMILES: COc1ccc(-n2c(=O)c(-c3ccc(OC)cn3)cc3cnc(NCC(F)(F)F)nc32)cc1] |
| 176 | 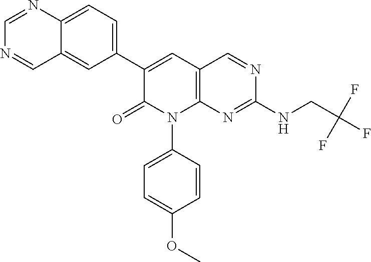 [SMILES: COc1ccc(-n2c(=O)c(-c3ccc4ncncc4c3)cc3cnc(NCC(F)(F)F)nc32)cc1] |
| 177 | 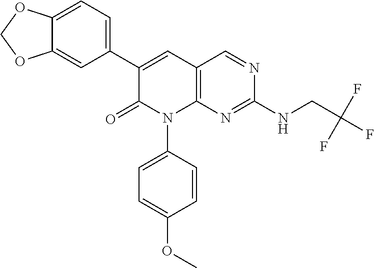 [SMILES: COc1ccc(-n2c(=O)c(-c3ccc4c(c3)OCO4)cc3cnc(NCC(F)(F)F)nc32)cc1] |
| 178 | 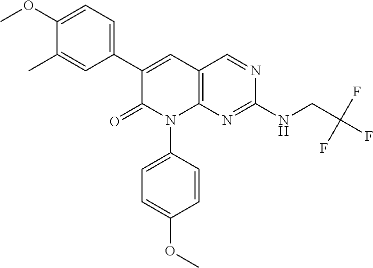 [SMILES: COc1ccc(-n2c(=O)c(-c3ccc(OC)c(C)c3)cc3cnc(NCC(F)(F)F)nc32)cc1] |
| 179 | 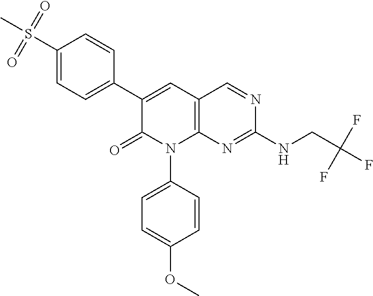 [SMILES: COc1ccc(-n2c(=O)c(-c3ccc(S(C)(=O)=O)cc3)cc3cnc(NCC(F)(F)F)nc32)cc1] |
| 180 | 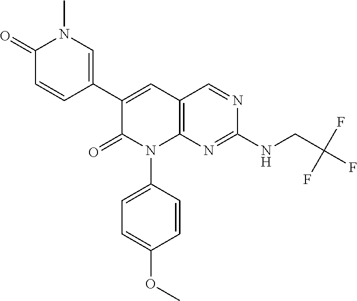 [SMILES: COc1ccc(-n2c(=O)c(-c3ccc(=O)n(C)c3)cc3cnc(NCC(F)(F)F)nc32)cc1] |
| 181 | 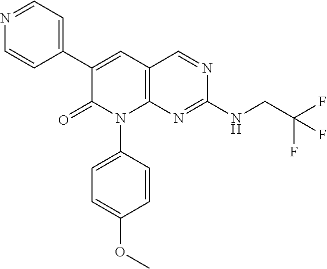 [SMILES: COc1ccc(-n2c(=O)c(-c3ccncc3)cc3cnc(NCC(F)(F)F)nc32)cc1] |
| 182 | 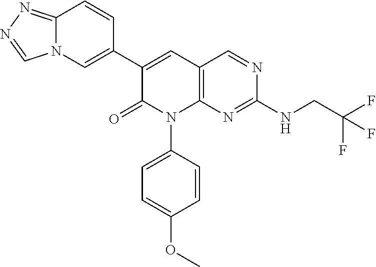 [SMILES: COc1ccc(-n2c(=O)c(-c3ccc4nncn4c3)cc3cnc(NCC(F)(F)F)nc32)cc1] |
| 183 | 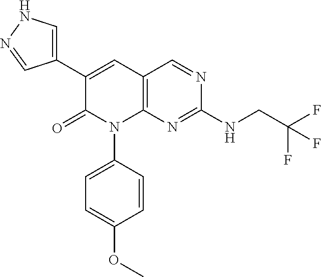 [SMILES: COc1ccc(-n2c(=O)c(-c3cn[nH]c3)cc3cnc(NCC(F)(F)F)nc32)cc1] |
| 184 | 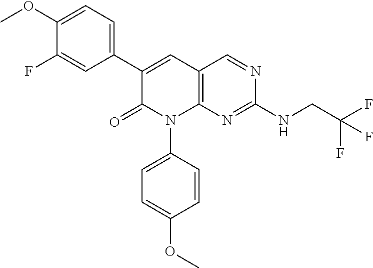 [SMILES: COc1ccc(-n2c(=O)c(-c3ccc(OC)c(F)c3)cc3cnc(NCC(F)(F)F)nc32)cc1] |
| 185 | 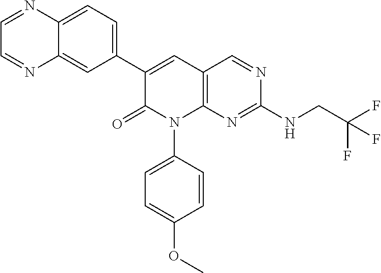 [SMILES: COc1ccc(-n2c(=O)c(-c3ccc4nccnc4c3)cc3cnc(NCC(F)(F)F)nc32)cc1] |
| 186 | 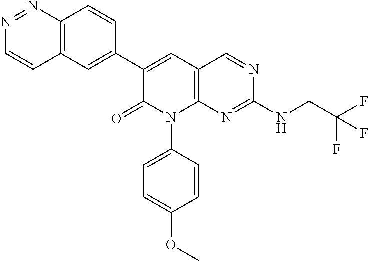 [SMILES: COc1ccc(-n2c(=O)c(-c3ccc4nnccc4c3)cc3cnc(NCC(F)(F)F)nc32)cc1] |
| 187 | 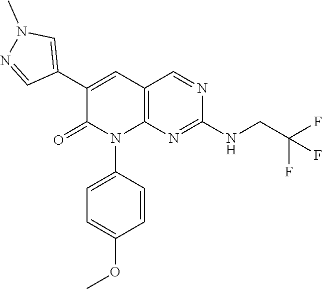 [SMILES: COc1ccc(-n2c(=O)c(-c3cnn(C)c3)cc3cnc(NCC(F)(F)F)nc32)cc1] |
| 188 | 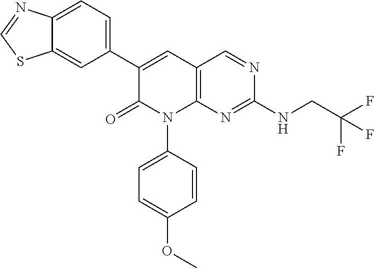 [SMILES: COc1ccc(-n2c(=O)c(-c3ccc4ncsc4c3)cc3cnc(NCC(F)(F)F)nc32)cc1] |
| 189 | 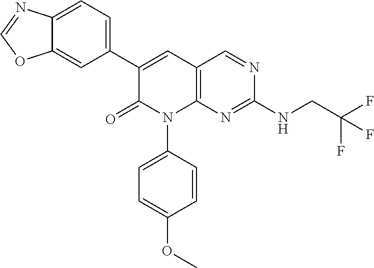 [SMILES: COc1ccc(-n2c(=O)c(-c3ccc4ncoc4c3)cc3cnc(NCC(F)(F)F)nc32)cc1] |
| 190 | 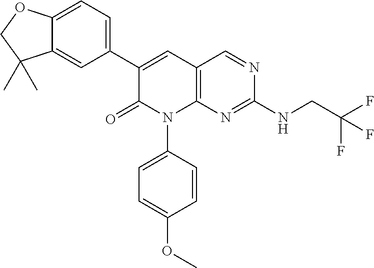 [SMILES: COc1ccc(-n2c(=O)c(-c3ccc4c(c3)C(C)(C)CO4)cc3cnc(NCC(F)(F)F)nc32)cc1] |
| 191 | 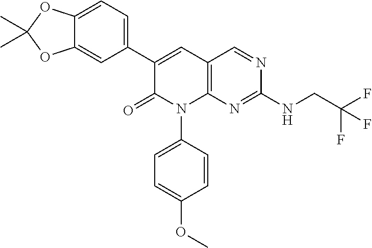 [SMILES: COc1ccc(-n2c(=O)c(-c3ccc4c(c3)OC(C)(C)O4)cc3cnc(NCC(F)(F)F)nc32)cc1] |
| 192 | 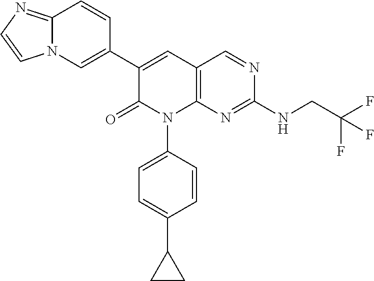 [SMILES: O=c1c(-c2ccc3nccn3c2)cc2cnc(NCC(F)(F)F)nc2n1-c1ccc(C2CC2)cc1] |
| 193 | 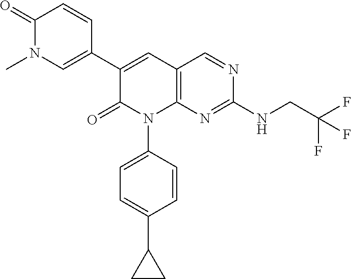 [SMILES: Cn1cc(-c2cc3cnc(NCC(F)(F)F)nc3n(-c3ccc(C4CC4)cc3)c2=O)ccc1=O] |
| 194 | 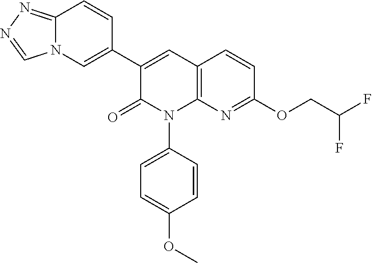 [SMILES: COc1ccc(-n2c(=O)c(-c3ccc4nncn4c3)cc3ccc(OCC(F)F)nc32)cc1] |
| 195 | 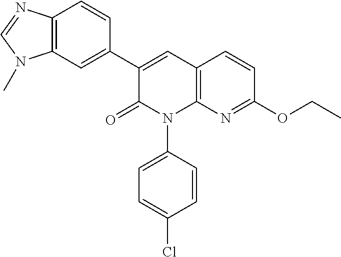 [SMILES: CCOc1ccc2cc(-c3ccc4ncn(C)c4c3)c(=O)n(-c3ccc(Cl)cc3)c2n1] |
| 196 | 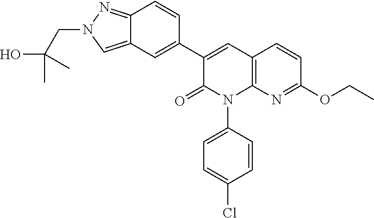 [SMILES: CCOc1ccc2cc(-c3ccc4nn(CC(C)(C)O)cc4c3)c(=O)n(-c3ccc(Cl)cc3)c2n1] |
| 197 | 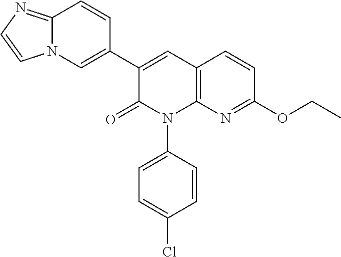 [SMILES: CCOc1ccc2cc(-c3ccc4nccn4c3)c(=O)n(-c3ccc(Cl)cc3)c2n1] |
| 198 | 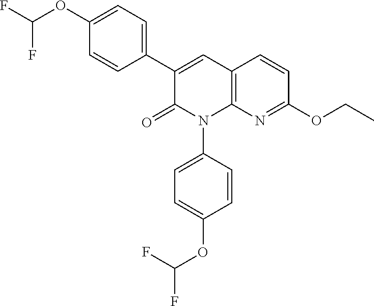 [SMILES: CCOc1ccc2cc(-c3ccc(OC(F)F)cc3)c(=O)n(-c3ccc(OC(F)F)cc3)c2n1] |
| 199 | 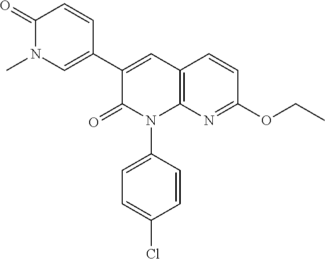 [SMILES: CCOc1ccc2cc(-c3ccc(=O)n(C)c3)c(=O)n(-c3ccc(Cl)cc3)c2n1] |
| 200 | 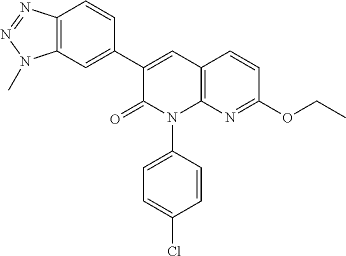 [SMILES: CCOc1ccc2cc(-c3ccc4nnn(C)c4c3)c(=O)n(-c3ccc(Cl)cc3)c2n1] |
| 201 | 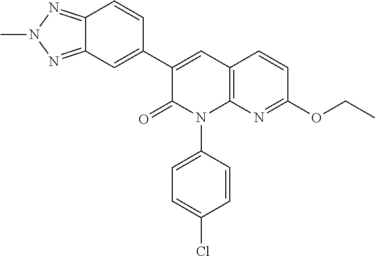 [SMILES: CCOc1ccc2cc(-c3ccc4nn(C)nc4c3)c(=O)n(-c3ccc(Cl)cc3)c2n1] |
| 202 | 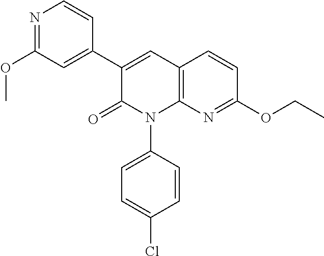 [SMILES: CCOc1ccc2cc(-c3ccnc(OC)c3)c(=O)n(-c3ccc(Cl)cc3)c2n1] |
| 203 | 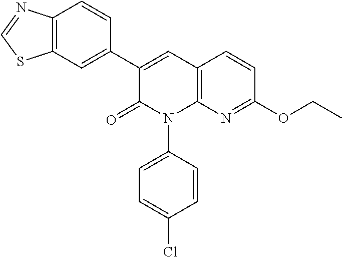 [SMILES: CCOc1ccc2cc(-c3ccc4ncsc4c3)c(=O)n(-c3ccc(Cl)cc3)c2n1] |
| 204 | 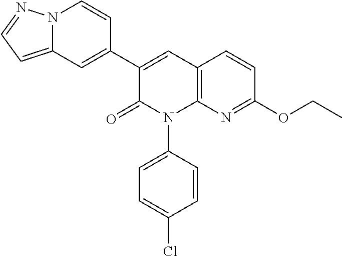 [SMILES: CCOc1ccc2cc(-c3ccn4nccc4c3)c(=O)n(-c3ccc(Cl)cc3)c2n1] |
| 205 | 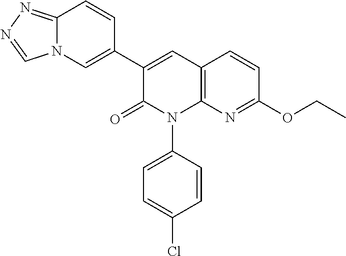 [SMILES: CCOc1ccc2cc(-c3ccc4nncn4c3)c(=O)n(-c3ccc(Cl)cc3)c2n1] |
| 206 | 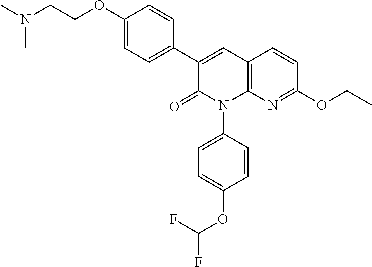 [SMILES: CCOc1ccc2cc(-c3ccc(OCCN(C)C)cc3)c(=O)n(-c3ccc(OC(F)F)cc3)c2n1] |
| 207 | 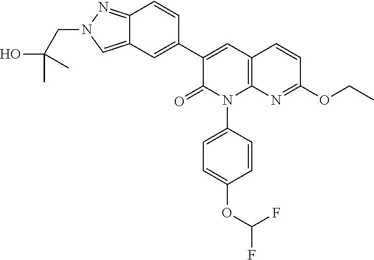 [SMILES: CCOc1ccc2cc(-c3ccc4nn(CC(C)(C)O)cc4c3)c(=O)n(-c3ccc(OC(F)F)cc3)c2n1] |
| 208 | 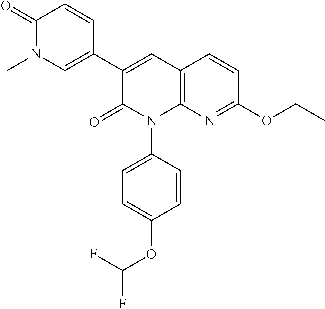 [SMILES: CCOc1ccc2cc(-c3ccc(=O)n(C)c3)c(=O)n(-c3ccc(OC(F)F)cc3)c2n1] |
| 209 | 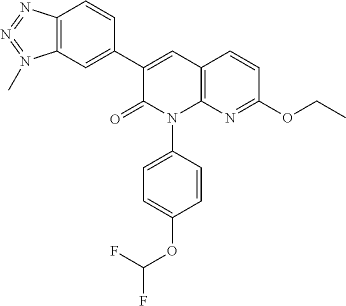 |
| 210 | 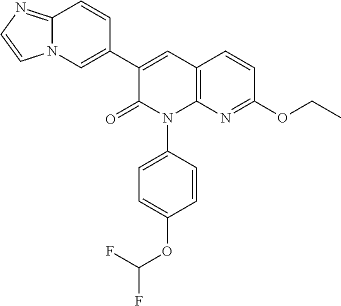 [SMILES: CCOc1ccc2cc(-c3ccc4nccn4c3)c(=O)n(-c3ccc(OC(F)F)cc3)c2n1] |
| 211 | 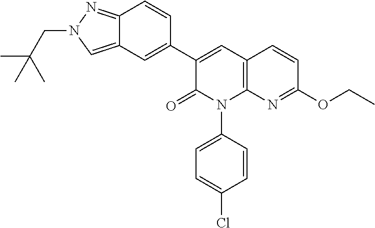 [SMILES: CCOc1ccc2cc(-c3ccc4nn(CC(C)(C)C)cc4c3)c(=O)n(-c3ccc(Cl)cc3)c2n1] |
| 212 | 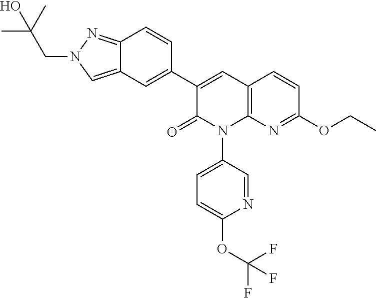 [SMILES: CCOc1ccc2cc(-c3ccc4nn(CC(C)(C)O)cc4c3)c(=O)n(-c3ccc(OC(F)(F)F)nc3)c2n1] |
| 213 | 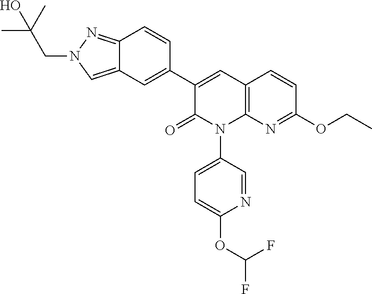 [SMILES: CCOc1ccc2cc(-c3ccc4nn(CC(C)(C)O)cc4c3)c(=O)n(-c3ccc(OC(F)F)nc3)c2n1] |
| 214 | 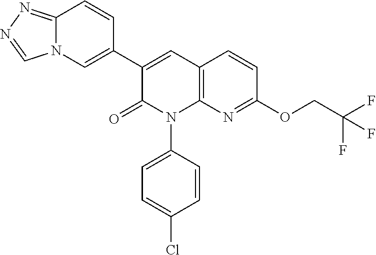 [SMILES: O=c1c(-c2ccc3nncn3c2)cc2ccc(OCC(F)(F)F)nc2n1-c1ccc(Cl)cc1] |
| 215 | 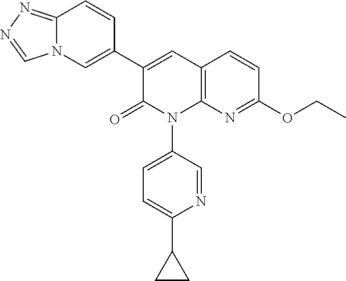 [SMILES: CCOc1ccc2cc(-c3ccc4nncn4c3)c(=O)n(-c3ccc(C4CC4)nc3)c2n1] |
| 216 | 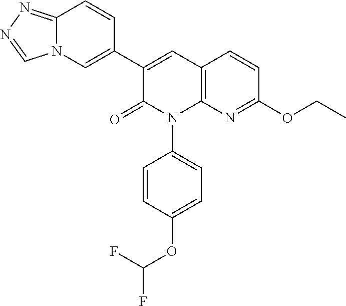 [SMILES: CCOc1ccc2cc(-c3ccc4nncn4c3)c(=O)n(-c3ccc(OC(F)F)cc3)c2n1] |
| 217 | 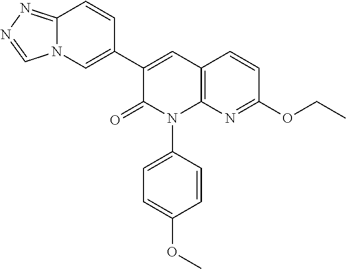 [SMILES: CCOc1ccc2cc(-c3ccc4nncn4c3)c(=O)n(-c3ccc(OC)cc3)c2n1] |
| 218 | 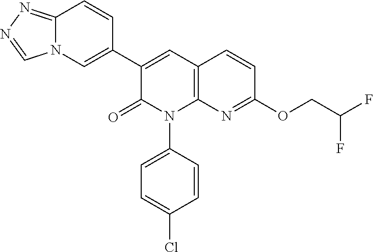 [SMILES: O=c1c(-c2ccc3nncn3c2)cc2ccc(OCC(F)F)nc2n1-c1ccc(Cl)cc1] |
| 219 | 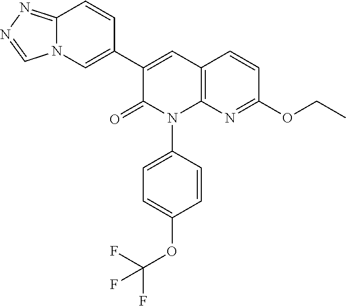 [SMILES: CCOc1ccc2cc(-c3ccc4nncn4c3)c(=O)n(-c3ccc(OC(F)(F)F)cc3)c2n1] |
| 220 | 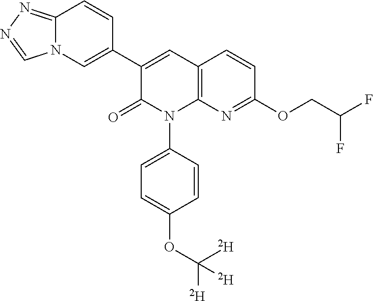 [SMILES: [2H]C([2H])([2H])Oc1ccc(-n2c(=O)c(-c3ccc4nncn4c3)cc3ccc(OCC(F)F)nc32)cc1] |
| 221 | 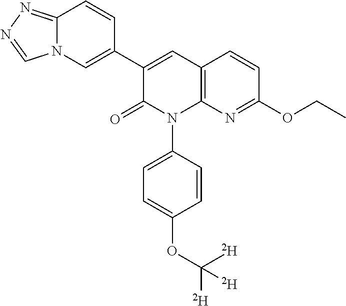 [SMILES: [2H]C([2H])([2H])Oc1ccc(-n2c(=O)c(-c3ccc4nncn4c3)cc3ccc(OCC)nc32)cc1] |
| 232 | 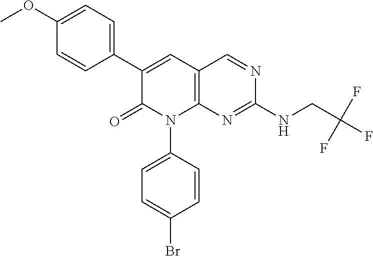 [SMILES: COc1ccc(-c2cc3cnc(NCC(F)(F)F)nc3n(-c3ccc(Br)cc3)c2=O)cc1] |
| 233 | 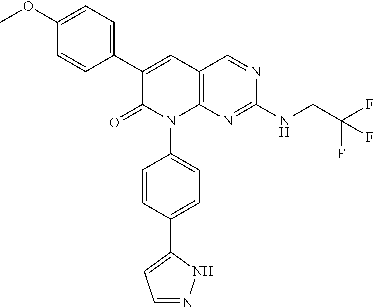 [SMILES: COc1ccc(-c2cc3cnc(NCC(F)(F)F)nc3n(-c3ccc(-c4ccn[nH]4)cc3)c2=O)cc1] |
| 234 | 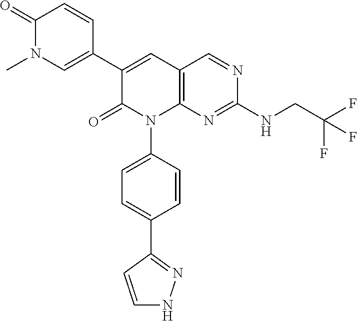 [SMILES: Cn1cc(-c2cc3cnc(NCC(F)(F)F)nc3n(-c3ccc(-c4cc[nH]n4)cc3)c2=O)ccc1=O] |
| 235 | 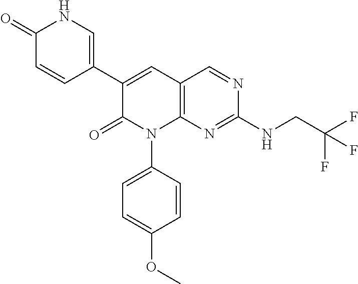 [SMILES: COc1ccc(-n2c(=O)c(-c3ccc(=O)[nH]c3)cc3cnc(NCC(F)(F)F)nc32)cc1] |
| 168 | 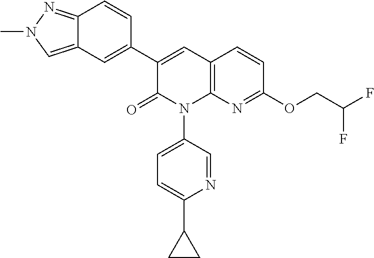 [SMILES: Cn1cc2cc(-c3cc4ccc(OCC(F)F)nc4n(-c4ccc(C5CC5)nc4)c3=O)ccc2n1] |
| 157 | 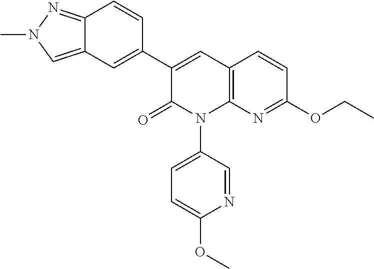 [SMILES: CCOc1ccc2cc(-c3ccc4nn(C)cc4c3)c(=O)n(-c3ccc(OC)nc3)c2n1] |
| 144 | 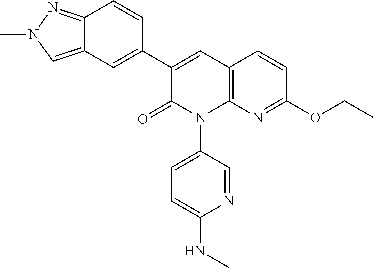 [SMILES: CCOc1ccc2cc(-c3ccc4nn(C)cc4c3)c(=O)n(-c3ccc(NC)nc3)c2n1] |
| 221 | 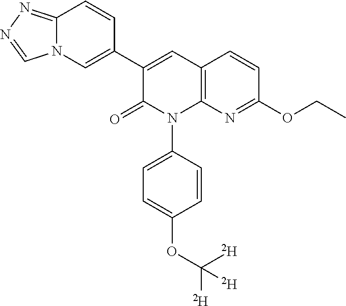 [SMILES: [2H]C([2H])([2H])Oc1ccc(-n2c(=O)c(-c3ccc4nncn4c3)cc3ccc(OCC)nc32)cc1] |
| 123 | 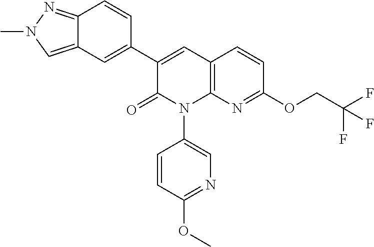 [SMILES: COc1ccc(-n2c(=O)c(-c3ccc4nn(C)cc4c3)cc3ccc(OCC(F)(F)F)nc32)cn1] |
| 205 | 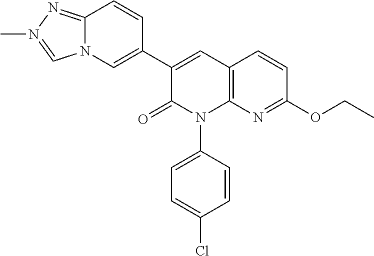 |
| 148 | 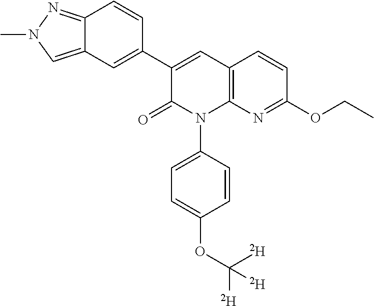 [SMILES: [2H]C([2H])([2H])Oc1ccc(-n2c(=O)c(-c3ccc4nn(C)cc4c3)cc3ccc(OCC)nc32)cc1] |
| 220 | 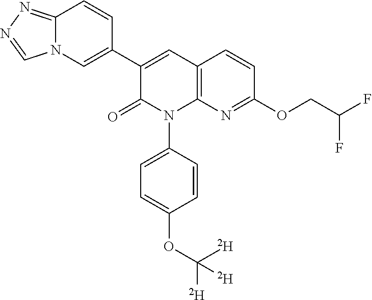 [SMILES: [2H]C([2H])([2H])Oc1ccc(-n2c(=O)c(-c3ccc4nncn4c3)cc3ccc(OCC(F)F)nc32)cc1] |
| 301 | 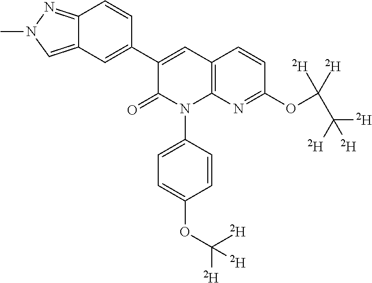 [SMILES: [2H]C([2H])([2H])Oc1ccc(-n2c(=O)c(-c3ccc4nn(C)cc4c3)cc3ccc(OC([2H])([2H])C([2H])([2H])[2H])nc32)cc1] |
| 318 | 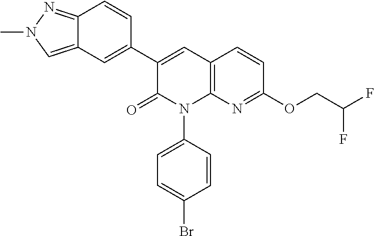 [SMILES: Cn1cc2cc(-c3cc4ccc(OCC(F)F)nc4n(-c4ccc(Br)cc4)c3=O)ccc2n1] |
| 194 | 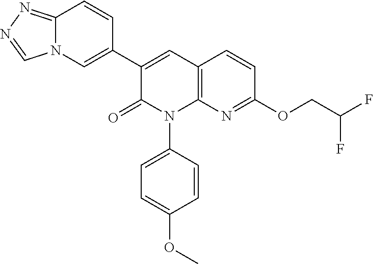 [SMILES: COc1ccc(-n2c(=O)c(-c3ccc4nncn4c3)cc3ccc(OCC(F)F)nc32)cc1] |
| 316 | 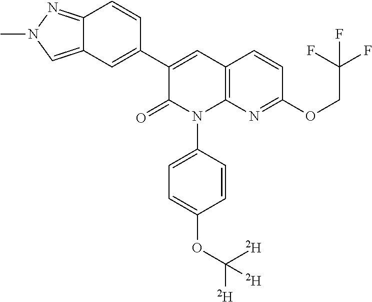 [SMILES: [2H]C([2H])([2H])Oc1ccc(-n2c(=O)c(-c3ccc4nn(C)cc4c3)cc3ccc(OCC(F)(F)F)nc32)cc1] |
| 319 | 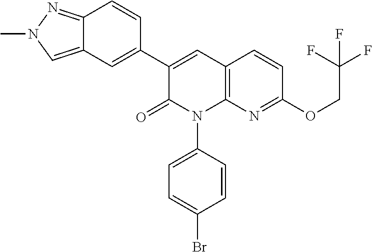 [SMILES: Cn1cc2cc(-c3cc4ccc(OCC(F)(F)F)nc4n(-c4ccc(Br)cc4)c3=O)ccc2n1] |
| 217 | 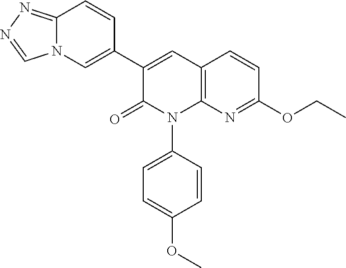 [SMILES: CCOc1ccc2cc(-c3ccc4nncn4c3)c(=O)n(-c3ccc(OC)cc3)c2n1] |
| 204 | 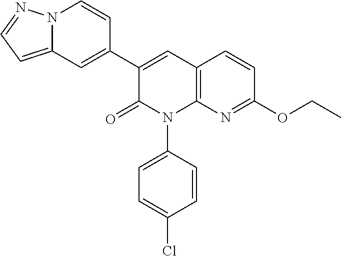 [SMILES: CCOc1ccc2cc(-c3ccn4nccc4c3)c(=O)n(-c3ccc(Cl)cc3)c2n1] |
| 200 | 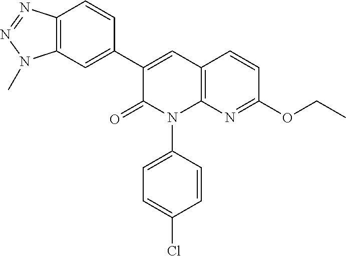 [SMILES: CCOc1ccc2cc(-c3ccc4nnn(C)c4c3)c(=O)n(-c3ccc(Cl)cc3)c2n1] |
| 218 | 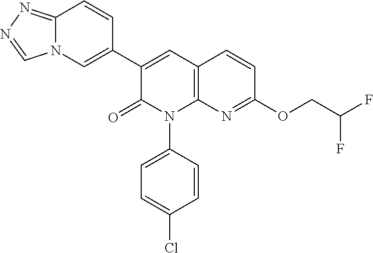 [SMILES: O=c1c(-c2ccc3nncn3c2)cc2ccc(OCC(F)F)nc2n1-c1ccc(Cl)cc1] |
| 306 | 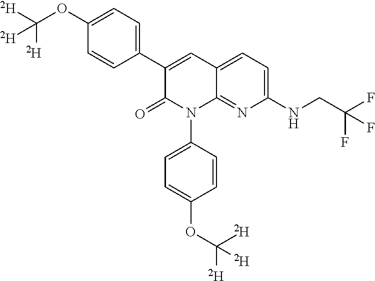 [SMILES: [2H]C([2H])([2H])Oc1ccc(-c2cc3ccc(NCC(F)(F)F)nc3n(-c3ccc(OC([2H])([2H])[2H])cc3)c2=O)cc1] |
| 312 | 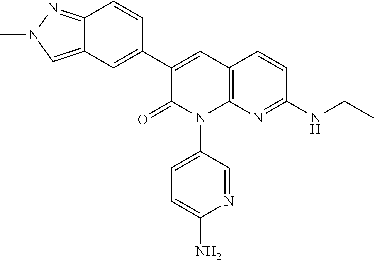 [SMILES: CCNc1ccc2cc(-c3ccc4nn(C)cc4c3)c(=O)n(-c3ccc(N)nc3)c2n1] |
| 315 | 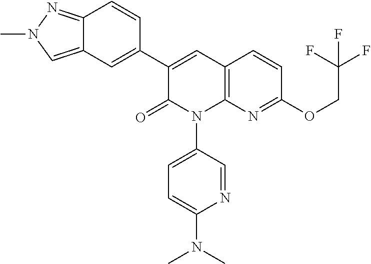 [SMILES: CN(C)c1ccc(-n2c(=O)c(-c3ccc4nn(C)cc4c3)cc3ccc(OCC(F)(F)F)nc32)cn1] |
| 317 | 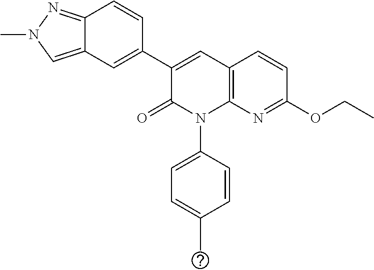 [SMILES: CCOc1ccc2cc(-c3ccc4nn(C)cc4c3)c(=O)n(-c3ccc(C)cc3)c2n1] |
| 320 | 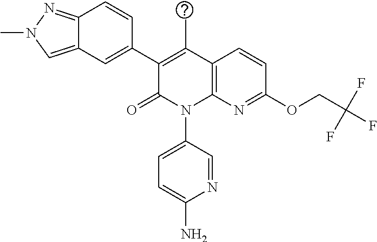 [SMILES: Cc1c(-c2ccc3nn(C)cc3c2)c(=O)n(-c2ccc(N)nc2)c2nc(OCC(F)(F)F)ccc12] |
| 321 | 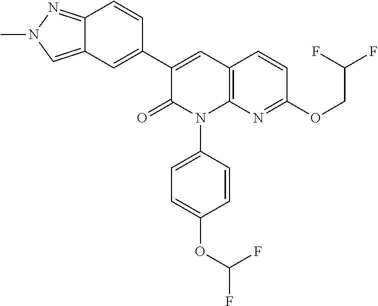 [SMILES: Cn1cc2cc(-c3cc4ccc(OCC(F)F)nc4n(-c4ccc(OC(F)F)cc4)c3=O)ccc2n1] |
| 323 | 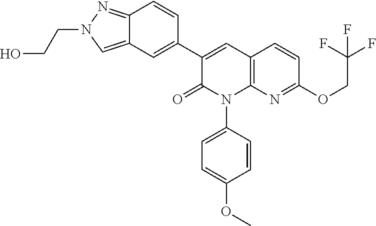 [SMILES: COc1ccc(-n2c(=O)c(-c3ccc4nn(CCO)cc4c3)cc3ccc(OCC(F)(F)F)nc32)cc1] |
| 353 | 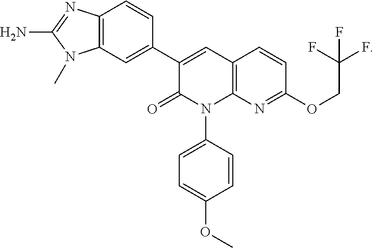 [SMILES: COc1ccc(-n2c(=O)c(-c3ccc4nc(N)n(C)c4c3)cc3ccc(OCC(F)(F)F)nc32)cc1] |

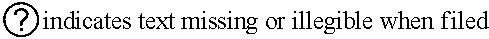

34. (canceled)

35. The compound or pharmaceutically acceptable salt thereof according to claim 2, wherein the compound is

| | |
|---|---|
| 222 | 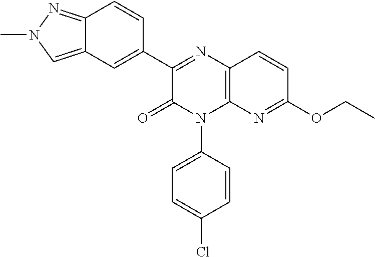 [SMILES: CCOc1ccc2nc(-c3ccc4nn(C)cc4c3)c(=O)n(-c3ccc(Cl)cc3)c2n1] |
| 223 | 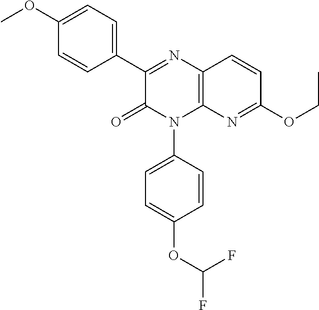 [SMILES: CCOc1ccc2nc(-c3ccc(OC)cc3)c(=O)n(-c3ccc(OC(F)F)cc3)c2n1] |
| 224 | 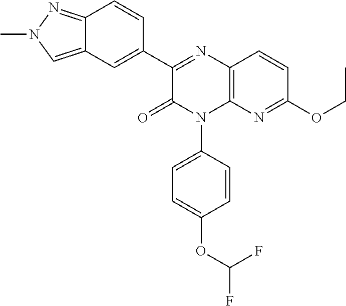 [SMILES: CCOc1ccc2nc(-c3ccc4nn(C)cc4c3)c(=O)n(-c3ccc(OC(F)F)cc3)c2n1] |
| 225 | 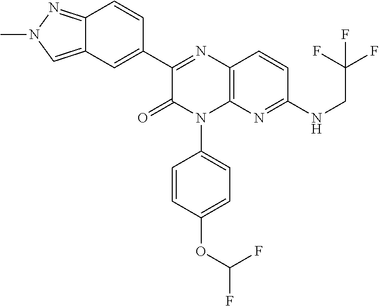 [SMILES: Cn1cc2cc(-c3nc4ccc(NCC(F)(F)F)nc4n(-c4ccc(OC(F)F)cc4)c3=O)ccc2n1] |
| 226 | 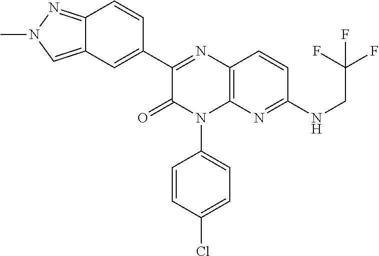 [SMILES: Cn1cc2cc(-c3nc4ccc(NCC(F)(F)F)nc4n(-c4ccc(Cl)cc4)c3=O)ccc2n1] |
| 227 | 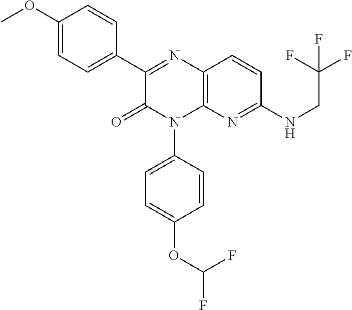 [SMILES: COc1ccc(-c2nc3ccc(NCC(F)(F)F)nc3n(-c3ccc(OC(F)F)cc3)c2=O)cc1] |
| 228 | 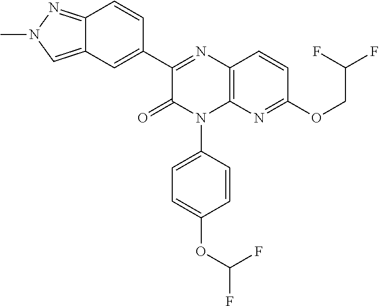 [SMILES: Cn1cc2cc(-c3nc4ccc(OCC(F)F)nc4n(-c4ccc(OC(F)F)cc4)c3=O)ccc2n1] |
| 229 | 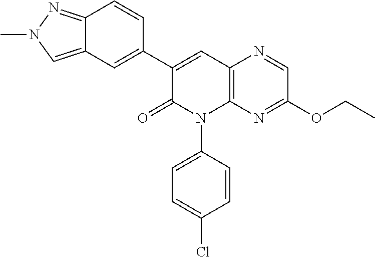 [SMILES: CCOc1cnc2cc(-c3ccc4nn(C)cc4c3)c(=O)n(-c3ccc(Cl)cc3)c2n1] |
| 230 | 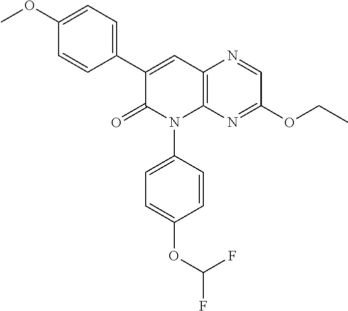 [SMILES: CCOc1cnc2cc(-c3ccc(OC)cc3)c(=O)n(-c3ccc(OC(F)F)cc3)c2n1] |
| 231 | 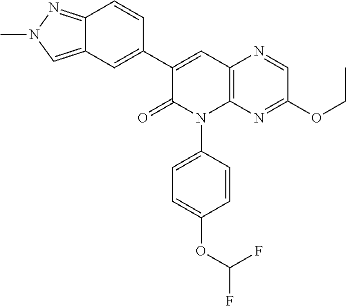 [SMILES: CCOc1cnc2cc(-c3ccc4nn(C)cc4c3)c(=O)n(-c3ccc(OC(F)F)cc3)c2n1] |
| 237 | 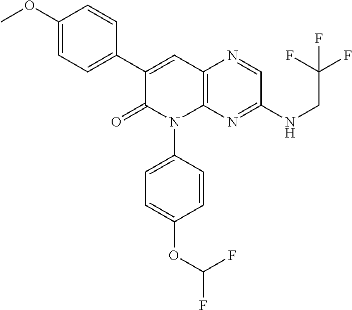 [SMILES: COc1ccc(-c2cc3ncc(NCC(F)(F)F)nc3n(-c3ccc(OC(F)F)cc3)c2=O)cc1] |
| 326 | 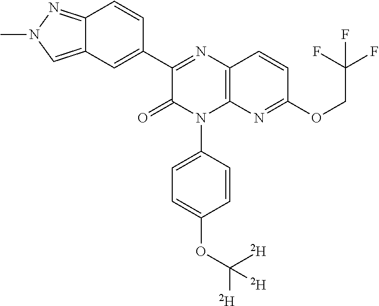 [SMILES: [2H]C([2H])([2H])Oc1ccc(-n2c(=O)c(-c3ccc4nn(C)cc4c3)nc3ccc(OCC(F)(F)F)nc32)cc1] |
| 342 | 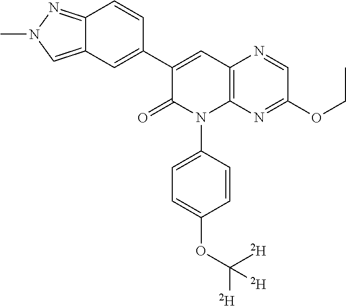 [SMILES: [2H]C([2H])([2H])Oc1ccc(-n2c(=O)c(-c3ccc4nn(C)cc4c3)cc3ncc(OCC)nc32)cc1] |
| 344 | 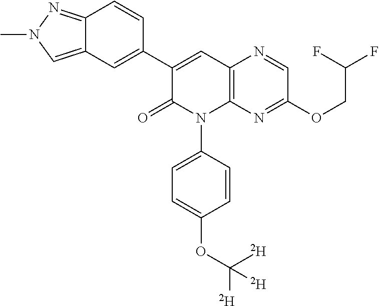 [SMILES: [2H]C([2H])([2H])Oc1ccc(-n2c(=O)c(-c3ccc4nn(C)cc4c3)cc3ncc(OCC(F)F)nc32)cc1] |
| 331 | 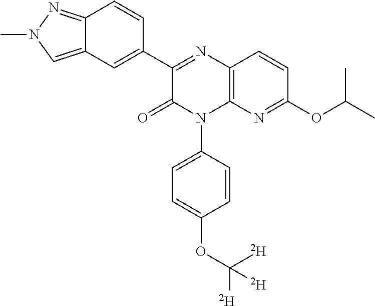 [SMILES: [2H]C([2H])([2H])Oc1ccc(-n2c(=O)c(-c3ccc4nn(C)cc4c3)nc3ccc(OC(C)C)nc32)cc1] |
| 329 | 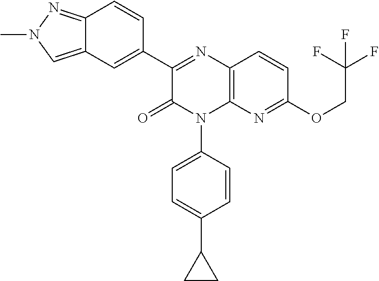 [SMILES: Cn1cc2cc(-c3nc4ccc(OCC(F)(F)F)nc4n(-c4ccc(C5CC5)cc4)c3=O)ccc2n1] |
| 328 | 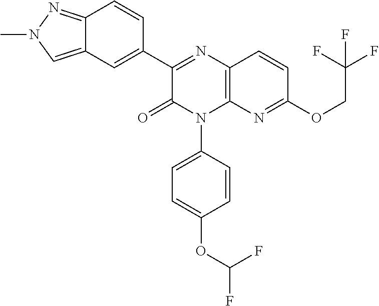 [SMILES: Cn1cc2cc(-c3nc4ccc(OCC(F)(F)F)nc4n(-c4ccc(OC(F)F)cc4)c3=O)ccc2n1] |
| 330 | 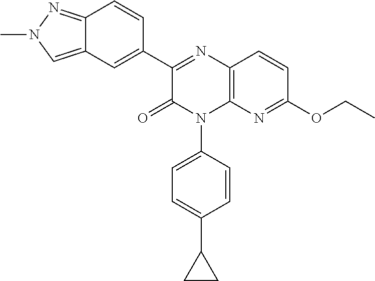 [SMILES: CCOc1ccc2nc(-c3ccc4nn(C)cc4c3)c(=O)n(-c3ccc(C4CC4)cc3)c2n1] |
| 334 | 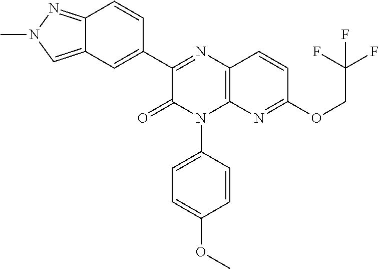 [SMILES: COc1ccc(-n2c(=O)c(-c3ccc4nn(C)cc4c3)nc3ccc(OCC(F)(F)F)nc32)cc1] |
| 343 | 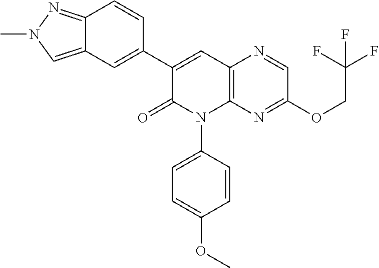 [SMILES: COc1ccc(-n2c(=O)c(-c3ccc4nn(C)cc4c3)cc3ncc(OCC(F)(F)F)nc32)cc1] |
| 345 | 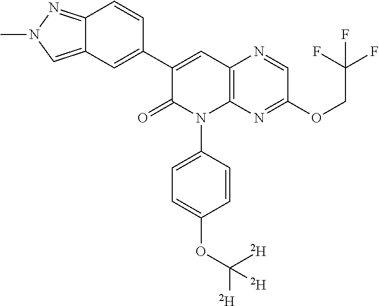 [SMILES: [2H]C([2H])([2H])Oc1ccc(-n2c(=O)c(-c3ccc4nn(C)cc4c3)cc3ncc(OCC(F)(F)F)nc32)cc1] |
| 324 | 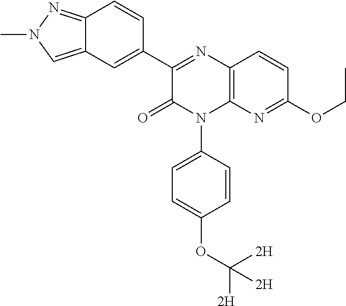 |
| 340 | 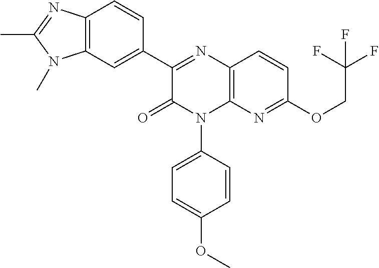 [SMILES: COc1ccc(-n2c(=O)c(-c3ccc4nc(C)n(C)c4c3)nc3ccc(OCC(F)(F)F)nc32)cc1] |
| 325 | 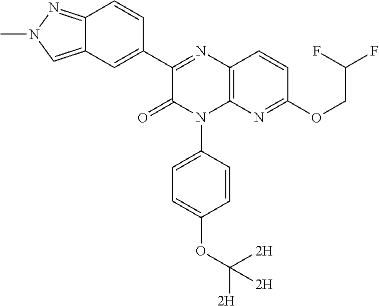 |
| 341 | 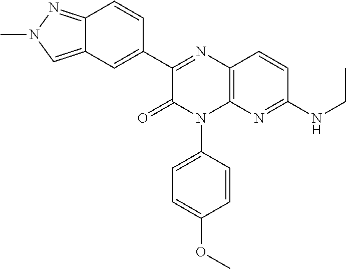 [SMILES: CCNc1ccc2nc(-c3ccc4nn(C)cc4c3)c(=O)n(-c3ccc(OC)cc3)c2n1] |
| 327 | 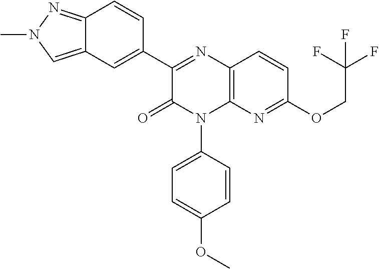 [SMILES: COc1ccc(-n2c(=O)c(-c3ccc4nn(C)cc4c3)nc3ccc(OCC(F)(F)F)nc32)cc1] |
| 346 | 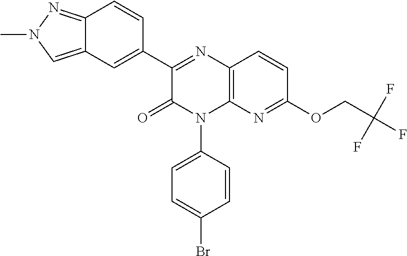 [SMILES: Cn1cc2cc(-c3nc4ccc(OCC(F)(F)F)nc4n(-c4ccc(Br)cc4)c3=O)ccc2n1] |
| 332 | 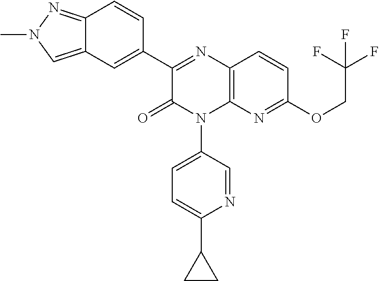 [SMILES: Cn1cc2cc(-c3nc4ccc(OCC(F)(F)F)nc4n(-c4ccc(C5CC5)nc4)c3=O)ccc2n1] |
| 347 | 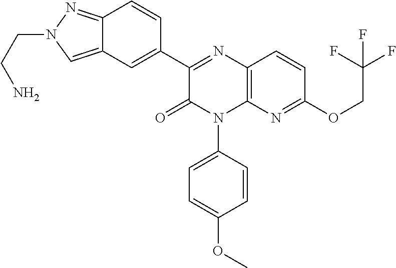 [SMILES: COc1ccc(-n2c(=O)c(-c3ccc4nn(CCN)cc4c3)nc3ccc(OCC(F)(F)F)nc32)cc1] |
| 333 | 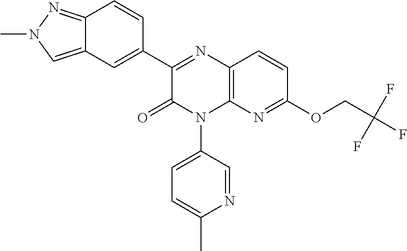 [SMILES: Cc1ccc(-n2c(=O)c(-c3ccc4nn(C)cc4c3)nc3ccc(OCC(F)(F)F)nc32)cn1] |
| 348 | 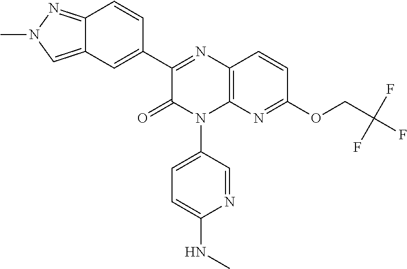 [SMILES: CNc1ccc(-n2c(=O)c(-c3ccc4nn(C)cc4c3)nc3ccc(OCC(F)(F)F)nc32)cn1] |
| 335 | 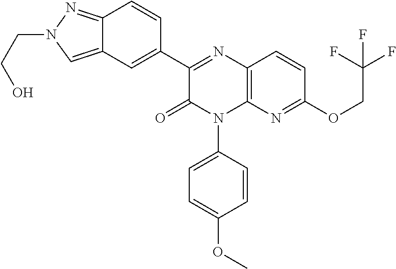 [SMILES: COc1ccc(-n2c(=O)c(-c3ccc4nn(CCO)cc4c3)nc3ccc(OCC(F)(F)F)nc32)cc1] |
| 349 | 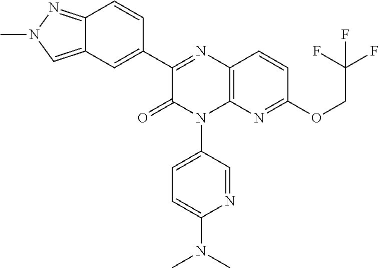 [SMILES: CN(C)c1ccc(-n2c(=O)c(-c3ccc4nn(C)cc4c3)nc3ccc(OCC(F)(F)F)nc32)cn1] |
| 336 | 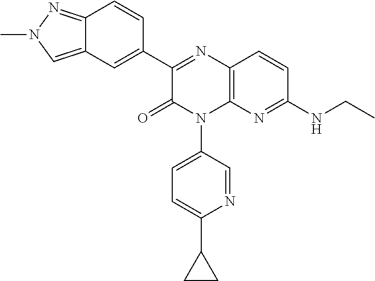 [SMILES: CCNc1ccc2nc(-c3ccc4nn(C)cc4c3)c(=O)n(-c3ccc(C4CC4)nc3)c2n1] |
| 350 | 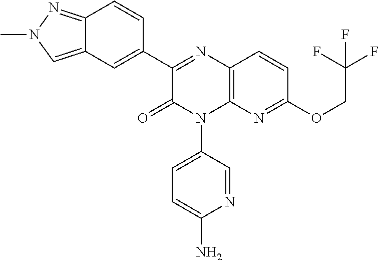 [SMILES: Cn1cc2cc(-c3nc4ccc(OCC(F)(F)F)nc4n(-c4ccc(N)nc4)c3=O)ccc2n1] |
| 337 | 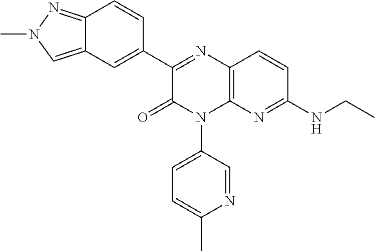 [SMILES: CCNc1ccc2nc(-c3ccc4nn(C)cc4c3)c(=O)n(-c3ccc(C)nc3)c2n1] |
| 351 | 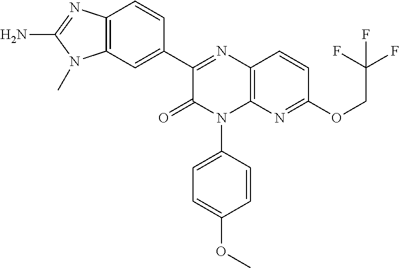 [SMILES: COc1ccc(-n2c(=O)c(-c3ccc4nc(N)n(C)c4c3)nc3ccc(OCC(F)(F)F)nc32)cc1] |
| 338 | 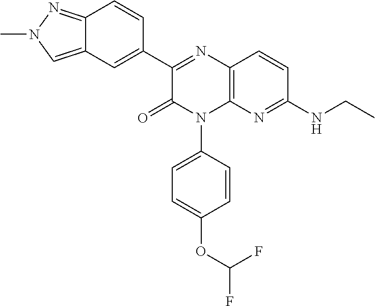 [SMILES: CCNc1ccc2nc(-c3ccc4nn(C)cc4c3)c(=O)n(-c3ccc(OC(F)F)cc3)c2n1] |
| 352 | 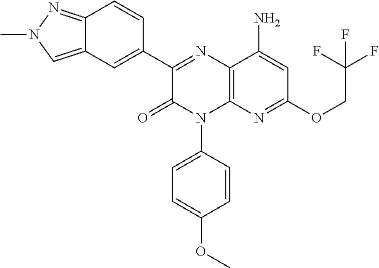 [SMILES: COc1ccc(-n2c(=O)c(-c3ccc4nn(C)cc4c3)nc3c(N)cc(OCC(F)(F)F)nc32)cc1] |
| 339 | 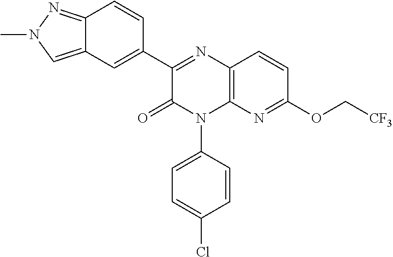 [SMILES: Cn1cc2cc(-c3nc4ccc(OCC(F)(F)F)nc4n(-c4ccc(Cl)cc4)c3=O)ccc2n1] |

36-38. (canceled)

39. A pharmaceutical composition comprising a therapeutically effective amount of a compound or pharmaceutically acceptable salt thereof according to claim 1 or a pharmaceutically acceptable salt thereof, and a pharmaceutically acceptable carrier.

40-41. (canceled)

42. A method for treating a cancer in a subject suffering therefrom, comprising administering to the subject an effective amount of a compound or pharmaceutically acceptable salt thereof according to claim 1.

43. The method according to claim 42, wherein the cancer is an MTAP-deleted cancer.

44. The method according to claim 42, wherein the cancer is (i) mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, or plasmocytoma; (ii B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, or breast cancer; (iii) a lung cancer selected from the group consisting of non-small cell lung cancer, small cell lung cancer, adenocarcinoma of the lung, and squamous cell carcinoma of the lung; (iv) a brain tumor selected from the group consisting of glioma, glioblastoma, astrocytoma, meningioma, medulloblastoma, peripheral neuroectodermal tumors, and craniopharyngioma; (v) triple negative breast cancer (TNBC); or (vi) a lymphoma selected from the group consisting of mantle cell lymphoma, Hodgkin lymphoma, non-Hodgkin lymphoma, Burkitt lymphoma, diffuse large B-cell lymphoma, and adult T-cell leukemia/lymphoma.

45-57. (canceled)

58. The compound or pharmaceutically acceptable salt thereof according to claim 2, wherein:
each of R⁴ and R⁵ is, independently, H or C₁-C₆-alkyl; and
R⁶ is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are, independently, H or C₁-C₆-alkyl).

59. The compound or pharmaceutically acceptable salt thereof according to claim 2, wherein at least one of R⁴, R⁵, and R⁶ is H.

60. The compound or pharmaceutically acceptable salt thereof according to claim 2, wherein R² is C₆-C₁₀-aryl.

61. The compound or pharmaceutically acceptable salt thereof according to claim 2, wherein R² is 5- to 10-membered heteroaryl, and wherein 1 ring member is N.

62. The compound or pharmaceutically acceptable salt thereof according to claim 2, wherein R³ is 5- to 10-membered heteroaryl.

63. The compound or pharmaceutically acceptable salt thereof according to claim 2, wherein R³ is C₆-C₁₀-aryl.

64. The compound or pharmaceutically acceptable salt thereof according to claim 2, wherein L is O or NR.

65. The compound or pharmaceutically acceptable salt thereof according to claim 2, wherein R¹ is C₁-C₆-alkyl optionally substituted by 1-3 fluoro or C₃-C₅-carbocyclyl.

66. The compound or pharmaceutically acceptable salt thereof according to claim 2, wherein:
L is O or NR and R is H;
R¹ is C₁-C₃-alkyl that is optionally substituted by 1-3 fluoro;
R2 is 5- to 10-membered heteroaryl (wherein 1 heteroaryl member is N) or C₆-C₁₀-aryl;
R³ is 5- to 10-membered heteroaryl (wherein 1 to 3 heteroaryl members are, independently, N, O, or S) or C₆-C₁₀-aryl; and
each of R⁴, R⁵, and R6 is H.

67. The compound or pharmaceutically acceptable salt thereof according to claim 2, wherein:
L is O or NR and R is H;
R¹ is C₁-C₃-alkyl that is optionally substituted by 1-3 fluoro;
R² is substituted phenyl or substituted pyridyl;
R³ is substituted benzimidazolyl, substituted indazolyl or substituted phenyl;
each of R⁴, R⁵, and R⁶ is H.

68. A pharmaceutical composition comprising a therapeutically effective amount of a compound or pharmaceutically acceptable salt thereof according to claim 2 or a pharmaceutically acceptable salt thereof, and a pharmaceutically acceptable carrier.

69. A method for treating a cancer in a subject suffering therefrom, comprising administering to the subject an effective amount of a compound or pharmaceutically acceptable salt thereof according to claim 2.

70. The method according to claim 69, wherein the cancer is an MTAP-deleted cancer.

71. The method according to claim 69, wherein the cancer is (i) mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, or plasmocytoma; (ii) B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, or breast cancer; (iii) a lung cancer selected from the group consisting of non-small cell lung cancer, small cell lung cancer, adenocarcinoma of the lung, and squamous cell carcinoma of the lung; (iv) a brain tumor selected from the group consisting of glioma, glioblastoma, astrocytoma, meningioma, medulloblastoma, peripheral neuroectodermal tumors, and craniopharyngioma; (v) triple negative breast cancer (TNBC); or (vi) a lymphoma selected from the group consisting of mantle cell lymphoma, Hodgkin lymphoma, non-Hodgkin lymphoma, Burkitt lymphoma, diffuse large B-cell lymphoma, and adult T-cell leukemia/lymphoma.
